SEQUENCE LISTING (34 sequences)

SEQ ID NO: 1 - DNA, 1421 bp, Bacillus amyloliquefaciens
  source 1..1421: mol_type = other DNA
cggcggctggctccataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcagacaaccatccggtattagccccggtttcccggagttatcccagtcttacaggcaggttacccacgtgttactcacccgtccgccgctaacatcagggagcaagctcccatctgtccgctcgactgcatg

SEQ ID NO: 2 - DNA, 1476 bp, Bacillus amyloliquefaciens
  source 1..1476: mol_type = other DNA
tcaccccaatcatctgtcccacttcggcggctggctccataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcagacaaccatccggtattagccccggtttcccggagttatcccagtcttacaggcaggttacccacgtgttactcacccgtccgccgctaacatcagggagcaagctcccatctgtccgctcgacttgcatgtattaggcacgccgccagcgttcgtcctga

SEQ ID NO: 3 - DNA, 569 bp, synthetic construct
  misc_feature 1..569: note = iturin gene sequence
  source 1..569: mol_type = other DNA
caaagacaatgaacgcgcagcccgctattttaacggtcagtgttattgcttttcaagtgtatatgcaggaaataggggtgaagccccgcttcctggcaggccatagcttaggcgaatattcagcgcttgtctgtgccggcgccctttcttttcaggatgccgttacacttgtaaggcagcggggaattcttatgcagaatgcggatcctcagcagcaggggacgatggccgccgtgactcacctctctcttcaaacgttgcaggaaatatgttcgaaagtgtcgacggaagactttccggcaggagtagcctgcatgaattcagaacagcagcatgtgatttccggacaccggcaagctgtggaacgtgtcatcaagatggcggaggaaaagggagcggcatacacttatttgaatgtcagtgcgccttttcacagttcgctgatacgatcagcatctgaacaattccagactgtattacaccggtattccttccgggaagccgcatggccgatcatttcaaatgtcactgcacgcccttacagcagcggaaattcgatcagcgaacat

SEQ ID NO: 4 - DNA, 413 bp, synthetic construct
  misc_feature 1..413: note = surfactin gene sequence
  source 1..413: mol_type = other DNA
cantttttcagtttcacttaatatgaacatgtggccgtccgaaaactcatgaaatacgggattgtcggcccattttttccatccgtccgcatctttgatacattttttatctttccgcccgttaaaaatatggacgggtgattgaatcatgtgagaatcagacggacggaagctttcaagcgcgcggtagtcggacctgaaagaaggcaggaaaaatgacatgacctccttattctccactaactcctgcggcattccgcccagctcgataatatgggcgagaaatttttcatcatcaagatgagacacttttttcctttcaacatgcggcggctgaatggctgagatgatcacggcctgcgggtaaatcccctcccgctccagcttttgcgccagcctgaaagcgaccatgc

SEQ ID NO: 5 - DNA, 547 bp, synthetic construct
  misc_feature 1..547: note = RecA gene sequence
  source 1..547: mol_type = other DNA
ttaagcaaatagaaaaacaattcggcaaaggttccatcatgaagctcggagaaaaaacggatacaagaatttcaacggtgccaagcggctcccttgcacttgatactgctctcggaataggcggatacccgcgcggacggattattgaagtatacggacctgaaagttcaggtaaaacgactgtagcgcttcacgcaatcgctgaggttcaggaaaaaggcggacaggccgcatttattgatgctgagcatgctcttgatcctgtttacgcgcaaaagctcggtgtcaatatcgaagagcttctgctttctcagccggatacgggagagcaggcgcttgagattgctgaagcgctggtgcgaagcggagctgttgatatcgtagtcgttgactctgttgcggcgcttgttccaaaagctgaaattgaaggtgacatgggtgattcacacgtcggtttacaggcgcgtcttatgtctcaggcgctccgtaagctttccggcgctatcaataaatctaaaacaatcgcaatctttattaaccagattcg

SEQ ID NO: 6 - DNA, 940 bp, synthetic construct
  misc_feature 1..940: note = GyrA gene sequence
  source 1..940: mol_type = other DNA
cgttatcgtatcccgggcgcttccggatgtgcgtgacggtctgaagccggttcacagacggattttgtacgcaatgaatgatttaggcatgaccagtgacaaaccatataaaaaatctgcccgtatcgtcggtgaagttatcggtaagtaccacccgcacggtgactcagcggtttacgaatcaatggtcagaatggcgcaggattttaactaccgctacatgcttgttgacggacacggcaacttcggttcggttgacggcgactcagcggccgcgatgcgttacacagaagcgagaatgtcaaaaatcgcaatggaaattctgcgtgacattacgaaagacacgattgactatcaagataactatgacggttcagaaagagagcctgccgtcatgccttcgagatttccgaatctgctcgtaaacggggctgccggtattgcggtcggaatggcgacaaacattcccccgcatcagcttggggaagtcattgaaggcgtgcttgccgtaagtgagaatcctgagattacaaaccaggagctgatggaatacatcccgggcccggattttccgactgcaggtcagattttgggccggagcggcatccgcaaggcatatgaatccggacggggatcaatcacgatccgggctaaggctgaaatcgaagagacttcatcgggaaaagaaagaattattgtcacggaacttccttatcaggtgaacaaagcgagattaattgaaaaaatcgcggatcttgtccgagacaaaaaaatcgaaggaattaccgatctgcgagacgaatccgaccgtaacggaatgagaatcgtcattgagatccgccgtgacgccaatgctcacgtcattttgaataacctgtacaaacaaacggccctgcagacgtctttcggaatcaatctgctggcgctcgttgacgaaagc

SEQ ID NO: 7 - DNA, 468 bp, Curtobacterium oceanosedimentum
  source 1..468: mol_type = other DNA
tgggtggattagtggcgaacgggtgagtaacacgtgagtaacctgcccctgactctgggataagcgttggaaacgacgtctaatactggatacgactgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaag

SEQ ID NO: 8 - DNA, 1424 bp, Curtobacterium oceanosedimentum
  source 1..1424: mol_type = other DNA
ctcaggacgaacgctggcggcgtgcttaacacatgcaagtcgaacgatgatgcccagcttgctgggtggattagtggcgaacgggtgagtaacacgtgagtaacctgcccctgactctgggataagcgttggaaacgacgtctaatactggatacgactgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtc

SEQ ID NO: 9 - DNA, 1341 bp, Pseudomonas oryzihabitans
  source 1..1341: mol_type = other DNA
actagctacttctggagcaacccactcccatggtgtgacgggcggtgtgtacaaggcccgggaacgtattcaccgtgacgttctgattcacgattactagcgattccgacttcacgcagtcgagttgcagactgcgatccggactacgatcggttttatgggattagctccacctcgcggcttggcaaccctttgtaccgaccattgtagcacgtgtgtagccctggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtgtctgagctcccgaaggcaccaatccatctctggaaagttctcagcatgtcaaggccaggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaagatctcaaggatcccaacggctagttgacatcgtttacggcgtggactaccagggtatctaatcctgtttgctccccacgctttcgcacctcagtgtcagtgtcagtccaggtagtcgccttcgccactggtgttccttccaatatctacgcatttcaccgctacactggaaattccactaccctctaccgcactctagccagacagttttggatgcagttcccaggttgagcccggggatttcacatccaacttatcaagccacctacgcgcgctttacgcccagtaattccgattaacgcttgcacccttcgtattaccgcggctgctggcacgaagttagccggtgcttattctgttggtaacgtcaaaactcacaggtattcgctatgagcccttcctcccaacttaaagtgctttacgacccgaaggccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtaggcctttaccctaccaactagctaatccgacctaggctcatctaatagcgtgaggtccgaagatcccccactttctcccgtaggacgtatgcggtattagcgttcctttcgaaacgttgtcccccactactaggcagattcctaggcattactcacccgtccgcc

SEQ ID NO: 10 - DNA, 1341 bp, Pseudomonas oryzihabitans
  source 1..1341: mol_type = other DNA
ggcggacgggtgagtaatgcctaggaatctgcctagtagtgggggacaacgtttcgaaaggaacgctaataccgcatacgtcctacgggagaaagtgggggatcttcggacctcacgctattagatgagcctaggtcggattagctagttggtagggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggccttcgggtcgtaaagcactttaagttgggaggaagggctcatagcgaatacctgtgagttttgacgttaccaacagaataagcaccggctaacttcgtgccagcagccgcggtaatacgaagggtgcaagcgttaatcggaattactgggcgtaaagcgcgcgtaggtggcttgataagttggatgtgaaatccccgggctcaacctgggaactgcatccaaaactgtctggctagagtgcggtagagggtagtggaatttccagtgtagcggtgaaatgcgtagatattggaaggaacaccagtggcgaaggcgactacctggactgacactgacactgaggtgcgaaagcgtggggagcaaacaggattagataccctggtagtccacgccgtaaacgatgtcaactagccgttgggatccttgagatcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggagctcagacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaacgtcacggtgaatacgttcccgggccttgtacacaccgcccgtcacaccatgggagtgggttgctccagaagtagctagt

SEQ ID NO: 11 - DNA, 653 bp, Pseudomonas oryzihabitans
  source 1..653: mol_type = other DNA
tcgagcggatgagaggagcttgctcttcgattcagcggcggacgggtgagtaatgcctaggaatctgcctagtagtgggggacaacgtttcgaaaggaacgctaataccgcatacgtcctacgggagaaagtgggggatcttcggacctcacgctattagatgagcctaggtcggattagctagttggtagggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggccttcgggtcgtaaagcactttaagttgggaggaagggctcatagcgaatacctgtgagttttgacgttaccaacagaataagcaccggctaacttcgtgccagcagccgcggtaatacgaagggtgcaagcgttaatcggaattactgggcgtaaagcgcgcgtaggtggcttgataagttggatgtgaaatccccgggctcaacctgggaactgcatccaaaactgtctggctagagtgcggtagagggtagtggaatttccagtgtagcggtgaaatgcgtagatattggaaggaacacc

SEQ ID NO: 12 - DNA, 1466 bp, Pseudomonas oryzihabitans
  source 1..1466: mol_type = other DNA
gctcagattgaacgctggcggcaggcctaacacatgcaagtcgagcggatgagaggagcttgctcttcgattcagcggcggacgggtgagtaatgcctaggaatctgcctagtagtgggggacaacgtttcgaaaggaacgctaataccgcatacgtcctacgggagaaagtgggggatcttcggacctcacgctattagatgagcctaggtcggattagctagttggtagggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggccttcgggtcgtaaagcactttaagttgggaggaagggctcatagcgaatacctgtgagttttgacgttaccaacagaataagcaccggctaacttcgtgccagcagccgcggtaatacgaagggtgcaagcgttaatcggaattactgggcgtaaagcgcgcgtaggtggcttgataagttggatgtgaaatccccgggctcaacctgggaactgcatccaaaactgtctggctagagtgcggtagagggtagtggaatttccagtgtagcggtgaaatgcgtagatattggaaggaacaccagtggcgaaggcgactacctggactgacactgacactgaggtgcgaaagcgtggggagcaaacaggattagataccctggtagtccacgccgtaaacgatgtcaactagccgttgggatccttgagatcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaacgtcacggtgaatacgttcccgggccttgtacacaccgcccgtcacaccatgggagtgggttgctccagaagtagctagtctaaccttcgggaggacggttaccacggagtgattcatgactggggtga

SEQ ID NO: 13 - DNA, 720 bp, Achromobacter xylosoxidans
  source 1..720: mol_type = other DNA
gtatcggaacgtgcccagtagcgggggataactacgcgaaagcgtagctaataccgcatacgccctacgggggaaagcaggggatcgcaagaccttgcactattggagcggccgatatcggattagctagttggtggggtaacggctcaccaaggcgacgatccgtagctggtttgagaggacgaccagccacactgggactgagacacggcccagactcctacgggaggcagcagtggggaattttggacaatgggggaaaccctgatccagccatcccgcgtgtgcgatgaaggccttcgggttgtaaagcacttttggcaggaaagaaacgtcgtgggttaataccccgcgaaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagagcttaactttggaactgcatttttaactaccgggctagagtgtgtcagagggaggtggaattccgcgtgtagcagtgaaatgcgtagatatgcggaggaacaccgatggcgaangcagcctcctgggataacactgacgctcatgcacgaaagcgtggggagcaaacaggattagataccctggtagtccacgccctaaacnatgtcaactagctgttggggcct

SEQ ID NO: 14 - DNA, 1458 bp, Achromobacter xylosoxidans
  source 1..1458: mol_type = other DNA
tcagattgaacgctagcgggatgccttacacatgcaagtcgaacggcagcacggacttcggtctggtggcgagtggcgaacgggtgagtaatgtatcggaacgtgcccagtagcgggggataactacgcgaaagcgtagctaataccgcatacgccctacgggggaaagcaggggatcgcaagaccttgcactattggagcggccgatatcggattagctagttggtggggtaacggctcaccaaggcgacgatccgtagctggtttgagaggacgaccagccacactgggactgagacacggcccagactcctacgggaggcagcagtggggaattttggacaatgggggaaaccctgatccagccatcccgcgtgtgcgatgaaggccttcgggttgtaaagcacttttggcaggaaagaaacgtcgtgggttaataccccgcgaaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagagcttaactttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttggtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaatgccgaagagatttggcagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcgggacagagggtcgccaacccgcgagggggagccaatcccagaaacccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggttttaccagaagtagttagcctaaccgcaaggggggcgattaccacggtaggattcatgactggggtga

SEQ ID NO: 15 - DNA, 477 bp, Pantoea dispersa
  source 1..477: mol_type = other DNA
agtgggggaccttcgggccacacaccatcggatgtgcccggatgggattagctagtaggtggggtaatggctcacctaggcgacgatccctagctggtctgagaggatgaccagccacactggaactgagacacggtccagactcctacgggagtangcagtggggaatattgcacaatgggcgcaagcctgatgcagccatgccgcgtgtatgaagaaggccttcgggttgtaaagtactttcagcggggaggaaggcggtgaggttaataaccttgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggta

SEQ ID NO: 16 - DNA, 1467 bp, Pantoea dispersa
  source 1..1467: mol_type = other DNA
ctcagattgaacgctggcggcaggcctaacacatgcaagtcgaacggcagcacagaagagcttgctctttgggtggcgagtggcggacgggtgagtaatgtctgggaaactgcccgatggagggggataactactggaaacggtagctaataccgcataacgtcgcaagaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcggtgaggttaataaccttgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgctaccactttgtgattcatgactggggt

SEQ ID NO: 17 - DNA, 616 bp, Enterobacter cloacae
  source 1..616: mol_type = other DNA
atgtctgggaaactgcctgatggagggggataactactggaaacggtagctaataccgcataatgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccacagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggc

SEQ ID NO: 18 - DNA, 1470 bp, Enterobacter cloacae
  source 1..1470: mol_type = other DNA
tcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataatgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccacagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaacttaccagagatgctttggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgagtc

SEQ ID NO: 19 - DNA, 20 bp, synthetic construct
  misc_feature 1..20: note = primer 27F
  source 1..20: mol_type = other DNA
agagtttgatcmtggctcag

SEQ ID NO: 20 - DNA, 16 bp, synthetic construct
  misc_feature 1..16: note = primer 1492R
  source 1..16: mol_type = other DNA
taccttgttacgactt

SEQ ID NO: 21 - DNA, 18 bp, synthetic construct
  misc_feature 1..18: note = primer GhPerox-F
  source 1..18: mol_type = other DNA
gcgtgacgtgctcaaacg

SEQ ID NO: 22 - DNA, 18 bp, synthetic construct
  misc_feature 1..18: note = primer GhPerox-R
  source 1..18: mol_type = other DNA
cattgagggcgccatgtt

SEQ ID NO: 23 - DNA, 20 bp, synthetic construct
  misc_feature 1..20: note = primer GhPR1-F
  source 1..20: mol_type = other DNA
agttgcgcagcaggtaaggt

SEQ ID NO: 24 - DNA, 17 bp, synthetic construct
  misc_feature 1..17: note = primer GhPR1-R
  source 1..17: mol_type = other DNA
tcgcgccaaaccacttg

SEQ ID NO: 25 - DNA, 21 bp, synthetic construct
  misc_feature 1..21: note = primer GhExpansin-F
  source 1..21: mol_type = other DNA
ctggcagagcaacacctacct

SEQ ID NO: 26 - DNA, 20 bp, synthetic construct
  misc_feature 1..20: note = primer GhExpansin-R
  source 1..20: mol_type = other DNA
cgcttgtggtgacttggaaa

SEQ ID NO: 27 - DNA, 21 bp, synthetic construct
  misc_feature 1..21: note = primer GhNT-F
  source 1..21: mol_type = other DNA
gaccatcaaggtccagctttg

SEQ ID NO: 28 - DNA, 18 bp, synthetic construct
  misc_feature 1..18: note = primer GhNT-R
  source 1..18: mol_type = other DNA
gccatgcacggtcgactt

SEQ ID NO: 29 - DNA, 21 bp, synthetic construct
  misc_feature 1..21: note = primer GhNR-F
  source 1..21: mol_type = other DNA
gaccatcaaggtccagctttg

SEQ ID NO: 30 - DNA, 18 bp, synthetic construct
  misc_feature 1..18: note = primer GhNR-R
  source 1..18: mol_type = other DNA
gccatgcacggtcgactt

SEQ ID NO: 31 - DNA, 19 bp, synthetic construct
  misc_feature 1..19: note = primer GhActinB-F
  source 1..19: mol_type = other DNA
ccggtgacggtgtttctca

SEQ ID NO: 32 - DNA, 20 bp, synthetic construct
  misc_feature 1..20: note = primer GhActinB-R
  source 1..20: mol_type = other DNA
gcatgtggaagggcatatcc

SEQ ID NO: 33 - DNA, 20 bp, synthetic construct
  misc_feature 1..20: note = primer ITS1
  source 1..20: mol_type = other DNA
tccgtaggtgaaccttgcgg

SEQ ID NO: 34 - DNA, 20 bp, synthetic construct
  misc_feature 1..20: note = primer ITS4
  source 1..20: mol_type = other DNA
tcctccgcttattgatatgc

(19) United States
(12) Patent Application Publication
(10) Pub. No.: US 2023/0332168 A1
(43) Pub. Date: Oct. 19, 2023

(54) COMPOSITIONS AND METHODS COMPRISING ENDOPHYTIC BACTERIUM FOR APPLICATION TO TARGET PLANTS TO INCREASE PLANT GROWTH, AND INCREASE RESISTANCE TO ABIOTIC AND BIOTIC STRESSORS

(71) Applicant: RUTGERS, THE STATE UNIVERSITY OF NEW JERSEY, New Brunswick, NJ (US)

(72) Inventors: James F. White, JR., South River, NJ (US); Ivelisse Irizarry Caraballo, San Juan, PR (US)

(73) Assignee: RUTGERS, THE STATE UNIVERSITY OF NEW JERSEY, New Brunswick, NJ (US)

(21) Appl. No.: 18/194,271

(22) Filed: Mar. 31, 2023

Related U.S. Application Data
(63) Continuation of application No. 16/636,565, filed on Feb. 4, 2020, now abandoned.
(60) Provisional application No. 62/541,395, filed on Aug. 4, 2017.

(51) Int. Cl. C12N 15/82; A01H 6/54; A01H 6/60; C12N 1/20

(52) U.S. Cl. CPC C12N 15/8241; A01H 6/542; A01H 6/604; C12N 1/205; C12R 2001/38

(57) ABSTRACT

Endophytic bacteria, compositions comprising the same, and methods of use thereof are disclosed which increase the root and shoot growth of cotton host plants, suppress growth of soil borne fungal pathogens of host plants, and increase resistance of the plant to salt stress and other abiotic stressors.

### CROSS REFERENCE TO RELATED APPLICATION

The present application is a Continuation of U.S. patent application Ser. No. 16/636,565, filed Feb. 4, 2020, which is a § 371 of International Application No. PCT/US18/45173, filed Aug. 3, 2018, which claims priority to U.S. Provisional Application No. 62/541,395 filed Aug. 4, 2017. The entire disclosure of each of the aforesaid applications is incorporated by reference in the present application.

### SEQUENCE LISTING

Incorporated herein by reference in its entirety is the sequence listing submitted via EFS-Web as a text file named RUT-P06501US02_SEQLIST.xml, created, Mar. 31, 2023 and having a size of 56,677 bytes.

### FIELD OF THE INVENTION

This invention relates to the fields of plant biology and endophytic bacteria. More specifically, the invention provides new strains of endophytic bacteria isolated from seeds of wild relatives of cotton which provide beneficial features to a target plant upon colonization of the same.

### BACKGROUND OF THE INVENTION

Several publications and patent documents are cited throughout the specification in order to describe the state of the art to which this invention pertains. Each of these citations is incorporated by reference herein as though set forth in full.

Plant microbiomes consist of communities of epiphytic, endophytic, rhizospheric, and seed-transmitted microbes. Microbes that make up the plant microbiome originate from various sources including seeds, soil, pollinators and other animals, as well as the environment. Some of these plant-associated microbes are beneficial to their plant hosts while others are disease causing agents.

Beneficial microbes can be used to enhance plant growth and decrease the demand for inorganic nitrogenous fertilizers. Plant associated bacteria, such as rhizobacteria, are being widely investigated for their plant growth promoting properties and potential to be developed into biofertilizers (Banerjee et al 2006, Mia et al 2010, Ashrafuzzaman et al 2009, Abbasi et al 2011, Bhardwaj et al 2014). In addition to enhancing growth in favorable conditions, certain microbes also alleviate stress in plants. Beneficial, growth promoting bacteria have been demonstrated to enhance the ability of plants to tolerate a variety stresses such as drought, salinity, low nutrient fertility, pollutants, and disease (Burd et al 2000, Ma et al 2011, Glick 1995, East 2013). Thus, identifying microbes (or communities of microbes) that enhance growth and increase host tolerance to various abiotic and biotic stresses is key to developing more resistant crops.

Indeed, harnessing such plant microbes and exploiting the benefits of plant growth promoting microbes has recently become a focus of many agrochemical companies. Seeking microbes and developing bioproducts based on plant growth promoting microorganisms can help support sustainable agriculture and is partly driven by the increased demand in organic agricultural products (Berg 2009, Berg et al 2013, Bhardwaj et al 2014). The demand for biofertilizers and their use will likely continue to increase in order to support a growing global population, coupled with environmental issues such as climate change, soil nutrient depletion, and the need to protect environmental resources from pollutants such as inorganic nitrogenous fertilizers.

### SUMMARY OF THE INVENTION

The present invention involves the restoration of beneficial cottonseed microbiomes through the use of beneficial microbes from seeds of non-cultivated relatives of cotton (e.g, portia tree (Thespesia populnea) and wild cotton (Gossypium hirsutum)) that are applied onto agricultural seeds with damaged microbiomes. The heterologously inoculated beneficial cottonseed microbiomes are able to: 1) stimulate seed germination, 2) increase seedling root growth, 3) increase absorption of nutrients (e.g., nitrogen) into seedling roots, 4) improve abiotic stress (e.g., high soil salt levels) tolerance in seedlings, and 5) protect seedlings from soil pathogens (e.g., Fusarium spp., Lasiodiplodia spp., Alternaria spp., etc.). The microbes (bacteria) that may be used to construct/rebuild cottonseed microbiomes include: 1) Bacillus amyloliquefaciens (strain Bamy), 2) Curtobacterium oceanosedimentum (strain WCB1), 3) Pseudomonas oryzihabitans (strain WCB2), 4) Pseudomonas oleovorans (strain Poryz), 5) Achromobacter xylosidans (strain Achromo), 6) Pantoea dispersa (strain Pdisp), and 7) Enterobacter cloacae (strain Entero). In the present invention the sybplication of strains involves use of strains in combination on cottonseeds. Synergistic combinations can include a strain mixture including 1) Bacillus amyloliquefaciens (strain Bamy), 2) Curtobacterium oceanosedimentum (strain WCB1) and 3) Pseudomonas oryzihabitans (strain WCB2). This combination of seed microbes maximizes seed microbiome functionality in that strain capabilities compliment one another (see Table 1) to maximize beneficial effects on seedlings, including: 1) fungal disease suppression, 2) enhancement of seed germination rate, 3) enhancement of root growth, 4) enhancement of abiotic stress tolerance, and 5) increased nutrient absorption (including nitrogen and phosphorus). Thus, strains Bamy, WCB1 and WCB2 are the core of the rebuilt cottonseed microbiome with the other strains (Poryz, Achromo, Pdisp, and Entero) used individually or together to augment functionality in increasing cotton seedling protection from fungal pathogens.

In one embodiment, a method of improving a plant phenotype is provided. An exemplary method comprising inoculating a plant element with a formulation comprising one or more biologically pure Bacillus spp., Curtobacterium ssp., Pseudomonas ssp, Achromobacter ssp, Pantoea ssp, or Enterobacter ssp. endophyte strains isolated from Thespesia populnea and Gossypium hirsutum which are heterologously disposed to said plant element, wherein said endophyte strains are present in the formulation in an amount capable of modulating a trait of agronomic importance in a plant comprising or derived from said plant element, as compared to a reference plant grown under the same conditions. The plant to be treated may be a monocot or dicot.

Target plants include, without limitation, cotton, okra, soybean, cacao, kenaf and kola nut, coffee, tobacco, potato, tomato, sweet potato, sunflower, rapeseed, wheat, corn, rice, barley, sorghum, grass, sugarcane, bamboo, buckwheat, snap bean, dry bean, canola, peas, peanuts, safflower, sunflower, alfalfa hay, clover, vetch, and trefoil, blackberry, blueberry, currant, elderberry, gooseberry, huckleberry, loganberry, raspberry, strawberry, grape, garlic, leek, onion, shallot, citrus hybrid, grapefruit, kumquat, lime, orange, pummelo, cucumber, melon, gourd, pumpkin, squash, eggplant, sweet pepper, hot pepper, tomatillo, herb, spice, mint, arugula, celery, chervil, endive, fennel, lettuce, parsley, radicchio, rhubarb, spinach, swiss chard, broccoli, brussels sprout, cabbage, cauliflower, collard, kale, kohlrabi, mustard green, asparagus, pear, quince, beet, sugarbeet, red beet, carrot, celeriac, chicory, horseradish, parsnip, radish rutabaga, salsify, and turnips, maple, pine, rye, wheat, sorghum, millet, apricot, cherry, nectarine, peach, plum, prune, almond, beech nut, Brazil nut, butternut, cashew, chestnut, filbert, hickory nut, macadamia nut, pecan, pistachio, walnut, artichoke, cassava, and ginger plants.

In a preferred aspect, the one or more endophyte strains can be selected from Bacillus amyloliquefaciens, Curtobacterium oceanosedimentum, Pseudomonas oryzihabitans, Pseudomonas oleovorans, Achromobacter xylosidans, Pantoea dispersa, and Enterobacter cloacae. In certain embodiments, the one or more endophyte strains comprise a nucleic acid sequence having at least 97% identity to a nucleic acid sequence selected from the group consisting of SEQ ID NO: 1, 2 and 7 to 18.

In certain embodiments, one or more endophyte strains are selected from the group of endophyte strains consisting of Bacillus amyloliquefaciens (strain Bamy) as deposited under NRRL Culture Deposit No. B-67479, Curtobacterium oceanosedimentum (strain WCB1) as deposited under NRRL Culture Deposit No. B-67478, Pseudomonas oryzihabitans (strain WCB2) as deposited under NRRL Culture Deposit No. B-67475, Pseudomonas oleovorans (strain Poryz) as deposited under NRRL Culture Deposit No. ______, Achromobacter xylosidans (strain Achromo) as deposited under NRRL Culture Deposit No. B-67480, Pantoea dispersa (strain Pdisp) as deposited under NRRL Culture Deposit No. B-67476, and Enterobacter cloacae (strain Entero) as deposited under NRRL Culture Deposit No. B-67477.

Traits of agronomic importance improved by treatment of the one or more endophytic strains include without limitation, suppression of growth of soil borne fungal pathogens, increased resistance to nematode disease, increased resistance to salt stress, increased biotic stress resistance, and increased abiotic stress resistance.

In a preferred embodiment, the plant is cotton or okra and the trait of agronomic importance is increased resistance to salt stress.

In yet another aspect, the one or more endophyte strains in the formulation are present in a synthetic seed ball or are used in a seed treatment. The endophyte strains may be present in a formulation, which may be a liquid or solid formulation. In certain embodiments the formulation is a liquid, and is sprayed on the plant or seeds. Alternatively, the liquid formulation is applied to the plant as a root dunk. The formulation may additionally comprise a controlled release fertilizer formulation. The formulation may be in the form of a synthetic combination which may also comprise an insecticide or fungicide.

In another aspect of the invention, a synthetic combination is provided. An exemplary synthetic combination comprises, a plant element and a formulation comprising one or more biologically pure Bacillus spp., Curtobacterium ssp., Pseudomonas ssp, Achromobacter ssp, Pantoea ssp., or Enterobacter ssp. endophyte strains isolated from Thespesia populnea and Gossypium hirsutuma, wherein said endophyte strains are present in the formulation in an amount capable of modulating a trait of agronomic importance in the plant comprising or derived from said plant element, as compared to a reference plant grown under the same conditions. The plant may be a monocot or a dicot. Plants to be treated with the synthetic combination include, without limitation cotton, okra, soybean, cacao, kenaf and kola nut, coffee, tobacco, potato, tomato, sweet potato, sunflower, rapeseed, wheat, corn, rice, barley, sorghum, grass, sugarcane, bamboo, buckwheat, snap bean, dry bean, canola, peas, peanuts, safflower, sunflower, alfalfa hay, clover, vetch, and trefoil, blackberry, blueberry, currant, elderberry, gooseberry, huckleberry, loganberry, raspberry, strawberry, grape, garlic, leek, onion, shallot, citrus hybrid, grapefruit, kumquat, lime, orange, pummelo, cucumber, melon, gourd, pumpkin, squash, eggplant, sweet pepper, hot pepper, tomatillo, herb, spice, mint, arugula, celery, chervil, endive, fennel, lettuce, parsley, radicchio, rhubarb, spinach, swiss chard, broccoli, brussels sprout, cabbage, cauliflower, collard, kale, kohlrabi, mustard green, asparagus, pear, quince, beet, sugarbeet, red beet, carrot, celeriac, chicory, horseradish, parsnip, radish rutabaga, salsify, and turnips, maple, pine, rye, wheat, sorghum, millet, apricot, cherry, nectarine, peach, plum, prune, almond, beech nut, Brazil nut, butternut, cashew, chestnut, filbert, hickory nut, macadamia nut, pecan, pistachio, walnut, artichoke, cassava, and ginger plants.

The synthetic combination preferably comprises one or more endophyte strains selected from Bacillus amyloliquefaciens, Curtobacterium oceanosedimentum, Pseudomonas oryzihabitans, Pseudomonas oleovorans, Achromobacter xylosidans, Pantoea dispersa, and Enterobacter cloacae.

The synthetic combination can be used to advantage to improve a plant trait selected from suppression of growth of soil borne fungal pathogens, increased resistance to nematode disease, increased resistance to salt stress, increased biotic stress resistance, and increased abiotic stress resistance.

In one aspect, the synthetic combination comprising one or more endophyte strains is present in a synthetic seed ball.

In a preferred embodiment, the synthetic combination comprises one or more endophyte strains selected from the group of endophyte strains consisting of Bacillus amyloliquefaciens (strain Bamy), Curtobacterium oceanosedimentum (strain WCB1) and Pseudomonas oryzihabitans and said plant to be treated is selected from the group of plants consisting of Cotton (Gossypium spp.), Okra Abelmoschus esculentus, Cacao (Theobroma cacao), Kenaf (Hibiscus cannabinus) and Kola nut (Cola spp.); Coffee (Coffea spp.), Tobacco (Nicotiana tabacum), Potato (Solanum tuberosum), Tomato (Solanum lycopsersicum), Sweet potato (Ipomoea batatas), Beans (Phaseolus spp.), Soybeans (Glycine max), Sunflowers (Helianthus spp.) and Rapeseed (Brassica napus).

Also provided is a method for enhancing nitrogen assimilation in a target host plant comprising introducing a plant expression vector encoding at least one of a plasma membrane HA-nitrate transporter, nitrate reductase, glutamine synthase, Wat-1, asparagine synthase, and asparaginase protein, thereby creating a transgenic plant, said transgenic plant exhibiting enhanced nitrogen assimilation relative to host plants lacking said plant transformation vector. This method can optionally further comprise, introducing a siRNA construct which down-modulates glutamate dehydrogenase 2, thereby creating a transgenic plant, said transgenic plant exhibiting enhanced nitrogen assimilation relative to host plants lacking said vector and said siRNA construct.

In another embodiment, a method for enhancing nitrogen assimilation in a target host plant comprising introducing a siRNA construct which down-modulates glutamate dehydrogenase 2, thereby creating a transgenic plant, said transgenic plant exhibiting enhanced nitrogen assimilation relative to host plants lacking said siRNA construct.

In another aspect a method for increasing salt tolerance in a target host plant comprising introducing a plant expression vector encoding at least one of a blue copper protein, lipid transfer protein DIR1, expansin A8, and laccase 4, thereby creating a transgenic plant, said transgenic plant exhibiting increased salt tolerance relative to host plants lacking said plant transformation vector. This method may optionally entail introducing a siRNA construct which down-modulates one or more of expansin B1, cucumisin, UDP-glycosyltransferase, alcohol dehydrogenase, and zinc finger protein ZAT11, thereby creating a transgenic plant, said transgenic plant exhibiting increased salt tolerance relative to host plants lacking said vector and said siRNA construct.

In yet another aspect, a method for increasing salt tolerance in a target host plant comprising introducing a siRNA construct which down-modulates one or more of expansin B1, cucumisin, UDP-glycosyltransferase, alcohol dehydrogenase, and zinc finger protein ZAT11,

In another embodiment, a method for increasing resistance to fungal infections in a target host plant comprising introducing a plant expression vector encoding at least one of EREB1, PR protein (PR1), ERF114, and peroxidase, thereby creating a transgenic plant, said transgenic plant exhibiting increased resistance to fungal infections relative to host plants lacking said plant transformation vector. This method may optionally entail introducing a siRNA construct which down-modulates one or more of metacaspase 9, metacaspase 3, and salicylate carboxymethyltransferase, thereby creating a transgenic plant, said transgenic plant exhibiting increased resistance to fungal infections relative to host plants lacking said vector and said siRNA construct.

In another aspect, a method for increasing resistance to fungal infections in a target host plant comprising introducing a siRNA construct which down-modulates one or more of metacaspase 9, metacaspase 3, and salicylate carboxymethyltransferase, thereby creating a transgenic plant, said transgenic plant exhibiting increased resistance to fungal infections relative to host plants lacking said siRNA construct.

Also provided are transgenic plants produced by any of methods described above.

### BRIEF DESCRIPTION OF THE DRAWINGS

FIG. 1. Microbial growth on Gossypium hirsutum seeds after 72 hours of inoculating on PDA. FIG. 1A) Non-surface sterilized seeds of non-cultivated cotton. FIG. 1B) Non-surface sterilized seeds of acid delinted, cultivated cotton. FIG. 1C) Seeds of wild, non-cultivated cotton seeds delinted with 25% sulfuric acid for 5 minutes.

FIG. 2. Percentage of germinated seeds of Gossypium hirsutum seeds inoculated with various bacterial suspensions (n=18). Ba=Bacillus amyloliquefaciens (strain Bamy), Co=Curtobacterium oceanosedimentum (strain WCB1), Po=Pseudomonas oryzihabitans (strain WCB2).

FIG. 3. Length of cotton seedling radicles inoculated with various bacterial suspensions (n=10). Ba=Bacillus amyloliquefaciens (strain Bamy), Co=Curtobacterium oceanosedimentum (strain WCB1), Po=Pseudomonas oryzihabitans (strain WCB2). FIG. 4. Inoculation of cotton seeds with Bacillus amyloliquefaciens (strain Bamy) led to the inoculation of the soil surrounding emerging radicles in soil treated with 150 mM NaCl (upper left) and water (upper right). Seeds in uninoculated control treatments did not have evidence of bacterial growth in the soil surrounding seedlings in soil treated with 150 mM NaCl (lower left) and water (lower right).

FIG. 5. Systemic colonization of Bacillus amyloliquefaciens (strain Bamy) in 7 day old Gossypium hirsutum seedlings. FIG. 5A) Colonization of cotyledons, FIG. 5B) colonization of seedling stems, FIG. 5C) Colonization of seedling roots.

FIG. 6. Frequency of Gossypium hirsutum seeds that developed into seedlings with expanded cotyledons in salt stressed and non-stressed conditions and inoculated with Bacillus amyloliquefaciens (strain Bamy) (n=21).

FIG. 7. Growth promotion of non-stressed and salt stressed cotton seedlings inoculated with Bacillus amyloliquefaciens (strain Bamy).

FIG. 8. Growth promotion and changes in root architecture of 7-day-old cotton seedlings inoculated with Bacillus amyloliquefaciens (strain Bamy). FIG. 8A) Cotton seedling roots grown in soil treated with 150 mM NaCl, FIG. 8B) seedling roots treated with water, FIG. 8C) seedling roots treated with 150 mM NaCl and inoculated with Bacillus amyloliquefaciens (strain Bamy), FIG. 8D) seedling roots treated with water and inoculated with Bacillus amyloliquefaciens (strain Bamy). (bar=1 cm).

FIG. 9. Distribution of differentially expressed genes (DEGs) in non-stressed cotton seedling roots inoculated with Bacillus amyloliquefaciens (strain Bamy) (n=252).

FIG. 10. Distribution of up-regulated DEGs in non-stressed cotton seedling roots inoculated with Bacillus amyloliquefaciens (strain Bamy) (n=139).

FIG. 11. Distribution of down-regulated DEGs in cotton seedling roots inoculated with Bacillus amyloliquefaciens (strain Bamy) (n=113).

FIG. 12. Distribution of total DEGs in salt stressed cotton seedling roots inoculated with Bacillus amyloliquefaciens (strain Bamy) (n=108).

FIG. 13. Distribution of up-regulated DEGs in salt stressed cotton seedling roots inoculated with Bacillus amyloliquefaciens (strain Bamy) (n=76).

FIG. 14. Distribution of down-regulated DEGs in salt stressed cotton seedling roots inoculated with Bacillus amyloliquefaciens (strain Bamy) (n=32).

FIG. 15. Exemplary DEGs identified to be involved in the nitrogen assimilation pathway in cotton seedling roots inoculated with Bacillus amyloliquefaciens (strain Bamy). Up-regulated genes encode for proteins in green and down-regulated genes encode for proteins indicated in red.

FIG. 16. Exemplary MapMan overview of DEGs related to metabolic pathways in non-stressed and inoculated cotton seedling roots.

FIG. 17. Exemplary MapMan visualization of nitrogen metabolism-related DEGs in inoculated cotton seedling roots in non-stressed conditions.

FIG. 18. Exemplary MapMan overview of DEGs related to metabolic pathways in salt stressed and inoculated cotton seedling roots.

FIG. 19. Exemplary MapMan summary of DEGs related to biotic stress pathways in non-stressed cotton seedling roots inoculated with Bacillus amyloliquefaciens (strain Bamy).

FIG. 20. Exemplary MapMan summary of DEGs related to biotic stress pathways in salt stressed cotton seedling roots inoculated with Bacillus amyloliquefaciens (strain Bamy).

FIG. 21. Inhibition zones formed between fungal isolates and Bacillus amyloliquefaciens (strain Bamy). FIG. 21A) Lasiodiplodia theobromae, FIG. 21B) Bionectria ochroleuca, FIG. 21C) Diaporthe sp., FIG. 21D) Cladosporium sp., FIG. 21E) Curvularia lunata, FIG. 21F) Fusarium sp., FIG. 21G) Fusarium brachygibbosum, FIG. 21H) Setosphaeria rostrata, FIG. 21I) Neofusicoccum australe, FIG. 21J) Diaporthe sp. FIG. 21K) Phoma sp., FIG. 21 L) Fusarium sp., FIG. 21M) Phoma sp., FIG. 21N) Neofusicoccum parvum, FIG. 21O) Diaporthe sp., FIG. 21P) Fusarium sp.

FIG. 22. Average width of inhibition zones (mm) formed between various fungi and Bacillus amyloliquefaciens (strain Bamy) when cultured on PDA for 7 days (n=3). The source of the fungal isolate is indicated in parentheses. Tp=Thespesia populnea, Gh=Gossypium hirsutum.

FIG. 23. The effects of endophytic bacteria on the colony diameter of various filamentous fungi after 72 hours of co-culturing on PDA (n=3).

FIG. 24A-24E. Growth rate of filamentous fungi colonies on PDA after 24, 48, and 72 hours t 25° C. when co-cultured with endophytic bacteria isolated from Thespesia populnea. FIG. 24A) Growth rate of Lasiodiplodia theobromae (Lt) colonies co-cultured with endophytic bacteria FIG. 24B) Growth rate of Diaporthe sp. (Dia) colonies co-cultured with endophytic bacteria FIG. 24C) Growth rate of Bionectria ochroleuca (Bo) colonies co-cultured with endophytic bacteria FIG. 24D) Growth rate of Curvularia lunata (Cl) colonies co-cultured with endophytic bacteria FIG. 24E) Growth rate of Fusarium sp. (Fus) colonies co-cultured with endophytic bacteria. Each point is the average of three replicates±the standard deviation. Po=Pseudomonas oryzihabitans (strain WCB2), Pd=Pantoea dispersa (strain Pdisp), Ax=Achromobacter xylosoxidans (strain Achromo), Ec=Enterobacter cloacae (strain Entero), Ba=Bacillus amyloliquefaciens (strain Bamy).

FIG. 25. Average hyphal width of seed-transmitted fungi of Thespesia populnea (Tp) and Gossypium hirsutum (Gh) when co-cultured with various endophytic bacteria from Thespesia populnea (n=20).

FIG. 26. Exemplary effects of endophytic bacteria described herein on the severity of Lasiodiplodia theobromae fruit rot in bananas. The top 3 panels from left to right provide exemplary depictions of 1) negative control (holes); 2) positive control (Lasiodiplodia theobromae); and 3) necrotic lesions on bananas pericarps inoculated with the pathogen Lasiodiplodia theobromae and the endophytic bacteria, B. amyloliquefaciens (strain Bamy). The bottom 4 panels from left to right provide exemplarydepictions of necrotic lesions on banas pericarps inoculated with the pathogen Lasiodiplodia theobromae and the endophytic bacteria 1) P. dispersa (strain Pdisp); 2) P. oryzihabitans (strain WCB2); 3) E. cloacae (strain Entero); and 4) A. xylosoxidans (strain Achromo).

FIG. 27. The effects of endophytic bacteria on the average size of necrotic lesions of Lasiodiplodia theobromae on banana pericarps (n=10). Lt=Lasiodiplodia theobromae, Ba=Bacillus amyloliquefaciens (strain Bamy), Pd=Pantoea dispersa (strain Pdisp), Po=Pseudomonas oryzihabitans (strain WCB2), Ec=Enterobacter cloacae (strain Entero), Ax=Achromobacter xylosoxidans (strain Achromo).

FIG. 28. Antifungal activity of a crude lipopeptide extract from Bacillus amyloliquefaciens (strain Bamy) grown on potato dextrose broth for 48 hours. FIG. 28A) Lasiodiplodia theobromae inhibited by crude lipopeptides extracted from B. amyloliquefaciens (strain Bamy). FIG. 28B) Control treatment with disks treated with methanol alone.

FIG. 29. HPLC profile of a crude lipopeptide extract from a liquid culture of Bacillus amyloliquefaciens (strain Bamy). The peaks recovered are possibly lipopeptides.

FIG. 30. HPLC fractions induce chlamydospore formation in Fusarium sp. Fractions #59 (A) and #60 (B) obtained through HPLC induced chlamydospore production in cultures of Fusarium sp. Samples were stained with aniline blue and viewed at 400× magnification.

FIG. 31. MALDI-TOF mass spectra of fraction #59. The green circle indicates a peak predicted to correspond to the lipopeptide fengycin (1435 m/z).

FIG. 32. MALDI-TOF mass spectra of fraction #60. The green circle indicates a peak predicted to correspond to the lipopeptide fengycin (1465 m/z).

FIG. 33 is an exemplary image demonstrating the ability of the bacteria of the present invention to inhibit the growth of the fungal pathogen Fusarium oxysporum 47 (Fo47) in the antibiosis assay described in Example IV. This plate had been incubated for 4 days at room temperature. The letters appear directly below the spots where the compound or bacteria to be tested was applied. Nothing was applied at position A as a negative control. The fungicidal compound Amphotericin B was applied at position B as a positive control. The carrier DMSO was applied at position C. The strain Achromo was applied at position D. The strain Bamy was applied at position E; this strain produced a distinct zone of inhibition indicating its strong anti-Fusarium activity. The strain WCB1 was applied at position F. The strain Entero was applied at position G and showed strong anti-Fusarium activity as denoted by the distinct and large zone of inhibition around the application site. The strain Pdisp was applied at position H. The strain Poryz was applied at position I. The strain WCB2 was applied at position J. A strain with established anti-Fusarium activity was applied at position N as a positive biological control.

FIG. 34 shows exemplary roots which were scored using the split-root scoring system at 45 days post planting. A exemplifies a root receiving a 0 score. B exemplifies a root receiving a 1 score. C exemplifies a root receiving a 2 score. D exemplifies a root receiving a 3 score.

FIG. 35 shows the percent survival of soybean plants in the presence of Fusarium virguliforme and Rotylenchulus reniformis. Soybean plants treated with strain Achromo (Ax) showed a 10% increase in survival relative to formulation controls. The bar height represents the mean value, the error bars represent 95% confidence interval from the mean. UTC=untreated control, Formulation=formulation control. The field methods are described in Example XI, survival was calculated at 46 days after planting.

FIG. 36 shows the height of soybean plants in the presence of Fusarium virguliforme and Rotylenchulus reniformis. Soybean plants treated with strain Entero and showed a greater than 10% increase height relative to formulation controls. The error bars represent 95% confidence interval from the mean. UTC=untreated control, Formulation=formulation control. The field methods are described in Example XI, plant height was measured at 49 days after planting.

FIG. 37 shows the fresh shoot weight of soybean plants in the presence of Fusarium virguliforme and Rotylenchulus remformis. Soybean plants treated with strains Entero, Bamy, and Achromo (Ax) showed a greater than 10% increase in fresh shoot weight relative to formulation controls. The error bars represent 95% confidence interval from the mean. UTC=untreated control, Formulation=formulation control. The field methods are described in Example XI, fresh shoot weight was measured at 49 days after planting.

FIG. 38 shows the number of Rotylenchulus reniformis (Reniform) eggs detected per plant for plants selected from each replicate for each treatment. Soybean plants treated with strains Entero and WCB1 showed reduced numbers of Reniform eggs per plant compared the formulation control and similar reductions to Strain 3 and Strain 4 which are known to have biocontrol activity against Reniform. The bar height represents the mean value, the error bars represent 95% confidence interval from the mean. Formulation=formulation control. The field methods are described in Example XI, egg count was measured at 49 days after planting.

FIG. 39 shows the soybean sudden death syndrome (SDS) disease score of soybean plants grown in the presence of Fusarium virguliforme and Rotylenchulus remformis. Treatment with Pdisp or WCB1 conferred improved SDS resistance. The error bars represent 95% confidence interval from the mean. UTC=untreated control, Formulation=formulation control. The field methods are described in Example XI. Disease symptoms were scored on a 0-3 scale (3 denotes strong disease symptoms) using the split-root scoring system at 45 days post planting.

FIG. 40 shows average yield as percent gain of Bamy treated cotton seeds over control seeds not treated with an endophyte for multiple trialing locations and varieties of cotton.

FIG. 41 shows average yield as percent gain of Bamy treated corn seeds over control seeds not treated with an endophyte for multiple trialing locations and varieties of corn.

FIG. 42 shows average yield as percent gain of M oxydans B2 treated corn seeds over control seeds not treated with an endophyte for multiple trialing locations and varieties of corn.

### DETAILED DESCRIPTION OF THE INVENTION

Beneficial bacterial endophytes are capable of promoting growth and alleviating abiotic and biotic stress in plants. Non-cultivated relatives of cotton in stressed environments as described hereinbelow possess beneficial bacteria capable of promoting growth and alleviating stress in cultivated plants. Using cotton as a model involves bacteria in non-cultivated relatives in the cotton family (Malvaceae). Cultivated cotton provides a useful model for these studies because cotton seeds are acid delinted. Acid delinting is a century-old process that involves treating seeds with diluted sulfuric or hydrochloric acid to remove fuzzy lint covering seeds which facilitates cotton seed mass planting and reduces the prevalence of seed-borne diseases. This practice disturbs the seed-transmitted cotton microbiome and thus, also affects the communities of beneficial microbes that are vertically transmitted to developing cotton seedlings.

In the Examples described herein, non-cultivated, wild plants in the Malvaceae family collected from saline and arid areas in Puerto Rico promoted growth, alleviated salt stress, and protected cotton seedlings against seed-borne fungal diseases. Endophytic bacteria of the present invention enhanced cotton seed germination and altered the growth of various fungi. In one aspect, the present invention includes strains of bacteria isolated from wild relatives of cotton and found to improve growth of cultivated cotton which include, without limitation, Achromobacter xylosidans (strain Achromo), Bacillus amyloliquefaciens (strain Bamy), Curtobacterium oceanosedimentum (strain WCB1), Pseudomonas oleovorans (strain Poryz), Pseudomonas oryzihabitans (strain WCB2), Pantoea dispersa (strain Pdisp) and Enterobacter cloacae (strain Entero). Among the endophytic bacteria described herein, Bacillus amyloliquefaciens (strain Bamy) was further demonstrated to promote growth, alleviate salt stress, and alter root architecture of cotton and okra seedlings. Using a GeneChip microarray gene expression analysis, it was demonstrated that inoculating cotton seedling roots with B. amyloliquefaciens led to the differential expression of hundreds of genes in both non-stressed and salt stressed conditions. Many of the differentially expressed genes appear to contribute to the phenotypic effects observed on inoculated cotton seedlings, including selective upregulation of stress tolerance genes, for example antioxidant genes, as well as nitrogen assimilation genes, e.g., increase nutrient assimilation in the plant. Moreover, B. amyloliquefaciens (strain Bamy) inhibited growth of numerous fungi and produced lipopeptides with antifungal and chlamydospore-inducing properties. Data supported that B. amyloliquefaciens (strain Bamy) promoted growth and alleviated biotic and abiotic stress of multiple hosts making it a suitable candidate to be used as a biofertilizer and biocontrol agent. Biological agents that enhance plant growth and health should be effective to decrease the demand for nitrogenous fertilizers and fungicides that are costly and detrimental to the environment.

Delinting of cotton seed with sulfuric or hydrochloric acid is a common practice in the process of cotton seed mass production that has been used for over a century. It is desirable to remove lint from cotton seeds to prevent seeds from clumping together and increase the flow of seeds during planting (Delouche 1986).

There are various methods to remove fibers from cotton seed that involve using acids such as applying wet acid, gas acid, and dilute wet acid. Aside from removing lint, treating seeds with acid, leads to enhanced germination and decreased infection by seed-transmitted pathogens (Brown 1933). Archibald (1927) documented that sulfuricacid delinting was used to prevent against diseases such as leaf spot, black arm, boll rot, and anthracnose. Unfortunately, acid delinting not only eliminates seed-transmitted pathogens of cotton, but also eliminates vertically transmitted beneficial bacteria of the seed microbiome. Thus, sterilizing cotton seeds through the process acid delinting eliminates the cotton seed microbiome as an initial source of beneficial microbes capable of promoting growth of young seedlings.

Table 1A provides a listing of the bacteria described herein, strain names and abbreviations used for the strains, the GenBank Accession number for rDNA sequences present in said bacteria, along with SEQ ID NOS and NRRL deposit information.

Table 1B provides the rDNA sequences and corresponding SEQ ID NOS for preferred isolates.

TABLE 1A
Beneficial bacterial endophytes of the present invention

| | | | | rDNA | NRRL | |
|---|---|---|---|---|---|---|
| | | Source | GenBank | SEQ | Culture | Anti- |
| Strain | Species | host | Nos. | ID NOS: | Deposit No. | Fungal |
| Bamy | Bacillus | Thespesia | KX622565 | SEQ ID | B-67479 | ✓* |
| (or Pb1)^{#} | amyloliquefaciens | populnea | | NO: 1 | | |
| | | (leaves, | | SEQ ID | | |
| | | seeds) | | NO: 2 | | |
| WCB1 | Curtobacterium | Wild cotton | KX622563 | SEQ ID | B-67478 | ✓ |
| (or Cb1) | oceanosedimentum | (Gossypium | | NO: 7 | | |
| | | hirsutum) | | SEQ ID | | |
| | | (seeds) | | NO: 8 | | |
| WCB2 | Pseudomonas | Wild cotton | KX622564 | SEQ ID | B-67475 | ✓ |
| (or Cb2) | oryzihabitans | (Gossypium | | NO:9 | | |
| | | hirsutum) | | SEQ ID | | |
| | | (seeds) | | NO: 10 | | |
| Poryz | Pseudomonas | Thespesia | KY471285 | SEQ ID | B-XXX | ✓ |
| (or Pb7) | oleovorans | populnea | | NO: 11 | | |
| | | (seeds) | | SEQ ID | | |
| | | | | NO: 12 | | |
| Achromo | Achromobacter | Thespesia | KY471283 | SEQ ID | B-67480 | ✓ |
| (Pb5 | xylosidans | populnea | | NO: 13 | | |
| or Ax) | | (seeds) | | SEQ ID | | |
| | | | | NO: 14 | | |
| Pdisp | Pantoea dispersa | Thespesia | KY471284 | SEQ ID | B-67476 | ✓ |
| (Pb6) | | populnea | | NO: 15 | | |
| | | (seeds) | | SEQ ID | | |
| | | | | NO: 16 | | |
| Entero | Enterobacter | Thespesia | KY471282 | SEQ ID | B-67477 | ✓ |
| (Pb4) | cloacae | populnea | | NO: 17 | | |
| | | (seeds) | | SEQ ID | | |
| | | | | NO: 18 | | |

| | Increase | Increase | | | Increase | |
|---|---|---|---|---|---|---|
| | root | salt | Phos. | | germ | Auxin |
| Strain | growth | tolerance | solubil. | Protease | rate | production |
| Bamy | ✓ | ✓ | ✓ | ✓ | ✓ | No |
| (or Pb1)^{#} | | | | | | |
| WCB1 | ✓ | — | ✓ | ✓ | ✓ | ✓ |
| (or Cb1) | | | | | | |
| WCB2 | ✓ | — | ✓ | No | ✓ | ✓ |
| (or Cb2) | | | | | | |
| Poryz | — | — | — | — | — | — |
| (or Pb7) | | | | | | |
| Achromo | — | — | — | — | — | — |
| (Pb5 | | | | | | |
| or Ax) | | | | | | |
| Pdisp | ✓ | ✓ | — | — | — | — |
| (Pb6) | | | | | | |
| Entero | — | — | — | — | — | — |
| (Pb4) | | | | | | |

TABLE 1B
16S rDNA sequences shown unless otherwise indicated
Sequences SEQ ID NO: 1 Bacillus amyloliquefaciens (strain Bamy)
CGGCGGCTGGCTCCATAAAGGTTACCTCACCGACTTCGGGTGTTACAAACTCTCGTGGTGTGACGGGCGGTGTGTAC
AAGGCCCGGGAACGTATTCACCGCGGCATGCTGATCCGCGATTACTAGCGATTCCAGCTTCACGCAGTCGAGTTGCA
GACTGCGATCCGAACTGAGAACAGATTTGTGGGATTGGCTTAACCTCGCGGTTTCGCTGCCCTTTGTTCTGTCCATT
GTAGCACGTGTGTAGCCCAGGTCATAAGGGGCATGATGATTTGACGTCATCCCCACCTTCCTCCGGTTTGTCACCGG
CAGTCACCTTAGAGTGCCCAACTGAATGCTGGCAACTAAGATCAAGGGTTGCGCTCGTTGCGGGACTTAACCCAACA
TCTCACGACACGAGCTGACGACAACCATGCACCACCTGTCACTCTGCCCCCGAAGGGGACGTCCTATCTCTAGGATT
GTCAGAGGATGTCAAGACCTGGTAAGGTTCTTCGCGTTGCTTCGAATTAAACCACATGCTCCACCGCTTGTGCGGGC
CCCCGTCAATTCCTTTGAGTTTCAGTCTTGCGACCGTACTCCCCAGGCGGAGTGCTTAATGCGTTAGCTGCAGCACT
AAGGGGCGGAAACCCCCTAACACTTAGCACTCATCGTTTACGGCGTGGACTACCAGGGTATCTAATCCTGTTCGCTC
CCCACGCTTTCGCTCCTCAGCGTCAGTTACAGACCAGAGAGTCGCCTTCGCCACTGGTGTTCCTCCACATCTCTACG
CATTTCACCGCTACACGTGGAATTCCACTCTCCTCTTCTGCACTCAAGTTCCCCAGTTTCCAATGACCCTCCCCGGT
TGAGCCGGGGGCTTTCACATCAGACTTAAGAAACCGCCTGCGAGCCCTTTACGCCCAATAATTCCGGACAACGCTTG
CCACCTACGTATTACCGCGGCTGCTGGCACGTAGTTAGCCGTGGCTTTCTGGTTAGGTACCGTCAAGGTGCCGCCCT
ATTTGAACGGCACTTGTTCTTCCCTAACAACAGAGCTTTACGATCCGAAAACCTTCATCACTCACGCGGCGTTGCTC
CGTCAGACTTTCGTCCATTGCGGAAGATTCCCTACTGCTGCCTCCCGTAGGAGTCTGGGCCGTGTCTCAGTCCCAGT
GTGGCCGATCACCCTCTCAGGTCGGCTACGCATCGTCGCCTTGGTGAGCCGTTACCTCACCAACTAGCTAATGCGCC
GCGGGTCCATCTGTAAGTGGTAGCCGAAGCCACCTTTTATGTCTGAACCATGCGGTTCAGACAACCATCCGGTATTA
GCCCCGGTTTCCCGGAGTTATCCCAGTCTTACAGGCAGGTTACCCACGTGTTACTCACCCGTCCGCCGCTAACATCA
GGGAGCAAGCTCCCATCTGTCCGCTCGACTGCATG
SEQ ID NO: 2 Bacillus amyloliquefaciens (strain Bamy)
TCACCCCAATCATCTGTCCCACTTCGGCGGCTGGCTCCATAAAGGTTACCTCACCGACTTCGGGTGTTACAAACTCT
CGTGGTGTGACGGGCGGTGTGTACAAGGCCCGGGAACGTATTCACCGCGGCATGCTGATCCGCGATTACTAGCGATT
CCAGCTTCACGCAGTCGAGTTGCAGACTGCGATCCGAACTGAGAACAGATTTGTGGGATTGGCTTAACCTCGCGGTT
TCGCTGCCCTTTGTTCTGTCCATTGTAGCACGTGTGTAGCCCAGGTCATAAGGGGCATGATGATTTGACGTCATCCC
CACCTTCCTCCGGTTTGTCACCGGCAGTCACCTTAGAGTGCCCAACTGAATGCTGGCAACTAAGATCAAGGGTTGCG
CTCGTTGCGGGACTTAACCCAACATCTCACGACACGAGCTGACGACAACCATGCACCACCTGTCACTCTGCCCCCGA
AGGGGACGTCCTATCTCTAGGATTGTCAGAGGATGTCAAGACCTGGTAAGGTTCTTCGCGTTGCTTCGAATTAAACC
ACATGCTCCACCGCTTGTGCGGGCCCCCGTCAATTCCTTTGAGTTTCAGTCTTGCGACCGTACTCCCCAGGCGGAGT
GCTTAATGCGTTAGCTGCAGCACTAAGGGGCGGAAACCCCCTAACACTTAGCACTCATCGTTTACGGCGTGGACTAC
CAGGGTATCTAATCCTGTTCGCTCCCCACGCTTTCGCTCCTCAGCGTCAGTTACAGACCAGAGAGTCGCCTTCGCCA
CTGGTGTTCCTCCACATCTCTACGCATTTCACCGCTACACGTGGAATTCCACTCTCCTCTTCTGCACTCAAGTTCCC
CAGTTTCCAATGACCCTCCCCGGTTGAGCCGGGGGCTTTCACATCAGACTTAAGAAACCGCCTGCGAGCCCTTTACG
CCCAATAATTCCGGACAACGCTTGCCACCTACGTATTACCGCGGCTGCTGGCACGTAGTTAGCCGTGGCTTTCTGGT
TAGGTACCGTCAAGGTGCCGCCCTATTTGAACGGCACTTGTTCTTCCCTAACAACAGAGCTTTACGATCCGAAAACC
TTCATCACTCACGCGGCGTTGCTCCGTCAGACTTTCGTCCATTGCGGAAGATTCCCTACTGCTGCCTCCCGTAGGAG
TCTGGGCCGTGTCTCAGTCCCAGTGTGGCCGATCACCCTCTCAGGTCGGCTACGCATCGTCGCCTTGGTGAGCCGTT
ACCTCACCAACTAGCTAATGCGCCGCGGGTCCATCTGTAAGTGGTAGCCGAAGCCACCTTTTATGTCTGAACCATGC
GGTTCAGACAACCATCCGGTATTAGCCCCGGTTTCCCGGAGTTATCCCAGTCTTACAGGCAGGTTACCCACGTGTTA
CTCACCCGTCCGCCGCTAACATCAGGGAGCAAGCTCCCATCTGTCCGCTCGACTTGCATGTATTAGGCACGCCGCCA
GCGTTCGTCCTGA
SEQ ID NO: 3 Iturin Gene Sequence (Strain Bamy)
CAAAGACAATGAACGCGCAGCCCGCTATTTTAACGGTCAGTGTTATTGCTTTTCAAGTGTATATGCAGGAAATAGGG
GTGAAGCCCCGCTTCCTGGCAGGCCATAGCTTAGGCGAATATTCAGCGCTTGTCTGTGCCGGCGCCCTTTCTTTTCA
GGATGCCGTTACACTTGTAAGGCAGCGGGGAATTCTTATGCAGAATGCGGATCCTCAGCAGCAGGGGACGATGGCCG
CCGTGACTCACCTCTCTCTTCAAACGTTGCAGGAAATATGTTCGAAAGTGTCGACGGAAGACTTTCCGGCAGGAGTA
GCCTGCATGAATTCAGAACAGCAGCATGTGATTTCCGGACACCGGCAAGCTGTGGAACGTGTCATCAAGATGGCGGA
GGAAAAGGGAGCGGCATACACTTATTTGAATGTCAGTGCGCCTTTTCACAGTTCGCTGATACGATCAGCATCTGAAC
AATTCCAGACTGTATTACACCGGTATTCCTTCCGGGAAGCCGCATGGCCGATCATTTCAAATGTCACTGCACGCCCT
TACAGCAGCGGAAATTCGATCAGCGAACAT
SEQ ID NO: 4 Surfactin Gene Sequence (Strain Bamy)
CANTTTTTCAGTTTCACTTAATATGAACATGTGGCCGTCCGAAAACTCATGAAATACGGGATTGTCGGCCCATTTTT
TCCATCCGTCCGCATCTTTGATACATTTTTTATCTTTCCGCCCGTTAAAAATATGGACGGGTGATTGAATCATGTGA
GAATCAGACGGACGGAAGCTTTCAAGCGCGCGGTAGTCGGACCTGAAAGAAGGCAGGAAAAATGACATGACCTCCTT
ATTCTCCACTAACTCCTGCGGCATTCCGCCCAGCTCGATAATATGGGCGAGAAATTTTTCATCATCAAGATGAGACA
CTTTTTTCCTTTCAACATGCGGCGGCTGAATGGCTGAGATGATCACGGCCTGCGGGTAAATCCCCTCCCGCTCCAGC
TTTTGCGCCAGCCTGAAAGCGACCATGC
SEQ ID NO: 5 RecA (Recombinase) Gene Sequence (Strain Bamy)
TAAGCAAATAGAAAAACAATTCGGCAAAGGTTCCATCATGAAGCTCGGAGAAAAAACGGATACAAGAATTTCAACGG
TGCCAAGCGGCTCCCTTGCACTTGATACTGCTCTCGGAATAGGCGGATACCCGCGCGGACGGATTATTGAAGTATAC
GGACCTGAAAGTTCAGGTAAAACGACTGTAGCGCTTCACGCAATCGCTGAGGTTCAGGAAAAAGGCGGACAGGCCGC
ATTTATTGATGCTGAGCATGCTCTTGATCCTGTTTACGCGCAAAAGCTCGGTGTCAATATCGAAGAGCTTCTGCTTT
CTCAGCCGGATACGGGAGAGCAGGCGCTTGAGATTGCTGAAGCGCTGGTGCGAAGCGGAGCTGTTGATATCGTAGTC
GTTGACTCTGTTGCGGCGCTTGTTCCAAAAGCTGAAATTGAAGGTGACATGGGTGATTCACACGTCGGTTTACAGGC
GCGTCTTATGTCTCAGGCGCTCCGTAAGCTTTCCGGCGCTATCAATAAATCTAAAACAATCGCAATCTTTATTAACC
AGATTCG
SEQ ID NO: 6 GyrA (Gyrase Subunit A) Gene Sequence (Strain Bamy)
CGTTATCGTATCCCGGGCGCTTCCGGATGTGCGTGACGGTCTGAAGCCGGTTCACAGACGGATTTTGTACGCAATGA
ATGATTTAGGCATGACCAGTGACAAACCATATAAAAAATCTGCCCGTATCGTCGGTGAAGTTATCGGTAAGTACCAC
CCGCACGGTGACTCAGCGGTTTACGAATCAATGGTCAGAATGGCGCAGGATTTTAACTACCGCTACATGCTTGTTGA
CGGACACGGCAACTTCGGTTCGGTTGACGGCGACTCAGCGGCCGCGATGCGTTACACAGAAGCGAGAATGTCAAAAA
TCGCAATGGAAATTCTGCGTGACATTACGAAAGACACGATTGACTATCAAGATAACTATGACGGTTCAGAAAGAGAG
CCTGCCGTCATGCCTTCGAGATTTCCGAATCTGCTCGTAAACGGGGCTGCCGGTATTGCGGTCGGAATGGCGACAAA
CATTCCCCCGCATCAGCTTGGGGAAGTCATTGAAGGCGTGCTTGCCGTAAGTGAGAATCCTGAGATTACAAACCAGG
AGCTGATGGAATACATCCCGGGCCCGGATTTTCCGACTGCAGGTCAGATTTTGGGCCGGAGCGGCATCCGCAAGGCA
TATGAATCCGGACGGGGATCAATCACGATCCGGGCTAAGGCTGAAATCGAAGAGACTTCATCGGGAAAAGAAAGAAT
TATTGTCACGGAACTTCCTTATCAGGTGAACAAAGCGAGATTAATTGAAAAAATCGCGGATCTTGTCCGAGACAAAA
AAATCGAAGGAATTACCGATCTGCGAGACGAATCCGACCGTAACGGAATGAGAATCGTCATTGAGATCCGCCGTGAC
GCCAATGCTCACGTCATTTTGAATAACCTGTACAAACAAACGGCCCTGCAGACGTCTTTCGGAATCAATCTGCTGGC
GCTCGTTGACGAAAGC
SEQ ID NO: 7 Curtobacterium oceanosedimentum (strain WCB1)
TGGGTGGATTAGTGGCGAACGGGTGAGTAACACGTGAGTAACCTGCCCCTGACTCTGGGATAAGCGTTGGAAACGAC
GTCTAATACTGGATACGACTGCCGGCCGCATGGTCTGGTGGTGGAAAGATTTTTTGGTTGGGGATGGACTCGCGGCC
TATCAGCTTGTTGGTGAGGTAATGGCTCACCAAGGCGACGACGGGTAGCCGGCCTGAGAGGGTGACCGGCCACACTG
GGACTGAGACACGGCCCAGACTCCTACGGGAGGCAGCAGTGGGGAATATTGCACAATGGGCGAAAGCCTGATGCAGC
AACGCCGCGTGAGGGATGACGGCCTTCGGGTTGTAAACCTCTTTTAGTAGGGAAGAAGCGAAAGTGACGGTACCTGC
AGAAAAAGCACCGGCTAACTACGTGCCAGCAGCCGCGGTAATACGTAGGGTGCAAGCGTTGTCCGGAATTATTGGGC
GTAAAG
SEQ ID NO: 8 Curtobacterium oceanosedimentum (strain WCB1)
CTCAGGACGAACGCTGGCGGCGTGCTTAACACATGCAAGTCGAACGATGATGCCCAGCTTGCTGGGTGGATTAGTGG
CGAACGGGTGAGTAACACGTGAGTAACCTGCCCCTGACTCTGGGATAAGCGTTGGAAACGACGTCTAATACTGGATA
CGACTGCCGGCCGCATGGTCTGGTGGTGGAAAGATTTTTTGGTTGGGGATGGACTCGCGGCCTATCAGCTTGTTGGT
GAGGTAATGGCTCACCAAGGCGACGACGGGTAGCCGGCCTGAGAGGGTGACCGGCCACACTGGGACTGAGACACGGC
CCAGACTCCTACGGGAGGCAGCAGTGGGGAATATTGCACAATGGGCGAAAGCCTGATGCAGCAACGCCGCGTGAGGG
ATGACGGCCTTCGGGTTGTAAACCTCTTTTAGTAGGGAAGAAGCGAAAGTGACGGTACCTGCAGAAAAAGCACCGGC
TAACTACGTGCCAGCAGCCGCGGTAATACGTAGGGTGCAAGCGTTGTCCGGAATTATTGGGCGTAAAGAGCTCGTAG
GCGGTTTGTCGCGTCTGCTGTGAAATCCCGAGGCTCAACCTCGGGCTTGCAGTGGGTACGGGCAGACTAGAGTGCGG
TAGGGGAGATTGGAATTCCTGGTGTAGCGGTGGAATGCGCAGATATCAGGAGGAACACCGATGGCGAAGGCAGATCT
CTGGGCCGTAACTGACGCTGAGGAGCGAAAGCGTGGGGAGCGAACAGGATTAGATACCCTGGTAGTCCACGCCGTAA
ACGTTGGGCGCTAGATGTAGGGACCTTTCCACGGTTTCTGTGTCGTAGCTAACGCATTAAGCGCCCCGCCTGGGGAG
TACGGCCGCAAGGCTAAAACTCAAAGGAATTGACGGGGGCCCGCACAAGCGGCGGAGCATGCGGATTAATTCGATGC
AACGCGAAGAACCTTACCAAGGCTTGACATACACCGGAAACGGCCAGAGATGGTCGCCCCCTTGTGGTCGGTGTACA
GGTGGTGCATGGTTGTCGTCAGCTCGTGTCGTGAGATGTTGGGTTAAGTCCCGCAACGAGCGCAACCCTCGTTCTAT
GTTGCCAGCGCGTTATGGCGGGGACTCATAGGAGACTGCCGGGGTCAACTCGGAGGAAGGTGGGGATGACGTCAAAT
CATCATGCCCCTTATGTCTTGGGCTTCACGCATGCTACAATGGCCGGTACAAAGGGCTGCGATACCGTAAGGTGGAG
CGAATCCCAAAAAGCCGGTCTCAGTTCGGATTGAGGTCTGCAACTCGACCTCATGAAGTCGGAGTCGCTAGTAATCG
CAGATCAGCAACGCTGCGGTGAATACGTTCCCGGGCCTTGTACACACCGCCCGTCAAGTCATGAAAGTCGGTAACAC
CCGAAGCCGGTGGCCTAACCCTTGTGGAAGGAGCCGTC
SEQ ID NO: 9 Pseudomonas oryzihabitans (Strain WCB2)
ACTAGCTACTTCTGGAGCAACCCACTCCCATGGTGTGACGGGCGGTGTGTACAAGGCCCGGGAACGTATTCACCGTG
ACGTTCTGATTCACGATTACTAGCGATTCCGACTTCACGCAGTCGAGTTGCAGACTGCGATCCGGACTACGATCGGT
TTTATGGGATTAGCTCCACCTCGCGGCTTGGCAACCCTTTGTACCGACCATTGTAGCACGTGTGTAGCCCTGGCCGT
AAGGGCCATGATGACTTGACGTCATCCCCACCTTCCTCCGGTTTGTCACCGGCAGTCTCCTTAGAGTGCCCACCATA
ACGTGCTGGTAACTAAGGACAAGGGTTGCGCTCGTTACGGGACTTAACCCAACATCTCACGACACGAGCTGACGACA
GCCATGCAGCACCTGTGTCTGAGCTCCCGAAGGCACCAATCCATCTCTGGAAAGTTCTCAGCATGTCAAGGCCAGGT
AAGGTTCTTCGCGTTGCTTCGAATTAAACCACATGCTCCACCGCTTGTGCGGGCCCCCGTCAATTCATTTGAGTTTT
AACCTTGCGGCCGTACTCCCCAGGCGGTCAACTTAATGCGTTAGCTGCGCCACTAAGATCTCAAGGATCCCAACGGC
TAGTTGACATCGTTTACGGCGTGGACTACCAGGGTATCTAATCCTGTTTGCTCCCCACGCTTTCGCACCTCAGTGTC
AGTGTCAGTCCAGGTAGTCGCCTTCGCCACTGGTGTTCCTTCCAATATCTACGCATTTCACCGCTACACTGGAAATT
CCACTACCCTCTACCGCACTCTAGCCAGACAGTTTTGGATGCAGTTCCCAGGTTGAGCCCGGGGATTTCACATCCAA
CTTATCAAGCCACCTACGCGCGCTTTACGCCCAGTAATTCCGATTAACGCTTGCACCCTTCGTATTACCGCGGCTGC
TGGCACGAAGTTAGCCGGTGCTTATTCTGTTGGTAACGTCAAAACTCACAGGTATTCGCTATGAGCCCTTCCTCCCA
ACTTAAAGTGCTTTACGACCCGAAGGCCTTCTTCACACACGCGGCATGGCTGGATCAGGCTTTCGCCCATTGTCCAA
TATTCCCCACTGCTGCCTCCCGTAGGAGTCTGGACCGTGTCTCAGTTCCAGTGTGACTGATCATCCTCTCAGACCAG
TTACGGATCGTCGCCTTGGTAGGCCTTTACCCTACCAACTAGCTAATCCGACCTAGGCTCATCTAATAGCGTGAGGT
CCGAAGATCCCCCACTTTCTCCCGTAGGACGTATGCGGTATTAGCGTTCCTTTCGAAACGTTGTCCCCCACTACTAG
GCAGATTCCTAGGCATTACTCACCCGTCCGCC
SEQ ID NO: 10 Pseudomonas oryzihabitans (Strain WCB2)
GGCGGACGGGTGAGTAATGCCTAGGAATCTGCCTAGTAGTGGGGGACAACGTTTCGAAAGGAACGCTAATACCGCAT
ACGTCCTACGGGAGAAAGTGGGGGATCTTCGGACCTCACGCTATTAGATGAGCCTAGGTCGGATTAGCTAGTTGGTA
GGGTAAAGGCCTACCAAGGCGACGATCCGTAACTGGTCTGAGAGGATGATCAGTCACACTGGAACTGAGACACGGTC
CAGACTCCTACGGGAGGCAGCAGTGGGGAATATTGGACAATGGGCGAAAGCCTGATCCAGCCATGCCGCGTGTGTGA
AGAAGGCCTTCGGGTCGTAAAGCACTTTAAGTTGGGAGGAAGGGCTCATAGCGAATACCTGTGAGTTTTGACGTTAC
CAACAGAATAAGCACCGGCTAACTTCGTGCCAGCAGCCGCGGTAATACGAAGGGTGCAAGCGTTAATCGGAATTACT
GGGCGTAAAGCGCGCGTAGGTGGCTTGATAAGTTGGATGTGAAATCCCCGGGCTCAACCTGGGAACTGCATCCAAAA
CTGTCTGGCTAGAGTGCGGTAGAGGGTAGTGGAATTTCCAGTGTAGCGGTGAAATGCGTAGATATTGGAAGGAACAC
CAGTGGCGAAGGCGACTACCTGGACTGACACTGACACTGAGGTGCGAAAGCGTGGGGAGCAAACAGGATTAGATACC
CTGGTAGTCCACGCCGTAAACGATGTCAACTAGCCGTTGGGATCCTTGAGATCTTAGTGGCGCAGCTAACGCATTAA
GTTGACCGCCTGGGGAGTACGGCCGCAAGGTTAAAACTCAAATGAATTGACGGGGGCCCGCACAAGCGGTGGAGCAT
GTGGTTTAATTCGAAGCAACGCGAAGAACCTTACCTGGCCTTGACATGCTGAGAACTTTCCAGAGATGGATTGGTGC
CTTCGGGAGCTCAGACACAGGTGCTGCATGGCTGTCGTCAGCTCGTGTCGTGAGATGTTGGGTTAAGTCCCGTAACG
AGCGCAACCCTTGTCCTTAGTTACCAGCACGTTATGGTGGGCACTCTAAGGAGACTGCCGGTGACAAACCGGAGGAA
GGTGGGGATGACGTCAAGTCATCATGGCCCTTACGGCCAGGGCTACACACGTGCTACAATGGTCGGTACAAAGGGTT
GCCAAGCCGCGAGGTGGAGCTAATCCCATAAAACCGATCGTAGTCCGGATCGCAGTCTGCAACTCGACTGCGTGAAG
TCGGAATCGCTAGTAATCGTGAATCAGAACGTCACGGTGAATACGTTCCCGGGCCTTGTACACACCGCCCGTCACAC
CATGGGAGTGGGTTGCTCCAGAAGTAGCTAGT
SEQ ID NO: 11 Pseudomonas oleovorans (Strain Poryz)
TCGAGCGGATGAGAGGAGCTTGCTCTTCGATTCAGCGGCGGACGGGTGAGTAATGCCTAGGAATCTGCCTAGTAGTG
GGGGACAACGTTTCGAAAGGAACGCTAATACCGCATACGTCCTACGGGAGAAAGTGGGGGATCTTCGGACCTCACGC
TATTAGATGAGCCTAGGTCGGATTAGCTAGTTGGTAGGGTAAAGGCCTACCAAGGCGACGATCCGTAACTGGTCTGA
GAGGATGATCAGTCACACTGGAACTGAGACACGGTCCAGACTCCTACGGGAGGCAGCAGTGGGGAATATTGGACAAT
GGGCGAAAGCCTGATCCAGCCATGCCGCGTGTGTGAAGAAGGCCTTCGGGTCGTAAAGCACTTTAAGTTGGGAGGAA
GGGCTCATAGCGAATACCTGTGAGTTTTGACGTTACCAACAGAATAAGCACCGGCTAACTTCGTGCCAGCAGCCGCG
GTAATACGAAGGGTGCAAGCGTTAATCGGAATTACTGGGCGTAAAGCGCGCGTAGGTGGCTTGATAAGTTGGATGTG
AAATCCCCGGGCTCAACCTGGGAACTGCATCCAAAACTGTCTGGCTAGAGTGCGGTAGAGGGTAGTGGAATTTCCAG
TGTAGCGGTGAAATGCGTAGATATTGGAAGGAACACC
SEQ ID NO: 12 Pseudomonas oleovorans (Strain Poryz)
GCTCAGATTGAACGCTGGCGGCAGGCCTAACACATGCAAGTCGAGCGGATGAGAGGAGCTTGCTCTTCGATTCAGCG
GCGGACGGGTGAGTAATGCCTAGGAATCTGCCTAGTAGTGGGGGACAACGTTTCGAAAGGAACGCTAATACCGCATA
CGTCCTACGGGAGAAAGTGGGGGATCTTCGGACCTCACGCTATTAGATGAGCCTAGGTCGGATTAGCTAGTTGGTAG
GGTAAAGGCCTACCAAGGCGACGATCCGTAACTGGTCTGAGAGGATGATCAGTCACACTGGAACTGAGACACGGTCC
AGACTCCTACGGGAGGCAGCAGTGGGGAATATTGGACAATGGGCGAAAGCCTGATCCAGCCATGCCGCGTGTGTGAA
GAAGGCCTTCGGGTCGTAAAGCACTTTAAGTTGGGAGGAAGGGCTCATAGCGAATACCTGTGAGTTTTGACGTTACC
AACAGAATAAGCACCGGCTAACTTCGTGCCAGCAGCCGCGGTAATACGAAGGGTGCAAGCGTTAATCGGAATTACTG
GGCGTAAAGCGCGCGTAGGTGGCTTGATAAGTTGGATGTGAAATCCCCGGGCTCAACCTGGGAACTGCATCCAAAAC
TGTCTGGCTAGAGTGCGGTAGAGGGTAGTGGAATTTCCAGTGTAGCGGTGAAATGCGTAGATATTGGAAGGAACACC
AGTGGCGAAGGCGACTACCTGGACTGACACTGACACTGAGGTGCGAAAGCGTGGGGAGCAAACAGGATTAGATACCC
TGGTAGTCCACGCCGTAAACGATGTCAACTAGCCGTTGGGATCCTTGAGATCTTAGTGGCGCAGCTAACGCATTAAG
TTGACCGCCTGGGGAGTACGGCCGCAAGGTTAAAACTCAAATGAATTGACGGGGGCCCGCACAAGCGGTGGAGCATG
TGGTTTAATTCGAAGCAACGCGAAGAACCTTACCTGGCCTTGACATGCTGAGAACTTTCCAGAGATGGATTGGTGCC
TTCGGGAACTCAGACACAGGTGCTGCATGGCTGTCGTCAGCTCGTGTCGTGAGATGTTGGGTTAAGTCCCGTAACGA
GCGCAACCCTTGTCCTTAGTTACCAGCACGTTATGGTGGGCACTCTAAGGAGACTGCCGGTGACAAACCGGAGGAAG
GTGGGGATGACGTCAAGTCATCATGGCCCTTACGGCCAGGGCTACACACGTGCTACAATGGTCGGTACAAAGGGTTG
CCAAGCCGCGAGGTGGAGCTAATCCCATAAAACCGATCGTAGTCCGGATCGCAGTCTGCAACTCGACTGCGTGAAGT
CGGAATCGCTAGTAATCGTGAATCAGAACGTCACGGTGAATACGTTCCCGGGCCTTGTACACACCGCCCGTCACACC
ATGGGAGTGGGTTGCTCCAGAAGTAGCTAGTCTAACCTTCGGGAGGACGGTTACCACGGAGTGATTCATGACTGGGG
TGA
SEQ ID NO: 13 Achromobacter xylosoxidans (Strain Achromo)
GTATCGGAACGTGCCCAGTAGCGGGGGATAACTACGCGAAAGCGTAGCTAATACCGCATACGCCCTACGGGGGAAAG
CAGGGGATCGCAAGACCTTGCACTATTGGAGCGGCCGATATCGGATTAGCTAGTTGGTGGGGTAACGGCTCACCAAG
GCGACGATCCGTAGCTGGTTTGAGAGGACGACCAGCCACACTGGGACTGAGACACGGCCCAGACTCCTACGGGAGGC
AGCAGTGGGGAATTTTGGACAATGGGGGAAACCCTGATCCAGCCATCCCGCGTGTGCGATGAAGGCCTTCGGGTTGT
CTAACTACGTGCCAGCAGCCGCGGTAATACGTAGGGTGCAAGCGTTAATCGGAATTACTGGGCGTAAAGCGTGCGCA
GGCGGTTCGGAAAGAAAGATGTGAAATCCCAGAGCTTAACTTTGGAACTGCATTTTTAACTACCGGGCTAGAGTGTG
TCAGAGGGAGGIGGAATTCCGCGTGTAGCAGTGAAATGCGTAGATATGCGGAGGAACACCGATGGCGAANGCAGCCT
CCTGGGATAACACTGACGCTCATGCACGAAAGCGTGGGGAGCAAACAGGATTAGATACCCTGGTAGTCCACGCCCTA
AACNATGTCAACTAGCTGTTGGGGCCT
SEQ ID NO: 14 Achromobacter xylosoxidans (Strain Achromo)
TCAGATTGAACGCTAGCGGGATGCCTTACACATGCAAGTCGAACGGCAGCACGGACTTCGGTCTGGTGGCGAGTGGC
GAACGGGTGAGTAATGTATCGGAACGTGCCCAGTAGCGGGGGATAACTACGCGAAAGCGTAGCTAATACCGCATACG
CCCTACGGGGGAAAGCAGGGGATCGCAAGACCTTGCACTATTGGAGCGGCCGATATCGGATTAGCTAGITGGTGGGG
TAACGGCTCACCAAGGCGACGATCCGTAGCTGGTTTGAGAGGACGACCAGCCACACTGGGACTGAGACACGGCCCAG
ACTCCTACGGGAGGCAGCAGTGGGGAATTTTGGACAATGGGGGAAACCCTGATCCAGCCATCCCGCGTGTGCGATGA
AGGCCTTCGGGTTGTAAAGCACTTTTGGCAGGAAAGAAACGTCGTGGGTTAATACCCCGCGAAACTGACGGTACCTG
CAGAATAAGCACCGGCTAACTACGTGCCAGCAGCCGCGGTAATACGTAGGGTGCAAGCGTTAATCGGAATTACTGGG
CGTAAAGCGTGCGCAGGCGGTTCGGAAAGAAAGATGTGAAATCCCAGAGCTTAACTTTGGAACTGCATTTTTAACTA
CCGGGCTAGAGTGTGTCAGAGGGAGGTGGAATTCCGCGTGTAGCAGTGAAATGCGTAGATATGCGGAGGAACACCGA
TGGCGAAGGCAGCCTCCTGGGATAACACTGACGCTCATGCACGAAAGCGTGGGGAGCAAACAGGATTAGATACCCTG
GTAGTCCACGCCCTAAACGATGTCAACTAGCTGTTGGGGCCTTCGGGCCTTGGTAGCGCAGCTAACGCGTGAAGTTG
ACCGCCTGGGGAGTACGGTCGCAAGATTAAAACTCAAAGGAATTGACGGGGACCCGCACAAGCGGTGGATGATGTGG
ATTAATTCGATGCAACGCGAAAAACCTTACCTACCCTTGACATGTCTGGAATGCCGAAGAGATTTGGCAGTGCTCGC
AAGAGAACCGGAACACAGGTGCTGCATGGCTGTCGTCAGCTCGTGTCGTGAGATGTTGGGTTAAGTCCCGCAACGAG
CGCAACCCTTGTCATTAGTTGCTACGAAAGGGCACTCTAATGAGACTGCCGGTGACAAACCGGAGGAAGGTGGGGAT
GACGTCAAGTCCTCATGGCCCTTATGGGTAGGGCTTCACACGTCATACAATGGTCGGGACAGAGGGTCGCCAACCCG
CGAGGGGGAGCCAATCCCAGAAACCCGATCGTAGTCCGGATCGCAGTCTGCAACTCGACTGCGTGAAGTCGGAATCG
CTAGTAATCGCGGATCAGCATGTCGCGGTGAATACGTTCCCGGGTCTTGTACACACCGCCCGTCACACCATGGGAGT
GGGTTTTACCAGAAGTAGTTAGCCTAACCGCAAGGGGGGCGATTACCACGGTAGGATTCATGACTGGGGTGA
SEQ ID NO: 15 Pantoea dispersa (Strain Pdisp)
AGTGGGGGACCTTCGGGCCACACACCATCGGATGTGCCCGGATGGGATTAGCTAGTAGGTGGGGTAATGGCTCACCT
AGGCGACGATCCCTAGCTGGTCTGAGAGGATGACCAGCCACACTGGAACTGAGACACGGTCCAGACTCCTACGGGAG
TANGCAGTGGGGAATATTGCACAATGGGCGCAAGCCTGATGCAGCCATGCCGCGTGTATGAAGAAGGCCTTCGGGTT
GTAAAGTACTTTCAGCGGGGAGGAAGGCGGTGAGGTTAATAACCTTGCCGATTGACGTTACCCGCAGAAGAAGCACC
GGCTAACTCCGTGCCAGCAGCCGCGGTAATACGGAGGGTGCAAGCGTTAATCGGAATTACTGGGCGTAAAGCGCACG
CAGGCGGTCTGTTAAGTCAGATGTGAAATCCCCGGGCTTAACCTGGGAACTGCATTTGAAACTGGCAGGCTTGAGTC
TCGTAGAGGGGGGTA
SEQ ID NO: 16 Pantoea dispersa (Strain Pdisp)
CTCAGATTGAACGCTGGCGGCAGGCCTAACACATGCAAGTCGAACGGCAGCACAGAAGAGCTTGCTCTTTGGGTGGC
GAGTGGCGGACGGGTGAGTAATGTCTGGGAAACTGCCCGATGGAGGGGGATAACTACTGGAAACGGTAGCTAATACC
GCATAACGTCGCAAGACCAAAGTGGGGGACCTTCGGGCCTCACACCATCGGATGTGCCCAGATGGGATTAGCTAGTA
GGTGGGGTAATGGCTCACCTAGGCGACGATCCCTAGCTGGTCTGAGAGGATGACCAGCCACACTGGAACTGAGACAC
GGTCCAGACTCCTACGGGAGGCAGCAGTGGGGAATATTGCACAATGGGCGCAAGCCTGATGCAGCCATGCCGCGTGT
ATGAAGAAGGCCTTCGGGTTGTAAAGTACTTTCAGCGGGGAGGAAGGCGGTGAGGTTAATAACCTTGCCGATTGACG
TTACCCGCAGAAGAAGCACCGGCTAACTCCGTGCCAGCAGCCGCGGTAATACGGAGGGTGCAAGCGTTAATCGGAAT
TACTGGGCGTAAAGCGCACGCAGGCGGTCTGTTAAGTCAGATGTGAAATCCCCGGGCTTAACCTGGGAACTGCATTT
GAAACTGGCAGGCTTGAGTCTCGTAGAGGGGGGTAGAATTCCAGGTGTAGCGGTGAAATGCGTAGAGATCTGGAGGA
ATACCGGTGGCGAAGGCGGCCCCCTGGACGAAGACTGACGCTCAGGTGCGAAAGCGTGGGGAGCAAACAGGATTAGA
TACCCTGGTAGTCCACGCCGTAAACGATGTCGACTTGGAGGTTGTGCCCTTGAGGCGTGGCTTCCGGAGCTAACGCG
TTAAGTCGACCGCCTGGGGAGTACGGCCGCAAGGTTAAAACTCAAATGAATTGACGGGGGCCCGCACAAGCGGTGGA
GCATGTGGTTTAATTCGATGCAACGCGAAGAACCTTACCTGGCCTTGACATCCAGAGAACTTAGCAGAGATGCTTTG
GTGCCTTCGGGAACTCTGAGACAGGTGCTGCATGGCTGTCGTCAGCTCGTGTTGTGAAATGTTGGGTTAAGTCCCGC
AACGAGCGCAACCCTTATCCTTTGTTGCCAGCGGTTCGGCCGGGAACTCAAAGGAGACTGCCGGTGATAAACCGGAG
GAAGGTGGGGATGACGTCAAGTCATCATGGCCCTTACGGCCAGGGCTACACACGTGCTACAATGGCGCATACAAAGA
GAAGCGACCTCGCGAGAGCAAGCGGACCTCATAAAGTGCGTCGTAGTCCGGATTGGAGTCTGCAACTCGACTCCATG
AAGTCGGAATCGCTAGTAATCGTAGATCAGAATGCTACGGTGAATACGTTCCCGGGCCTTGTACACACCGCCCGTCA
CACCATGGGAGTGGGTTGCAAAAGAAGTAGGTAGCTTAACCTTCGGGAGGGCGCTACCACTTTGTGATTCATGACTG
GGGT
SEQ ID NO: 17 Enterobacter cloacae (strain Entero)
ATGTCTGGGAAACTGCCTGATGGAGGGGGATAACTACTGGAAACGGTAGCTAATACCGCATAATGTCGCAAGACCAA
AGAGGGGGACCTTCGGGCCTCTTGCCATCAGATGTGCCCAGATGGGATTAGCTAGTAGGTGGGGTAACGGCTCACCT
AGGCGACGATCCCTAGCTGGTCTGAGAGGATGACCAGCCACACTGGAACTGAGACACGGTCCAGACTCCTACGGGAG
GCAGCAGTGGGGAATATTGCACAATGGGCGCAAGCCTGATGCAGCCATGCCGCGTGTATGAAGAAGGCCTTCGGGTT
GTAAAGTACTTTCAGCGGGGAGGAAGGTGTTGTGGTTAATAACCACAGCAATTGACGTTACCCGCAGAAGAAGCACC
GGCTAACTCCGTGCCAGCAGCCGCGGTAATACGGAGGGTGCAAGCGTTAATCGGAATTACTGGGCGTAAAGCGCACG
CAGGCGGTCTGTCAAGTCGGATGTGAAATCCCCGGGCTCAACCTGGGAACTGCATTCGAAACTGGCAGGCTAGAGTC
TTGTAGAGGGGGGTAGAATTCCAGGTGTAGCGGTGAAATGCGTAGAGATCTGGAGGAATACCGGTGGCGAAGGCGGC
SEQ ID NO: 18 Enterobacter cloacae (strain Entero)
TCAGATTGAACGCTGGCGGCAGGCCTAACACATGCAAGTCGAACGGTAGCACAGAGAGCTTGCTCTCGGGTGACGAG
TGGCGGACGGGTGAGTAATGTCTGGGAAACTGCCTGATGGAGGGGGATAACTACTGGAAACGGTAGCTAATACCGCA
TAATGTCGCAAGACCAAAGAGGGGGACCTTCGGGCCTCTTGCCATCAGATGTGCCCAGATGGGATTAGCTAGTAGGT
GGGGTAACGGCTCACCTAGGCGACGATCCCTAGCTGGTCTGAGAGGATGACCAGCCACACTGGAACTGAGACACGGT
CCAGACTCCTACGGGAGGCAGCAGTGGGGAATATTGCACAATGGGCGCAAGCCTGATGCAGCCATGCCGCGTGTATG
AAGAAGGCCTTCGGGTTGTAAAGTACTTTCAGCGGGGAGGAAGGTGTTGTGGTTAATAACCACAGCAATTGACGTTA
CCCGCAGAAGAAGCACCGGCTAACTCCGTGCCAGCAGCCGCGGTAATACGGAGGGTGCAAGCGTTAATCGGAATTAC
TGGGCGTAAAGCGCACGCAGGCGGTCTGTCAAGTCGGATGTGAAATCCCCGGGCTCAACCTGGGAACTGCATTCGAA
ACTGGCAGGCTAGAGTCTTGTAGAGGGGGGTAGAATTCCAGGTGTAGCGGTGAAATGCGTAGAGATCTGGAGGAATA
CCGGTGGCGAAGGCGGCCCCCTGGACAAAGACTGACGCTCAGGTGCGAAAGCGTGGGGAGCAAACAGGATTAGATAC
CCTGGTAGTCCACGCCGTAAACGATGTCGATTTGGAGGTTGTGCCCTTGAGGCGTGGCTTCCGGAGCTAACGCGTTA
AATCGACCGCCTGGGGAGTACGGCCGCAAGGTTAAAACTCAAATGAATTGACGGGGGCCCGCACAAGCGGTGGAGCA
TGTGGTTTAATTCGATGCAACGCGAAGAACCTTACCTGGTCTTGACATCCACAGAACTTACCAGAGATGCTTTGGTG
CCTTCGGGAACTGTGAGACAGGTGCTGCATGGCTGTCGTCAGCTCGTGTTGTGAAATGTTGGGTTAAGTCCCGCAAC
GAGCGCAACCCTTATCCTTTGTTGCCAGCGGTCCGGCCGGGAACTCAAAGGAGACTGCCAGTGATAAACTGGAGGAA
GGTGGGGATGACGTCAAGTCATCATGGCCCTTACGACCAGGGCTACACACGTGCTACAATGGCGCATACAAAGAGAA
GCGACCTCGCGAGAGCAAGCGGACCTCATAAAGTGCGTCGTAGTCCGGATTGGAGTCTGCAACTCGACTCCATGAAG
TCGGAATCGCTAGTAATCGTAGATCAGAATGCTACGGTGAATACGTTCCCGGGCCTTGTACACACCGCCCGTCACAC
CATGGGAGTGGGTTGCAAAAGAAGTAGGTAGCTTAACCTTCGGGAGGGCGCTTACCACTTTGTGATTCATGACTGGG
GTGAGTC

### I. Definitions

An “endophyte” or “endophytic microbe” is an organism that lives within a plant or is otherwise associated therewith. Endophytes can occupy the intracellular or intercellular spaces of plant tissue, including the leaves, stems, flowers, fruits, seeds, or roots. An endophyte can be either a bacterial or a fungal organism that can confer a beneficial property to a plant such as an increase in yield, biomass, resistance, or fitness in its host plant. As used herein, the term “microbe” or “bacteria” is sometimes used to describe an endophyte.

Several strains of the bacteria described herein can be identified by their distinct ribosomal 16S sequences. 16S ribosomal RNA (or 16S rRNA) is the component of the 30S small subunit of a prokaryotic ribosome that binds to the Shine-Dalgarno sequence. The genes coding for it are referred to as 16S rRNA gene and are used in reconstructing phylogenies, due to the slow rates of evolution of this region of the gene. Ribosomal RNA sequences from the bacteria described herein are provided in SEQ ID NOS: 1, 2 and 7-18.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, and the like) available through the worldwide web at sites including but not limited to GENBANK (on the world wide web at ncbi.nlm.nih.gov/genbank/) and EMBL-EBI (on the world wide web at ebi.ac.uk.). Sequence identity between two polynucleotides or two polypeptide sequences is generally calculated using the standard default parameters of the various methods or computer programs. A high degree of sequence identity, as used herein, between two polynucleotides or two polypeptides is typically between about 90% identity and 100% identity, for example, about 90% identity or higher, preferably about 95% identity or higher, more preferably about 98% identity or higher. A moderate degree of sequence identity, as used herein, between two polynucleotides or two polypeptides is typically between about 80% identity to about 85% identity, for example, about 80% identity or higher, preferably about 85% identity. A low degree of sequence identity, as used herein, between two polynucleotides or two polypeptides is typically between about 50% identity and 75% identity, for example, about 50% identity, preferably about 60% identity, more preferably about 75% identity.

The terms “promoting plant growth” and “stimulating plant growth” are used interchangeably herein, and refer to the ability to enhance or increase at least one of the plant's height, weight, leaf size, root size, shoot length, stem size, competition with competitor plants, resistance to fungal infection, increased protein yield from the plant or increased grain yield of the plant.

Particular formulations to be applied in spraying forms such as water dispersible concentrates or wettable powders may contain surfactant such as wetting and dispersing agents, e.g., the condensation product of formaldehyde with naphthalene sulphonate, an alkyl-aryl-sulphonate, a lignin sulphonate, a fatty alkyl sulphate an ethoxylated alkylphenol and an ethoxylated fatty alcohol.

As used herein the terms “spray” or “spraying” include the technique of applying to an exterior surface an ejected liquid material.

As used herein, the terms “coat” or “coating” include application, typically of a liquid or flowable solid, to an exterior surface such as a seed.

As used herein, a “stabilizer” includes a chemical compound that can be added to a formulation to prolong the stability and/or viability of components of the formulation, a critical aspect of product shelf-stability. A stabilizer can be one of a variety of compounds, such as a dessicant.

As used herein, a “preservative” includes any chemical compound and/or physical conditions that prevent the decomposition of organic constituents of seeds treated with formulations. Chemical preservatives could include, for example, synthetic or non-synthetic antioxidants and physical preservatives could include, for example, refrigeration, freeze-drying or drying.

According to an embodiment the at least one dispersing agent can be in the range of about 2% to about 60% on a dry weight by weight basis. Various dispersing agents are commercially available for use in agricultural compositions, such as those marketed by Rhone Poulenc, Witco, Westvaco, International Speciality products, Croda chemicals, Borregaard, BASF, Rhodia, etc. According to an embodiment the dispersing agents which can be used in the agricultural composition can be chosen from a group comprising polyvinylpyrrolidone, polyvinylalcohol, lignosulphonates, phenyl naphthalene sulphonates, ethoxylated alkyl phenols, ethoxylated fatty acids, alkoxylated linear alcohols, polyaromatic sulfonates, sodium alkyl aryl sulfonates, glyceryl esters, maleic anhydride copolymers, phosphate esters, condensation products of aryl sulphonic acids and formaldehyde, condensation products of alkylaryl sulphonic acids and formaldehyde, addition products of ethylene oxide and fatty acid esters, salts of addition products. of ethylene oxide and fatty acid esters, sulfonates of condensed naphthalene, addition products of ethylene oxide and fatty acid esters, salts of addition products of ethylene oxide and fatty acid esters, lignin derivatives, naphthalene formaldehyde condensates, sodium salt of isodecylsulfosuccinic acid half ester, polycarboxylates, sodium alkylbenzenesulfonates, sodium salts of sulfonated naphthalene, ammonium salts of sulfonated naphthalene, salts of polyacrylic acids, salts of phenolsulfonic acids and salts of naphthalene sulfonic acids. However, those skilled in the art will appreciate that it is possible to utilize other dispersing agents known in the art without departing from the scope of the claims of the present invention.

In some embodiments, a bacterial endophyte is a seed-origin bacterial endophyte. As used herein, a “seed-origin” or “seed-vectored” bacterial endophyte” refers to a population of bacteria associated with or derived from the seed of a host plant. For example, a seed-origin bacterial endophyte can be found in mature, dry, undamaged (e.g., no cracks, visible fungal infection, or prematurely germinated) seeds. The bacteria can be associated with or derived from the surface of the seed; alternatively, or in addition, it can be associated with or derived from the interior seed compartment (e.g., of a surface-sterilized seed) or from a seedling. In some cases, a seed-origin bacterial endophyte is capable of replicating within the plant tissue, for example, the interior of the seed. Also, in some cases, the seed-origin bacterial endophyte is capable of surviving desiccation.

Seed-origin or seed-vectored means that the bacterial entity is obtained directly or indirectly from the seed surface or seed interior compartment or is obtainable from a seed surface or seed interior compartment. For example, a seed-origin bacterial entity can be obtained directly or indirectly from a seed surface or seed interior compartment when it is isolated, or isolated and purified, from a seed preparation; in some cases, the seed-origin bacterial entity which has been isolated, or isolated and purified, may be cultured under appropriate conditions to produce a purified bacterial population consisting essentially of a seed-origin bacterial endophyte. A seed-origin bacterial endophyte can be considered to be obtainable from a seed surface, a seedling, or seed interior compartment if the bacteria can be detected on or in, or isolated from, a seed surface or seed interior compartment of a plant.

In some embodiments, the present invention contemplates methods of manually or mechanically combining an endophyte described herein with one or more plant elements, such as a seed, a leaf, or a root, in order to confer an improved agronomic trait or improved agronomic trait potential to said plant element or host plant. In some embodiments, the present invention contemplates methods of manually or mechanically combining a plurality of endophytes described herein with one or more plant elements.

As used herein, a “synthetic combination” is the combination of a plant element, seedling, or whole plants and a plurality of endophytes, combined by human endeavor, in which one or more of the plurality of endophytes are heterologously disposed, said combination which is not found in nature. In some embodiments, the synthetic combination includes two or more endophytes that synergistically interact providing a benefit to an agricultural seed, seedling, or plant derived thereby. In some embodiments, a synthetic combination is used to refer to a treatment formulation comprising an isolated, purified population of endophytes heterologously disposed to a plant element. In some embodiments of the present invention, “synthetic combination” refers to a purified population of endophytes in a treatment formulation comprising additional compositions with which said endophytes are not found associated in nature.

As used herein, an endophyte is “heterologously disposed” when mechanically or manually applied, artificially inoculated or disposed onto or into a plant element, seedling, plant or onto or into a plant growth medium or onto or into a treatment formulation so that the endophyte exists on or in said plant element, seedling, plant, plant growth medium, or treatment formulation in a manner not found in nature prior to the application of the endophyte, e.g., said combination which is not found in nature. In some embodiments, such a manner is contemplated to include: the presence of the endophyte; presence of the endophyte in a different number, concentration, or amount; the presence of the endophyte in or on a different plant element, tissue, cell type, or other physical location in or on the plant; the presence of the endophyte at different time period, e.g. developmental phase of the plant or plant element, time of day, time of season, and combinations thereof. In some embodiments, plant growth medium is soil, a hydroponic apparatus, or artificial growth medium such as commercial potting mix. In some embodiments, the plant growth medium is soil in an agricultural field. In some embodiments, the plant growth medium is commercial potting mix. In some embodiments, the plant growth medium is an artificial growth medium such as germination paper. As a non-limiting example, if the plant element or seedling or plant has an endophyte normally found in the root tissue but not in the leaf tissue, and the endophyte is applied to the leaf, the endophyte would be considered to be heterologously disposed. As a non-limiting example, if the endophyte is naturally found in the mesophyll layer of leaf tissue but is applied to the epithelial layer, the endophyte would be considered to be heterologously disposed. As a non-limiting example, an endophyte is heterologously disposed at a concentration that is at least 1.5 times, between 1.5 and 2 times, 2 times, between 2 and 3 times, 3 times, between 3 and 5 times, 5 times, between 5 and 7 times, 7 times, between 7 and 10 times, 10 times greater, or even greater than 10 times higher number, amount, or concentration than that which is naturally present. As a non-limiting example, an endophyte is heterologously disposed on a seedling if that endophyte is normally found at the flowering stage of a plant and not at a seedling stage.

The compositions provided herein are preferably stable. The seed-origin bacterial endophyte is optionally shelf stable, where at least 10% of the CFUs are viable after storage in desiccated form (i.e., moisture content of 30% or less) for 1, 2, 3, 4, 5, 6, 7, 8, 9, 10 or greater than 10 weeks at 4° C. or at room temperature. Optionally, a shelf stable formulation is in a dry formulation, a powder formulation, or a lyophilized formulation. In some embodiments, the formulation is formulated to provide stability for the population of bacterial endophytes. In one embodiment, the formulation is substantially stable at temperatures between about 0° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In another embodiment, the formulation is substantially stable at temperatures between about 4° C. and about 37° C. for at least about 5, 10, 15, 20, 25, 30 or greater than 30 days.

In some embodiments, plants (including seeds and other plant elements) treated in accordance with the present invention are monocots. In some embodiments, plants (including seeds or other plant elements) treated in accordance with the present invention are dicots. In some embodiments, plants treated in accordance with the present invention include, but are not limited to: agricultural row, agricultural grass plants or other field crops: wheat, rice, barley, buckwheat, beans (soybean, snap, dry), corn (grain, seed, sweet corn, silage, popcorn, high oil), cotton, canola, peas (dry, succulent), peanuts, safflower, sunflower, alfalfa hay, forage crops (alfalfa, clover, vetch, and trefoil), berries and small fruits (blackberries, blueberries, currants, elderberries, gooseberries, huckleberries, loganberries, raspberries, strawberries, bananas and grapes), bulb crops (garlic, leeks, onions, shallots, and ornamental bulbs), citrus fruits (citrus hybrids, grapefruit, kumquat, lines, oranges, and pummelos), cucurbit vegetables (cucumbers, melons, gourds, pumpkins, and squash), flowers, bedding plants, ornamentals, fruiting vegetables (eggplant, sweet and hot peppers, tomatillos, and tomatoes), herbs, spices, mints, hydroponic crops (cucumbers, tomatoes, lettuce, herbs, and spices), leafy vegetables and cole crops (arugula, celery, chervil, endive, fennel, lettuce (head and leaf), parsley, radicchio, rhubarb, spinach, Swiss chard, broccoli, Brussels sprouts, cabbage, cauliflower, collards, kale, kohlrabi, and mustard greens), asparagus, legume vegetable and field crops (snap and dry beans, lentils, succulent and dry peas, and peanuts), pome fruit (pears and quince), root crops (beets, sugarbeets, red beets, carrots, celeriac, chicory, horseradish, parsnip, radish rutabaga, salsify, and turnips), deciduous trees (maple and oak), pine, small grains (rye, wheat, sorghum, millet, stone fruits (apricots, cherries, nectarines, peaches, plums, and prunes), tree nuts (almonds, beech nuts, Brazil nuts, butternuts, cashews, chestnuts, filberts, hickory nuts, macadamia nuts, pecans, pistachios, and walnuts), tuber crops (potatoes, sweet potatoes, yams, artichoke, cassava, and ginger), and turfgrass (turf, sports fields, parks, established and new preparation of golf course tees, greens, fairways and roughs, seed production and sod production).

Preferred target species of agricultural plants include species of Malvaceae (cotton family): Cotton (Gossypium spp.), Okra Abelmoschus esculentus, Cacao (Theobroma cacao), Kenaf (Hibiscus cannabinus) and Kola nut (Cola spp.). Target species also include other dicot crops, including but not limited to, Coffee (Coffea spp.), Tobacco (Nicotianatabacum), Potato (Solanum tuberosum), Tomato (Solanum lycopsersicum), Sweet potato (Ipomoea batatas), Beans (Phaseolus spp.), Soybeans (Glycine max), Sunflowers (Helianthus spp.) and Rapeseed (Brassica napus).

As used herein, an agricultural grass plant includes, but is not limited to, maize (Zea mays), common wheat (Triticum aestivum), spelt (Triticum spelta), einkorn wheat (Triticum monococcum), emmer wheat (Triticum dicoccum), durum wheat (Triticum durum), Asian rice (Oryza sativa), African rice (Oryza glabaerreima), wild rice (Zizania aquatica, Zizania latifolia, Zizania palustris, Zizania texana), barley (Hordeum vulgare), Sorghum (Sorghum bicolor), Finger millet (Eleusine coracana), Proso millet (Panicum miliaceum), Pearl millet (Pennisetum glaucum), Foxtail millet (Setaria italic), Oat (Avena sativa), Triticale (Triticosecale), rye (Secale cereal), Russian wild rye (Psathyrostachys juncea), bamboo (Bambuseae), grasses, including Agrostis spp., Poa spp., Festuca spp., Lolium spp., Cynodon spp., Zoysia spp., Koleria spp., Danthonia spp., or sugarcane (e.g., Saccharum arundinaceum, Saccharum barberi, Saccharum bengalense, Saccharum edule, Saccharum munja, Saccharum officinarum, Saccharum procerum, Saccharum ravennae, Saccharum robustum, Saccharum sinense, or Saccharum spontaneum).

A “host plant” includes any plant, particularly an agricultural plant, which an endophytic microbe such as a bacterial endophyte can colonize. As used herein, a microbe is said to “colonize” a plant or seed when it can be stably detected within the plant or seed over a period time, such as one or more days, weeks, months or years; in other words, a colonizing microbe is not transiently associated with the plant or seed.

As used herein, a “reference agricultural plant” is an agricultural plant of the same species, strain, or cultivar to which a treatment, formulation, composition or endophyte preparation as described herein is not administered/contacted. Exemplary reference agricultural plants are described herein. A reference agricultural plant, therefore, is identical to the treated plant with the exception of the presence of the endophyte and can serve as a control for detecting the effects of the endophyte that is conferred to the plant.

A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, kelkis, shoot, bud. As used herein, a “plant element” is synonymous to a “portion” of a plant, and refers to any part of the plant, and can include distinct tissues and/or organs, and may be used interchangeably with the term “tissue” throughout.

“Biomass” means the total mass or weight (fresh or dry), at a given time, of a plant tissue, plant tissues, an entire plant, or population of plants. Biomass is usually given as weight per unit area. The term may also refer to all the plants or species in the community (community biomass).

A “bacterial network” means a plurality of endophyte entities (e.g., bacteria, fungi, or combinations thereof) co-localized in an environment, such as on or within a grass agricultural plant. Preferably, a bacterial network includes two or more types of endophyte entities that synergistically interact, such synergistic endophytic populations capable of providing a benefit to the agricultural seed, seedling, or plant derived thereby.

An “increased yield” can refer to any increase in biomass or seed or fruit weight, seed size, seed number per plant, seed number per unit area, bushels per acre, tons per acre, kilo per hectare, or carbohydrate yield. Typically, the particular characteristic is designated when referring to increased yield, e.g., increased grain yield or increased seed size.

As used herein, a microbe or plant or plant element is “modified” when it comprises an artificially introduced genetic or epigenetic modification. In some embodiments, the modification is introduced by a genome engineering technology. In some embodiments, the modification is introduced by a targeted nuclease. In some embodiments, targeted nucleases include, but are not limited to, Transcription Activator-Like Effector Nuclease (TALEN), zinc finger nuclease (ZNF), Clustered Regulatory Interspaced Short Palindromic Repeats (CRISPR), CRISPR/Cas9, CRISPR/CPF1, and combinations thereof. In some embodiments, the modification is an epigenetic modification. In some embodiments, the modification is introduced by treatement with a DNA methyltransferase inhibitor such as 5-azacytidine, or a histone deacetylase inhibitor such as trichostatin A. In some embodiments, the modification is introduced via tissue culture. In some embodiments, a modified microbe or plant or plant element comprises a transgene.

A “transgenic plant” refers to a plant whose genome has been altered by the introduction of at least one heterologous nucleic acid molecule.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, phage or virus, that is capable of replication largely under its own control. A replicon may be either RNA or DNA and may be single or double stranded.

A “vector” is any vehicle to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15 to 25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15 to 25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “promoter region” refers to the 5′ regulatory regions of a gene (e.g., CaMV 35S promoters and/or tetracycline repressor/operator gene promoters).

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, gene editing, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “DNA construct” refers to a genetic sequence used to transform plants and generate progeny transgenic plants. These constructs may be administered to plants in a viral or plasmid vector. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

The phrase “double-stranded RNA mediated gene silencing” refers to a process whereby target gene expression is suppressed in a plant cell via the introduction of nucleic acid constructs encoding molecules which form double-stranded RNA structures with target gene encoding mRNA which are then degraded.

The term “co-suppression” refers to a process whereby expression of a gene, which has been transformed into a cell or plant (transgene), causes silencing of the expression of endogenous genes that share sequence identity with the transgene. Silencing of the transgene also occurs.

As used herein, “transgenic plant” includes a plant that comprises within its genome a heterologous polynucleotide. The heterologous polynucleotide can be either stably integrated into the genome, or can be extra-chromosomal. Preferably, the polynucleotide of the present invention is stably integrated into the genome such that the polynucleotide is passed on to successive generations. Direct and indirect progeny of transformed plants or plant cells that also contain the heterologous polynucleotide are also considered transgenic.

Disclosed herein are transgenic plants having an improved growth or stress resistant (IGSR) phenotype. Transgenic plants with an IGSR phenotype may include an improved biomass quantity and/or an improved resistance to one or more abiotic or pathogenic stressors.

The IGSR phenotype in a transgenic plant may include improved root length or shoot length, increased leaf size, increased biomass, increased germination rates or enhanced resistance to various stressors. In some embodiments of a transgenic plant, the ISGR phenotype may be an increase in biomass relative to control, non-transgenic, or wild-type plants.

In certain embodiments, the disclosed transgenic plants comprise a transformation vector comprising an IGSR nucleotide sequence that encodes or is complementary to a sequence that encodes an “IGSR” polypeptide. In particular embodiments, expression of an IGSR polypeptide in a transgenic plant causes an altered growth rate, an altered biomass content, and/or an altered stress resistant phenotype in the transgenic plant. In certain embodiments, the transgenic plant is selected from the group consisting of plants of the Brassica species, including canola and rapeseed, soy, corn, sunflower, cotton, cocoa, safflower, oil palm, coconut palm, flax, castor, peanut, wheat, oat and rice.

As mentioned above, various methods for the introduction of a desired polynucleotide sequence encoding the desired protein into plant cells are available and known to those of skill in the art and include, but are not limited to: (1) physical methods such as microinjection, electroporation, and microprojectile mediated delivery (biolistics or gene gun technology); (2) virus mediated delivery methods; and (3) Agrobacterium-mediated transformation methods (see, for example, WO 2007/053482 and WO 2005/107437, which are incorporated herein by reference in their entirety).

The most commonly used methods for transformation of plant cells are the Agrobacterium-mediated DNA transfer process and the biolistics or microprojectile bombardment mediated process (i.e., the gene gun). Typically, nuclear transformation is desired but where it is desirable to specifically transform plastids, such as chloroplasts or amyloplasts, plant plastids may be transformed utilizing a microprojectile-mediated delivery of the desired polynucleotide.

Agrobacterium-mediated transformation is achieved through the use of a genetically engineered soil bacterium belonging to the genus Agrobacterium. A number of wild-type and disarmed strains of Agrobacterium tumefaciens and Agrobacterium rhizogenes harboring Ti or Ri plasmids can be used for gene transfer into plants. Gene transfer is done via the transfer of a specific DNA known as “T-DNA” that can be genetically engineered to carry any desired piece of DNA into many plant species.

Agrobacterium-mediated genetic transformation of plants involves several steps. The first step, in which the virulent Agrobacterium and plant cells are first brought into contact with each other, is generally called “inoculation.” Following the inoculation, the Agrobacterium and plant cells/tissues are permitted to be grown together for a period of several hours to several days or more under conditions suitable for growth and T-DNA transfer. This step is termed “co-culture.” Following co-culture and T-DNA delivery, the plant cells are treated with bactericidal or bacteriostatic agents to kill the Agrobacterium remaining in contact with the explant and/or in the vessel containing the explant. If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step. When a “delay” is used, it is typically followed by one or more “selection” steps.

With respect to microprojectile bombardment (U.S. Pat. Nos. 5,550,318; 5,538,880, 5,610,042; and PCT Publication WO 95/06128; each of which is specifically incorporated herein by reference in its entirety), particles are coated with nucleic acids and delivered into cells by a propelling force. Exemplary particles include those comprised of tungsten, platinum, and preferably, gold.

An illustrative embodiment of a method for delivering DNA into plant cells by acceleration is the Biolistics Particle Delivery System (BioRad, Hercules, Calif.), which can be used to propel particles coated with DNA or cells through a screen, such as a stainless steel or Nytex screen, onto a filter surface covered with monocot plant cells cultured in suspension.

Microprojectile bombardment techniques are widely applicable, and may be used to transform virtually any plant species. Examples of species that have been transformed by microprojectile bombardment include monocot species such as maize (PCT Publication No. WO 95/06128), barley, wheat (U.S. Pat. No. 5,563,055, incorporated herein by reference in its entirety), rice, oat, rye, sugarcane, and sorghum, as well as a number of dicots including tobacco, soybean (U.S. Pat. No. 5,322,783, incorporated herein by reference in its entirety), sunflower, peanut, cotton, tomato, and legumes in general (U.S. Pat. No. 5,563,055, incorporated herein by reference in its entirety).

To select or score for transformed plant cells regardless of transformation methodology, the DNA introduced into the cell contains a gene that functions in a regenerable plant tissue to produce a compound that confers upon the plant tissue resistance to an otherwise toxic compound. Genes of interest for use as a selectable, screenable, or scorable marker would include but are not limited to GUS, green fluorescent protein (GFP), luciferase (LUX), antibiotic or herbicide tolerance genes. Examples of antibiotic resistance genes include the penicillins, kanamycin (and neomycin, G418, bleomycin), methotrexate (and trimethoprim), chloramphenicol, and tetracycline. Polynucleotide molecules encoding proteins involved in herbicide tolerance are known in the art, and include, but are not limited to a polynucleotide molecule encoding 5-enolpyruvylshikimate-3-phosphate synthase (EPSPS) described in U.S. Pat. Nos. 5,627,061, 5,633,435, and 6,040,497 and aroA described in U.S. Pat. No. 5,094,945 for glyphosate tolerance; a polynucleotide molecule encoding bromoxynil nitrilase (Bxn) described in U.S. Pat. No. 4,810,648 for Bromoxynil tolerance; a polynucleotide molecule encoding phytoene desaturase (crtl) described in Misawa et al., (Plant J. 4:833-840, 1993) and Misawa et al., (Plant J. 6:481-489, 1994) for norflurazon tolerance; a polynucleotide molecule encoding acetohydroxyacid synthase (AHAS, also known as ALS) described in Sathasiivan et al. (Nucl. Acids Res. 18:2188-2193, 1990) for tolerance to sulfonylurea herbicides; and the bar gene described in DeBlock, et al., (EMBO J. 6:2513-2519, 1987) for glufosinate and bialaphos tolerance.

The regeneration, development, and cultivation of plants from various transformed explants are well documented in the art. This regeneration and growth process typically includes the steps of selecting transformed cells and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Cells that survive the exposure to the selective agent, or cells that have been scored positive in a screening assay, may be cultured in media that supports regeneration of plants. Developing plantlets are transferred to soil less plant growth mix, and hardened off, prior to transfer to a greenhouse or growth chamber for maturation.

The present invention can be used with any transformable cell or tissue. By transformable as used herein is meant a cell or tissue that is capable of further propagation to give rise to a plant. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves.

Any suitable plant culture medium can be used. Examples of suitable media would include but are not limited to MS-based media (Murashige and Skoog, Physiol. Plant, 15:473-497, 1962) or N6-based media (Chu et al., Scientia Sinica 18:659, 1975) supplemented with additional plant growth regulators including but not limited to auxins, cytokinins, ABA, and gibberellins. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Those of skill in the art are aware that media and media supplements such as nutrients and growth regulators for use in transformation and regeneration and other culture conditions such as light intensity during incubation, pH, and incubation temperatures that can be optimized for the particular variety of interest.

One of ordinary skill will appreciate that, after an expression cassette is stably incorporated in transgenic plants and confirmed to be operable, it can be introduced into other plants by sexual crossing. Any of a number of standard breeding techniques can be used, depending upon the species to be crossed.

The terms “pathogen” and “pathogenic” in reference to a bacterium includes any such organism that is capable of causing or affecting a disease, disorder or condition of a host containing the organism.

As used herein, an “agricultural seed” is a seed used to grow a plant typically used in agriculture (an “agricultural plant”). The seed may be of a monocot or dicot plant, and may be planted for the production of an agricultural product, for example grain, food, fiber, etc. As used herein, an agricultural seed is a seed that is prepared for planting, for example, in farms for growing.

In some cases, the present invention contemplates the use of microbes that are “compatible” with agricultural chemicals, for example, a fungicide, an anti-bacterial compound, or any other agent widely used in agricultural which has the effect of killing or otherwise interfering with optimal growth of microbes. As used herein, a microbe is “compatible” with an agricultural chemical when the microbe is modified, such as by genetic modification, e.g., contains a transgene that confers resistance to an herbicide, or is adapted to grow in, or otherwise survive, the concentration of the agricultural chemical used in agriculture. For example, a microbe disposed on the surface of a seed is compatible with the fungicide metalaxyl if it is able to survive the concentrations that are applied on the seed surface.

In some embodiments, an agriculturally compatible carrier can be used to formulate an agricultural formulation or other composition that includes a purified bacterial preparation. As used herein an “agriculturally compatible carrier” refers to any material, other than water, which can be added to a seed or a seedling without causing or having an adverse effect on the seed (e.g., reducing seed germination) or the plant that grows from the seed, or the like.

As used herein, a “portion” of a plant refers to any part of the plant, and can include distinct tissues and/or organs, and is used interchangeably with the term “tissue” throughout.

A “population” of plants, as used herein, can refer to a plurality of plants that were subjected to the same inoculation methods described herein, or a plurality of plants that are progeny of a plant or group of plants that were subjected to the inoculation methods. In addition, a population of plants can be a group of plants that are grown from coated seeds. The plants within a population will typically be of the same species, and will also typically share a common genetic derivation.

A “reference environment” refers to the environment, treatment or condition of the plant in which a measurement is made. For example, production of a compound in a plant associated with a purified bacterial population (e.g., a seed-origin bacterial endophyte) can be measured in a reference environment of drought stress, and compared with the levels of the compound in a reference agricultural plant under the same conditions of drought stress. Alternatively, the levels of a compound in plant associated with a purified bacterial population (e.g., a seed-origin bacterial endophyte) and reference agricultural plant can be measured under identical conditions of no stress.

As used herein, a “colony-forming unit” (“CFU”) is used as a measure of viable microorganisms in a sample. A CFU is an individual viable cell capable of forming on a solid medium a visible colony whose individual cells are derived by cell division from one parental cell.

In part, the present invention describes preparations of novel endophytes, and the creation of synthetic combinations of agricultural seeds and/or seedlings with heterologous endophytes and formulations containing the synthetic combinations, as well as the recognition that such synthetic combinations display a diversity of beneficial properties present in the agricultural plants and the associated endophyte populations newly created by the present inventors. Such beneficial properties include metabolism, transcript expression, proteome alterations, morphology, and the resilience to a variety of environmental stresses, and the combination of a plurality of such properties.

Provided herein are novel compositions, methods, and products related to our invention's ability to overcome the limitations of the prior art in order to provide reliable increases in crop yield, biomass, germination, vigor, stress resilience, and other properties to agricultural crops.

In some embodiments, microbes can confer beneficial properties across a range of concentrations.

In some embodiments, combinations of one or more heterologously disposed endophytes confer additive advantages to plants, including multiple functional properties and resulting in seed, seedling, and plant hosts that display single or multiple improved agronomic properties. In some embodiments, combinations of heterologously disposed endophytes confer syngergistic advantages to plants, including multiple functional properties and resulting in seed, seedling, and plant hosts that display single or multiple improved agronomic properties.

In one aspect, the present invention contemplates a synthetic combination of a plant element of a plant that is coated with an endophyte on its surface. The plant element can be any agricultural plant element, for example an agricultural seed. In one embodiment, the plant element of the first plant is from a monocotyledonous plant. For example, the plant element of the first plant is from a cereal plant. The plant element of the first plant can be selected from the group consisting of a maize plant, a wheat plant, a barley plant, an onion plant, a sorghum plant, or a rice plant. In an alternative embodiment, the plant element of the first plant is from a dicotyledonous plant. The plant element of the first plant can be selected from the group consisting of a cotton plant, a Brassica napus plant, a tomato plant, a pepper plant, a cabbage plant, a lettuce plant, a melon plant, a strawberry plant, a turnip plant, a watermelon plant, a peanut plant, or a soybean plant. In still another embodiment, the seed of the first plant can be from a genetically modified plant. In another embodiment, the seed of the first plant can be a hybrid seed.

The synthetic combination of the present invention contemplates the presence of an endophyte on the surface of the seed of the first plant. In one embodiment, the seed of the first plant is coated with at least 10 CFU or spores of the endophyte per seed, for example, at least 20 CFU or spores, at least 50 CFU or spores, at least 100 CFU or spores, at least 200 CFU or spores, at least 300 CFU or spores, at least 500 CFU or spores, at least 1,000 CFU or spores, at least 3,000 CFU or spores, at least 10,000 CFU or spores, at least 30,000 CFU or spores or more per plant element. In another embodiment, the plant element is coated with at least 10, for example, at least 20, at least 50, at least 100, at least 200, at least 300, at least 500, at least 1,000, at least 3,000, at least 10,000, at least 30,000, at least 100,000, at least 300,000, at least 1,000,000 or more of the endophyte as detected by the number of copies of a particular endophyte gene detected, for example, by quantitative PCR.

In some embodiments of the present invention, it is contemplated that combinations of endophytes can provide an increased benefit to the host plant, as compared to that conferred by a single endophyte, by virtue of additive effects. For example, one endophyte strain that induces a benefit in the host plant may induce such benefit equally well in a plant that is also colonized with a different endophyte strain that also induces the same benefit in the host plant. The host plant thus exhibits the same total benefit from the combination of different endophyte strains as the additive benefit to individual plants colonized with each individual endophyte of the combination. In one example, a plant is colonized with two different endophyte strains: one provides a 1× increase in biomass when associated with the plant, and the other provides a 2× increase in biomass when associated with a different plant. When both endophyte strains are associated with the same plant, that plant would experience a 3× (additive of 1×+2× single effects) increase in auxin biomass. Additive effects are a surprising embodiment of the present invention, as non-compatibility of endophytes may result in a cancelation of the beneficial effects of both endophytes.

In some embodiments of the present invention, it is contemplated that a combination of endophytes can provide an increased benefit to the host plant, as compared to that conferred by a single endophyte, by virtue of synergistic effects. For example, one endophyte strain that induces a benefit in the host plant may induce such benefit beyond additive effects in a plant that is also colonized with a different endophyte strain that also induces that benefit in the host plant. The host plant thus exhibits the greater total benefit from the combination of different endophyte strains than could be seen from the additive benefit of individual plants colonized with each individual endophyte of the combination. In one example, a plant is colonized with two different endophyte strains: one provides a 1× increase in biomass when associated with a plant, and the other provides a 2× increase in biomass when associated with a different plant. When both endophyte strains are associated with the same plant, that plant would experience a 5× (greater than an additive of 1×+2× single effects) increase in biomass. Synergistic effects are a surprising embodiment of the present invention.

In another embodiment, the present invention contemplates methods of coating a plant element, e.g., a seed of a plant, with a plurality of endophytes, as well as synthetic compositions comprising a plurality of endophytes on and/or in the plant element. The methods according to this embodiment can be performed in a manner similar to those described herein for single endophyte coating. In one example, multiple endophytes can be prepared in a single preparation that is coated onto the plant element, e.g., a seed.

Where a plurality of endophytes are coated onto the plant element, any or all of the endophytes may be capable of conferring a beneficial trait onto the host plant. In some cases, all of the endophytes are capable of conferring a beneficial trait onto the host plant. The trait conferred by each of the endophytes may be the same (e.g., both improve the host plant's tolerance to a particular biotic stress), or may be distinct (e.g., one improves the host plant's tolerance to drought, while another improves phosphate utilization). In other cases the conferred trait may be the result of interactions between the endophytes.

In one embodiment, an agricultural plant is contacted with a formulation comprising at least two endophytic microbial entities. Specific examples of pairs of endophytic microbial entities that can be applied to an agricultural plant include, for example, a pair of endophytic microbes containing nucleic acid sequences that are each at least 97% identical to the nucleic acid sequence selected from the group consisting of SEQ ID NO: 1, SEQ ID NO: 2, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18.

In some cases, a single endophyte strain, a plurality of endophytes, or each individual type of endophytes of that plurality, may produce one or more compounds and/or have one or more activities, e.g., one or more of the following: production of a metabolite, production of a phytohormone such as auxin, production of acetoin, production of an antimicrobial compound, production of a siderophore, production of a cellulase, production of a pectinase, production of a chitinase, production of a xylanase, nitrogen fixation, or mineral phosphate solubilization. For example, an endophyte can produce a phytohormone selected from the group consisting of an auxin, a cytokinin, a gibberellin, ethylene, a brassinosteroid, and abscisic acid. In some embodiments, the endophyte produces auxin (e.g., indole-3-acetic acid (IAA)).

In some embodiments, a single endophyte strain, a plurality of endophytes, or each individual type of endophytes of that plurality, can produce a compound with antimicrobial properties. In some embodiments, the compound with antibacterial properties shows bacteriostatic or bactericidal activity against E. coli and/or Bacillus sp. In other embodiments, the endophyte produces a compound with antifungal properties, for example, fungicidal or fungistatic activity against Pythium, Fusarium, Rhizoctonia and/or Lasiodiplodia theobromae. In other embodiments, the endophyte produces a compound with anti-nematode properties, for example, nematocidal activity against Meloidogyne incognita and/or Rotylenchulus remformis.

In some embodiments, a single endophyte strain, a plurality of endophytes, or each individual type of endophytes of that plurality, is capable of nitrogen assimiliation, and is thus capable of increasing nutrient assimilation in the inoculated host plant.

Also described herein is a preparation comprising one or more isolated modified endophytes described above. The preparation further comprises an agriculturally acceptable carrier, and the preparation comprises an amount of endophytes sufficient to improve an agronomic trait of the population of seeds. In one embodiment, the isolated endophyte is cultured, for example, on semi-synthetic or synthetic growth medium. In one embodiment, the endophyte is provided as a powder, for example, a lyophilized powder. In another embodiment, the endophyte is applied in suspension at a suitable concentration. The preparation of microbes can be an aqueous solution, an oil-in-water emulsion or water-in-oil emulsion containing a minimum concentration of a microbe. Microbes are present as live cells, viable cells, spores, or mycelia. Typically, the concentration is at least 10⁴ CFU/ml, for example at least 3×10⁴ CFU/mL, at least 10⁵ CFU/mL, at least 3×10⁵ CFU/mL, at least 10⁶ CFU/mL, at least 3×10⁶ CFU/mL, at least 10⁷ CFU/ml, at least 3×10⁷ CFU/mL, at least 10⁸ CFU/mL, 10⁹ CFU/mL, or more. In one embodiment, the preparation is a solution containing a microbe at a concentration between about 10⁵ CFU/mL and about 10⁹ CFU/mL. In another embodiment, the preparation contains a microbe at a concentration between about 10⁶ CFU/mL and about 10⁸ CFU/mL.

The synthetic preparation can also contain any number of other components. In one embodiment, the synthetic preparation may contain growth media or constituents required for the growth and propagation of the microbe.

The synthetic preparation can be of a defined pH range. In one embodiment, the pH of the preparation can be between pH 5.5-6.0, pH 5.75-6.25, pH 6.0-6.5, pH 6.25-6.75, pH 6.5-7.0, pH 6.75-7.25, and pH 7.0-7.5. The pH of the medium can be adjusted using any biologically compatible buffering agent.

The synthetic preparation can also comprise a carrier, such as diatomaceous earth, clay, or chitin, which act to complex with chemical agents, such as control agents.

The synthetic preparation can also comprise an adherent. Such agents are useful for combining the microbes of the invention with carriers that can contain other compounds (e.g., control agents that are not biologic), to yield a coating composition. Such compositions help create coatings around the plant or seed to maintain contact between the microbe and other agents with the plant or plant part. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. Other examples of adherent compositions that can be used in the synthetic preparation include those described in EP 0818135, CA 1229497, WO 2013090628, EP 0192342, WO 2008103422 and CA 1041788, each of which is incorporated by reference in its entirety.

The synthetic preparation can also contain one or more reagents that promote internalization of the microbe into the plant, and can include any one of the following classes of compounds: a surfactant, an abrasive, an osmoticum, and a plant signaling molecule.

The preparation can also contain a surfactant. Non-limiting examples of surfactants include nitrogen-surfactant blends such as Prefer 28 (Cenex), Surf-N (US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

The synthetic preparation of a defined osmolality can also be used. In one embodiment, the synthetic preparation has an osmolality of less than about 100 mOsm, for example less than about 75 mOsm, less than about 50 mOsm, or less than about 25 mOsm. In another embodiment, the synthetic preparation has an osmolality of at least 250 mOsm, for example at least 300 mOsm, at least 400 mOsm, at least 500 mOsm, at least 600 mOsm, at least 700 mOsm, at least 800 mOsm, 900 mOsm or greater. The osmolality of the preparation can be adjusted by addition of an osmoticum: the osmoticum can be any commonly used osmoticum, and can selected from the group consisting of: mannitol, sorbitol, NaCl, KCl, CaCl₂, MgSO4, sucrose, or any combination thereof.

The endophyte can be obtained from growth in culture, for example, using semi-synthetic or synthetic growth medium. In addition, the microbe can be cultured on solid media, for example on petri dishes, scraped off and suspended into the preparation. Microbes at different growth phases can be used. For example, microbes at lag phase, early-log phase, mid-log phase, late-log phase, stationary phase, early death phase, or death phase can be used.

In another aspect, the seeds according to the present invention provide a substantially uniform population of seeds with a uniform endophyte composition. The uniform population of seeds can be of a predefined weight. For example, a substantially uniform population of seeds containing at least 100 g seeds, for example at least 1 kg seeds, at least 5 kg seeds, at least 10 kg seeds, can be provided by the method according to the present invention that contains—as a whole product—more than 1%, for example more than 5%, more than 10%, more than 20%, more than 30%, more than 40%, especially more than 50%, of the endophytic microorganism, i.e., the strain that is coated onto the surface of the seeds. According to a preferred embodiment, the present invention provides a marketable seed product containing at least 100 g seeds, for example, at least 1 kg seeds, for example at least 5 kg seeds, at least 10 kg seeds, wherein—as a whole product—more than 50%, for example, more than 60%, more than 70%, more than 80%, more than 90%, more than 95%, more than 99%, or 100% of the seeds contain the microbe, i.e., the inoculant strain. Each of the seeds can also contain a uniform number of microbes (for example, viable endophytes): for example, at least 50% of the seeds, for example at least 60%, at least 70%, at least 80%, at least 90%, at least 95%, at least 99%, or more of the seeds in the population can contain at least 100 CFU or spores, at least 300 CFU or spores, at least 1,000 CFU or spores, at least 3,000 CFU or spores, at least 10,000 CFU or spores, at least 30,000 CFU or spores or more, of the endophytic microorganism. In some embodiments, at least 50% of the seeds, for example at least 60%, at least 70%, at least 80%, at least 90%, at least 95%, at least 99%, or more of the seeds in the population contains a single endophyte or a plurality of endophytes at a concentration between about 100 CFU or spores and about 30,000 CFU or spores, between about 100 CFU or spores and about 300 CFU or spores, between about 100 CFU or spores and about 1,000 CFU or spores, between about 100 CFU or spores and about 3,000 CFU or spores, between about 100 CFU or spores and about 10,00 CFU or spores, between about 100 CFU or spores and about 30,000 CFU or spores, between about 300 CFU or spores and about 1,000 CFU or spores, between about 300 CFU or spores and about 3,000 CFU or spores, between about 300 CFU or spores and about 10,00 CFU or spores, between about 300 CFU or spores and about 30,000 CFU or spores, between about 1,000 CFU or spores and about 3,000 CFU or spores, between about 1,000 CFU or spores and about 10,00 CFU or spores, between about 1,000 CFU or spores and about 30,00 CFU or spores, between about 3,000 CFU or spores and about 10,000 CFU or spores, between about 3,000 CFU or spores and about 30,00 CFU or spores, or between about 10,000 CFU or spores and about 30,000 CFU or spores. The endophyte can also be quantitated using other means, for example, using quantitative PCR, to detect the total number of endophyte present on each seed.

The uniformity of the microbes within the seed population can be measured in several different ways. In one embodiment, a substantial portion of the population of seeds, for example at least 10%, at least 20%, at least 30%, at least 40%, at least 50%, at least 60%, at least 70%, at least 75%, at least 80%, at least 90%, at least 95% or more of the seeds in a population, contains a viable endophyte on its surface. In another embodiment, a substantial portion of the population of seeds, for example at least 10%, at least 20%, at least 30%, at least 40%, at least 50%, at least 60%, at least 70%, at least 75%, at least 80%, at least 90%, at least 95% or more of the seeds in a population contain on its surface a threshold number of viable microbe that is at least 1 CFU or spore per seed, at least 10 CFU or spores per seed, for example, at least 100 CFU or spores, at least 300 CFU or spores, at least 1,000 CFU or spores, at least 3,000 CFU or spores, or more, of the microbe per seed. In some embodiments, a substantial portion of the population of seeds, for example at least 10%, at least 20%, at least 30%, at least 40%, at least 50%, at least 60%, at least 70%, at least 75%, at least 80%, at least 90%, at least 95% or more of the seeds in a population contain on its surface a threshold number of viable microbe that is between 1 CFU or spore per seed and about 3,000 CFU or spores per seed, between 1 CFU or spore per seed and about 10 CFU or spores per seed, between 1 CFU or spore per seed and about 100 CFU or spores per seed, between 1 CFU or spore per seed and about 300 CFU or spores per seed, between 1 CFU or spore per seed and about 1,000 CFU or spores per seed, between 1 CFU or spore per seed and about 3,000 CFU or spores per seed, between about 10 CFU or spore per seed and about 100 CFU or spores per seed, between about 10 CFU or spore per seed and about 300 CFU or spores per seed, between about 10 CFU or spore per seed and about 1,000 CFU or spores per seed, between about 10 CFU or spore per seed and about 3,000 CFU or spores per seed, between about 100 CFU or spore per seed and about 300 CFU or spores per seed, between about 100 CFU or spore per seed and about 1,000 CFU or spores per seed, between about 100 CFU or spore per seed and about 3,000 CFU or spores per seed, between about 300 CFU or spore per seed and about 1,000 CFU or spores per seed, between about 300 CFU or spore per seed and about 3,000 CFU or spores per seed, or between about 1,000 CFU or spore per seed and about 3,000 CFU or spores per seed.

In still another aspect, the present invention discloses a substantially uniform population of plants produced by growing the population of seeds described above. In one embodiment, at least 75%, at least 80%, at least 90%, at least 95% or more of the plants comprise in one or more tissues an effective amount of the endophyte or endophytes. In another embodiment, at least 1%, between 1% and 10%, for example, at least 10%, between 10% and 20%, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, at least 95% or more of the plants comprise a microbe population that is substantially similar.

Increased uniformity of the microbes' epigenetic status can also be used to detect increased uniformity of a population of seeds or plants derived from such seeds. For example, where a microbe that has been inoculated by a plant is also present in the plant (for example, in a different tissue or portion of the plant), or where the introduced microbe is sufficiently similar to a microbe that is present in some of the plants (or portion of the plant, including seeds), it is still possible to distinguish between the inoculated microbe and the native microbe, for example, by distinguishing between the two microbe types on the basis of their epigenetic status. Therefore, in one embodiment, the epigenetic status is detected in microbes across individual seeds or the plants that grow from such seeds.

Such uniformity in microbial composition is unique and is extremely advantageous for high-tech and/or industrial agriculture. It allows significant standardization with respect to qualitative endophyte load of seed products. Suitable volumes or weights are those that are currently used for plant seeds (e.g., the at least 100 g, at least 1, 5 or 10 kg; but also 25 or more, 40 or more, 50 kg or more, even 100 kg or more, 500 kg or more, 1 ton or more, etc.). Suitable containers or packages are those traditionally used in plant seed commercialization: however, other containers with more sophisticated storage capabilities (e.g., with microbiologically tight wrappings or with gas- or water-proof containments) can be used. The endophyte amount (qualitatively and quantitatively) contained in the seeds or in the marketable seed product as a whole can be determined by standard techniques in microbiology readily available to any person skilled in the art of plant endophyte analysis.

The methods described herein can also comprise a validating step. The validating step can entail, for example, growing some seeds collected from the inoculated plants into mature agricultural plants, and testing those individual plants for uniformity. Such validating step can be performed on individual seeds collected from cobs, individual plants, individual plots (representing plants inoculated on the same day) or individual fields, and tested as described above to identify pools meeting the required specifications.

In another aspect, described herein is an agricultural field, including a greenhouse, comprising the population of plants described above. In one embodiment, the agricultural field comprises at least 100 plants. In another embodiment, the population occupies at least about 100 square feet of space, wherein at least about 10%, 20%, 30%, 40%, 50%, 60%, 70%, 80%, 90% or more than 90% of the population comprises an effective amount of the microbe. In another embodiment, the population occupies at least about 100 square feet of space, wherein at least 1%, between 1% and 10%, for example, at least 10%, between 10% and 20%, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, at least 95% or more of the population comprises the microbe in reproductive tissue. In still another embodiment, the population occupies at least about 100 square feet of space, wherein at least about 10%, 20%, 30%, 40%, 50%, 60%, 70%, 80%, 90% or more than 90% of the population comprises at least 10 CFUs or spores, 100 CFUs or spores, 1,000 CFUs or spores, 10,000 CFUs or spores or more of the microbe. In still another embodiment, the population occupies at least about 100 square feet of space, wherein at least 1%, between 1% and 10%, for example, at least 10%, between 10% and 20%, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, at least 95% or more of the population comprises between about 10 CFU or spores and about 10,000 CFU or spores, between about 10 CFU or spores and about 100 CFU or spores, between about 10 CFU or spores and about 1,000 CFU or spores, between about 100 CFU or spores and about 1,000 CFU or spores, between about 100 CFU or spores and about 10,00 CFU or spores, or between about 1,000 CFU or spores and about 10,000 CFU or spores. In yet another embodiment, the population occupies at least about 100 square feet of space, wherein at least 1%, between 1% and 10%, for example, at least 10%, between 10% and 20%, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, at least 95% or more of the population comprises a exogenous microbe (i.e., the endophyte) of monoclonal origin.

Plants can be grown individually from the seeds coated with the endophytes to propagate the desired microbes in indoor or outdoor settings. An advantage of the present invention is that it allows multiple plants harboring endophytes to be grown under agricultural methods as a means of providing improved uniformity of microbe-derived benefits to farmers.

Therefore, in another aspect, provided herein are indoor arrangements of populations (e.g., greenhouse) of plants generated from the methods of the present invention. Such arrangements can include at least a defined number of plants of the present invention, such as at least 1, at least 2, at least 3, between 3 and 5, at least 5, between 5 and 10, at least 10, between 10 and 15, at least 15, between 15 and 20, at least 20, between 20 and 30, at least 30, between 30 and 50, at least 50, between 50 and 100, at least 100, between 100 and 200, at least 200, between 200 and 500, at least 500, between 500 and 1000, at least 1000, between 1000 and 5000, at least 5000, between 5000 and 10000, at least 10000 or more plants.

Also provided herein are agricultural fields that contain populations of plants generated from the seeds of the present invention. Agricultural fields can occupy as little as 100 square feet or less, or can occupy hundreds or thousands of acres. Area of field containing a population of microbe-associated plants can be measured in square feet, such as at least 100, 500, 1000, 5000, 10,000, 50,000 or greater than 50,000 square feet, or can be measured in acres, such as least 1, at least 2, at least 3, between 3 and 5, at least 5, between 5 and 10, at least 10, between 10 and 15, at least 15, between 15 and 20, at least 20, between 20 and 30, at least 30, between 30 and 50, at least 50, between 50 and 100, at least 100, between 100 and 200, at least 200, between 200 and 500, at least 500, between 500 and 1000, at least 1000, between 1000 and 5000, at least 5000, between 5000 and 10000, at least 10000, between 10000 and 50000, at least 50000 or greater acres. The field can also be measured in hectares, for example at least 1, at least 2, at least 3, between 3 and 5, at least 5, between 5 and 10, at least 10, between 10 and 15, at least 15, between 15 and 20, at least 20, between 20 and 30, at least 30, between 30 and 50, at least 50, between 50 and 100, at least 100, between 100 and 200, at least 200, between 200 and 500, at least 500, between 500 and 1000, at least 1000, between 1000 and 5000, at least 5000, between 5000 and 10000, at least 10000 or more hectares. Additionally, a field containing a population of microbe-associated plants can be characterized by the number of plants in the population, generally a field is at least two, such as at least 3, between 3 and 5, at least 5, between 5 and 10, at least 10, between 10 and 15, at least 15, between 15 and 20, at least 20, between 20 and 30, at least 30, between 30 and 50, at least 50, between 50 and 100, at least 100, between 100 and 200, at least 200, between 200 and 500, at least 500, between 500 and 1000, at least 1000, between 1000 and 5000, at least 5000, between 5000 and 10000, at least 10000, between 10000 and 25000, at least 250000, between 25000 and 50000, at least 500000, between 50000 and 75000, at least 750000, between 75000 and 100000, at least 1000000 or more plants. A field is generally a contiguous area but may be separated by geographical features such as roads, waterways, buildings, fences, and the like known to those skilled in the art. Because the microbe-associated plants described herein benefit from an increased level of uniformity of germination and other characteristics, it is desirable to maximize the percentage of plants containing microbes. For example, at least 10% (e.g., between 10% and 20%, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, between 95% and 99%, at least 99% or more) of the plants contain the microbes.

In certain embodiments, the endophyte is selected on the basis of its compatibility with commonly used agrichemicals. Agricultural plants can be treated with a vast array of agrichemicals, including fungicides, biocides (anti-bacterial agents), herbicides, insecticides, nematicides, rodenticides, fertilizers, and other agents.

In some cases, it can be important for the endophyte to be compatible with agrichemicals, particularly those with fungicidal or antibacterial properties, in order to persist in the plant although, as mentioned earlier, there are many such fungicidal or antibacterial agents that do not penetrate the plant, at least at a concentration sufficient to interfere with the endophyte. Therefore, where a systemic fungicide or antibacterial agent is used in the plant, compatibility of the endophyte to be inoculated with such agents will be an important criterion.

In one embodiment, natural isolates of endophytes that are compatible with agrichemicals can be used to inoculate the plants according to the methods described herein. For example, fungal endophytes which are compatible with agriculturally employed fungicides can be isolated by plating a culture of the endophytes on a petri dish containing an effective concentration of the fungicide, and isolating colonies of the endophyte that are compatible with the fungicide. In another embodiment, an endophyte that is compatible with a fungicide is used for the methods described herein. Fungicide compatible endophytes can also be isolated by selection on liquid medium. The culture of endophytes can be plated on petri dishes without any forms of mutagenesis; alternatively, the endophytes can be mutagenized using any means known in the art. For example, microbial cultures can be exposed to UV light, gamma-irradiation, or chemical mutagens such as ethylmethanesulfonate (EMS) prior to selection on fungicide containing media. Finally, where the mechanism of action of a particular fungicide is known, the target gene can be specifically mutated (either by gene deletion, gene replacement, site-directed mutagenesis, etc.) to generate an endophyte that is resilient against that particular fungicide.

It will also be appreciated by one skilled in the art that a plant may be exposed to multiple types of fungicides or antibacterial compounds, either simultaneously or in succession, for example at different stages of plant growth. Where the target plant is likely to be exposed to multiple fungicidal and/or antibacterial agents, an endophyte that is compatible with many or all of these agrichemicals can be used to inoculate the plant. An endophyte that is compatible with several fungicidal agents can be isolated, for example, by serial selection. An endophyte that is compatible with the first fungicidal agent is isolated as described above (with or without prior mutagenesis). A culture of the resulting endophyte can then be selected for the ability to grow on liquid or solid media containing the second antifungal compound (again, with or without prior mutagenesis). Colonies isolated from the second selection are then tested to confirm its compatibility to both antifungal compounds.

Endophytes that are compatible to biocides (including herbicides such as glyphosate or antibacterial compounds, whether bacteriostatic or bactericidal) that are agriculturally employed can be isolated using methods similar to those described for isolating fungicide compatible endophytes. In one embodiment, mutagenesis of the microbial population can be performed prior to selection with an antibacterial agent. In another embodiment, selection is performed on the microbial population without prior mutagenesis. In still another embodiment, serial selection is performed on an endophyte: the endophyte is first selected for compatibility to a first antibacterial agent. The isolated compatible endophyte is then cultured and selected for compatibility to the second antibacterial agent. Any colony thus isolated is tested for compatibility to each, or both antibacterial agents to confirm compatibility with these two agents.

Resistance, or compatibility with an antimicrobial agent can be determined by a number of means known in the art, including the comparison of the minimal inhibitory concentration (MIC) of the unmodified and modified endophyte. Therefore, in one embodiment, the present invention discloses an isolated modified endophyte derived from an endophyte isolated from within a plant or tissue thereof, wherein the endophyte is modified such that it exhibits at least 3 fold greater, for example, at least 5 fold greater, at least 10 fold greater, at least 20 fold greater, at least 30 fold greater or more MIC to an antimicrobial agent when compared with the unmodified endophyte.

Candidate isolates can be tested to ensure that the selection for agrichemical compatibility did not result in loss of a desired microbial bioactivity. Isolates of the endophyte that are compatible with commonly employed fungicides can be selected as described above. The resulting compatible endophyte can be compared with the parental endophyte on plants in its ability to promote germination.

The present invention contemplates the establishment of a microbial symbiont in a plant. In one embodiment, the microbial association results in a detectable change to the seed or plant. The detectable change can be an improvement in a number of agronomic traits (e.g., improved general health, increased response to biotic or abiotic stresses, or enhanced properties of the plant or a plant part, including fruits and grains). Alternatively, the detectable change can be a physiological or biological change that can be measured by methods known in the art. The detectable changes are described in more detail in the sections below. As used herein, an endophyte is considered to have conferred an improved agricultural trait whether or not the improved trait arose from the plant, the endophyte, or the concerted action between the plant and endophyte. Therefore, for example, whether a beneficial hormone or chemical is produced by the plant or endophyte, for purposes of the present invention, the endophyte will be considered to have conferred an improved agronomic trait upon the host plant.

In some embodiments, plant-endophyte combinations confer an agronomic benefit in agricultural plants. In some embodiments, the agronomic trait is selected from the group consisting of altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a transcript, relative to a reference plant. In other embodiments, at least two agronomic traits are improved in the agricultural plant.

For example, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, or at least 300% or more, when compared with uninoculated plants grown under the same conditions.

The method of the present invention can facilitate crop productivity by enhancing germination, seedling vigor and biomass in comparison with a non-treated control. Moreover, the introduction of the beneficial microorganisms to within the seed instead of by, e.g., seed coating, makes the endophytes less susceptible to environmental perturbation and more compatible with chemical seed coatings (e.g., pesticides and herbicides). Using endophyte colonized seeds, the plant growth and biomass increases are statistically similar to those obtained using conventional inoculation methods e.g., exogenous seed soaking and soil inoculation (that are more laborious and less practicable in certain circumstances).

Also described herein are plants, and fields of plants, that are associated with beneficial endophytes, such that the overall fitness, productivity or health of the plant or a portion thereof, is maintained, increased and/or improved over a period of time. Improvement in overall plant health can be assessed using numerous physiological parameters including, but not limited to, height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof. Improved plant health, or improved field health, can also be demonstrated through improved resistance or response to a given stress, either biotic or abiotic stress, or a combination of one or more abiotic stresses, as provided herein.

Disclosed herein are endophyte-associated plants with increased resistance to an abiotic stress. Exemplary abiotic stresses include, but are not limited to: drought, salt, high metal content, low nutrients, cold stress, and heat stress. In some embodiments, the plants comprise a single endophyte strain or a plurality of endophytes able to increase heat and/or drought-tolerance in sufficient quantity, such that increased growth or improved recovery from wilting under conditions of heat or drought stress is observed. For example, a plurality of endophyte populations described herein can be present in sufficient quantity in a plant, resulting in increased growth as compared to a plant that does not contain endophytes, when grown under drought conditions or heat shock conditions, or following such conditions. Increased heat and/or drought tolerance can be assessed with physiological parameters including, but not limited to, increased height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, wilt recovery, turgor pressure, or any combination thereof, as compared to a reference agricultural plant grown under similar conditions. For example, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions.

In other embodiments, a single endophyte strain or plurality of endophytes able to confer increased tolerance to salinity stress can be introduced into plants. The resulting plants comprising endophytes can exhibit increased resistance to salt stress, whether measured in terms of survival under saline conditions, or overall growth during, or following salt stress. The physiological parameters of plant health recited above, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., isogenic plants without the endophytes) grown under identical conditions. For example, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions.

In some embodiments, a plant resulting from seeds containing an endophyte able to confer salt tolerance described herein exhibits an increase in the inhibitory sodium concentration by at least 10 mM, for example at least 15 mM, at least 20 mM, at least 30 mM, at least 40 mM, at least 50 mM, at least 60 mM, at least 70 mM, at least 80 mM, at least 90 mM, at least 100 mM or more, when compared with the reference agricultural plants.

In other embodiments, a single endophyte strain or plurality of endophytes protects the plant from a biotic stress, for example, insect infestation, nematode infestation, complex infection, fungal infection, oomycete infection, protozoal infection, viral infection, and herbivore grazing, or a combination thereof. For example, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions.

In some embodiments, endophytes described herein confer upon the host plant the ability to repel insect herbivores. In other cases, the endophytes may produce, or induce the production in the plant of, compounds which are insecticidal or insect repellant. The insect may be any one of the common pathogenic insects affecting plants, particularly agricultural plants. Examples include, but are not limited to: Leptinotarsa spp. (e.g., L. decemlineata (Colorado potato beetle), L. juncta (false potato beetle), or L. texana (Texan false potato beetle)); Nilaparvata spp. (e.g., N. lugens (brown planthopper)); Laode/phax spp. (e.g., L. striatellus (small brown planthopper)); Nephotettix spp. (e.g., N. virescens or N. cincticeps (green leafhopper), or N. nigropictus (rice leafhopper)); Sogatella spp. (e.g., S. furcifera (white-backed planthopper)); Chilo spp. (e.g., C. suppressalis (rice striped stem borer), C. auricilius (gold-fringed stem borer), or C. polychrysus (dark-headed stem borer)); Sesamia spp. (e.g., S. inferens (pink rice borer)); Tryporyza spp. (e.g., T. innotata (white rice borer), or T. incertulas (yellow rice borer)); Anthonomus spp. (e.g., A. grandis (boll weevil)); Phaedon spp. (e.g., P. cochleariae (mustard leaf beetle)); Epilachna spp. (e.g., E. varivetis (Mexican bean beetle)); Tribolium spp. (e.g., T. castaneum (red floor beetle)); Diabrotica spp. (e.g., D. virgifera (western corn rootworm), D. barberi (northern corn rootworm), D. undecimpunctata howardi (southern corn rootworm), D. virgifera zeae (Mexican corn rootworm); Ostrinia spp. (e.g., O. nubilalis (European corn borer)); Anaphothrips spp. (e.g., A. obscrurus (grass thrips)); Pectinophora spp. (e.g., P. gossypiella (pink bollworm)); Heliothis spp. (e.g., H. virescens (tobacco budworm)); Trialeurodes spp. (e.g., T. abutiloneus (banded-winged whitefly) T. vaporariorum (greenhouse whitefly)); Bemisia spp. (e.g., B. argentifoii (silverleaf whitefly)); Aphis spp. (e.g., A. gossypii (cotton aphid)); Lygus spp. (e.g., L. lineolaris (tarnished plant bug) or L. hesperus (western tarnished plant bug)); Euschistus spp. (e.g., E. conspersus (consperse stink bug)); Chlorochroa spp. (e.g., C. sayi (Say stinkbug)); Nezara spp. (e.g., N. viridula (southern green stinkbug)); Thrips spp. (e.g., T. tabaci (onion thrips)); Frankliniella spp. (e.g., F. fusca (tobacco thrips), or F. occidentalis (western flower thrips)); Acheta spp. (e.g., A. domesticus (house cricket)); Myzus spp. (e.g., M. persicae (green peach aphid)); Macrosiphum spp. (e.g., M. euphorbiae (potato aphid)); Blissus spp. (e.g., B. leucopterus (chinch bug)); Acrosternum spp. (e.g., A. hilare (green stink bug)); Chilotraea spp. (e.g., C. polychrysa (rice stalk borer)); Lissorhoptrus spp. (e.g., L. oryzophilus (rice water weevil)); Rhopalosiphum spp. (e.g., R. maidis (corn leaf aphid)); Anuraphis spp. (e.g., A. maidiradicis (corn root aphid)), and combinations thereof.

The endophyte-associated plant can be tested for its ability to resist, or otherwise repel, pathogenic insects by measuring, for example, insect load, overall plant biomass, biomass of the fruit or grain, percentage of intact leaves, or other physiological parameters described herein, and comparing with a reference agricultural plant. In some embodiments, the endophyte-associated plant exhibits increased biomass as compared to a reference agricultural plant grown under the same conditions (e.g., grown side-by-side, or adjacent to, endophyte-associated plants). In other embodiments, the endophyte-associated plant exhibits increased fruit or grain yield as compared to a reference agricultural plant grown under the same conditions (e.g., grown side-by-side, or adjacent to, endophyte-associated plants). In any of the above, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions.

In some embodiments, the endophyte-associated plant has an increased resistance to a nematode when compared with a reference agricultural plant. As before with insect herbivores, biomass of the plant or a portion of the plant, or any of the other physiological parameters mentioned elsewhere, can be compared with the reference agricultural plant grown under the same conditions. Particularly useful measurements include overall plant biomass, biomass and/or size of the fruit or grain, and root biomass. In some embodiments, the endophyte-associated plant exhibits increased biomass as compared to a reference agricultural plant grown under the same conditions (e.g., grown side-by-side, or adjacent to, the endophyte-associated plants, under conditions of nematode challenge). In other embodiments, the endophyte-associated plant exhibits increased root biomass as compared to a reference agricultural plant grown under the same conditions (e.g., grown side-by-side, or adjacent to, the endophyte-associated plants, under conditions of nematode challenge). In still another embodiment, the endophyte-associated plant exhibits increased fruit or grain yield as compared to a reference agricultural plant grown under the same conditions (e.g., grown side-by-side, or adjacent to, the endophyte-associated plants, under conditions of nematode challenge). In any of the above, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions.

The present invention contemplates the use a single endophyte strain or of a plurality of endophytes that is able to confer resistance to fungal pathogens to the host plant. Increased resistance to fungal inoculation can be measured, for example, using any of the physiological parameters presented above, by comparing with reference agricultural plants. In some embodiments, the endophyte-associated plant exhibits increased biomass and/or less pronounced disease symptoms as compared to a reference agricultural plant grown under the same conditions (e.g., grown side-by-side, or adjacent to, the endophyte-associated plants, infected with the fungal pathogen). In still another embodiment, the endophyte-associated plant exhibits increased fruit or grain yield as compared to a reference agricultural plant grown under the same conditions (e.g., grown side-by-side, or adjacent to, the endophyte-associated plants, infected with the fungal pathogen). In other embodiments, the endophyte-associated plant exhibits decreased hyphal growth as compared to a reference agricultural plant grown under the same conditions (e.g., grown side-by-side, or adjacent to, the endophyte-associated plants, infected with the fungal pathogen). In any of the above, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions.

Plant viruses are estimated to account for 18% of global crop losses due to disease. There are numerous examples of viral pathogens affecting agricultural productivity. Examples include the American wheat striate mosaic virus (AWSMV) (wheat striate mosaic), Barley stripe mosaic virus (BSMV), Barley yellow dwarf virus (BYDV), Brome mosaic virus (BMV), Cereal chlorotic mottle virus (CCMV), Corn chlorotic vein banding virus (CCVBV), Brazilian maize mosaic virus, Corn lethal necrosis Virus complex from Maize chlorotic mottle virus, (MCMV), Maize dwarf mosaic virus (MDMV), A or B Wheat streak mosaic virus (WSMV), Cucumber mosaic virus (CMV), Cynodon chlorotic streak virus (CCSV), Johnsongrass mosaic virus (JGMV), Maize bushy stunt Mycoplasma-like organism (MLO) associated virus, Maize chlorotic dwarf Maize chlorotic dwarf virus (MCDV), Maize chlorotic mottle virus (MCMV), Maize dwarf mosaic virus (MDMV), strains A, D, E and F, Maize leaf fleck virus (MLFV), Maize line virus (MLV), Maize mosaic (corn leaf stripe, Maize mosaic virus (MMV), enanismo rayado), Maize mottle and chlorotic stunt virus, Maize pellucid ringspot virus (MPRV), Maize raya gruesa virus (MRGV), Maize rayado fino (fine striping) virus (MRFV), Maize red stripe virus (MRSV), Maize ring mottle virus (MRMV), Maize rio cuarto virus (MRCV), Maize rough dwarf virus (MRDV), Cereal tillering disease virus, Maize sterile stunt virus, barley yellow striate virus, Maize streak virus (MSV), Maize stripe virus, Maize chloroticstripe virus, maize hoja blanca virus, Maize stunting virus; Maize tassel abortion virus (MTAV), Maize vein enation virus (MVEV), Maize wallaby ear virus (MWEV), Maize white leaf virus, Maize white line mosaic virus (MWLMV), Millet red leaf virus (MRLV), Northern cereal mosaic virus (NCMV), Oat pseudorosette virus, (zakuklivanie), Oat sterile dwarf virus (OSDV), Rice black-streaked dwarf virus (RBSDV), Rice stripe virus (RSV), Sorghum mosaic virus (SrMV), Sugarcane mosaic virus (SCMV) strains H, 1 and M, Sugarcane Fiji disease virus (FDV), Sugarcane mosaic virus (SCMV) strains A, B, D, E, SC, BC, Sabi and MB (formerly MDMV-B), and Wheat spot mosaic virus (WSMV). In one embodiment, the endophyte-associated plant provides protection against viral pathogens such that there is at least 5% greater biomass, for example, at least 10%, at least 15%, at least 20%, at least 30%, at least 40%, at least 50%, at least 75%, at least 100% or more biomass, than the reference agricultural plant grown under the same conditions. In still another embodiment, the endophyte-associated plant exhibits at least 5% greater fruit or grain yield, for example, at least 10%, at least 15%, at least 20%, at least 30%, at least 40%, at least 50%, at least 75%, at least 100% or more fruit or grain yield when challenged with a virus, as compared to a reference agricultural plant grown under the same conditions. In yet another embodiment, the endophyte-associated plant exhibits at least 5% lower viral titer, for example, at least 10%, at least 15%, at least 20%, at least 30%, at least 40%, at least 50%, at least 75%, at least 100% lower viral titer when challenged with a virus, as compared to a reference agricultural plant grown under the same conditions.

Likewise, bacterial pathogens are a significant problem negatively affecting agricultural productivity and accounting for 27% of global crop losses due to plant disease. In one embodiment, the endophyte-associated plant described herein provides protection against bacterial pathogens such that there is at least 5% greater biomass, for example, at least 10%, at least 15%, at least 20%, at least 30%, at least 40%, at least 50%, at least 75%, at least 100% or more biomass, than the reference agricultural plant grown under the same conditions. In still another embodiment, the endophyte-associated plant exhibits at least 5% greater fruit or grain yield, for example, at least 10%, at least 15%, at least 20%, at least 30%, at least 40%, at least 50%, at least 75%, at least 100% or more fruit or grain yield when challenged with a bacterial pathogen, than the reference agricultural plant grown under the same conditions. In yet another embodiment, the endophyte-associated plant exhibits at least 5% lower bacterial count, for example, at least 10%, at least 15%, at least 20%, at least 30%, at least 40%, at least 50%, at least 75%, at least 100% lower bacterial count when challenged with a bacteria, as compared to a reference agricultural plant grown under the same conditions.

In other embodiments, the improved trait can be an increase in overall biomass of the plant or a part of the plant, including its fruit or seed. In some embodiments, a single endophyte strain or a plurality of endophytes is disposed on the surface or within a tissue of the plant element in an amount effective to increase the biomass of the plant, or a part or tissue of the plant grown from the plant element. The increased biomass is useful in the production of commodity products derived from the plant. Such commodity products include an animal feed, a fish fodder, a cereal product, a processed human-food product, a sugar or an alcohol. Such products may be a fermentation product or a fermentable product, one such exemplary product is a biofuel. The increase in biomass can occur in a part of the plant (e.g., the root tissue, shoots, leaves, etc.), or can be an increase in overall biomass. Increased biomass production, such an increase meaning at at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions. Such increase in overall biomass can be under relatively stress-free conditions. In other cases, the increase in biomass can be in plants grown under any number of abiotic or biotic stresses, including drought stress, salt stress, heat stress, cold stress, low nutrient stress, nematode stress, insect herbivory stress, fungal pathogen stress, bacterial pathogen stress, and viral pathogen stress. In some embodiments, a plurality of endophytes is disposed in an amount effective to increase root biomass by at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions, when compared with a reference agricultural plant.

In other cases, a plurality of endophytes is disposed on the plant element in an amount effective to increase the average biomass of the fruit or cob from the resulting plant at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, when compared with uninoculated plants grown under the same conditions.

The present invention contemplates a synthetic combination of a plant element that is associated with a single endophyte strain or a plurality of endophytes to confer an improved trait of agronomic importance to the host plant, or an improved agronomic trait potential to a plant element associated with the endophytes, that upon and after germination will confer said benefit to the resultant host plant.

In some embodiments, the plant element is a leaf, and the synthetic combination is formulated for application as a foliar treatment.

In some embodiments, the plant element is a seed, and the synthetic combination is formulated for application as a seed coating.

In some embodiments, the plant element is a root, and the synthetic combination is formulated for application as a root treatment.

In certain embodiments, the plant element becomes associated with a plurality of endophytes through delayed exposure. For example, the soil in which a plant element is to be introduced is first treated with a composition comprising a plurality of endophytes. In another example, the area around the plant or plant element is exposed to a formulation comprising a plurality of endophytes, and the plant element becomes subsequently associated with the endophytes due to movement of soil, air, water, insects, mammals, human intervention, or other methods.

The plant element can be obtained from any agricultural plant. In some embodiments, the plant element of the first plant is from a monocotyledonous plant. For example, the plant element of the first plant is from a cereal plant. The plant element of the first plant can be selected from the group consisting of a maize seed, a wheat seed, a barley seed, a rice seed, a sugarcane seed, a maize root, a wheat root, a barley root, a sugarcane root, a rice root, a maize leaf, a wheat leaf, a barley leaf, a sugarcane leaf, or a rice leaf. In an alternative embodiment, the plant element of the first plant is from a dicotyledonous plant. The plant element of the first plant can be selected from the group consisting of a cotton seed, a tomato seed, a canola seed, a pepper seed, a soybean seed, a cotton root, a tomato root, a canola root, a pepper root, a soybean root, a cotton leaf, a tomato leaf, a canola leaf, a pepper leaf, or a soybean leaf. In still another embodiment, the plant element of the first plant can be from a genetically modified plant. In other embodiments, the plant element of the first plant can be a hybrid plant element.

A single endophyte strain or a plurality of endophytes is intended to be useful in the improvement of agricultural plants, and as such, may be formulated with other compositions as part of an agriculturally compatible carrier. It is contemplated that such carriers can include, but not be limited to: seed treatment, root treatment, foliar treatment, soil treatment. The carrier composition with a plurality of endophytes, may be prepared for agricultural application as a liquid, a solid, or a gas formulation. Application to the plant may be achieved, for example, as a powder for surface deposition onto plant leaves, as a spray to the whole plant or selected plant element, as part of a drip to the soil or the roots, or as a coating onto the seed prior to planting. Such examples are meant to be illustrative and not limiting to the scope of the invention.

In some embodiments, the present invention contemplates plant elements comprising a single endophyte strain or a plurality of endophytes, and further comprising a formulation. The formulation useful for these embodiments generally comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient.

In some cases, a single endophyte strain or a plurality of endophytes is mixed with an agriculturally compatible carrier. The carrier can be a solid carrier or liquid carrier. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in a composition of the invention. Water-in-oil emulsions can also be used to formulate a composition that includes a plurality of endophytes. Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or plant growth medium. Other agricultural carriers that may be used include fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, leaf, root, plant elements, sugar cane bagasse, hulls or stalks from grain processing, ground plant material or wood from building site refuse, sawdust or small fibers from recycling of paper, fabric, or wood. Other suitable formulations will be known to those skilled in the art.

The formulation can also contain a surfactant, wetting agent, emulsifier, stabilizer, or anti-foaming agent. Non-limiting examples of surfactants include nitrogen-surfactant blends such as Prefer 28 (Cenex), Surf-N (US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision), polysorbate 20, polysorbate 80, Tween 20, Tween 80, Scattics, Alktest TW20, Canarcel, Peogabsorb 80, Triton X-100, Conco NI, Dowfax 9N, Igebapl CO, Makon, Neutronyx 600, Nonipol NO, Plytergent B, Renex 600, Solar NO, Sterox, Serfonic N, T-DET-N, Tergitol NP, Triton N, IGEPAL CA-630, Nonident P-40, and Pluronic. In some embodiments, the surfactant is present at a concentration of between 0.01% v/v to 10% v/v. In other embodiments, the surfactant is present at a concentration of between 0.1% v/v to 1% v/v. An example of an anti-foaming agent is Antifoam-C.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the endophytes used, and should promote the ability of the microbial population to survive application on the plant elements and to survive desiccation. Examples of suitable desiccants include one or more of trehalose, sucrose, glycerol, and Methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In the liquid form, for example, solutions or suspensions, a plurality of endophytes can be mixed or suspended in aqueous solutions. Suitable liquid diluents or carriers include aqueous solutions, petroleum distillates, or other liquid carriers.

Solid compositions can be prepared by dispersing a plurality of endophytes of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

In some embodiments, the formulation is ideally suited for coating of a plurality of endophytes onto plant elements. The plurality of endophytes is capable of conferring many agronomic benefits to the host plants. The ability to confer such benefits by coating the plurality of endophytes on the surface of plant elements has many potential advantages, particularly when used in a commercial (agricultural) scale.

The formulations comprising a plurality of endophytes of the present invention typically contains between about 0.1 to 95% by weight, for example, between about 1% and 90%, between about 3% and 75%, between about 5% and 60%, between about 10% and 50% in wet weight of a plurality of endophytes. In some embodiments, the formulation contains at least about 10^{A}2 per ml of formulation, at least about 10\hat{ }3 per ml of formulation, for example, at least about 10\hat{ }4, at least about 10\hat{ }5, at least about 10\hat{ }6, at least about 10\hat{ }7 CFU or spores, at least about 10\hat{ }8 CFU or spores per ml of formulation. In some embodiments, the formulation be applied to the plant element at about 10\hat{ }2 CFU/seed, between 10\hat{ }2 and 10\hat{ }3 CFU, at least about 10^{A}3 CFU, between 10^{A}3 and 10^{A}4 CFU, at least about 10^{A}4 CFU, between 10^{A}4 and 10^{A}5 CFU, at least about 10\hat{ }5 CFU, between 10\hat{ }5 and 10\hat{ }6 CFU, at least about 10\hat{ }6 CFU, between 10\hat{ }6 and 10\hat{ }7 CFU, at least about 10\hat{ }7 CFU, between 10\hat{ }7 and 10\hat{ }8 CFU, or even greater than 10^{A}8 CFU per seed.

The compositions provided herein are preferably stable. The endophyte may be shelf-stable, where at least 0.01%, of the CFU or spores are viable after storage in desiccated form (i.e., moisture content of 30% or less) for 1, 2, 3, 4, 5, 6, 7, 8, 9, 10 or greater than 10 weeks at 4° C. or at room temperature. Optionally, a shelf-stable formulation is in a dry formulation, a powder formulation, or a lyophilized formulation. In some embodiments, the formulation is formulated to provide stability for the population of endophytes. In one embodiment, the formulation is substantially stable at temperatures between about −20° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In another embodiment, the formulation is substantially stable at temperatures between about 4° C. and about 37° C. for at least about 5, 10, 15, 20, 25, 30 or greater than 30 days.

Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole.

Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some embodiments, the endophyte is compatible with at least one of the fungicides selected from the group consisting of: 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, benalaxyl, benomyl, benthiavalicarb-isopropyl, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fentin, fentin acetate, fentin hydroxide, ferbam, ferimzone, fluazinam, fludioxonil, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), ipconazole, iprobenfos, iprodione, iprovalicarb, isoprothiolane, isopyrazam, isotianil, kasugamycin, kasugamycin hydrochloride hydrate, kresoxim-methyl, mancopper, mancozeb, mandipropamid, maneb, mepanipyrim, mepronil, mercuric chloride, mercuric oxide, mercurous chloride, metalaxyl, mefenoxam, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z071, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)-N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, 5-fluorocytosine and profungicides thereof, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, picolinamide UK-2A and derivatives thereof, prothiocarb; prothiocarb hydrochloride, pyracarbolid, pyridinitril, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, XRD-563, and zarilamide, IK-1140. In still another embodiment, an endophyte that is compatible with an antibacterial compound is used for the methods described herein. For example, the endophyte is compatible with at least one of the antibiotics selected from the group consisting of: Amikacin, Gentamicin, Kanamycin, Neomycin, Netilmicin, Tobramycin, Paromomycin, Spectinomycin, Geldanamycin, Herbimycin, Rifaximin, streptomycin, Loracarbef, Ertapenem, Doripenem, Imipenem/Cilastatin, Meropenem, Cefadroxil, Cefazolin, Cefalotin or Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Teicoplanin, Vancomycin, Telavancin, Clindamycin, Lincomycin, Daptomycin, Azithromycin, Clarithromycin, Dirithromycin, Erythromycin, Roxithromycin, Troleandomycin, Telithromycin, Spiramycin, Aztreonam, Furazolidone, Nitrofurantoin, Linezolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), Sulfonamidochrysoidine (archaic), Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampicin (Rifampin in US), Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, and Trimethoprim.

A fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used as a control agent in a seed composition.

Preferred nematode-antagonistic biocontrol agents include ARF18; Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium virens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

In some embodiments, the endophytes of the present invention display tolerance to an agrichemical selected from the group consisting of: Aeris®, Avicta® DuoCot 202, Cruiser®, Syntenta CCB® (A), Clariva®, Albaugh, Dynasty®, Apron®, Maxim®, Gaucho®, Provoke@ST, Syngenta CCB®, Trilex®, WG Purple, WG Silver, Azoxystrobin, Carboxin, Difenoconazole, Fludioxonil, fluxapyroxad, Ipconazole, Mefenoxam, Metalaxyl, Myclobutanil, Penflufen, pyraclostrobin, Sedaxane, TCMTB, Tebuconazole, Thiram, Triadimenol (Baytan®), Trifloxystrobin, Triticonazole, Tolclofos-methyl, PCNB, Abamectin, Chlorpyrifos, Clothianidin, Imidacloprid, Thiamethoxam, and Thiodicarb.

In certain embodiments, a composition described herein may be in the form of a liquid, a slurry, a solid, or a powder (wettable powder or dry powder). In another embodiment, a composition may be in the form of a seed coating. Compositions in liquid, slurry, or powder (e.g., wettable powder) form may be suitable for coating plant elements. When used to coat plant elements, the composition may be applied to the plant elements and allowed to dry. In embodiments wherein the composition is a powder (e.g., a wettable powder), a liquid, such as water, may need to be added to the powder before application to a seed.

In still another embodiment, the methods can include introducing into the soil an inoculum of one or more of the endophyte populations described herein. Such methods can include introducing into the soil one or more of the compositions described herein. The inoculum(s) or compositions may be introduced into the soil according to methods known to those skilled in the art. Non-limiting examples include in-furrow introduction, spraying, coating seeds, foliar introduction, etc. In a particular embodiment, the introducing step comprises in-furrow introduction of the inoculum or compositions described herein.

In one embodiment, plant elements may be treated with composition(s) described herein in several ways but preferably via spraying or dripping. Spray and drip treatment may be conducted by formulating compositions described herein and spraying or dripping the composition(s) onto a seed(s) via a continuous treating system (which is calibrated to apply treatment at a predefined rate in proportion to the continuous flow of seed), such as a drum-type of treater. Batch systems, in which a predetermined batch size of seed and composition(s) as described herein are delivered into a mixer, may also be employed.

### II. Methods of the Invention

The invention relates to a process and method for the production and use of endophytes as plant inoculants products that provide unique inoculant feature/benefits for the promotion of plant vigor, health, growth and yield comprising bacteria isolated from non-cultivated members of the cotton family from the family Malvaceae, e.g., endophytic bacteria Bacillus amyloliquefaciens (strain Bamy), Pantoea dispersa (strain Pdisp), Pseudomonas oleovorans (strain Poryz, isolated from T. populnea; Pseudomonas oryzihabitans, strain WCB2, isolated from G. hirsute), Enterobacter cloacae (strain Entero), Curtobacterium oceanosedimentum (strain WCB1) and Achromobacter xylosoxidans (strain Achromo) described in Table 1.

Several of these new strains of bacteria have been deposited with the NRRL Agriculture Research Service Culture Collection (herein after “NRRL”) under the terms of the Budapest Treaty, under accession numbers provided herein.

The invention also relates to a process and method for producing economically acceptable quantities of preparations of the aforementioned bacteria. The invention further relates to an endophyte product(s) produced by such processes and methods. The endophyte product(s) may comprise a solid substrate of, for example, certain cereals e.g. rye, which contain sufficient natural emulsifiers in the form of various proteins, lignin, to provide the bacteria with excellent natural dispersing/wetting/sticker agents that allows for rapid site occupation on/in plants. The product formulation allows/enables practical use and application of the product(s) to roots, stems, leaves, flowers, bulbs, etc of plants as a water-based sprayable formulations or as dusts for other uses e.g. seed treatment, or as dusts for insect/mite vectors. The product when applied to seeds, roots, stems, leaves, flowers, wounds or cut surfaces of plants enables the endophyte to act as an inoculant within the tissues of plants. The product provides improved roots, leaf, stem and or vegetative bud (flowers) growth to plants and or enhances/improves the germination and emergence of seeds and also causes mortality in neighboring competitor species. The product provides for reduction of environmental or cultural stress to plants e.g. root loss due to trimming, pruning, cutting or other stresses. The product provides improved crop quality and faster development to marketability of the crop. The product provides for a reduction in the dependency on chemical pesticides for pest control e.g. control of Botrytis, Fusarium, Pythium and the like. The product can be used for the production of a variety of greenhouse, horticultural and agronomic field crops. The composition of the invention can be a plant inoculant composition comprising the bacteria described above in admixture with an agrochemically acceptable diluent or carrier. The invention also relates to a method of enhancing growth, health vigor or yield of a plant which method comprises applying the plant inoculant composition of the invention to a plant or plant locus. The invention further relates to a method of combating a plant fungus which method comprises applying an antifungal effective amount of the composition.

Lyophilization Procedure

Freeze drying bacteria (lyophilization) is a very well established method for the archiving and long-term storage. Initial reports of freeze drying bacteria can be found in the middle of last century. The approaches used vary widely, but they all following the standard process associated with lyophilization, namely the freezing of the sample, application of a high vacuum, warming of the sample while under vacuum which causes water sublimation, driving off excess water through a drying phase, and finally sealing of the sample to prevent water uptake. This general process is used to preserve bacteria, fungi, yeasts, proteins, nucleic acids, and any other molecules which may be degraded due to the presence of water.

Thus in one aspect of the invention, one or more of the endophytic bacteria will be applied to a plant or a plant part (such as seeds) as a lyophilized (freeze-dried) powder. In brief, the liquid culture will be: centrifuged, re-suspended in a lyophilization medium which will optionally include cryoprotectants and biological- and/or chemical-oxygen scavengers, transferred to a shelf lyophilizer, lyophilized, and packaged for transport and storage.

In an alternative approach, one or more bacteria may be encapsulated in alginate beads enriched with humic acid as described by Young C C et al., Biotechnol Bioeng. 2006 Sep. 5; 95(1):76-83. Also see “Alginate beads as a storage, delivery and containment system for genetically modified PCB degrader and PCB biosensor derivatives of Pseudomonas fluorescens F113 B” by Power et al., Journal of Applied Microbiology 110, 1351-1358, 2011.

Other approaches include coating seeds with preparations comprising the endophytic bacteria of the invention. For example, the endophytic bacteria Bacillus amyloliquefaciens, Pantoea dispersa, Pseudomonas oryzihabitans, Pseudomonas oleovorans, Enterobacter cloacae, and Achromobacter xylosoxidans, alone or in combination, can be incorporated into a carrier, which include without limitation, alginate (micro-bead formation), chitosan, carboxymethylcellulose-starch, clay, finely-ground peat mixed with calcium carbonate, methacrylic acid, bio-char and biogels. In certain embodiments, carrier and Bacillus amyloliquefaciens, for example, may be mixed with additives (including: adhesives, nutrients, surfactants and stabilizers).

The carrier and Bacillus amyloliquefaciens, for example, (with additives) can be applied to seeds of cotton crops using a commercial seed dressing machine (e.g., MAYJOY High Speed Seeds Dressing Machine/Corn Seed Dresser).

In some embodiments, endophytic bacteria described herein may also be used as an additive to create seed balls. In this approach, clay mixed with freeze-dried preparation of an endophytic bacteria and seeds (2-3) are added to the center of a small clay ball. The seed balls are then dried and stored for future use. In some embodiments, the plant element is associated with a single endophyte strain or a plurality of endophytes on its surface. Such association is contemplated to be via a mechanism selected from the group consisting of: spraying, immersion, coating, encapsulating, dusting, dripping, aerosolizing, seed treatment, root wash, seedling soak, foliar application, soil inocula, in-furrow application, sidedress application, soil pre-treatement, wound inoculation, drip tape irrigation, vector-mediation via a pollinator, injection, osmopriming, hydroponics, aquaponics, and aeroponics.

In some embodiments, the endophytic bacteria is Bacillus amyloliquefaciens, Pantoea dispersa, Pseudomonas oryzihabitans, Pseudomonas oleovorans, Enterobacter cloacae, or Achromobacter xylosoxidans, alone or in combination.

Any endophytic bacteria described herein can also be applied to host plants in soil drenching approaches. For example, freeze dried preparation of Bacillus amyloliquefaciens can be mixed with a liquid carrier (comprising water, buffers, plant nutrients, and microbial nutrients,). This liquid preparation of bacterium and carrier (with additives) may be applied to the soil around plant or seed or in the alternative be applied to soil and plants using a commercial sprayer.

The following materials and methods are provided to facilitate the practice of Example I.

Host Collection and Isolation of Bacteria

Seeds were collected in order to isolate microorganisms from non-cultivated plants in the Malvaceae family. Seeds of Thespesia populnea trees were collected at Luquillo, PR and Rincón, PR. Fibers and seeds of wild, non-cultivated Gossypium hirsutum plants were collected from Guayama, PR. Thespesia populnea seeds were surface sterilized with 4% NaOCl for 20 minutes and placed on potato dextrose agar (PDA). Germinating seeds were grown on potting soil. Leaves from T. populnea seedlings were surface sterilized with 4% NaOCl and inoculated on PDA to isolate endophytic bacteria. Fibers and seeds from non-cultivated Gossypium hirsutum plants were inoculated on PDA and incubated at room temperature (25° C.).

Seeds of Thespesia populnea, non-cultivated Gossypium hirsutum, purchased delinted cultivated cotton and Clemson Spineless okra seeds were used to determine the frequency of seeds with culturable microbes. A primary goal of these experiments was to determine whether acid delinted cotton seeds had fewer microbes than seeds from wild, non-cultivated plants in the Malvaceae family. Seeds from wild, non-cultivated cotton were removed from bulk fibers, treated with 25% sulfuric acid for 5 minutes, and washed 3× with sterile distilled water. Acid delinted seeds were placed on PDA, incubated at 25° C. and observed after 72 hours for evidence of microbial growth. This experiment was also designed to determine whether treating seeds with 4% NaOCl for 20 minutes was sufficient to decrease the frequency of seeds with culturable surface microbes to generate seeds for future inoculations and testing. Seeds of Thespesia populnea, delinted cultivated cotton, and Clemson Spineless okra seeds were surface sterilized with 4% NaOCl for 20 minutes and washed 3× with sterile water. Seeds were placed on PDA and incubated at room temperature for 72 hours.

Identification of Bacteria

Bacterial DNA was extracted using GenElute™ Bacterial Genomic DNA Kit (Sigma-Aldrich, St. Louis, MO). The 16S rRNA gene was amplified using the primers 27F (5′-AGAGTTTGATCMTGGCTCAG; SEQ ID NO: 19) and 1492R (5′-TACCTTGTTACGACTT; SEQ ID NO: 20). Polymerase chain reactions (PCR) were initially denatured for 5 minutes at 95° C. followed by 30 cycles of 1 minute at 94° C., 1 minute at 55° C., and 1.5 minutes at 72° C. along with a final extension step of 10 minutes at 72° C. PCR reactions were purified using the QIAquick PCR Purification Kit (Qiagen, Valencia, CA) and sent to GENEWIZ (South Plainfield, NJ) for sequencing. Sequences were analyzed using BLAST (Altschul et al. 1990).

Plant Growth Promoting Potential of Bacteria from Non-Cultivated Malvaceae Plants

Bacteria were grown using a variety of culture media to determine their plant growth promoting potential. Bacteria were inoculated onto PDA with 5% and 10% NaCl to determine their salt tolerance. Potato dextrose agar was used to utilize a plant-based medium throughout these experiments. In addition, bacteria were grown on skim milk agar, Pikovskaya's agar, and nitrogen free liquid media to determine their abilities to secrete proteases, solubilize phosphate, and fix nitrogen, respectively. Bacteria were also tested for their ability to produce indole acetic acid (IAA). Bacteria were grown on potato dextrose broth with 1 mg/ml of tryptophan (Acuna et al 2011). After 5 days of growth, 1 ml of each bacterial culture was centrifuged and 2 ml of Salkowski reagent was added to the supernatant of each sample. Reactions were incubated in the dark for 30 minutes. Reactions that developed into a dark magenta color were considered positive for the production of IAA.

Germination and Radicle Development of Cultivated Cotton Seeds Inoculated with Bacteria from Non-Cultivated Plants

Cotton seeds were surface sterilized with 50% bleach for 20 minutes and washed 3× with sterile distilled water. Seeds were placed in Petri dishes containing 0.7% agarose and treated with 10 μl of different bacterial suspensions. Suspensions had approximately 10⁸ cells/ml of Bacillus amyloliquefaciens (strain Bamy), Curtobacterium oceanosedimentum (strain WCB1), or Pseudomonas oryzihabitans (strain WCB2) or combinations of equal parts of these three species. Seed germination was observed after 24, 48, 72 hours (n=18). In addition, the length of cotton seedling radicles was measured after 24, 48, 72, and 96 hours to determine if bacteria promoted early radicle growth (n=10).

Application of Bacillus amyloliquefaciens on Cotton Seeds as a Source of Rhizobacteria

Experiments were performed to ascertain whether the beneficial bacteria applied to seeds could be successfully vectored to soil and seedling rhizosphere. Cotton seeds were surface sterilized and soaked for 1 hour in a bacterial suspension of 10⁸ cell/ml of Bacillus amyloliquefaciens (strain Bamy) in water or 150 mM NaCl. Seeds were air dried and placed into magenta vessels containing 20 g twice-sterilized soil. After 36 hours and upon seed germination and radicle emergence, a sterile micropipette tip was used as a probe to harvest soil surrounding the emerging seedling radicle. The soil collected was spread onto PDA. Petri dishes were observed after 48 hours for evidence of bacterial growth.

Growth of Salt Stressed and Non-Stressed Cotton Seedlings Inoculated with Bacillus amyloliquefaciens

Delinted, cultivated cotton seeds were inoculated with Bacillus amyloliquefaciens to evaluate whether the bacteria promoted growth of cotton seedlings. Magenta vessels containing 20 grams of soil were twice sterilized, once every 24 hours. Suspensions containing 10⁸ cells/ml of Bacillus amyloliquefaciens (strain Bamy) were prepared in sterile water and in a 150 mM NaCl solution. Cotton seeds were surface sterilized in 4% NaOCl for 20 minutes, soaked in the bacterial suspensions for 1 hour, placed in the sterile soil, and kept at room temperature (25° C.) for the duration of the growth period. Uninoculated seeds were included as controls in both salt stressed and non-stressed conditions. Eleven cotton seedlings per treatment were grown for 10 days under grow lamps set to a 16 h/8 h light cycle. After 10 days, the soil was removed and roots were washed. The shoot height (mm), primary root length (mm), number of lateral roots, and length of the longest lateral root (mm) of cotton seedlings were measured. In addition, seedlings were placed in an incubator at 60° C. for 72 hours and the dry weights of seedling roots and shoots were used to calculate the root to shoot ratio.

Evaluating the Effects of Bacillus amyloliquefaciens on the Root Architecture of Cotton Seedlings

The root imaging analysis software WinRHIZO® (Regent Instruments Inc., Quebec, Canada) was used to further study the effects of inoculating cotton seedlings with Bacillus amyloliquefaciens (strain Bamy) on their root architecture. Inoculated and salt stressed cotton seedlings were grown as previously described for 7 days. Ten seedlings per treatment were removed from magenta vessels, washed, and scanned at 400 dpi in an Epson Expression 1680 scanner. Images of scanned roots were analyzed with WinRHIZO®. The software was used to measure the total root length (cm) which includes the length of both the primary and lateral roots, total root surface area (cm³), number of tips, and root diameter (cm) of 7-day-old cotton seedlings.

Growth Promotion of Cotton Seedlings Inoculated with B. amyloliquefaciens in Non-Sterile Conditions

Experiments were performed to determine whether Bacillus amyloliquefaciens (strain Bamy) enhanced cotton seedling growth in non-sterile conditions. Cotton seeds were treated with a solution of 10⁸ cells/ml of Bacillus amyloliquefaciens (strain Bamy) for 1 hour and sown onto non-sterile soil treated with water or a 150 mM NaCl solution. Control samples consisted of seeds that were not inoculated with the bacteria. Sixteen cotton seeds were placed in a large pot for each treatment and placed under a growth lamp with a 16 h/8 h light cycle. A total of 64 seeds were used for this experiment. Pots were treated with water or saline solution daily. The frequency of cotton seed germination was calculated at 5 and 7 days.

Growth Promotion and Root Architecture of Non-Stressed and Salt Stressed Okra Seedlings Inoculated with B. amyloliquefaciens

It was also determined whether B. amyloliquefaciens (strain Bamy) also enhanced growth and altered root architecture of okra seedlings. Seeds were soaked in a solution containing 10⁸ cells/ml of Bacillus amyloliquefaciens (strain Bamy) in sterile water or 100 mM NaCl. Control samples consisted of seeds that were not inoculated with the bacteria. Okra seedlings were grown in magenta vessels containing 20 g of twice sterilized soil. Growth promotion was evaluated by measuring shoot height and primary root length. In addition, WinRHIZO® was used to measure total root length (cm), total root surface area (cm³), number of tips, and root diameter (cm). Ten, 6-day-old okra Clemson seedlings were analyzed per treatment.

Statistical Analysis

Significant difference between the means of treated and control samples was determined by carrying out two tailed Student's T-tests. Significant difference between samples was attributed when p<0.05.

The following examples are provided to illustrate certain embodiments of the invention. They are not intended to limit the invention in any way.

### Example I

### Host Promotional Properties of Endophytic Bacteria from Seeds of Non-Cultivated Cotton (Malvaceae Spp.)

Bacteria were isolated from non-cultivated cotton bolls and fibers sampled at the southern coastal municipality of Guayama, Puerto Rico. Gossypium hirsutum is a member of the Malvaceae family and is a native plant of Puerto Rico.

Colonization Frequency of Culturable Seed-Transmitted Bacteria

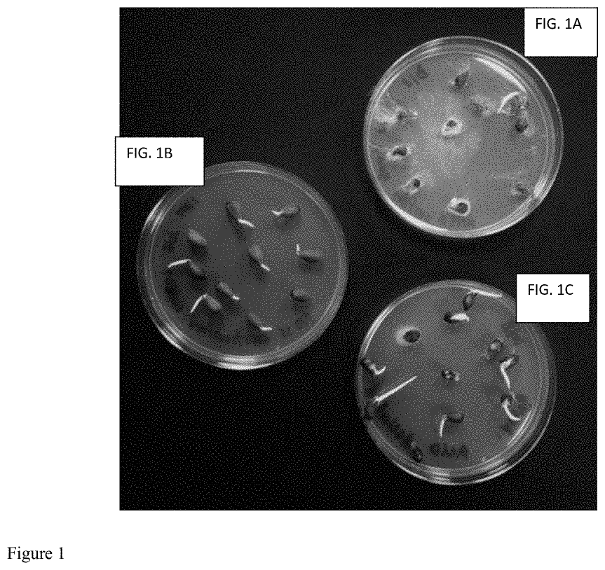

The frequency of seeds containing culturable bacteria was determined (Table 2). The non-surface sterilized seeds with the greatest frequency of culturable bacteria were collected from Thespesia populnea (100%), Clemson Spineless okra (100%), and wild, non-cultivated cotton (45%). Acid delinted, non-sterilized, cultivated cotton seeds had 0% frequency of culturable bacteria when inoculated on PDA. Acid delinting non-cultivated cotton seeds with 25% sulfuric acid greatly reduced the frequency of seeds with culturable bacteria. Non-surface sterilized and non-cultivated cotton seeds had 45% of bacterial colonization while 4.5% of cotton seeds delinted with 25% sulfuric acid had culturable bacteria. Acid delinting or treating seeds with 4% NaOCl reduced the amount of culturable microbes on seeds (Table 2, FIG. 1). In addition to reducing the growth of bacteria, acid delinting and commercially purchased cotton seeds had decreased growth of fungi as observed in FIG. 1.

TABLE 2
Frequency of seeds from various hosts in the Malvaceae family
colonized by culturable seed-transmitted bacteria.

| | | % of seeds with | | % of seeds with | |
|---|---|---|---|---|---|
| | | superficial bacteria | | endophytic bacteria | |
| | | (non-surface | | (surface | |
| Seed | Seed source | sterilized) | | sterilized) | |
| Thespesia populnea | Rincón, Puerto | 100% | (n = 25) | 6.7% | (n = 30) |
| | Rico | | | | |
| Wild, non-cultivated | Guayama, | 45% | (n = 20) | 4.5% | (n = 22) |
| Gossypium hirsutum | Puerto Rico | | | | |
| Cultivated cotton | Unknown, | 0% | (n = 20) | 0% | (n = 60) |
| | Purchased | | | | |
| Clemson Spineless | Unknown, | 100% | (n = 20) | 0% | (n = 20) |
| okra | Purchased | | | | |

*Seeds of non-cultivated Gossypium hirsutum were delinted and surface sterilized with 25% sulfuric acid for 5 minutes. Seeds of Thespesia populnea, cultivated cotton, and Clemson Spineless okra were surface sterilized using 4% NaOCl for 20 minutes.

Three different bacteria were selected from Thespesia populnea and Gossypium hirsutum and identified through 16S rRNA gene sequencing. B. amyloliquefaciens (strain Bamy), Curtobacterium oceanosedimentum (strain WCB 1) and Pseudomonas oryzihabitans (strain WCB2) grew on PDA with 5% NaCl. Furthermore, Bacillus amyloliquefaciens grew on PDA containing 10% NaCl. Both Bacillus amyloliquefaciens (strain Bamy) and Curtobacterium oceanosedimentum (strain WCB1) produced proteases on skim milk agar and all three species solubilized phosphate on Pikovskaya's agar. Pseudomonas oryzihabitans (strain WCB2) tested negative for the production of proteases on skim milk agar. The three species grew on nitrogen free liquid media. In addition, C. oceanosedimentum (strain WCB1), and P. oryzihabitans (strain WCB2), tested positive for IAA production upon adding the Salkowski reagent. Bacillus amyloliquefaciens (strain Bamy) tested negative for IAA production in repeated experiments.

TABLE 3
Bacteria isolated from non-cultivated plants in the Malvaceae
family and their growth promoting potential.

| | | Accession | Plant | PDA + | PDA + | | | | |
|---|---|---|---|---|---|---|---|---|---|
| Taxonomy | Strain | No. | source | 5% NaCl | 10% NaCl | SMA | NFM | PVK | IAA |
| Bacillus | Bamy | KX622565 | T. | + | + | + | + | + | − |
| amyloliquefaciens | | | populnea | | | | | | |
| Curtobacterium | WCB1 | KX622563 | G. | + | − | + | + | + | + |
| oceanosedimentum | | | hirsutum | | | | | | |
| Pseudomonas | WCB2 | KX622564 | G. | + | − | − | + | + | + |
| oryzihabitans | | | hirsutum | | | | | | |

Germination of Cotton Seeds Inoculated with Bacteria

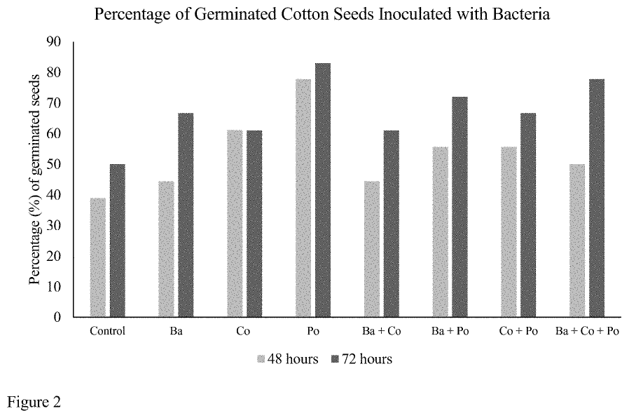

Inoculating surface sterilized cotton seeds with bacterial suspensions increased germination after 48 and 72 hours on 0.7% agarose plates (FIG. 2). Pseudomonas oryzihabitans (strain WCB2) isolated from wild, non-cultivated Gossypium hirsutum fibers increased the germination of cultivated cotton seeds the most out of the treatments applied. After 72 hours, 83% of cotton seeds inoculated with P. oryzihabitans (strain WCB2) had germinated whereas only 50% of seeds germinated in uninoculated control treatments. The second most effective treatment at enhancing cotton seed germination after 72 hours consisted of a suspension containing equal parts of Bacillus amyloliquefaciens (strain Bamy), Curtobacterium oceanosedimentum (strain WCB 1), and Pseudomonas oryzihabitans (strain WCB2). Seeds treated with an equal part mixture of these three isolates of bacteria had a 77.8% germination rate after 72 hours.

Length of Emerging Cotton Seedling Radicles Inoculated with Bacteria

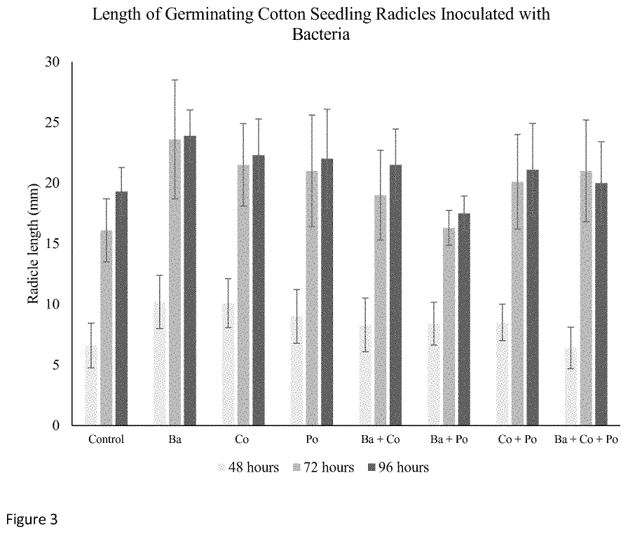

Upon germination, surface sterilized cotton seeds treated with bacterial suspensions had longer radicles than radicles from uninoculated seeds (FIG. 3). Seeds treated with Bacillus amyloliquefaciens (strain Bamy) had the longest radicles after 48, 72, and 96 hours and T-tests supported that they were significantly longer than uninoculated controls (p<0.00). The average length of cotton seedling radicles inoculated with Bacillus amyloliquefaciens (strain Bamy) was 24% greater than the length of the radicles from seedlings in the control treatment after 96 hours. In addition, seeds treated with a suspension of Curtobacterium oceanosedimentum (strain WCB1) had significantly longer radicles at 48, 72, and 96 hours (p≤0.016) and increased the length of the radicles by 15% after 96 hours. Furthermore, cotton seeds treated with Pseudomonas oryzihabitans (strain WCB2) also had significantly longer (p≤0.016) radicles and their length was enhanced by 14%. Cotton seeds treated with a mixture of Bacillus amyloliquefaciens (strain Bamy) and Pseudomonas oryzihabitans (strain WCB2) had longer radicles than control samples after 48 hours (p=0.04).

Bacteria on Cotton Seeds as a Source for Rhizosphere Bacteria

Bacillus amyloliquefaciens (strain Bamy) was recovered from the soil immediately surrounding cotton seedling radicles 48 hours after inoculated seeds had been placed into twice sterilized soil. Bacteria were recovered from soil treated with sterile water and from soil treated with a sterile solution of 150 mM NaCl. No bacteria were recovered from the soil surrounding radicles of surface sterilized seeds in uninoculated control treatments (FIG. 4). Aside from demonstrating that applying bacteria onto seeds leads to soil colonization, this experiment confirmed that the autoclaving method used in preparing magenta vessels and soil for enclosed experiments was effective.

Systemic Colonization of Bacillus amyloliquefaciens in Cotton Seedlings and Enhanced Cotyledon Expansion

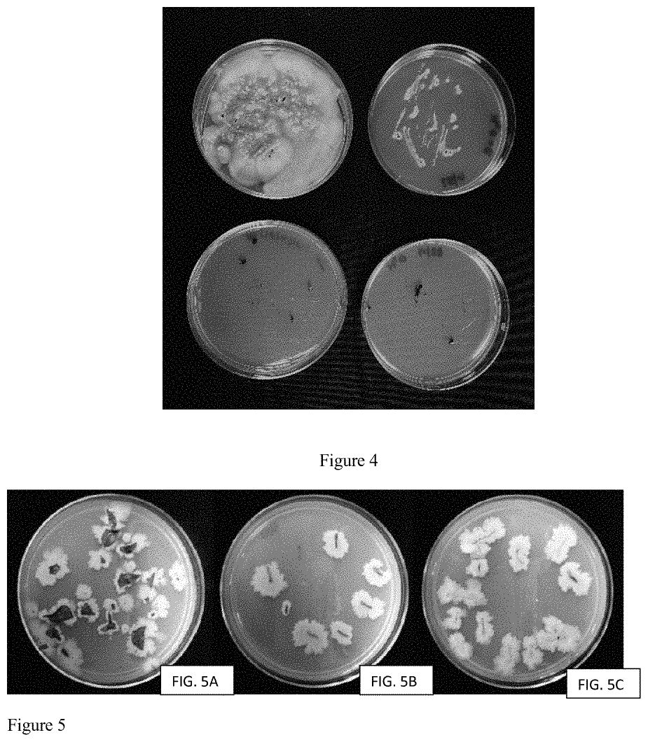

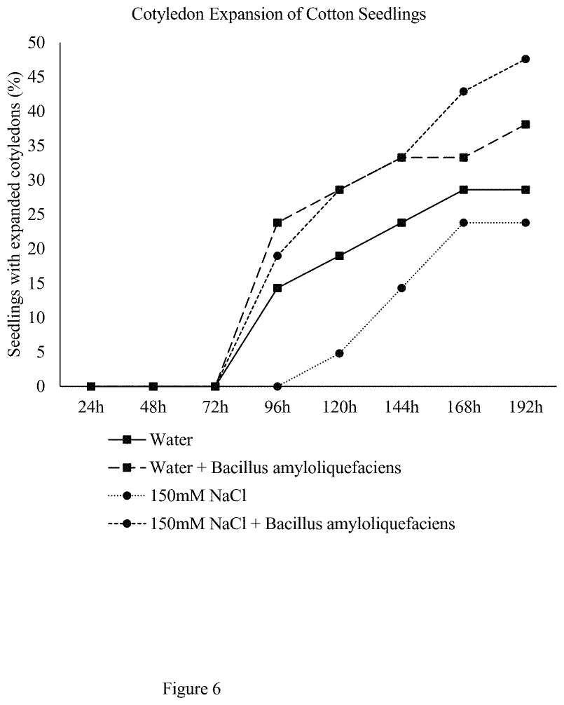

Bacillus amyloliquefaciens (strain Bamy) systemically colonized 7-day-old cotton seedlings and was re-isolated from surface sterilized cotyledons, stems, and roots (FIG. 5). During the course of an 8-day experiment, a greater percentage of seeds treated with B. amyloliquefaciens developed into seedlings with fully expanded cotyledons than uninoculated seedlings growing in both non-stressed and salt stressed conditions (FIG. 6). At the eighth day, 47.6% of inoculated cotton seeds in salt stressed conditions had developed into seedlings with expanded cotyledons compared to 23.8% of uninoculated seeds. In addition, 38.1% of inoculated cotton seeds in non-stressed conditions developed into seedlings with expanded cotyledons compared to 28.6% of uninoculated seeds.

Growth Promotion of Salt Stressed and Non-Stressed Cotton Seedlings Inoculated with Bacillus amyloliquefaciens

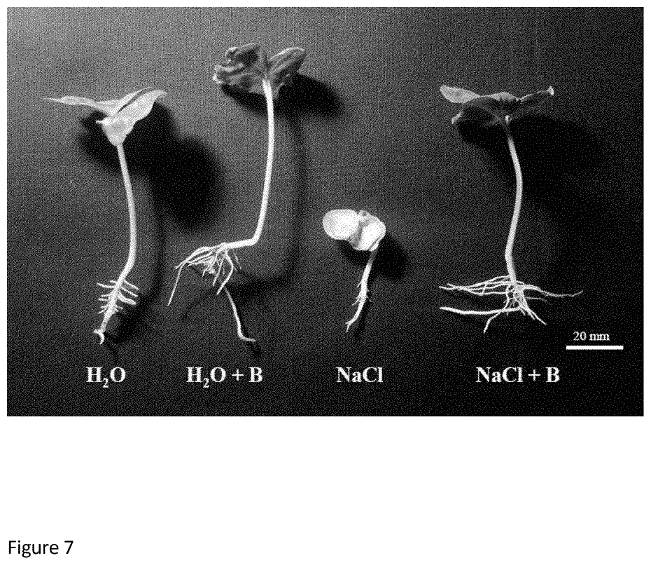

Growth promotion was observed in 10-day-old, non-stressed and salt stressed cotton seedlings inoculated with Bacillus amyloliquefaciens (strain Bamy) (Table 4, FIG. 7). The shoot height of inoculated and salt stressed cotton seedlings was significantly greater (p=0.04) than the shoot height of uninoculated seedlings. The shoot height was also slightly greater in non-stressed and inoculated seedlings but the increase in height was not significant after 10 days of growth (p=0.27).

The primary root length of non-stressed and salt stressed inoculated cotton seedlings was also significantly greater than uninoculated seedlings (p=0.00). The length of cotton seedling primary roots inoculated with Bacillus amyloliquefaciens (strain Bamy) was over 3× greater than the length of primary roots of uninoculated cotton seedlings in both non-stressed and salt stressed conditions. In addition to increasing the primary root length, cotton seedlings treated with B. amyloliquefaciens (strain Bamy) had a greater number of lateral roots and longer lateral roots compared to uninoculated seedlings. The dry weight of shoots and roots of 10-day-old cotton seedlings were used to calculate the average root to shoot ratio of seedlings in each treatment.

The root to shoot ratio of inoculated cotton seedlings was significantly greater than the root to shoot ratio of uninoculated seedlings in both salt stressed and non-stressed conditions (both p=0.00).

TABLE 4
Growth promotion of 10-day-old cotton seedlings inoculated with Bacillus amyloliquefaciens
(n = 11). Ba = Bacillus amyloliquefaciens (strain Bamy)

| | | | Number of | Length of the | |
|---|---|---|---|---|---|
| | Shoot height | Primary root | lateral roots | longest lateral | Root to shoot |
| Treatment | (mm) | length (mm) | (mm) | root (mm) | ratio |
| Water | 62.82 ± 6.84 a | 29.09 ± 6.56 a | 20 ± 4.40 a | 14.64 ± 5.30 a | 0.21 ± 0.04 a |
| Water + Ba | 67.64 ± 12.22 a | 93.55 ± 13.80 b | 34.55 ± 8.05 b | 40.45 ± 7.49 b | 0.35 ± 0.06 b |
| 150 mM NaCl | 59.18 ± 9.35 a | 34.18 ± 6.81 a | 22.55 ± 4.37 a | 14.45 ± 6.22 a | 0.20 ± 0.04 a |
| 150 mM NaCl + Ba | 67.36 ± 7.85 b | 106.27 ± 12.75 c | 30.64 ± 5.54 b | 28.27 ± 9.22 c | 0.25 ± 0.05 c |

Root Architecture and Development of Cotton Seedlings Inoculated with Bacillus amyloliquefaciens

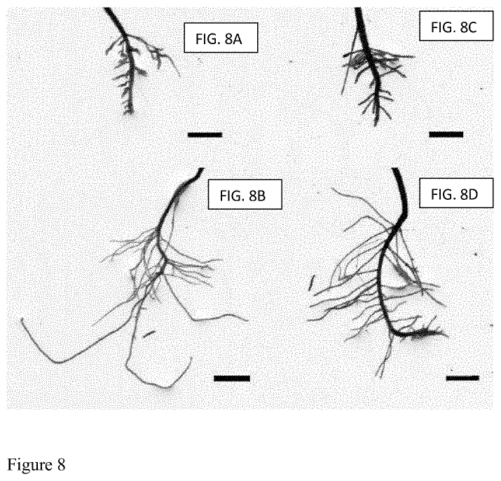

Differences in root growth and development were observed in 7-day-old cotton seedlings inoculated with B. amyloliquefaciens (strain Bamy) grown under salt stressed and non-stressed conditions (FIG. 8). Inoculating cotton seeds with bacteria led the development of seedlings with greater total root length, total root surface area, and number of tips compared to uninoculated cotton seedlings. In contrast, inoculating the seedlings with the bacteria led to a decrease in root diameter (Table 5). The total root surface area of cotton seedlings inoculated with the bacteria was significantly greater than that of uninoculated salt stressed and non-stressed seedlings (p=0.00 and p=0.01). Inoculating Bacillus amyloliquefaciens (strain Bamy) onto cotton seeds led to the development of seedlings with a greater amount of root branching as supported by an increased number of tips (Table 5). The number of tips on salt stressed cotton seedlings roots inoculated with B. amyloliquefaciens (strain Bamy) was 96% greater than that of uninoculated seedlings (p=0.02). Inoculating cotton seeds with the bacteria also led to the development of cotton seedlings with thinner roots in both salt stressed and non-stressed growth conditions (both p=0.00).

TABLE 5
Effects of inoculating Bacillus amyloliquefaciens on the root architecture of 7-
day-old cotton seedlings (n = 10). Ba = Bacillus amyloliquefaciens (strain Bamy)

| | | Total root | | Root |
|---|---|---|---|---|
| | Total root | surface area | Number of | diameter |
| Treatment | length (cm) | (cm²) | tips | (mm) |
| Water | 9.4 ± 3.01 a | 3.48 ± 0.73 a | 25.7 ± 5.12 a | 1.22 ± 0.13 a |
| Water + Ba | 19.71 ± 3.35 b | 6.03 ± 0.77 b | 49 ± 9.65 b | 1.00 ± 0.06 b |
| 150 mM NaCl | 11.26 ± 3.71 a | 3.80 ± 1.04 a | 23.5 ± 3.54 a | 1.19 ± 0.24 a |
| 150 mM NaCl + Ba | 27.75 ± 6.86 c | 6.67 ± 0.56 c | 46.6 ± 11.93 b | 0.80 ± 0.09 c |

*Different letters indicate significant difference between means according to Student's T-tests (p < 0.05).

Germination of Cotton Seeds Inoculated with Bacillus amyloliquefaciens in Non-Sterile Conditions

The percentage of germinated cotton seeds inoculated with Bacillus amyloliquefaciens (strain Bamy) was greater than that of uninoculated seeds in both salt stressed and non-stressed conditions (Table 6). The percentage of germinated seeds per treatment was calculated after 5 and 7 days of inoculation. After 5 days of growth in non-stressed conditions, 50% of cotton seeds inoculated with B. amyloliquefaciens (strain Bamy) germinated while 31.3% of uninoculated seeds germinated. In addition, cotton seed germination was greater in inoculated seeds compared to uninoculated seeds in salt stressed conditions. After 5 days of being inoculated with B. amyloliquefaciens (strain Bamy) and sown in non-sterile soil, 12.5% of cotton seeds had germinated while 6.3% of uninoculated seeds germinated in salt stressed conditions. Germination of inoculated cotton seeds was also greater than seeds in control treatments after 7 days of growth (Table 6).

TABLE 6
Germination of cotton seeds inoculated with Bacillus amyloliquefaciens
in non-stressed and salt stressed conditions. Seeds were sown
in non-sterile soil and grown in open containers (n = 16).
Ba = Bacillus amyloliquefaciens (strain Bamy)

| | Treatment | % of germinated seeds |
|---|---|---|
| 5 days | Water | 31.3 |
| | Water + Ba | 50 |
| | 150 mM NaCl | 6.3 |
| | 150 mM NaCl + Ba | 12.5 |
| 7 days | Water | 31.3 |
| | Water + Ba | 50 |
| | 150 mM NaCl | 12.5 |
| | 150 mM NaCl + Ba | 18.8 |

Growth Promotion and Altered Root Architecture of Okra Seedlings Inoculated with Bacillus amyloliquefaciens

Inoculating okra seeds with Bacillus amyloliquefaciens (strain Bamy) enhanced growth and altered root architecture of okra seedlings (Table 7). Inoculated okra seedling roots in salt stressed conditions had longer shoots than uninoculated seedlings. In addition, inoculating okra seeds with B. amyloliquefaciens (strain Bamy) led to the development of seedlings with greater total root length, surface area, and number of tips compared to uninoculated seedlings in both salt stressed and non-stressed conditions. Furthermore, seeds inoculated with B. amyloliquefaciens (strain Bamy) produced seedlings with thinner root diameters than those developing from uninoculated seeds.

TABLE 7
Growth promotion and changes in root architecture of okra Clemson seedlings inoculated with
Bacillus amyloliquefaciens in non-stressed and salt stressed conditions (n = 10).

| | Shoot | Primary | Total root | Total root | | Root |
|---|---|---|---|---|---|---|
| | height | root length | length | surface area | Number | diameter |
| | (mm) | (mm) | (cm) | (cm³) | of tips | (mm) |
| Water | 68.1 ± 12.98 a | 23.50 ± 2.88 a | 3.04 ± 0.60 a | 2.02 ± 0.16 a | 18.2 ± 7.73 a | 1.22 ± 0.1 a |
| Water + Ba | 74 ± 20.07 a | 67.40 ± 15.42 b | 10.93 ± 3.13 b | 5 ± 1.21 b | 79.9 ± 34.35 b | 0.95 ± 0.13 b |
| 100 mM NaCl | 12.4 ± 5.02 b | 19.50 ± 6.57 a | 1.73 ± 0.90 c | 1.42 ± 0.51 c | 14.4 ± 5.58 a | 1.25 ± 0.23 a |
| 100 mM NaCl + Ba | 18 ± 4.94 c | 51 ± 8.69 c | 5.81 ± 1.49 d | 2.67 ± 0.76 d | 46.1 ± 22.28 c | 1.01 ± 0.09 b |

Ba = Bacillus amyloliquefaciens (strain Bamy)
*Different letters indicate significant difference between means according to Student's T-tests (p < 0.05).

### Discussion

Acid Delinting Disturbs the Cotton Seed Microbiome

The colonization frequency survey (Table 2) demonstrated that wild, non-cultivated plants in the Malvaceae family such as Thespesia populnea and non-cultivated Gossypium hirsutum had a greater frequency of bacteria on seeds than those of cultivated and acid delinted cotton seeds. It was also demonstrated that using 25% sulfuric acid to remove fibers from cotton seeds decreased the amount of culturable bacteria present on the seed surface. Seeds of cultivated okra yielded bacteria from a high percentage of seeds. However, okra seeds are not acid treated and microbes may be present on the seed surface. The data presented herein support the hypothesis that delinting cotton seeds with sulfuric acid affects the native cotton seed microbiome.

As demonstrated herein, bioprospecting for microbes in non-cultivated relatives of crop plants in stressful environments is an effective strategy to isolate growth promoting microbes. Applying growth promoting and stress alleviating microbes as crop seed inoculants provides an effective strategy for enhancing plant growth and stress tolerance.

### Example II

### Bacillus amyloliquefaciens Alters Gene Expression, ROS Production, and Lignin Synthesis of Cotton Seedling Roots

The plant growth promoting and salt stress alleviating ability of Bacillus amyloliquefaciens (strain Bamy) on cotton seedlings was demonstrated by the data provided in Example I. This salt tolerant bacterium was originally isolated from Thespesia populnea and has various growth promoting properties such as phosphate solubilization and protease secretion.

The responses elicited in cotton seedling roots as a result of being inoculated with the PGPB B. amyloliquefaciens (strain Bamy) were further investigated. The changes in gene expression that occur when plants are inoculated with plant growth promoting bacteria (PGPB) are largely unknown.

Experiments were designed to determine the changes in gene expression that occur when cotton seedling roots are inoculated with the PGPB Bacillus amyloliquefaciens (strain Bamy). Microarrays and qRT-PCR were used to study gene expression of inoculated cotton seedling roots, thereby identifying enriched pathways that are induced upon inoculating cotton with Bacillus amyloliquefaciens (strain Bamy). Finally, seedlings were stained for ROS production and lignin synthesis to determine whether inoculating cotton seeds with B. amyloliquefaciens (strain Bamy) had an effect on ROS and lignin accumulation.

The following materials and methods are provided to facilitate the practice of Example II.

Seed Inoculation and Growth of Cotton Seedlings

Cotton seeds were surface sterilized with 4% NaOCl for 20 minutes and washed 3× with sterile distilled water. Seeds were submerged in a solution containing 10⁸ cells/ml of Bacillus amyloliquefaciens (strain Bamy). Seeds were sown into 20 g of twice-autoclaved soil in magenta vessels. Seedlings were grown at room temperature (25° C.) for 10 days under a grow lamp with a 16 h/8 h light cycle.

RNA Extraction of Cotton Seedling Roots

Seedlings were removed from magenta vessels and carefully washed to remove soil from roots. Approximately 0.1 g of a 10-day-old cotton seedling root from each treatment was ground in liquid nitrogen. A biological replicate was included and a total of 8 seedling roots were used. Total RNA of each root was extracted using an RNeasy Plant Mini Kit (Qiagen, Valencia, CA). RNA purity was evaluated using a Caliper LabChip GX and HT RNA Kit (Perkin Elmer, Waltham, MA). RNA concentrations were measured using a Trinean DropSense96 UV/VIS reader (Trinean, Gentbrugge, Belgium).

GeneChip Hybridization and Data Analysis

A GeneChip Cotton Genome Array (Affymetrix, Santa Clara, CA) was used for each sample. Each microarray consisted of 239,777 probe sets from 21,854 transcripts. In total, 8 cotton arrays were used. To prepare the samples for hybridization, 100 ng of total RNA from each sample were used to generate cRNA using the 3′IVT Plus amplification kit (Affymetrix, Santa Clara, CA). Amplification was carried out for 16 hours. The concentration of cRNA was verified and 12 g of each sample was added to each fragmentation reaction. Then, 10 g were loaded onto the GeneChip arrays. Chips were incubated in a hybridization oven for 16 hours at 45° C. and a 60 rpm rotation. Arrays were washed in a fluidics station and scanned using Affy GeneChip Scanner 3000 7G (Affymetrix, Santa Clara, CA). The data was normalized using Bioconductor and scatter plots were generated to determine if samples were replicable. The correlation between sample pairs were determined using Pearson's method.

Transcriptome Analysis Console 3.0 (Affymetrix, Santa Clara, CA) was used to identify DEGs in inoculated cotton seedling roots under both non-stressed and saline conditions. For each set, the log 2 scale of the average signal of both duplicates per sample was used to determine the fold-change of each probe. Genes with a 2-fold or greater change in expression and p-values≤0.05 were considered for further analysis. The DEGs were identified by searching the accession number for each corresponding probe using BLAST. Each gene was categorized based on data in Uniprot. The up-regulated Affymetrix probe IDs were also used for singular enrichment analysis (SEA) in agriGO (Du et al 2010). The background reference was selected as the cotton Affymetrix genome array. Fisher's exact test and the Hochberg (FDR) multi-test adjustment method were selected with 0.05 significance level. The minimum number of mapping entries was selected as 1. The transcript IDs and log 2 fold-change values of up and down-regulated genes were also used in MapMan to visualize changes in gene expression (Thimm et al 2004, Usadel et al 2009).

Microarray Validation and Gene Expression Analysis Using qRT-PCR

Quantitative RT-PCR was used to test the expression of up-regulated genes identified through the microarray analysis. RNA of cotton seedlings was extracted as previously described. The concentration and purity of the samples was measured using a NanoDrop® ND1000 spectrophotometer (Thermo Fisher Scientific, Wilmington, DE). A total of 6 genes were amplified by qRT-PCR using the actin gene was chosen as a control. Primers were designed using Primer Express@ Software (Thermo Fisher Scientific, Wilmington, DE), purchased and validated using a standard curve (Table 8). A Step One Plus Cycler (Applied Biosystems, Foster City, CA) was used to carry out RT-qPCR reactions. Reverse transcription reactions were carried out using the High Capacity cDNA Reverse Transcription Kit (Applied Biosystems, Foster City, CA) and qPCR reactions were prepared using Power SYBR Green Master Mix in 20 L reactions (Thermo Fisher Scientific, Wilmington, DE). Reverse transcription reactions were carried out at 50° C. for 30 minutes. qPCR conditions included an initial 10 minute step at 95° C., 40 cycles of 94° C. for 15 s and 60° C. for 60 s followed by a melt curve analysis. Data was analyzed in the Step One Plus Software and the relative expression of genes compared to the expression of the actin gene as a control was determined using the ΔΔCt method.

TABLE 8
Primers sets used for qRT-PCR validation
of microarray results.

| | | Tm | SEQ ID |
|---|---|---|---|
| Primer name | Sequence | (° C.) | NO. |
| GhPerox-F | GCGTGACGTGCTCAAACG | 59 | 21 |
| GhPerox-R | CATTGAGGGCGCCATGTT | 59 | 22 |
| GhPR1-F | AGTTGCGCAGCAGGTAAGGT | 59 | 23 |
| GhPR1-R | TCGCGCCAAACCACTTG | 59 | 24 |
| GhExpansin-F | CTGGCAGAGCAACACCTACCT | 58 | 25 |
| GhExpansin-R | CGCTTGTGGTGACTTGGAAA | 58 | 26 |
| GhNT-F | GACCATCAAGGTCCAGCTTTG | 58 | 27 |
| GhNT-R | GCCATGCACGGTCGACTT | 59 | 28 |
| GhNR-F | GACCATCAAGGTCCAGCTTTG | 59 | 29 |
| GhNR-R | GCCATGCACGGTCGACTT | 59 | 30 |
| GhActinB-F | CCGGTGACGGTGTTTCTCA | 59 | 31 |
| GhActinB-R | GCATGTGGAAGGGCATATCC | 58 | 32 |

Phloroglucinol-HCl Staining of Lignin in Cotton Seedling Roots Inoculated with B. amyloliquefaciens

Cotton seedlings were stained with phloroglucinol-HCl stain to determine whether inoculating seeds with B. amyloliquefaciens (strain Bamy) altered lignin synthesis in cotton seedling roots. Cotton seedlings were inoculated, grown, and treated as previously described above. Seedlings were removed from the soil and roots were washed with distilled water. Seedlings were placed in 50 ml sterile tubes with 20 ml phloroglucinol-HCl stain. Cotton seedling roots were observed using a light microscope after 4 hours of staining.

Cotton Microarray Analysis

Cotton genome arrays were used to determine the DEGs in non-stressed and salt stressed cotton seedling roots inoculated with Bacillus amyloliquefaciens (strain Bamy). The microarray analysis included two biological replicates from each treatment. The reproducibility between two sample replicates was determined using scatter plots. Sample replicates demonstrated to be highly reproducible in each of the four treatments. Correlation coefficients for all samples and their duplicates were greater than or equal to 0.98.

DEGs with a 2-fold change in expression or above and p-values<0.05 were selected for further analysis. Inoculated roots in favorable conditions had 252 DEGs. Out of those 252 DEGs, 139 genes were up-regulated and 113 were down-regulated. Inoculated cotton seedling roots in saline conditions had a total of 108 DEGs, out of which 76 were up-regulated and 32 were down-regulated.

DEGs in Non-Stressed Cotton Seedling Roots Inoculated with Bacillus amyloliquefaciens

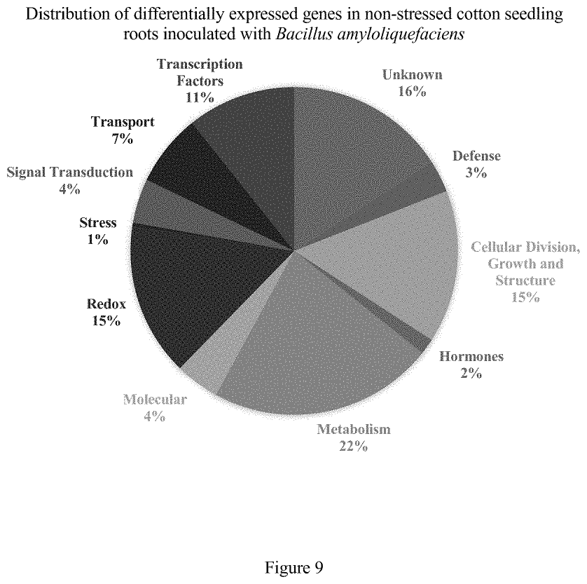

Most of the genes that were differentially expressed in non-stressed cotton seedling roots inoculated with bacteria were categorized under metabolism functions (22%) followed by those of unknown function (16%), cellular division, growth, and structure (15%), redox reactions (15%), transcription factors (11%), and transport (7%). It was observed that 3% of DEGs were related to defense mechanisms in cotton seedlings. Only 1% of DEGs in non-stressed and inoculated cotton seedling roots were categorized under stress-related genes (FIG. 9).

Up-Regulated Genes in Non-Stressed Cotton Seedling Roots Inoculated with Bacillus amyloliquefaciens

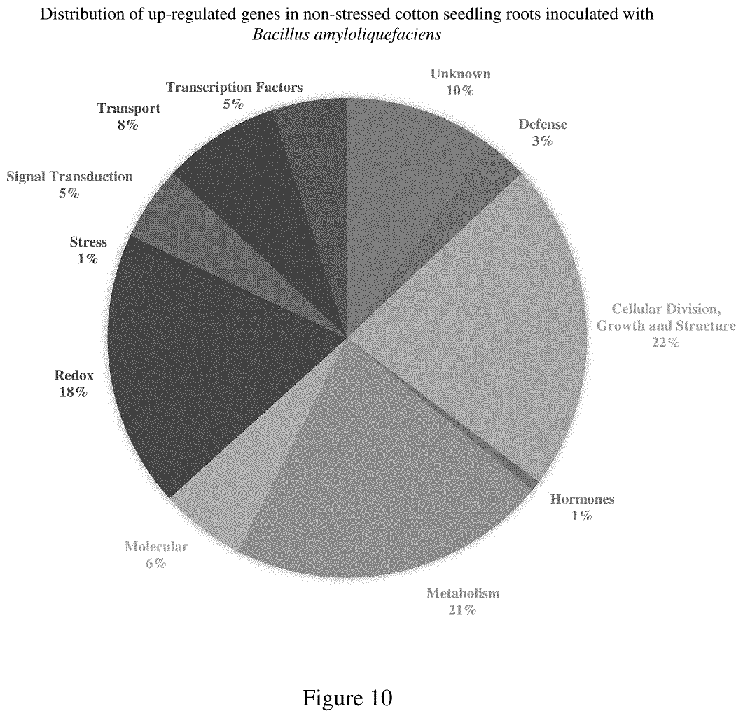

Of the 139 up-regulated genes in non-stressed cotton seedling roots, most belonged to the cellular division, growth, and structure category (22%) followed by the metabolism category (21%) (FIG. 10). The single most up-regulated gene in cotton seedling roots inoculated with B. amyloliquefaciens (strain Bamy) encoded for a WAT1-related protein which had a 14.75-fold change increase in expression (Table 9). Some up-regulated genes in the cell division, growth, and structure category encoded for predicted proteins such as tubulin, xyloglucan galactosyltransferase, fasciclin-like arabinogalactan protein, expansins, and pectinesterase, among others (Table 9). Some up-regulated genes in the metabolism category encoded for the predicted proteins glutamine synthetase, asparaginase, proteases, asparagine synthetase, and methyltransferases, among others. The most up-regulated gene in the metabolism category was asparagine synthetase with a 9.69-fold increase in expression followed by nitrate reductase with a 7.54-fold increase (Table 9). Inoculating cotton seedling roots with B. amyloliquefaciens (strain Bamy) also led to the increased expression of genes involved in oxidation-reduction reactions. Of the genes within the redox reaction category, the most up-regulated gene encoded for a predicted blue copper containing glycoprotein with a 7.41-fold increase in expression followed by a predicted peroxidase gene with a 6.73-fold increase. Additional genes in the redox category whose expression was up-regulated encoded for the predicted proteins ascorbate oxidase, monodehydroascorbate reductase, glucose-6-phoshate 1-dehydrogenase, and malate dehydrogenase (Table 9).

Down-Regulated Genes in Non-Stressed Cotton Seedling Roots Inoculated with Bacillus amyloliquefaciens

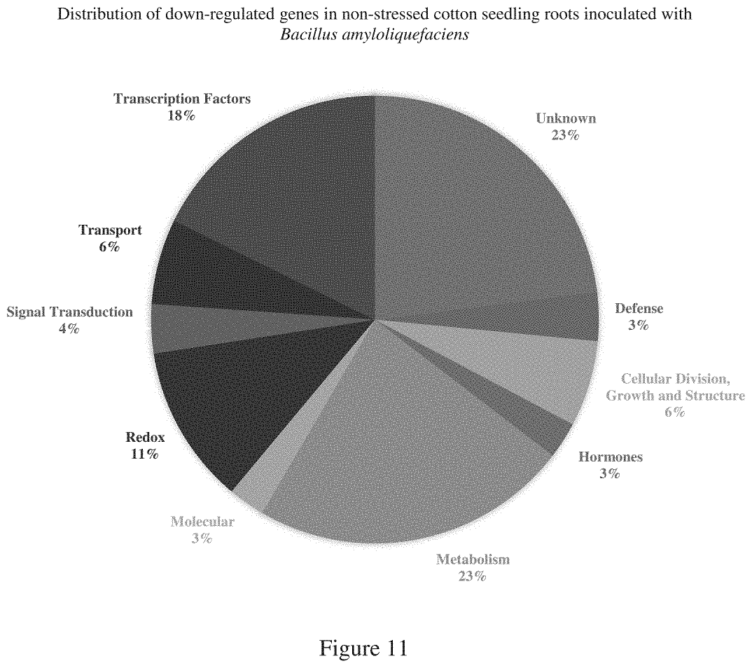

A total of 113 genes were down-regulated in cotton seedling roots inoculated with Bacillus amyloliquefaciens (strain Bamy). Most of the genes whose expression was reduced belonged to the metabolism (23%) and unknown category (23%). Down-regulated genes also belonged to the category of transcription factors (18%), redox (11%), cellular division, growth and structure (6%), transport (6%), signal transduction (4%), defense (3%), hormones (3%), and molecular function (3%) (FIG. 11). The single most individually down-regulated gene in the metabolism category encoded for a predicted salicylate carboxy methyltransferase protein which had a 47.79-fold decrease in expression. The second most down-regulated gene in non-stressed cotton seedling roots inoculated with the bacteria encoded for metacaspase-9 with a 43.67-fold decrease in expression compared to uninoculated roots. The most down-regulated gene transcript in the transcription factor category was identified as a predicted zinc finger protein (ZAT10) and WRKY94, and WRKY70 with a 4.64, 3.88, and 3.69-fold decrease in expression, respectively (Table 10).

DEGs in Salt Stressed Cotton Seedling Roots Inoculated with Bacillus amyloliquefaciens

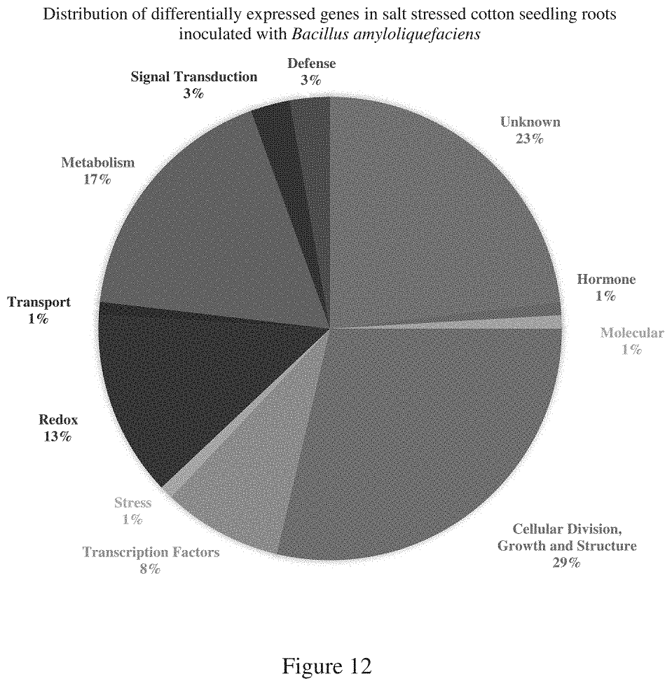

Out of the 108 DEGs in salt stressed cotton seedlings, most were placed within the category of cellular division, growth, and structure (29%) followed by those of unknown function (23%), metabolism (17%), and redox (13%), transcription factors (8%), defense (3%), and signal transduction (3%). Only 1% of DEGs were categorized under transport, hormone, stress and molecular functions (FIG. 12).

Up-Regulated Genes in Salt Stressed Cotton Seedling Roots

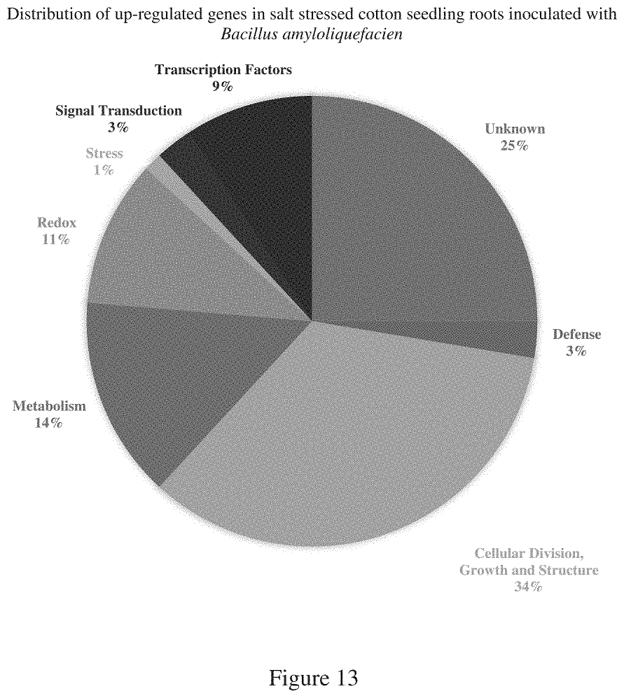

A total of 76 genes were up-regulated in inoculated and salt stressed cotton seedling roots (Table 9). The most up-regulated transcripts encoded for a predicted blue copper protein, expansin, and laccase with a 6.87, 6.71, and 5.39-fold increase in expression compared to uninoculated control samples (Table 11). Most of the up-regulated genes belonged to the cellular division, growth, and structure category (34%) followed by those categorized as unknown (25%), metabolism (14%), redox (11%), transcription factors (9%), defense (3%), signal transduction (3%), and stress (1%) (FIG. 13). Out of the up-regulated genes in the cellular division, growth, and structure category the most up-regulated gene transcripts encoded for the predicted proteins expansin, WLIM1, pectinesterase, and polygalacturonase (Table 9). It was also determined that 5 out of the 76 up-regulated genes in inoculated, salt-stressed cotton roots encoded for predicted ethylene-related transcription factors.

Down-Regulated Genes in Salt Stressed Cotton Seedling Roots

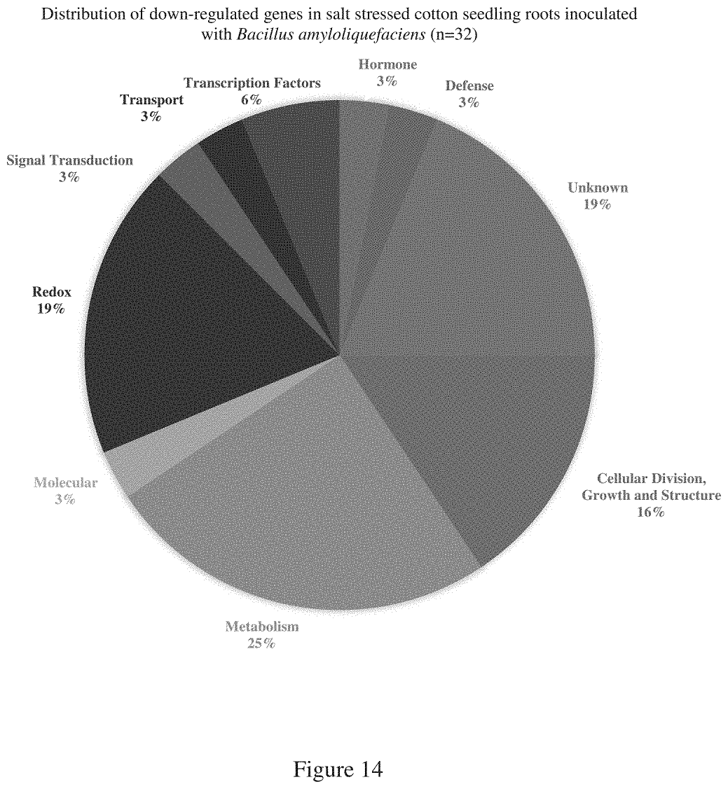

Thirty-two genes were down-regulated in inoculated cotton seedling roots in salt stress conditions. Most of the genes that were down-regulated in salt stressed and inoculated roots where grouped in the metabolism category (25%), followed by redox (19%), unknown (19%), cellular division, growth and structure (16%), and transcription factors (6%) (FIG. 14). The most down-regulated genes encoded for the predicted proteins expansin B1, cucumisin, and UDP-glycosyltransferase with 6.56, 5.98, and 4.4-fold decrease in expression, respectively (Table 10). The most down-regulated gene in the defense category encoded for a predicted metacaspase 3 with a 3.3-fold decrease in expression. It was also determined that the most down-regulated gene in the redox category encoded for a predicted alcohol dehydrogenase with a 3.23-fold decrease in expression. The most down-regulated transcript in the transcription factor category encoded for a predicted zinc finger protein ZAT11 with a 3.31-fold decrease in expression. The predicted transcript for the eceriferum gene had a 2.34-fold decrease in expression (Table 10).

TABLE 9
Exemplary up-regulated genes in cotton seedling roots in non-stressed conditions inoculated
with the growth promoting bacterium Bacillus amyloliquefaciens (strain Bamy).

| | | | Fold-Change | | |
|---|---|---|---|---|---|
| Transcript ID | Accession No. | Description | (linear) | Category | p-value |
| Gra.2669.1 | CO085938 | WAT1-related protein | 14.75 | T | 0.013 |
| Ghi.10557.1 | DN758244 | asparagine synthase | 9.69 | M | 0.023 |
| Gra.718.1 | CO085887 | nitrate reductase | 7.54 | Redox | 0.010 |
| GhiAffx.22444.1 | DT459384 | Mavicyanin | 7.41 | Redox | 0.007 |
| Ghi.6876.3 | DT461954 | metallothiol transferase FosB | 7.17 | M | 0.002 |
| Ghi.6234.2 | CO491431 | Peroxidase | 6.73 | Redox | 0.014 |
| GhiAffx.61409.1 | DW509160.1 | non-specific lipid-transfer protein A | 5.48 | T | 0.021 |
| Ghi.6234.1 | CO493806 | Peroxidase | 5.46 | Redox | 0.003 |
| Ghi.8419.1 | CO492888 | Unknown | 5.34 | — | 0.034 |
| Ghi.8448.1 | AF521240.1 | tubulin beta-7 chain | 5.34 | G | 0.044 |
| GraAffx.28607.1 | CO085993 | Unknown | 5.04 | — | 0.006 |
| Ghi.5484.1 | DT047572 | ascorbate oxidase | 4.82 | Redox | 0.000 |
| Ghi.3394.1 | DT465656 | cadmium resistance 6 | 4.63 | T | 0.013 |
| Ghi.2396.1 | DR455743 | ascorbate oxidase | 4.53 | Redox | 0.026 |
| Ghi.9446.1 | DT047992 | tubulin beta-2-chain | 4.51 | G | 0.036 |
| Ghi.7226.1 | AW186996 | Unknown | 4.5 | — | 0.026 |
| Ghi.716.1 | DR455728 | defensin like protein 1 | 3.97 | D | 0.015 |
| Ghi.9253.2 | DT466833 | ascorbate oxidase | 3.87 | Redox | 0.002 |
| Ghi.850.1 | DR454028 | yellow leaf senescence 9 | 3.84 | D | 0.029 |
| GhiAffx.40791.1 | DW499532.1 | RNA-binding protein 38 | 3.83 | Mol | 0.041 |
| Ghi.3151.1 | DT468993 | xyloglucan galactosyltransferase GT17 | 3.82 | G | 0.012 |
| Ghi.10775.1 | DN759794 | glutamine synthetase leaf isozyme, | 3.81 | M | 0.002 |
| | | chloroplastic like | | | |
| GhiAffx.6228.2 | DW498003.1 | glucose-6-phosphate 1-dehydrogenase | 3.79 | Redox | 0.003 |
| Ghi.4629.2 | CO499249 | isoflavone reductase | 3.77 | Redox | 0.050 |
| Gra.1351.1 | CO107103 | DNA-damage-repair/toleration protein | 3.73 | Mol | 0.024 |
| | | DRT100 | | | |
| GhiAffx.48851.1 | DW482562.1 | monodehydroascorbate reductase 5 | 3.63 | Redox | 0.037 |
| Ghi.3170.2 | DT468829 | ferredoxin-nitrite reductase | 3.5 | Redox | 0.005 |
| Ghi.8203.1 | AI729411 | basic leucine zipper 61 | 3.48 | TF | 0.015 |
| Ghi.4685.1 | DT051431 | glucose-6-phosphate 1-dehydrogenase | 3.47 | Redox | 0.002 |
| Ghi.4315.1 | DT455587 | transcription factor bHLH113 | 3.46 | TF | 0.045 |
| GhiAffx.25489.1 | DW232960.1 | malate dehydrogenase | 3.4 | Redox | 0.016 |
| Ghi.4828.1 | DT050294 | UTP-glucose-1-phosphate uridylyltransferase | 3.3 | M | 0.033 |
| Ghi.68.1 | DR176761 | fasciclin-like arabinogalactan protein 9 | 3.25 | G | 0.019 |
| Gra.1548.1 | CO123811 | isoaspartyl peptidase/L-asparaginase 2 | 3.23 | M | 0.006 |
| Ghi.3212.1 | DT467489 | Peroxidase | 3.2 | Redox | 0.044 |
| Gra.2653.1 | CO084400 | luminal-binding protein 5 | 3.19 | G | 0.007 |
| GraAffx.1872.1 | CO123596 | UTP-glucose-1-phosphate uridylyltransferase | 3.18 | M | 0.018 |
| Ghi.7383.1 | AI731186 | coatomer subunit zeta-2 | 3.18 | T | 0.022 |
| Ghi.1421.1 | DV848729 | Unknown | 3.17 | — | 0.038 |
| GhiAffx.20319.1 | DW493277.1 | cyclin U2-l-like | 3.16 | G | 0.002 |
| Ghi.5537.1 | DT047379 | proteasome subunit alpha type-5 | 3.11 | M | 0.049 |
| GhiAffx.11691.1 | DW229345.1 | basic 7S globulin | 3.1 | M | 0.007 |
| Ghi.4241.1 | DT456606 | beta-D-xylosidase 7 | 3.05 | G | 0.024 |
| GhiAffx.30987.1 | DW481679.1 | SNARE 11 | 3.03 | G | 0.033 |
| GraAffx.1029.1 | CO127686 | glucose-6-phosphate 1-dehydrogenase | 3.01 | Redox | 0.031 |
| GhiAffx.19944.1 | DW231640.1 | GPI-anchored protein | 3 | — | 0.011 |
| Gra.2669.1 | CO085938 | WAT1-related protein | 3 | T | 0.023 |
| Ghi.6433.1 | CK988080 | pathogenesis-related protein 1 | 2.99 | D | 0.019 |
| Ghi.7832.1 | DT573045 | tubulin alpha-3 chain | 2.98 | G | 0.037 |
| Ghi.9707.1 | DT051791 | tubulin beta-9 chain | 2.96 | G | 0.018 |
| Ghi.10183.1 | DT562914 | kinesin-like protein KIN-7D | 2.95 | G | 0.026 |
| Gra.2483.1 | CO074903 | asparagine synthetase | 2.94 | M | 0.000 |
| Ghi.4649.1 | DT047634 | ferredoxin--NADP reductase | 2.94 | Redox | 0.034 |
| Ghi.3295.2 | DT467569 | siroheme synthase | 2.93 | M | 0.024 |
| GhiAffx.62090.2 | DW501790.1 | snakin-2 | 2.91 | D | 0.007 |
| Ghi.6281.1 | DR456049 | transmembrane 9 superfamily member 12 | 2.89 | T | 0.042 |
| GhiAffx.58804.1 | DW496210.1 | SNARE 11 | 2.82 | G | 0.018 |
| Ghi.10297.1 | DR458723 | luminal-binding protein 5 | 2.79 | M | 0.035 |
| Ghi.8856.1 | DT466900 | Ferredoxin | 2.75 | Redox | 0.003 |
| Ghi.3375.1 | DT466692 | Unknown | 2.74 | — | 0.005 |
| Ghi.7874.1 | AY962572.1 | ethylene responsive transcription factor | 2.73 | TF | 0.049 |
| GhiAffx.15520.1 | DW496425.1 | deSI-like protein | 2.72 | M | 0.041 |
| GhiAffx.59931.1 | DW506084.1 | 14 kDa proline-rich protein DC2.15 | 2.71 | G | 0.020 |
| GhiAffx.14333.1 | CD486517 | expansin-A8 | 2.7 | G | 0.042 |
| GhiAffx.34037.1 | DW244974.1 | Calreticulin | 2.67 | M | 0.032 |
| Ghi.8128.1 | AY827548.1 | EREB1 transcription factor | 2.67 | TF | 0.044 |
| Ghi.5147.1 | DR462373 | monodehydroascorbate reductase 5 | 2.66 | Redox | 0.014 |
| GhiAffx.24591.1 | DW502202.1 | serine carboxypeptidase-like 40 | 2.64 | M | 0.018 |
| Ghi.7617.1 | AI728876 | receptor protein kinase TMK1 | 2.64 | ST | 0.022 |
| Ghi.7164.1 | AW187576 | gamma-interferon-inducible lysosomal thiol | 2.62 | Redox | 0.011 |
| | | reductase | | | |
| Ghi.6101.1 | DT457964 | transcription factor MYB36 | 2.62 | TF | 0.045 |
| GhiAffx.53638.1 | DW232569.1 | expansin A11 | 2.58 | G | 0.044 |
| GhiAffx.16299.1 | DW229097.1 | fasciclin-like arabinogalactan protein 9 | 2.57 | G | 0.003 |
| Ghi.10346.1 | DN758230 | luminal-binding protein 5 | 2.57 | M | 0.007 |
| Ghi.4685.1 | DT051431 | glucose-6-phosphate 1-dehydrogenase | 2.57 | Redox | 0.018 |
| Ghi.5188.1 | DT048700 | inactive receptor kinase | 2.57 | ST | 0.040 |
| GhiAffx.25508.1 | DW496030.1 | ethylene-responsive transcription factor | 2.57 | TF | 0.043 |
| | | ERF114 | | | |
| GhiAffx.22625.1 | DW501687.1 | acyl-lipid (9-3)-desaturase | 2.56 | M | 0.006 |
| GhiAffx.52253.1 | DR463559 | pyrophosphat--fructose 6-phosphate 1- | 2.56 | M | 0.037 |
| | | phosphotransferase subunit beta | | | |
| GhiAffx.50488.1 | DT456967 | DNA ligase 1 | 2.54 | Mol | 0.005 |
| Ghi.8186.1 | DT460349 | fasciclin-like arabinogalactan protein 9 | 2.53 | G | 0.003 |
| GhiAffx.6395.1 | DW484802.1 | auxin-responsive protein IAA14 | 2.53 | TF | 0.025 |
| Ghi.1893.1 | DV849529 | tubulin beta chain-like | 2.52 | G | 0.010 |
| GarAffx.24390.1 | BF273948 | non-symbiotic hemoglobin 2 | 2.52 | M | 0.027 |
| Ghi.7718.1 | AI726866 | ATP-citrate synthase beta chain protein 2 | 2.51 | M | 0.011 |
| GhiAffx.6254.1 | DW498306.1 | high-affinity nitrate transporter | 2.51 | T | 0.043 |
| Ghi.1253.1 | DT051546 | UMP-CMP kinase 3 | 2.5 | M | 0.016 |
| Ghi.1853.1 | DR462814 | methyltransferase PMT21 | 2.5 | M | 0.020 |
| Ghi.7542.1 | AI729532 | serine hydroxymethyltransferase 2 | 2.5 | M | 0.020 |
| GhiAffx.49935.1 | DW487642.1 | chaperonin CPN60-2 | 2.5 | S | 0.044 |
| Ghi.10754.1 | DV849226 | COBRA | 2.47 | G | 0.046 |
| Ghi.8669.1 | DR452575 | kinesin-related protein 11 | 2.46 | G | 0.019 |
| Ghi.669.2 | DT463066 | beta-galactosidase 3 | 2.43 | G | 0.015 |
| GhiAffx.31831.1 | DT456192 | ribonucleoside-diphosphate reductase | 2.41 | Mol | 0.012 |
| Ghi.3168.2 | DR463922 | beta-galactosidase 10 | 2.39 | G | 0.034 |
| Ghi.7657.1 | DR454717 | ERR receptor-like serine/threonine-protein | 2.38 | ST | 0.019 |
| | | kinase MRH1 | | | |
| GhiAffx.6062.1 | DW495992.1 | Unknown | 2.37 | — | 0.014 |
| Ghi.395.1 | DN817680 | Aquaporin | 2.37 | T | 0.043 |
| Ghi.8845.1 | DT456859 | Unknown | 2.35 | — | 0.037 |
| Ghi.2406.1 | DT052156 | Pectinesterase | 2.33 | G | 0.000 |
| GhiAffx.7836.1 | DW516257.1 | GMP synthase | 2.33 | M | 0.006 |
| Ghi.8359.1 | CA993401 | boron transporter 1 | 2.33 | T | 0.040 |
| Gra.2279.2 | CO111411 | 5-methyltetrahydropteroyltriglutamate-- | 2.3 | M | 0.016 |
| | | homocysteine methyltransferase | | | |
| GhiAffx.12945.1 | DT463227 | -like protein 8 | 2.29 | M | 0.013 |
| Ghi.3122.2 | DT460791 | chloroplast stem-loop binding protein of 41 | 2.29 | Mol | 0.037 |
| | | kDa | | | |
| Gra.1696.1 | CO085682 | TBC1 domain family member 15 | 2.28 | ST | 0.006 |
| GhiAffx.46616.1 | DR456388 | protein RALF-like 33 | 2.28 | ST | 0.018 |
| Gra.2199.2 | CO121107 | disulfide-isomerase like 2-2 | 2.27 | M | 0.049 |
| Ghi.1907.1 | DV849267 | proteasome subunit alpha type-5 | 2.26 | M | 0.046 |
| Ghi.6906.1 | CA992663 | Unknown | 2.23 | — | 0.013 |
| GhiAffx.16114.2 | DW236410.1 | pollen-specific leucine-rich repeat extensin- | 2.23 | G | 0.039 |
| | | like protein 2 | | | |
| GhiAffx.5071.1 | DW516454.1 | ubiquitin-conjugating enzyme E2 20 | 2.23 | M | 0.044 |
| Gra.431.2 | CO123490 | D-3-phosphoglycerate dehydrogenase 1 | 2.22 | Redox | 0.042 |
| GhiAffx.47164.1 | CO495471 | tetrahydrocannabinolic acid synthase | 2.21 | M | 0.006 |
| GhiAffx.7826.1 | DW516143.1 | kinesin-related protein 11 | 2.2 | G | 0.005 |
| Ghi.734.1 | DR455414 | Unknown | 2.19 | — | 0.017 |
| GhiAffx.9554.1 | DR456349 | xyloglucan glycosyltransferase 4 | 2.18 | G | 0.027 |
| GarAffx.29986.1 | AW725588 | non-specific lipid transfer protein GPI- | 2.18 | T | 0.045 |
| | | anchored 1 | | | |
| Ghi.3929.1 | DR461083 | 50S ribosomal protein L2 | 2.17 | Mol | 0.003 |
| Ghi.3170.2 | DT468829 | ferredoxin-nitrite reductase | 2.17 | Redox | 0.012 |
| GhiAffx.7622.1 | DW513729.1 | CBL-interacting protein kinase 32 | 2.17 | ST | 0.015 |
| Ghi.4321.1 | AW186956 | monocopper oxidase-like protein SKU5 | 2.16 | G | 0.027 |
| Ghi.7911.1 | AY476737.1 | xyloglucan endotransglucosylase/hydrolase | 2.14 | G | 0.003 |
| | | protein 22 | | | |
| GhiAffx.63257.1 | DW500546.1 | Unknown | 2.13 | — | 0.032 |
| GraAffx.23146.1 | CO101937 | pectinesterase 3 | 2.13 | G | 0.043 |
| Ghi.4007.1 | DR459308 | Unknown | 2.1 | — | 0.004 |
| Ghi.8798.1 | DT456078 | Unknown | 2.1 | — | 0.034 |
| GraAffx.28945.1 | CO085033 | fructokinase-6 | 2.1 | M | 0.044 |
| GarAffx.13408.1 | BM357988 | 39S ribosomal protein L45 | 2.09 | Mol | 0.007 |
| Ghi.10256.1 | DT567984 | pectate lyase | 2.06 | G | 0.048 |
| Ghi.9249.4 | DT461850 | indole-3-acetic acid-amido synthetase GH3.1 | 2.05 | H | 0.018 |
| Gra.294.1 | CO089366 | aquaporin TIP4-1 | 2.05 | T | 0.020 |
| Ghi.7047.1 | AI730734 | polyadenylate-binding protein 2 | 2.04 | Mol | 0.008 |
| Ghi.10401.1 | DT555004 | enoyl-reductase | 2.04 | Redox | 0.017 |
| Ghi.4236.1 | DT456638 | Unknown | 2.03 | — | 0.022 |
| GhiAffx.25281.1 | DW516167.1 | cellulose synthase A catalytic subunit 5 | 2.01 | G | 0.024 |
| | | [UDP-forming] | | | |
| Ghi.9045.1 | DT048514 | methylsterol monooxygenase 2-2 | 2.01 | Redox | 0.025 |
| Ghi.6559.1 | CD485748 | Peroxidase | 2.01 | Redox | 0.030 |
| Ghi.3291.1 | DT467607 | receptor-like protein kinase THESEUS 1 | 2.01 | ST | 0.043 |

TABLE 10
Exemplary down-regulated genes of non-stressed cotton seedling roots
inoculated with Bacillus amyloliquefaciens (strain Bamy).

| | | | Fold-Change | | |
|---|---|---|---|---|---|
| Transcript ID | Accession No. | Description | (linear) | Category | p-value |
| GhiAffx.43038.1 | DW497938.1 | salicylate carboxymethyltransferase | −47.79 | M | 0.010 |
| GhiAffx.25701.1 | DW517734.1 | metacaspase-9 | −43.67 | D | 0.009 |
| Ghi.6022.1 | CO490753 | Unknown | −5.5 | — | 0.007 |
| GhiAffx.10674.1 | CO498018 | calcium-binding protein CML10 | −5.27 | M | 0.039 |
| GhiAffx.40045.1 | DW495842.1 | DMR6-LIKE OXYGENASE 2 | −5.08 | D | 0.025 |
| Ghi.5602.1 | DT050039 | methionine gamma-lyase | −4.8 | M | 0.025 |
| Ghi.1080.1 | DV849085 | oxygen-evolving enhancer protein 1 | −4.68 | Redox | 0.046 |
| Ghi.1444.1 | DN781680 | light-regulated protein | −4.66 | M | 0.024 |
| Ghi.6901.1 | CA992707 | zinc finger protein ZAT10 | −4.64 | TF | 0.031 |
| GhiAffx.6177.1 | DW505740.1 | WRKY94 | −3.88 | TF | 0.049 |
| GhiAffx.7927.1 | DW499556.1 | translocator protein homolog | −3.84 | T | 0.024 |
| Ghi.6780.1 | CA993199 | chaperone protein dnaJ 11 | −3.72 | Mol | 0.026 |
| Ghi.10646.1 | DN780414 | P21 | −3.7 | G | 0.024 |
| Ghi.1660.1 | DN760124 | Unknown | −3.69 | — | 0.015 |
| Ghi.9193.2 | DT469110 | WRKY transcription factor 70 | −3.69 | TF | 0.027 |
| GhiAffx.46297.1 | AI054544 | expansin-like B1 | −3.64 | G | 0.015 |
| Ghi.8451.1 | CO496935 | Unknown | −3.57 | — | 0.006 |
| GhiAffx.13391.3 | AI055016 | BIG GRAIN 1 | −3.55 | T | 0.026 |
| GraAffx.23006.2 | CO126999 | WAT1-related protein | −3.51 | T | 0.004 |
| GraAffx.30924.2 | CO089587 | cytochrome b6-f complex | −3.45 | Redox | 0.019 |
| GhiAffx.10817.1 | DW517233.1 | F-box protein PP2-A13 | −3.4 | M | 0.025 |
| GhiAffx.48816.1 | DW505775.1 | Unknown | −3.36 | — | 0.007 |
| GhiAffx.14096.1 | CO494404 | TRANSPARENT TESTA 12 | −3.34 | T | 0.013 |
| GhiAffx.39386.1 | DW233404.1 | Unknown | −3.2 | — | 0.011 |
| Ghi.7797.1 | AI726250 | cysteine proteinase inhibitor B | −3.18 | M | 0.002 |
| GraAffx.15208.1 | CO125472 | serine/threonine-protein kinase HT1 | −3.18 | ST | 0.041 |
| Ghi.3788.1 | DT461699 | Unknown | −3.16 | — | 0.049 |
| Ghi.6465.2 | CD485906 | expansin-like B1 | −3.14 | G | 0.011 |
| Ghi.6613.1 | CA994335 | oxygen-evolving enhancer protein 3 | −3.14 | Redox | 0.018 |
| Ghi.8342.1 | CA992699 | Unknown | −3.11 | — | 0.013 |
| GhiAffx.53687.1 | DW232903.1 | Unknown | −3.09 | — | 0.030 |
| Ghi.10656.1 | DR463281 | photosystem I reaction center subunit II | −3.07 | Redox | 0.013 |
| GraAffx.31485.1 | CO077629 | Periaxin | −3.06 | G | 0.010 |
| GhiAffx.22386.1 | DW224724.1 | NRT1/PTR FAMILY 5.6 | −3.06 | T | 0.016 |
| Ghi.1650.1 | DN758296 | PAP-specific phosphatase HAL2 | −3.02 | M | 0.042 |
| GhiAffx.40824.1 | DW499731.1 | zinc finger protein ZAT11 | −2.93 | TF | 0.039 |
| Ghi.563.2 | DT463965 | Unknown | −2.89 | — | 0.034 |
| Ghi.5228.1 | DT048570 | Unknown | −2.88 | — | 0.003 |
| GhiAffx.20889.1 | DW233795.1 | ferredoxin-1 | −2.84 | Redox | 0.003 |
| Ghi.1165.1 | DT050989 | zinc finger protein CONSTANS-LIKE 1 | −2.84 | TF | 0.041 |
| Gra.2634.1 | CO125011 | chlorophyllide a oxygenase | −2.83 | Redox | 0.046 |
| GhiAffx.863.1 | DN817257 | magnesium-chelatase subunit ChlH | −2.78 | M | 0.004 |
| Ghi.10709.1 | DT050206 | Phytosulfokines | −2.73 | H | 0.001 |
| GhiAffx.3323.1 | DW224416.1 | flavin-dependent oxidoreductase FOX2 | −2.73 | Redox | 0.010 |
| GhiAffx.60596.1 | DW505179.1 | Unknown | −2.59 | — | 0.001 |
| GhiAffx.43407.1 | CA993283 | Unknown | −2.57 | — | 0.030 |
| Gra.2885.1 | CO100046 | probable strigolactone esterase D14 | −2.55 | M | 0.004 |
| GhiAffx.4924.2 | DW501614.1 | UDP-glycosyltransferase 74B1 | −2.55 | M | 0.007 |
| GhiAffx.58403.1 | DW236137.1 | cyclin-D1-1 | −2.54 | G | 0.013 |
| Ghi.10472.2 | DN758193 | zinc finger protein CONSTANS-LIKE 4 | −2.54 | TF | 0.039 |
| GhiAffx.15714.1 | DW224008.1 | chloroplast stem-loop binding protein of | −2.54 | Mol | 0.050 |
| | | 41 kDa | | | |
| GhiAffx.10273.1 | DW514588.1 | Unknown | −2.53 | — | 0.032 |
| Ghi.1134.1 | DR460878 | photosystem I reaction center subunit IV | −2.53 | M | 0.049 |
| | | B | | | |
| GraAffx.9623.1 | CO084784 | Unknown | −2.52 | — | 0.005 |
| GhiAffx.41348.1 | DW502424.1 | photosystem II reaction center W protein | −2.51 | M | 0.045 |
| Gra.2595.2 | CO088781 | Unknown | −2.5 | — | 0.025 |
| Gra.2751.1 | CO092060 | LOL1 | −2.48 | D | 0.004 |
| Ghi.4064.2 | DR461611 | cytokinin riboside 5′-monophosphate | −2.48 | H | 0.033 |
| | | phosphoribohydrolase LOG1 | | | |
| GhiAffx.3475.1 | DW514212.1 | glycerate dehydrogenase | −2.48 | Redox | 0.048 |
| Ghi.8389.1 | CO498953 | CASP-like protein 1D1 | −2.47 | G | 0.015 |
| GhiAffx.3473.1 | DW232059.1 | protein LHY | −2.44 | TF | 0.028 |
| Ghi.10242.1 | DT053185 | AT-hook motif nuclear-localized protein 9 | −2.43 | TF | 0.035 |
| Ghi.8113.1 | AY174160.1 | DRE binding protein 1 | −2.4 | TF | 0.003 |
| GhiAffx.30296.1 | DW509681.1 | Unknown | −2.39 | — | 0.002 |
| Ghi.5259.1 | DT048446 | glutamate dehydrogenase 2 | −2.39 | Redox | 0.004 |
| Ghi.6888.1 | DV848826 | geranylgeranyl diphosphate reductase | −2.39 | Redox | 0.021 |
| GhiAffx.4924.1 | DW501276.1 | UDP-glycosyltransferase 74B1 | −2.37 | M | 0.009 |
| GhiAffx.5828.1 | DW514056.1 | extra-large guanine nucleotide-binding | −2.36 | ST | 0.004 |
| | | protein 1 | | | |
| Ghi.3451.2 | DT465672 | alpha,alpha-trehalose-phosphate synthase | −2.34 | M | 0.006 |
| GraAffx.28214.1 | CO087188 | transcription factor MYB36 | −2.34 | TF | 0.041 |
| Gra.111.1 | CO097047 | thiosulfate sulfurtransferase 16 | −2.31 | M | 0.000 |
| GhiAffx.22025.1 | DW498464.1 | serine/threonine-protein phosphatase PP1 | −2.31 | M | 0.009 |
| | | isozyme 3 | | | |
| GhiAffx.3708.1 | DR457956 | transcription factor TCP2 | −2.31 | TF | 0.021 |
| Gra.429.1 | CO090033 | Unknown | −2.3 | — | 0.028 |
| Gra.1323.2 | CO117765 | nudix hydrolase 8 | −2.26 | M | 0.013 |
| GhiAffx.32507.1 | CO494420 | LOL1 | −2.25 | D | 0.017 |
| Ghi.1766.1 | AY138252.1 | phospholipase D delta | −2.25 | M | 0.041 |
| GhiAffx.24039.1 | DW509198.1 | zinc finger protein 4 | −2.25 | TF | 0.049 |
| GhiAffx.21780.1 | DW497160.1 | Unknown | −2.24 | — | 0.008 |
| GraAffx.28297.1 | CO086928 | Unknown | −2.21 | — | 0.036 |
| Ghi.836.1 | DR454138 | Unknown | −2.2 | — | 0.031 |
| GhiAffx.63986.1 | DW514188.1 | 4-hydroxyphenylpyruvate dioxygenase | −2.2 | Redox | 0.041 |
| Ghi.10826.1 | DN827439 | glucan endo-1,3-beta-glucosidase, basic | −2.19 | M | 0.048 |
| | | vacuolar isoform | | | |
| Ghi.1386.1 | DT051580 | receptor-like protein kinase HSL1 | −2.16 | ST | 0.008 |
| GhiAffx.27739.1 | DW506283.1 | dof zinc finger protein DOF4.6 | −2.16 | TF | 0.026 |
| Ghi.6847.1 | DN779665 | photosystem II 5 kDa protein | −2.15 | M | 0.022 |
| GhiAffx.25540.1 | DW225138.1 | cyclic dof factor 3 | −2.15 | TF | 0.022 |
| Ghi.4716.1 | DT051239 | ethylene-responsive transcription factor | −2.15 | TF | 0.031 |
| | | ERF119 | | | |
| GhiAffx.21685.1 | CO493081 | Unknown | −2.14 | — | 0.003 |
| Ghi.10332.1 | DT465694 | WRKY transcription factor 2 | −2.14 | TF | 0.033 |
| Ghi.279.1 | DR461178 | Unknown | −2.13 | — | 0.015 |
| GhiAffx.43289.1 | DW501066.1 | RING-H2 finger protein ATL78 | −2.13 | M | 0.038 |
| Ghi.1670.1 | DN760074 | homeobox protein knotted-1-like 6 | −2.13 | TF | 0.040 |
| GraAffx.33744.1 | CO107275 | guanine nucleotide exchange factor 7 | −2.12 | Mol | 0.004 |
| GhiAffx.61812.1 | DW511383.1 | CSC1-like protein ERD4 | −2.12 | T | 0.029 |
| GhiAffx.26447.1 | DW504068.1 | GPI-anchored CFEM domain protein A | −2.11 | G | 0.024 |
| Ghi.6041.1 | DV849896 | aspartyl protease family protein | −2.11 | M | 0.031 |
| Ghi.10259.1 | DT463881 | bZIP transcription factor 53 | −2.11 | TF | 0.050 |
| Ghi.6171.1 | DT048318 | auxin-responsive protein SAUR36 | −2.09 | H | 0.018 |
| Ghi.6751.1 | CA993334 | fructose-1,6-bisphosphatase | −2.09 | M | 0.020 |
| Ghi.1822.2 | DV850271 | Unknown | −2.08 | — | 0.042 |
| GhiAffx.59645.1 | DW511857.1 | high-light-induced protein | −2.07 | M | 0.001 |
| Ghi.6674.1 | CA993725 | WRKY transcription factor 40 | −2.07 | TF | 0.013 |
| Ghi.6538.1 | CD485949 | NAC transcription factor 29 | −2.07 | TF | 0.016 |
| GraAffx.31672.1 | CO077112 | Unknown | −2.06 | — | 0.011 |
| GraAffx.27948.1 | CO087928 | secoisolariciresinol dehydrogenase | −2.06 | Redox | 0.017 |
| Ghi.10388.2 | DT469076 | CBL-interacting serine/threonine-protein | −2.06 | ST | 0.018 |
| | | kinase 6 | | | |
| GhiAffx.22987.1 | DW503573.1 | Unknown | −2.05 | — | 0.033 |
| GhiAffx.10268.1 | DW501443.1 | 7-deoxyloganetin glucosyltransferase | −2.04 | M | 0.016 |
| Ghi.7225.1 | DR462056 | Mitochondria | −2.04 | M | 0.025 |
| Ghi.8342.1 | CA992699 | Unknown | −2.03 | — | 0.001 |
| GhiAffx.20424.1 | DW493894.1 | pyridoxal reductase | −2.01 | Redox | 0.038 |
| GhiAffx.6761.1 | DW504313.1 | protein NRT1/PTR FAMILY 8.1 | −2.01 | T | 0.045 |

TABLE 11
Exemplary up-regulated genes of salt stressed cotton roots inoculated
with Bacillus amyloliquefaciens (strain Bamy).

| | | | Fold-Change | | |
|---|---|---|---|---|---|
| Probe Set ID | Accession No. | Description | (linear) | Category | p-value |
| Ghi.10547.3 | DR458023 | blue copper protein | 6.87 | Redox | 0.041 |
| GhiAffx.1161.1 | DN759989 | expansin B2 | 6.71 | G | 0.015 |
| Ghi.1630.1 | DN760255 | laccase 17 | 5.39 | Redox | 0.050 |
| Ghi.10547.1 | DN758139 | blue copper protein | 5.1 | Redox | 0.026 |
| Ghi.10553.1 | DN760229 | lipid transfer protein DIR1 | 4.13 | D | 0.035 |
| GhiAffx.32420.1 | CO496669 | BAG chaperone regulator 4 | 4.01 | S | 0.031 |
| GhiAffx.15941.1 | AJ513880 | Unknown | 3.97 | — | 0.038 |
| Gra.20.2 | CO087377 | WLIM1 | 3.95 | G | 0.034 |
| GhiAffx.42278.1 | DW507308.1 | Unknown | 3.93 | — | 0.041 |
| Ghi.8139.1 | Z68154.1 | glucan endo-1,3-beta-glucosidase | 3.91 | D | 0.020 |
| GhiAffx.13781.1 | DW506341.1 | Unknown | 3.85 | — | 0.049 |
| Ghi.3017.1 | DN759882 | Pectinesterase | 3.57 | G | 0.033 |
| GhiAffx.30838.1 | DW504875.1 | Polygalacturonase | 3.55 | G | 0.001 |
| GhiAffx.34625.1 | DW227939.1 | COP1 interacting protein | 3.5 | G | 0.040 |
| Gra.1548.1 | CO123811 | isoaspartyl peptidase/L-asparaginase 2 | 3.44 | M | 0.011 |
| GbaAffx.196.1 | AY572462.1 | ethylene-responsive transcription factor | 3.43 | TF | 0.023 |
| | | RAP2-3 | | | |
| GhiAffx.25797.1 | DW518337.1 | pectinesterase inhibitor 61 | 3.41 | G | 0.014 |
| Ghi.4358.1 | DT053995 | Unknown | 3.35 | — | 0.026 |
| GhiAffx.64005.1 | DW496581.1 | glycine-rich cell wall structural protein 1 | 3.34 | G | 0.043 |
| Ghi.249.1 | DQ204496.1 | expansin A8 | 3.32 | G | 0.005 |
| GhiAffx.34523.1 | DW227339.1 | protein IQ-DOMAIN 14 | 3.31 | M | 0.046 |
| GhiAffx.58784.1 | DW496107.1 | transcription factor MYB86 | 3.29 | TF | 0.004 |
| GhiAffx.26018.1 | DW519688.1 | endoglucanase 8 | 3.24 | G | 0.016 |
| GhiAffx.1589.49 | DW234098.1 | kinesin light chain 4 | 3.24 | G | 0.018 |
| GbaAffx.196.1 | AY572462.1 | ethylene-responsive transcription factor | 3.24 | TF | 0.034 |
| | | RAP2-3 | | | |
| Ghi.294.2 | DR461090 | Unknown | 3.2 | — | 0.047 |
| Ghi.7874.1 | AY962572.1 | ethylene-responsive transcription factor | 3.15 | TF | 0.014 |
| | | RAP2-3 | | | |
| Ghi.9707.1 | DT051791 | tubulin beta-9-chain | 3.12 | G | 0.035 |
| GhiAffx.15745.1 | DW224242.1 | laccase 4 | 3.1 | Redox | 0.043 |
| Ghi.8095.1 | AY125487.1 | Unknown | 3.07 | — | 0.044 |
| Ghi.8638.2 | DT468706 | Polygalacturonase | 3.05 | G | 0.035 |
| Ghi.4877.1 | DT460085 | leucine-rich repeat extensin-like protein 6 | 3 | G | 0.029 |
| GhiAffx.7716.1 | DW514934.1 | Unknown | 2.99 | — | 0.003 |
| Ghi.9666.1 | DN759995 | fasciclin-like arabinogalactan protein 10 | 2.99 | G | 0.009 |
| Ghi.7294.1 | AI731843 | glucomannan 4-beta mannosyltransferase-2 | 2.95 | G | 0.024 |
| GraAffx.17891.1 | CO117605 | aspartic protease in guard cell 1 | 2.94 | M | 0.023 |
| GhiAffx.39841.1 | DW237452.1 | patellin 3 | 2.92 | G | 0.023 |
| Ghi.8128.1 | AY827548.1 | EREB1 transcription factor | 2.9 | TF | 0.020 |
| GhiAffx.53142.1 | DW224608.1 | cytochrome P450 | 2.86 | Redox | 0.046 |
| GhiAffx.7362.1 | DW237315.1 | patellin 3 | 2.83 | G | 0.012 |
| Ghi.10477.1 | DN758086 | laccase 4 | 2.74 | Redox | 0.036 |
| Ghi.10615.1 | DN759950 | Unknown | 2.69 | — | 0.004 |
| GhiAffx.4490.1 | CO497091 | transcription factor MYB86 | 2.69 | TF | 0.031 |
| Ghi.10354.1 | AI730528 | Polygalacturonase | 2.68 | G | 0.030 |
| Ghi.885.1 | AI728693 | Unknown | 2.64 | — | 0.032 |
| GhiAffx.15613.1 | DW514029.1 | E3 ubiquitin-protein ligase | 2.6 | M | 0.018 |
| GhiAffx.22837.1 | DW502760.1 | tyrosine-protein phosphatase | 2.54 | ST | 0.022 |
| Ghi.309.1 | DR460966 | aspartic protease in guard cell 1 | 2.53 | M | 0.030 |
| Ghi.5516.1 | DT047448 | reticulon protein B21 | 2.52 | M | 0.029 |
| Ghi.9268.1 | DT463525 | 2-oxoglutarate-dependent dioxygenase | 2.5 | Redox | 0.030 |
| GraAffx.33025.1 | CO118932 | Unknown | 2.48 | — | 0.026 |
| GhiAffx.23182.1 | DW504552.1 | UDP-glucuronate: xylan alpha- | 2.42 | G | 0.031 |
| | | glucuronosyltransferase 1 | | | |
| Gra.1095.1 | CO125355 | CBS domain-containing protein CBSX5 | 2.4 | M | 0.045 |
| Ghi.5727.1 | DT046456 | Unknown | 2.39 | — | 0.008 |
| Ghi.6322.1 | DN758059 | (R,S)-reticuline 7-O-methyltransferase | 2.36 | M | 0.003 |
| GhiAffx.7609.1 | DW513579.1 | Unknown | 2.35 | — | 0.033 |
| GhiAffx.25760.1 | DW518128.1 | Unknown | 2.33 | — | 0.037 |
| GhiAffx.6671.1 | DW503045.1 | Unknown | 2.31 | — | 0.026 |
| GhiAffx.33980.1 | DW514762.1 | myosin-2 heavy chain | 2.29 | G | 0.008 |
| GhiAffx.33585.1 | DW507662.1 | cell wall/vacuolar inhibitor of fructosidase 1 | 2.29 | M | 0.009 |
| Ghi.2060.2 | DN759860 | 36.4 kDa proline-rich protein | 2.28 | G | 0.012 |
| GhiAffx.36287.1 | DW501982.1 | cytosolic sulfotransferase 5 | 2.27 | M | 0.006 |
| Ghi.8009.1 | CD809330 | Unknown | 2.23 | — | 0.003 |
| Ghi.4510.1 | DT049560 | kinesin KP1 | 2.23 | G | 0.037 |
| GhiAffx.7861.1 | DW516540.1 | Unknown | 2.22 | — | 0.020 |
| Ghi.8145.1 | AY212968.1 | inactive receptor-like serine/threonine- | 2.21 | ST | 0.046 |
| | | protein kinase | | | |
| Ghi.4479.1 | DT051628 | Unknown | 2.18 | — | 0.040 |
| Ghi.8009.2 | DR460320 | Unknown | 2.16 | — | 0.019 |
| Ghi.680.1 | AF044205.1 | proline-rich protein precursor | 2.14 | G | 0.023 |
| Ghi.2255.1 | AY962571.1 | ethylene responsive element binding protein | 2.13 | TF | 0.045 |
| | | 2 | | | |
| Ghi.1933.1 | CA992810 | 7-deoxyloganetic acid glucosyltransferase | 2.12 | M | 0.019 |
| Gra.2722.2 | CO085482 | cytochrome P450 | 2.08 | Redox | 0.037 |
| Ghi.3072.1 | DT527412 | Unknown | 2.06 | — | 0.007 |
| Ghi.2060.1 | DT049026 | 36.4 kDa proline-rich protein | 2.06 | G | 0.018 |
| GhiAffx.4153.1 | DW224618.1 | Polygalacturonase | 2.06 | G | 0.046 |
| Gra.3004.2 | CO086920 | expansin A15 | 2.04 | G | 0.049 |

TABLE 12
Exemplary down-regulated genes in salt stressed cotton seedling roots
inoculated with Bacillus amyloliquefaciens (strain Bamy).

| | | | Fold-Change | | |
|---|---|---|---|---|---|
| Probe Set ID | Accession No. | Description | (linear) | Category | p-value |
| GhiAffx.46297.1 | AI054544 | expansin-like B1 | −6.56 | G | 0.038 |
| Ghi.3468.1 | DT465471 | Cucumisin | −6.26 | M | 0.007 |
| Ghi.6465.2 | CD485906 | expansin-like B1 | −5.98 | G | 0.034 |
| Gra.2963.1 | CO103741 | UDP-glycosyltransferase 73C6 | −4.4 | M | 0.045 |
| Ghi.5751.1 | DT046426 | histidine-rich glycoprotein | −4.22 | G | 0.014 |
| GhiAffx.7921.1 | DW517203.1 | Unknown | −3.43 | — | 0.032 |
| GhiAffx.45274.1 | DW241839.1 | mitoferrin-like | −3.43 | T | 0.040 |
| GhiAffx.40824.1 | DW499731.1 | zinc finger protein ZAT11 | −3.31 | TF | 0.017 |
| GhiAffx.23888.1 | DW508393.1 | metacaspase-3 | −3.3 | D | 0.049 |
| GarAffx.29310.2 | BE054959 | alcohol dehydrogenase | −3.23 | Redox | 0.029 |
| GhiAffx.24344.1 | DW510728.1 | LRR receptor-like serine/threonine-protein kinase | −3.1 | ST | 0.016 |
| | | GSO2 | | | |
| Gra.2743.1 | CO091320 | cytochrome P450 86B1 | −2.89 | Redox | 0.033 |
| Ghi.3600.1 | DT463857 | FAD-dependent urate hydroxylase | −2.83 | Redox | 0.041 |
| Ghi.2239.1 | DR459719 | lupeol synthase | −2.82 | G | 0.023 |
| Ghi.9768.1 | DN760794 | granule-bound starch synthase 1 | −2.78 | M | 0.032 |
| Ghi.8451.1 | CO496935 | Unknown | −2.75 | — | 0.026 |
| Ghi.1752.1 | DT463475 | Unknown | −2.63 | — | 0.018 |
| GhiAffx.25398.1 | DW516152.1 | auxin response factor 5 | −2.62 | TF | 0.028 |
| Ghi.6718.1 | CA993519 | peroxidase 11 | −2.59 | Redox | 0.026 |
| GhiAffx.22987.1 | DW503573.1 | Unknown | −2.57 | — | 0.008 |
| Ghi.9843.1 | AI728365 | 3-ketoacyl-CoA synthase 11 | −2.44 | M | 0.030 |
| GhiAffx.1119.2 | DW225844.1 | DNA-directed RNA polymerase subunit alpha | −2.43 | Mol | 0.038 |
| GhiAffx.29821.1 | DW520191.1 | ECERIFERUM 1 | −2.34 | G | 0.018 |
| Ghi.7225.1 | DR462056 | Mitochondria | −2.28 | M | 0.038 |
| Ghi.1660.1 | DN760124 | Unknown | −2.24 | — | 0.030 |
| GhiAffx.18012.1 | DW482227.1 | Mitochondria | −2.23 | M | 0.009 |
| GhiAffx.1557.2 | DT460784 | Mitochondria | −2.16 | M | 0.046 |
| GhiAffx.54078.1 | DW230253.1 | Mitochondria | −2.13 | M | 0.002 |
| Gra.3054.1 | CO116519 | FAD-dependent urate hydroxylase | −2.11 | Redox | 0.043 |
| Ghi.1760.1 | DN758667 | Unknown | −2.1 | — | 0.012 |
| Ghi.9241.4 | CO491852 | 5′-adenylylsulfate reductase 2 | −2.09 | Redox | 0.036 |
| Ghi.3425.2 | DT466037 | GEM-like protein 5 | −2.01 | H | 0.009 |

Gene Ontology and Enriched Pathways in Non-Stressed Cotton Seedling Roots

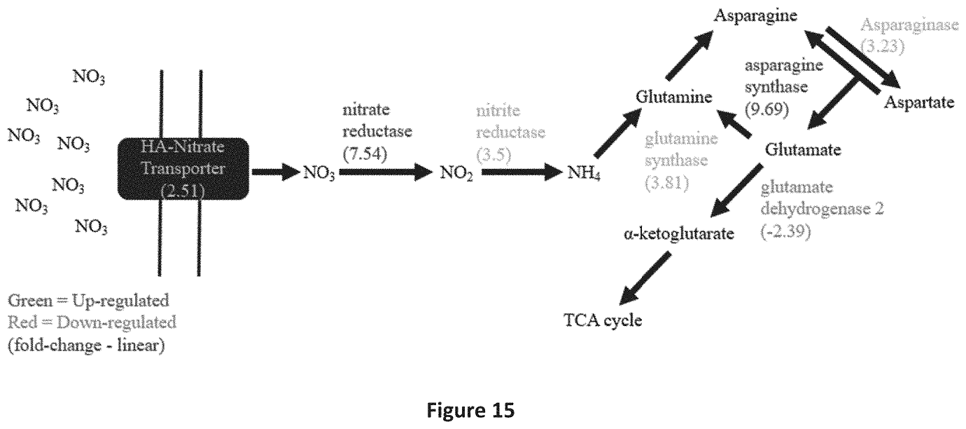

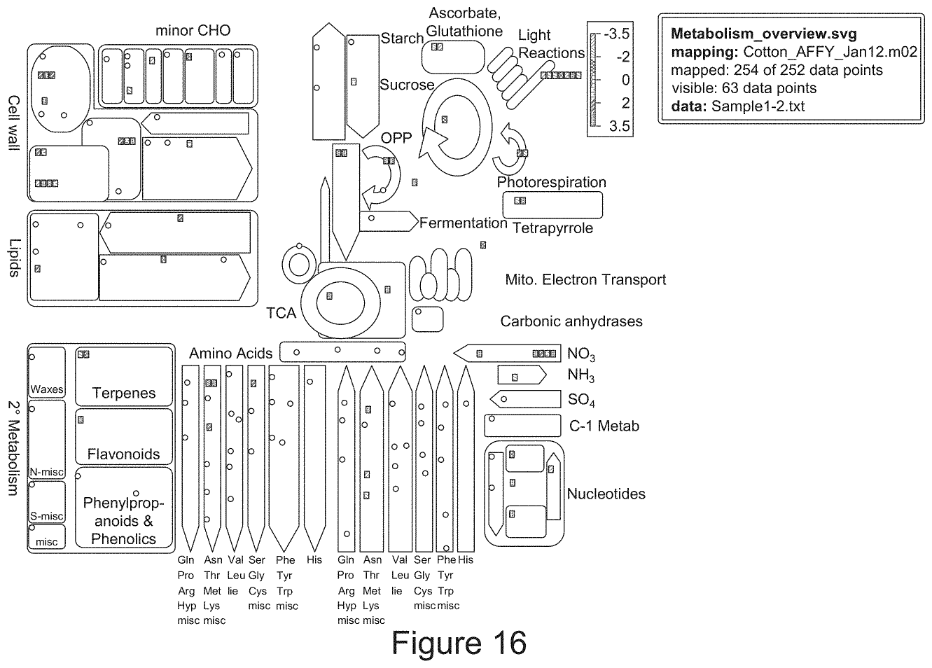

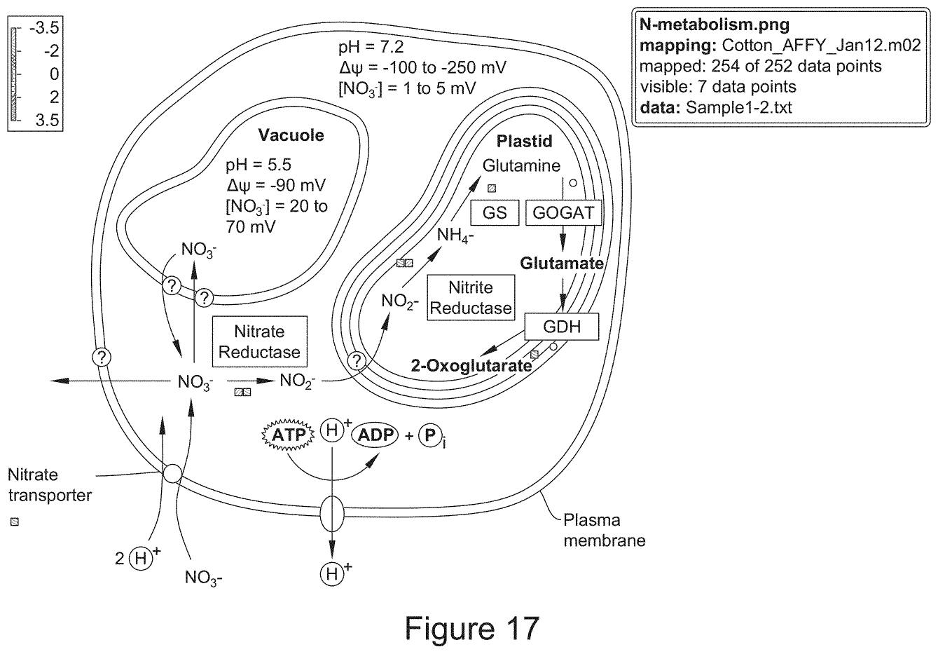

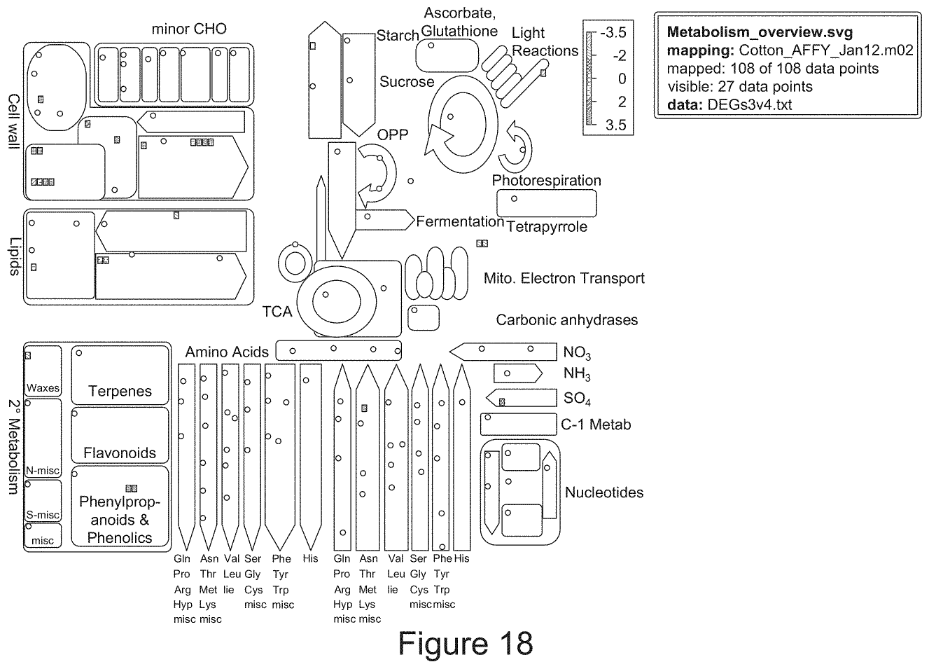

Gene ontology and enriched pathways were determined using SEA in agriGO. Twenty-four significantly enriched pathways (p<0.05) were identified through SEA analysis in inoculated cotton seedlings in non-stressed conditions. The three most enriched pathways are related to nitrogen assimilation, redox pathways and metabolism. (Table 13). Other nitrogen-related pathways that were enriched included asparagine metabolic process, oxidoreductase activity that acts on other nitrogenous compounds as donors, and ferredoxin-nitrite reductase activity (Table 13). The up-regulation of genes related to nitrogen related biological processes is also illustrated in FIG. 15 and FIG. 16. In addition to pathways related to nitrogen assimilation and metabolism, various pathways that were enriched were related to microtubule-based movement, vesicles, and microtubules in the cytoskeleton (Table 13). The up-regulation of genes related to cellular components is illustrated in FIG. 17. Additional enriched pathways were identified to be involved in molecular functions (FIG. 18). Genes involved in heme bin binding, copper ion binding, and tetrapyrrole binding pathways were also determined to be up-regulated using SEA (Table 13). In addition, genes encoding for UTP-uridylyltransferases and FAD binding processes were enriched (Table 13, FIG. 18).

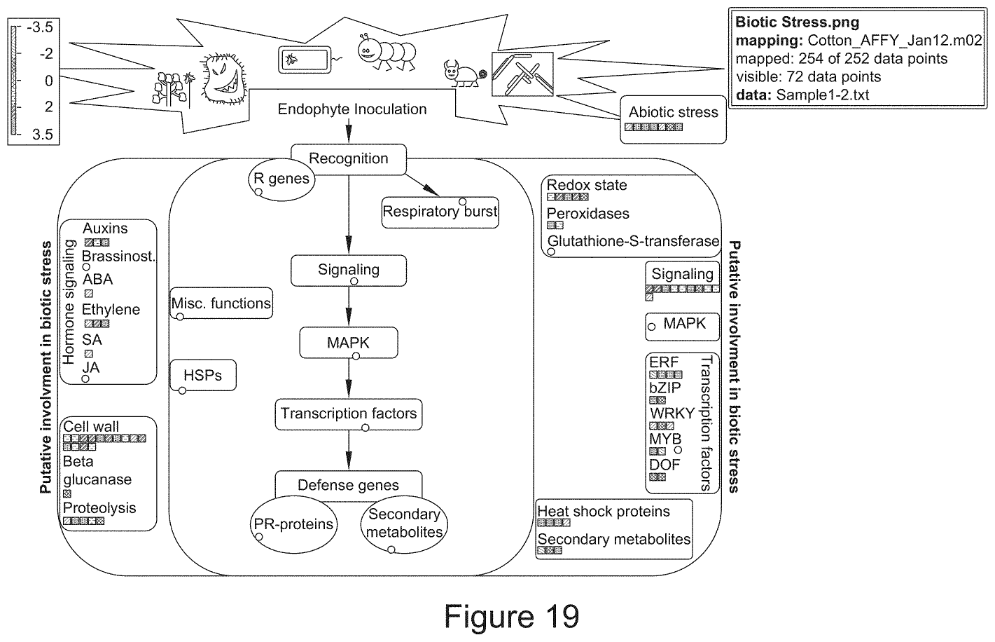

The gene ontology flash bar chart generated using SEA in agriGO illustrated that there was a greater percentage of expressed genes related to numerous processes compared to background data including: cellular component organization, immune system processes, growth, cellular component biogenesis, biological regulation, death, cellular processes, metabolic processes, establishment of localization, localization, response to stimulus, organelles, macromolecular complex, cell parts, cells, extracellular regions, structural molecule activity, transporter activity, antioxidant activity, catalytic activity, electron carrier activity and binding activity (FIG. 19).

TABLE 13
Exemplary enriched pathways in inoculated cotton seedling roots
in non-stressed conditions identified using SEA in agriGO.

| GO term | Description | p-value | FDR |
|---|---|---|---|
| GO:0042126 | nitrate metabolic process | 2.10E−05 | 0.003 |
| GO:0042128 | nitrate assimilation | 2.10E−05 | 0.003 |
| GO:0034641 | cellular nitrogen compound metabolic process | 1.40E−05 | 0.003 |
| GO:0006528 | asparagine metabolic process | 0.00023 | 0.025 |
| GO:0007018 | microtubule-based movement | 0.00037 | 0.031 |
| GO:0016661 | oxidoreductase activity, acting on other nitrogenous | 3.00E−05 | 0.006 |
| | compounds as donors | | |
| GO:0005507 | copper ion binding | 0.00026 | 0.013 |
| GO:0048307 | ferredoxin-nitrite reductase activity | 0.00024 | 0.013 |
| GO:0016664 | oxidoreductase activity, acting on other nitrogenous | 0.00024 | 0.013 |
| | compounds as donors, iron-sulfur protein as acceptor | | |
| GO:0016491 | oxidoreductase activity | 0.00067 | 0.026 |
| GO:0050660 | FAD binding | 0.00078 | 0.026 |
| GO:0070569 | uridylyltransferase activity | 0.0018 | 0.036 |
| GO:0020037 | heme binding | 0.0013 | 0.036 |
| GO:0051748 | UTP-monosaccharide-1-phosphate uridylyltransferase activity | 0.0018 | 0.036 |
| GO:0003983 | UTP:glucose-1-phosphate uridylyltransferase activity | 0.0014 | 0.036 |
| GO:0046906 | tetrapyrrole binding | 0.0026 | 0.047 |
| GO:0005576 | extracellular region | 2.20E−05 | 0.0024 |
| GO:0016023 | cytoplasmic membrane-bounded vesicle | 0.00016 | 0.0037 |
| GO:0031982 | Vesicle | 0.00017 | 0.0037 |
| GO:0031988 | membrane-bounded vesicle | 0.00016 | 0.0037 |
| GO:0031410 | cytoplasmic vesicle | 0.00017 | 0.0037 |
| GO:0031225 | anchored to membrane | 0.00037 | 0.0069 |
| GO:0015630 | microtubule cytoskeleton | 0.0022 | 0.034 |
| GO:0005874 | Microtubule | 0.0026 | 0.036 |

MapMan Functional Analysis and Pathway Mapping

MapMan was used to illustrate the pathways and functions of DEGs in non-stressed and salt stressed cotton seedling roots inoculated with Bacillus amyloliquefaciens (strain Bamy). An overview of DEGs involved in metabolism showed that non-stressed seedling roots had up-regulated nitrate metabolism and assimilation genes. In addition, a nitrogen metabolism pathway analysis demonstrated that various genes were involved in nitrogen assimilation (FIG. 17). MapMan analysis also demonstrated that genes related to cell walls, lipid and amino acid metabolism, and the oxidative pentose phosphate pathway were up-regulated in non-stressed cotton seedlings inoculated with B. amyloliquefaciens (strain Bamy) (FIG. 16). Some of the down-regulated genes in inoculated and non-stressed cotton seedlings were related to light reactions, nitrogen degradation, and carbohydrate metabolism (FIG. 16). MapMan was also used to generate a visual summary of metabolism-related genes that were differentially expressed in salt stressed and inoculated cotton seedling roots (FIG. 18). Gene transcripts related to cell walls, phenylpropanoids and phenolics, and lipids were up-regulated in inoculated and salt stressed cotton seedling roots. Some down-regulated transcripts in the metabolism category were related to the light reactions, electron transport, and wax synthesis.

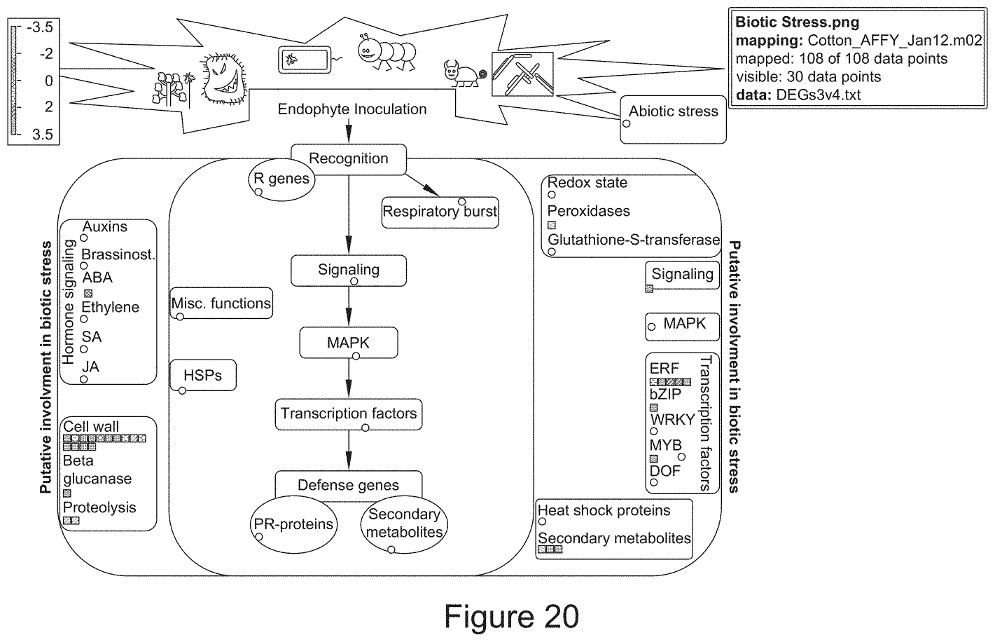

MapMan was also used to summarize and illustrate DEGs involved in biotic stress pathways in inoculated cotton seedling roots in non-stressed and salt stressed conditions. A visual summary of gene transcripts involved in biotic stress-related pathways showed that inoculating non-stressed cotton seedling roots with Bacillus amyloliquefaciens (strain Bamy) led to the differential expression of numerous genes. Various DEGs were related to hormonal signaling, redox state and antioxidants, signaling, transcription factors, cell walls, proteolysis, and heat shock proteins (FIG. 19). In addition, inoculating non-stressed cotton seedling roots with B. amyloliquefaciens (strain Bamy) led to the up-regulation of various genes related to abiotic stress. Some of the up-regulated transcripts related to biotic stress pathways were involved in auxin and ethylene pathways indicating that hormonal pathways were up-regulated in non-stressed inoculated seedlings. Inoculated seedling roots under salt stressed conditions also had differential expression of genes related to biotic stress pathways. Transcripts encoding for genes related to cell walls, betaglucanases, transcription factors, secondary metabolites, and signaling (FIG. 20).

Regulatory processes were also summarized and visualized using MapMan. Non-stressed cotton seedling roots inoculated with Bacillus amyloliquefaciens (strain Bamy) had DEGs related to processes including transcription factors, protein modification, protein degradation, hormones, receptor kinases, calcium regulation, and numerous redox genes. Inoculated and salt stressed cotton seedlings also had a number of DEGs involved in regulatory processes. DEGs were categorized under categories such as transcription factors, protein modification and degradation, and calcium regulation.

DEGs involved in sugar metabolism, specifically glycolysis and the TCA cycle, were visualized using MapMan. Six transcripts were identified to be involved in these pathways in non-stressed cotton seedling roots inoculated with Bacillus amyloliquefaciens (strain Bamy). The up-regulated genes were identified in MapMan as fructokinases, glucose-1-phosphate uridylyltransferase, citrate lyase, and malate dehydrogenase. A down-regulated transcript was identified as an NADPH dehydrogenase. Salt stressed cotton seedling roots inoculated with Bacillus amyloliquefaciens (strain Bamy, also referred to as strain PB1) had 2 DEGs related to glycolysis and the TCA cycle. These genes were identified in MapMan as NADPH dehydrogenases.

qRT-PCR Analysis and Comparison to Microarray Results

Reverse transcriptase quantitative PCR analysis of RNA extractions from non-stressed cotton seedling roots showed that various genes were up-regulated. Five genes that were identified to be up-regulated using GeneChip microarrays were also determined to be up-regulated using qRT-PCR (Table 14). The genes for peroxidase, PR1, expansin A, nitrate transporter, and nitrate reductase were up-regulated in non-stressed and inoculated cotton seedling roots.

TABLE 14
Exemplary changes in gene expression detected using microarrays
and qRT-PCR of RNA extracted from non-stressed cotton seedling roots
inoculated with Bacillus amyloliquefaciens (strain Bamy).

| Gene | Microarray Fold-Change | qPCR Fold-Change |
|---|---|---|
| (Microarray Probe) | (log₂) | (log₂) |
| Peroxidase | 6.73 | 2.62 |
| PR1 | 2.99 | 3.52 |
| Expansin A | 2.7 | 1.41 |
| High affinity nitrate | 2.51 | 4.3 |
| transporter | | |
| Nitrate reductase | 7.54 | 3.8 |

Auxin Pathways

The most up-regulated gene in cotton seedling roots inoculated with B. amyloliquefaciens (strain Bamy) in non-saline conditions encoded for a predicted WAT1-related protein with a 14.75 fold increase in expression. The WAT1 gene encodes for a transmembrane protein that was shown to be involved in growth and secondary cell wall development, auxin transport and homeostasis in Arabidopsis thaliana. In some embodiments, up-regulating the expression of genes encoding for proteins related to WAT1 influence auxin homeostasis and/or transport and thus alter growth.

The up-regulation of transcripts encoding for the predicted protein indole-3-acetic acid amido (IAA) synthetase GH3.1 suggests that inoculated cotton seedlings express genes related to auxin homeostasis. In some embodiments, up-regulation of the predicted protein indole-3-acetic acid amido (IAA) synthetase GH3.1 gene function to control elevated levels of auxin by conjugating IAA to amino acids.

Another up-regulated transcript related to the auxin pathway encoded for the predicted protein IAA14 which was previously shown to be involved in auxin signaling and lateral root formation in Arabidopsis (Fukaki et al 2005). The up-regulation of these IAA-related transcripts suggest that inoculating cotton seedling roots with B. amyloliquefaciens (strain Bamy) enhances the expression of certain genes in the auxin pathway which could have an effect on root growth, lateral root development, and defense. These genes were not shown to be up-regulated in inoculated cotton seedling roots in saline conditions.

Ethylene Pathways

Various transcripts that encoded for transcription factors related to ethylene pathways were up-regulated in non-stressed and salt stressed cotton seedling roots inoculated with Bacillus amyloliquefaciens (strain Bamy). MapMan analysis visualized and summarized these transcripts related to biotic stress pathways and regulatory processes (FIG. 19). Some up-regulated transcripts related to biotic stress pathways in inoculated and non-stressed cotton seedling roots were related to ethylene responsive transcription factors. For example, some of the up-regulated transcripts encoded for the ethylene responsive transcription factor EREB1. Inoculating cotton seedlings with Bacillus amyloliquefaciens (strain Bamy) led to an increased expression of these ethylene-related transcription factors which could lead to greater protection of cotton seedlings from subsequent infections by pathogens.

Cell Division, Growth, Expansion, and Lateral Root Development

Many of the up-regulated transcripts in inoculated seedling roots in both salt stressed and non-stressed conditions were related to plant cell growth, elongation and cell wall synthesis. The increased expression of some of these genes likely results in the loosening of cell walls and/or facilitating the elongation and division of cells in rapidly growing cotton roots.

Genes encoding for the predicted proteins cellulose synthase and COBRA were up-regulated in non-stressed cotton seedling roots inoculated with Bacillus amyloliuefaciens (strain Bamy). In addition, genes encoding for predicted extensins, expansins and fasciclin-like arabinogalactan proteins were up-regulated in inoculated cotton seedlings. Another growth-related gene, fasciclin-like arabinogalactan, was also up-regulated in inoculated and non-stressed cotton seedling roots. Fasciclin-like arabinogalactan proteins are a widely dispersed group of proteins that are predicted to be involved in cellular adhesion due to their fasciclin domains (Johnson et al 2003). Pectinesterases, which were also up-regulated, catalyze the de-esterification of polygalacturonans which are cell wall components. In addition to pectinesterase, another up-regulated growth-related transcript encoded for the predicted enzyme pectate lyase. Pectate lyases are enzymes that break down pectin and are produced by certain microbes (bacteria and fungi) and plants (Marín-Rodríguez et al 2002). Plant pectate lyases are involved in fruit ripening (Marin-Rodriguez et al 2002, Payasi and Sanwal 2003). The pathogenic microbes Erwinia carotovora and Erwinia chrysanthemi which cause soft-rot also produce the enzyme pectate lyase which enhances their virulence (Keen et al 1984, Lei et al 1987, Boccara et al 1988). The enzyme pectate lyase in a legume has been previously linked to the ability of nodule-forming rhizobacteria to successfully penetrate and colonize roots (Xie et al 2012).

The up-regulation of these genes may be a common host response to growth promoting endophytes. Aside from promoting growth, the up-regulation of cell wall loosening enzymes likely facilitates the systemic endophytic colonization of Bacillus amyloliquefaciens (strain Bamy) in cotton seedlings.

Nitrogen Metabolism

Microarray results, SEA analysis, and MapMan pathways demonstrated that the expression of genes related to nitrate uptake and assimilation were up-regulated in non-stressed cotton seedling roots inoculated with B. amyloliquefaciens (strain Bamy). Nitrate assimilation is an energetically costly process that is regulated by an elaborate network of pathways. Nitrate itself is a regulatory signal for the nitrate assimilation pathway and also acts directly as a nutrient by acting as a nitrogen source. Up-regulated transcripts encoding for proteins involved in nitrate assimilation suggest that non-stressed cotton seedlings inoculated with B. amyloliquefaciens (strain Bamy) could have an enhanced ability to import nitrate, transform nitrate to nitrite, nitrite to ammonium, and to subsequently form glutamine. The predicted enzymes asparagine synthetase and asparaginase were also up-regulated. The enhanced expression of nitrogen assimilation genes could ultimately contribute to enhanced root growth of cotton seedlings. Thus, use of endophytic bacteria to increase a plant's ability to uptake nitrogenous compounds could ultimately decrease the need to apply nitrogenous fertilizers.

Carbon Metabolism

Various genes related to glycolysis and the citric acid cycle were up-regulated in non-stressed and inoculated seedling roots. Genes encoding for enzymes such as malate dehydrogenase, glucose-6-phosphate dehydrogenase, ATP-citrate synthase, and fructokinase-6 were up-regulated in non-stressed cotton seedling roots inoculated with B. amyloliquefaciens (strain Bamy) (Table 10). None of these genes were determined to be up-regulated in salt stressed and inoculated cotton seedling roots. The up-regulation of genes involved in carbohydrate metabolism may support the demand of carbon compounds used as backbones for nitrogen assimilation.

The gene encoding for the enzyme malate dehydrogenase was determined to be up-regulated 3.4-fold in non-stressed cotton seedling roots inoculated with Bacillus amyloliquefaciens (strain Bamy) (Table 9). Malate dehydrogenase catalyzes the reversible reaction that facilitates the conversion of malate and oxaloacetate. Inoculating Bacillus amyloliquefaciens (strain Bamy) into cotton seedling roots may increase the expression of genes involved in the production of organic acids such as malic acid and could function to attract beneficial chemotactic bacteria such as Bacillus spp. towards the seedling rhizosphere.

Redox and Antioxidant Enzymes

Antioxidants and oxidoreductases were also up-regulated in inoculated cotton seedling roots in non-stressed and salt stressed conditions. In non-stressed and inoculated seedling roots, various genes transcripts encoding for the predicted enzymes peroxidase, ascorbate oxidase, and monodehydroascorbate peroxidase were up-regulated. Increased expression of genes encoding for antioxidants likely increases the host's tolerance to ROS which was demonstrated to increase upon inoculation with B. amyloliquefaciens (strain Bamy).

Genes encoding for the predicted enzyme laccase were up-regulated in salt stressed cotton seedling roots inoculated with B. amyloliquefaciens (strain Bamy). Laccases are oxidoreductase glycoproteins which are involved in the lignification of vascular tissue (Wang et al 2008, Zhao et al 2013). Furthermore, the staining of cotton seedling roots with phloroglucinol-HCl demonstrated that non-stressed and salt stressed seedlings inoculated with B. amyloliquefaciens (strain Bamy) had more lignin than noninoculated seedlings.

Defense-Related Transcripts

Various genes predicted to be involved in defense pathways were up-regulated in inoculated cotton seedling roots. The gene encoding for a predicted defensin-like protein was up-regulated in non-stressed and inoculated cotton seedling roots. Another up-regulated transcript encoded for a predicted pathogenesis-related protein 1 (PR1) with a 2.99-fold increase in expression (Table 9).

The second most down-regulated gene in non-stressed inoculated cotton seedling roots encoded for a predicted metacaspase 9-like protein with a 43.67-fold decrease in expression. Metacaspase 3 was determined to be down-regulated in inoculated cotton seedling roots in salt stress conditions. In some embodiments, down-regulation of the Metacaspase 3 gene in cotton seedling roots facilitates the endophytic and non-pathogenic behavior of B. amyloliquefaciens (strain Bamy) by preventing cell death symptoms in cotton seedling roots.

Transcription Factors

Various gene transcripts encoding for transcription factors were differentially expressed in non-stressed and salt stressed cotton seedling roots inoculated with Bacillus amyloliquefaciens (strain Bamy). Transcription factors related to biotic stress pathways were summarized in FIGS. 19 and 20. It was determined that genes encoding for ERF transcription factors were up-regulated in inoculated and non-stressed seedlings (Table 9). In contrast, the WRKY transcription factors WRKY70, WRKY75, and WRKY40 related to biotic stress pathways were down-regulated in inoculated cotton seedling roots in non-stressed conditions (FIG. 19). In some embodiments, inoculating cotton seedlings with Bacillus amyloliquefaciens (strain Bamy) in non-stressed conditions leads to the down-regulation of the expression of WRKY transcription factors in cotton seedling roots and thus can have an effect on the ability of cotton seedlings to resist certain pathogens while becoming more susceptible to other types of infections.

The regulation overview visual summaries generated in MapMan also demonstrated that transcripts encoding for transcription factors were differentially expressed in inoculated seedling roots in non-stressed and salt stressed conditions. In addition to the transcripts encoding for WRKY and ERF, there were other transcripts which encoded for transcription factors with possible defense-related functions including two down-regulated transcripts which encoded for LOL1 which are positive regulators of programmed cell death in plants. Furthermore, BLAST results showed that transcripts encoding for ZAT10 and ZAT11, two zinc-finger proteins, were down-regulated in inoculated cottons seedling roots in non-stressed conditions (Table 10).

Comparison of Differential Gene Expression Between Non-Stressed and Salt Stressed Inoculated Cotton Seedling Roots

Differences were observed between the differentially expressed genes identified in non-stressed and salt stressed inoculated cotton seedling roots. For example, genes encoding for proteins involved in nitrate assimilation were up-regulated in non-stressed seedling roots but were not up-regulated in salt stressed seedling roots. In addition to genes involved in nitrate assimilation, non-stressed cotton seedling roots had up-regulated genes related to flavonoid synthesis, oxidative pentose phosphate pathway, amino acid catabolism, and nucleotide synthesis. Instead, inoculated and salt stressed cotton seedling roots had up-regulated genes involved in the phenylpropanoid and phenolic synthesis pathways (FIG. 18). In addition, salt stressed and inoculated seedling roots had up-regulated expression of genes encoding for laccase and polygalacturonase while differential gene expression of these transcripts was not observed in non-stressed cotton seedling roots.

Similarities were also observed between the differentially expressed genes identified in non-stressed and salt stressed inoculated cotton seedling roots. Inoculated roots grown in both conditions had enhanced expression of genes related to cell wall processes including the cell wall loosening enzymes expansin, pectinesterase, and fasciclin-like arabinogalactan. In addition, the growth and microtubule related genes encoding for tubulins and kinesins were also up-regulated in inoculated roots growing in both conditions. Another similarity between both systems was that genes related to calcium regulation were up-regulated in inoculated cotton seedling roots in both non-stressed and salt stressed conditions. MapMan data showed that in non-stressed roots, genes encoding for calmodulin and calreticulin were up-regulated. In salt-stressed conditions, a different transcript encoding for a predicted calmodulin was up-regulated. Calmodulins are regulatory proteins that are involved in signal transduction of various processes.

### Conclusions

Acid delinted cotton seeds have disturbed seed microbiomes and could benefit from the application of beneficial bacteria. As described above, bacteria from wild, non-cultivated relatives of cotton altered gene expression of cotton seedling roots. The differential expression of genes involved in various functions including nitrogen assimilation, antioxidants, cell division and growth, defense, transcription factors, and transporters supports that the growth promotional effect of beneficial bacterial endophytes could be partially due to a complex genetic response by the host. This research demonstrated that hundreds of genes were differentially expressed in seedling roots inoculated with the plant growth promoting bacterium Bacillus amyloliquefaciens.

This information also provides new avenues for promoting the growth of target plants. For example, the skilled person may use molecular biological approaches based on the genetic profiles observed in the microbes described herein to up regulate and down regulate gene expression in target plants.

In one embodiment, where enhanced nitrogen absorption is desired, plants can be produced with increased capacity for nitrate assimilation. This can be achieved via introduction of nucleic acid sequences for overexpression of protein sequences or functional fragments thereof, encoding, for example, plasma membrane HA-nitrate transporter, nitrate reductase, glutamine synthase, Wat-1, asparagine synthase, and asparaginase. In an alternative or combinatorial approach, gene editing, siRNA or co-suppression approaches can be employed to down modulate expression of glutamate dehydrogenase 2 for example.

In embodiments for increasing salt tolerance, overexpression of one or more of blue copper protein, lipid transfer protein DIR1, expansin A8, and or laccase 4 is desirable. Alternatively, or in combination, gene editing, siRNA or co-suppression approaches can be employed to down modulate expression of expansin B1, cucumisin, UDP-glycosyltransferase, alcohol dehydrogenase, and zinc finger protein ZAT11.

In embodiments for increasing disease resistance in plants, overexpression of one or more of EREB1, PR protein (PR1), ERF114, and peroxidase is effective. As above this approach may be employed alone or in combination with gene editing, siRNA or co-suppression approaches for down regulating genes including, without limitation, metacaspase 9, metacaspase 3, and salicylate carboxymethyltransferase.

In an alternative approach, components of the bacterial cells themselves could be used to alter gene expression in a target plant. For example, cells wall components of bacteria could be used to induce expression of plant resistance genes and make plants resistant to pathogens or stresses.

### Example III

Defensive Properties of Bacterial Endophytes from Non-Cultivated Relatives of Cotton

Agricultural practices that alter the seed microbiome and reduce the community of vertically transmitted beneficial microbes have the potential to increase the vulnerability of crops to fungal pathogens. Acid delinting is a common practice used to remove lint from cotton seeds during seed mass production. The removal of beneficial microbes through acid delinting, further justifies the need to find effective biocontrol agents to protect vulnerable cotton seeds.

Bacterial endophytes use various mechanisms to enhance plant health and development. Some bacterial endophytes, including Bacillus spp., are capable of producing antifungal compounds such as lipopeptides that induce systemic resistance in plants and protect them against infections by fungal pathogens (Ongena et al 2007, Ongena et al 2005, Romero et al 2007, Zeriouh et al 2011, Gond et al 2015a).

As discussed above, novel bacteria from a non-cultivated, wild relative of cotton (Thespesia populnea) have been isolated and identified. These bacteria were also assessed for their ability to control the growth of seed-transmitted fungi and the underlying mechanisms contributing to antifungal activity. The effects of the bacteria on microscopic characteristics of the fungi including chlamydospore production were also determined. We also determined if bacteria were able to protect economically important crops such as Musa sp. and Gossypium hirsutum against Lasiodiplodia theobromae postharvest fruit rot and Fusarium sp. seed infections, respectively.

The following materials and methods are provided to facilitate the practice of Example III.

Isolation and Identification of Microbes

Bacteria and fungi associated with Thespesia populnea and Gossypium hirsutum were isolated as described above in Example I. To isolate seed-transmitted microbes, seeds were surface sterilized with 4% NaOCl for 15 minutes and inoculated onto PDA and TSA. Cultures were incubated at room temperature (25° C.). Microbes were isolated from surface sterilized stems and leaves of Thespesia populnea seedlings. Seed-transmitted fungi from surface sterilized Gossypium hirsutum were also isolated on PDA. Once isolates of cultures were obtained, stocks were prepared in 30% glycerol stored in cryotubes at −80° C.

Bacteria were identified based on genetic sequencing and fungi were identified using a combination of genetic sequencing and morphological characterization. DNA from the bacteria used in this study was extracted using GenElute™ Bacterial Genomic DNA Kit (Sigma-Aldrich, St. Louis, MO). The 16S rRNA gene was amplified using the primers 27F (5′-AGAGTTTGATCMTGGCTCAG; SEQ ID NO: 19) and 1492R (5′-TACCTTGTTACGACTT; SEQ ID NO: 20). Polymerase chain reactions (PCR) were initially denatured for 5 minutes at 95° C. followed by 30 cycles of 1 minute at 94° C., 1 minute at 55° C., and 1.5 minutes at 72° C. along with a final extension step of 10 minutes at 72° C. DNA from fungal isolates was extracted using the MoBio Power Plant DNA Isolation Kit (MoBio Laboratories, Carlsbad, CA). The ITS region was amplified using the primer set ITS1 (5′-TCCGTAGGTGAACCTTGCGG; SEQ ID NO: 33) and ITS4 (5′-TCCTCCGCTTATTGATATGC; SEQ ID NO: 34). PCR reactions for the ITS region were carried out using an initial denaturation step at 95° C. for 5 minutes and followed by 35 cycles of 30 seconds at 95° C., 30 s at 55° C., and 1 minute at 72° C. followed by an additional extension step of 10 minutes at 72° C. Positive PCR reactions were identified by 1% agarose gel electrophoresis and purified using the QIAquick PCR Purification Kit (Qiagen, Valencia, CA). Purified PCR reactions were sent to GENEWIZ (South Plainfield, NJ) for sequencing. Sequences were analyzed using BLAST (Altschul et al. 1990). Morphological identification of fungi consisted of macroscopic and microscopic observations of colonies grown on PDA.

Screening for Bacterial Endophytes Capable of Producing Antifungal Compounds

Bacteria isolated from surface sterilized seeds and tissue of Thespesia populnea seedlings were screened for antifungal activity against Lasiodiplodia theobromae, Bionectria ochroleuca, Diaporthe spp., Curvularia lunata, Cladosporium sp., Fusarium spp., Fusarium brachygibbosum, Setosphaeria rostrata, Neofusicoccum australe, Neofusicoccum parvum, and Phoma sp. The endophytic bacteria Bacillus amyloliquefaciens, Pantoea dispersa, Pseudomonas oleovorans, Achromobacter xylosoxidans, and Enterobacter cloacae were inoculated on PDA by streaking in a manner that would create three separate areas. The fungi were inoculated in the center of the three areas. The co-cultures were incubated at 25° C. and observed after 7 days for evidence of fungal inhibition. The width of inhibition zones formed between the bacterial and fungal colonies were measured. Three replicates of each treatment were measured.

Effects of Endophytic Bacteria on Seed-Transmitted Fungi of Thespesia populnea and Gossypium hirsutum

The effect endophytic bacteria have on the growth of fungal colonies was evaluated by co-inoculating bacteria and fungi on PDA and measuring the diameter of fungal colonies after 24, 48 and 72 hours. The fungi Lasiodiplodia theobromae, Bionectria ochroleuca, Diaporthe sp., Curvularia lunata, and Fusarium sp. were co-cultured with Bacillus amyloliquefaciens, Pantoea dispersa, Pseudomonas oleovorans (Strain Poryz), Achromobacter xylosoxidans, or Enterobacter cloacae. The bacteria were first streaked throughout the entire surface of the culture media and mycelium was then inoculated in the center of the plate. The cultures were incubated at room temperature (25° C.). The diameter of fungal colonies was recorded daily. Growth rates per day were calculated using the colony diameter data. T-tests were used to determine if there was statistical difference between the average fungal colony diameter inoculated with and without bacteria.

Effects of Endophytic Bacteria on the Hyphal Width of Seed-Transmitted Fungi from Thespesia populnea and Gossypium hirsutum

The hyphal width of Lasiodiplodia theobromae, Bionectria ochroleuca, Diaporthe sp., Curvularia lunata, and Fusarium sp. were measured to determine the effects endophytic bacteria had on the size of fungal hyphae. Microscope slides were prepared by excising a thin piece of agar from the area where the fungi and bacteria intersected. The excised piece of agar was placed on a microscope slide and stained with a solution of aniline blue and lactic acid. The specimens were viewed at 400× magnification using a Nikon Eclipse 80i microscope (Nikon Instruments Inc, Melville, NY). Hyphal width measurements were taken using NIS-Elements Imaging software (Nikon Instruments Inc., Melville, NY). The average hyphal width and standard deviation of each sample was calculated after taking 20 hyphal width measurements per sample. T-tests were used to compare the mean hyphal width from the control samples against the hyphal width of fungi co-cultured with the bacterial endophytes.

Effects of Endophytic Bacteria on the Severity of Lasiodiplodia theobromae Fruit Rot in Bananas

Endophytic bacteria isolated from Thespesia populnea were co-inoculated with Lasiodiplodia theobromae in bananas purchased at the local grocery store in order to screen for endophytes with antifungal activity that decrease the severity of fruit rot. Sterile toothpicks were used to bore small superficial openings into banana pericarps. These perforations did not fully penetrate into the fruit and attempted to mimic superficial wounding. Toothpicks were used to inoculate Bacillus amyloliquefaciens (strain Bamy), Pantoea dispersa (strain Pdisp), Pseudomonas oleovorans (strain Poryz), Enterobacter cloacae (strain Entero), and Achromobacter xylosoxidans (strain Achromo) into each of the wounds. After inoculating the bacteria, a small amount of hyphae from Lasiodiplodia theobromae was subsequently placed into the opening. Bananas treated with different species of bacteria were placed in separate covered transparent plastic containers and left at room temperature (25° C.). The diameter of ten necrotic lesions on the surface of the banana fruits was measured to determine if any of the endophytic bacteria decreased the severity of banana fruit necrosis. Control samples consisted of a banana containing only the superficial holes and another control sample inoculated only with Lasiodiplodia theobromae and lacking any bacterial inoculation. The average diameter of necrotic lesions caused by the fungal growth was calculated. T-tests were used to determine if the endophytic bacteria reduced the width of necrotic lesions on the surface of banana fruits compared to the control without bacteria.

Extraction of Lipopeptides Produced by Bacillus amyloliquefaciens

Lipopeptides were extracted from a liquid culture of Bacillus amyloliquefaciens (strain Bamy) to determine whether or not the bacteria produced antifungal lipopeptides using previously described methods (Smyth et al 2010; Gond et al 2015a). Bacillus amyloliquefaciens (strain Bamy) exhibited antifungal activity against all fungi tested during the screening assay. The bacteria were cultured in 1 L of potato dextrose broth (20 g/L) for 3 days at room temperature. The culture was divided into 250 ml subsamples and centrifuged at 8,000 rpm for 15 minutes at 4° C. To extract the lipopeptides, the supernatant from the subsamples was pooled and acidified using 5N HCl until the solution reached pH 2. The acidified solution was incubated overnight at 4° C. A pellet of precipitate was formed by centrifuging the solution at 10,000 rpm for 20 minutes at 4° C. The supernatant was discarded, and the pellet was dissolved in methanol. The methanol solution containing the contents of the pellet was filtered and the remaining solution was concentrated using a vapor evaporator at 30° C. The lipopeptides were re-dissolved in methanol.

Lipopeptide Disk Diffusion Assay for Antifungal Activity

A filter paper disk diffusion assay of the crude lipopeptide extract obtained from a liquid culture of Bacillus amyloliquefaciens (strain Bamy) was carried out to determine if the extract had antifungal properties against Lasiodiplodia theobromae (Chen et al 2010; Gond et al 2015a). Four sterile filter paper disks were dipped in the lipopeptide extract and placed on a petri dish containing PDA. The fungus was inoculated in between four disks containing the extract and incubated at room temperature (25° C.). Control samples consisted of filter paper disks dipped only in methanol. Colonies were observed for visible signs of growth inhibition.

Identification of Lipopeptides by MALDI-TOF

The crude extract obtained from a 1 L culture of Bacillus amyloliquefaciens (strain Bamy) was directly analyzed by MALDI-TOF to determine if antifungal lipopeptides were present using previously established methods (Gond et al 2015a). A 100 μg/l sample of the extract was diluted ten-fold with 2.5 mg/ml of α-cyano-4-hydroxycinnamic acid in 50% acetonitrile and 0.1% trifluoroacetic acid. Data was acquired in reflection and positive mode in an AB4700 instrument (Applied Biosystems, Foster City, CA). Data was collected between the masses of 800 and 4000 m/z. The MALDI-TOF analysis was carried out at the Center for Integrative Proteomics Research (Rutgers University, Piscataway, NJ). The masses detected were used to identify the types of lipopeptides produced by Bacillus amyloliquefaciens (strain Bamy) after culturing in 1 L of potato dextrose broth for 3 days at room temperature.

Evaluating the Effects of the Crude Lipopeptide Extract from Bacillus amyloliquefaciens on Chlamydospore Formation in Fungi

The effects of the crude lipopeptide extract on the microscopic features of Lasiodiplodia theobromae, Diaporthe sp., Fusarium sp., and Curvularia lunata were observed. The formation of chlamydospores was determined by counting the number of chlamydospores after four days of growth on PDA with filter paper disks dipped in the crude lipopeptide extract of Bacillus amyloliquefaciens (strain Bamy). The same lipopeptide extract was used to treat each of the filter paper disks in order to guarantee that all of them had the same concentration of the lipopeptide extract. Samples were stained with 5 mM of the fluorescent nucleic acid binding stain SYTO13® to confirm that the round structures observed were indeed spores (Life Technologies, Carlsbad, CA). To count chlamydospores, microscope slides were prepared by excising a piece of the fungal culture from the edge of filter paper disk that contained lipopeptides. The samples were stained with aniline blue and lactic acid and viewed at 200× using a light microscope. A 50 m by 50 m square grid was overlaid onto the image of each specimen using NIS-Elements Imaging Software and a number randomizer was used to select plots. The number of chlamydospores per random plot was counted in a total of five 2,500 μm² plots. The average number of chlamydospores was calculated and Student's T-tests were used to determine if there was significant difference between the means of both lipopeptide-treated and untreated samples at a 99% confidence interval.

Purification of Crude Lipopeptide Extract and Testing of Fractions Recovered by HPLC

Purification of the crude lipopeptide extract was carried out using HPLC as previously described (Smyth et al 2010). The methanol in the crude lipopeptide extract was evaporated and the remaining compound was dissolved in water and TFA. The sample was centrifuged at 13,000 rpm for 5 minutes. Five hundred microliters of the crude lipopeptide sample was loaded into the HPLC with a C18 column and a UV-VIS detector (Gilson, Middleton, WI). Mobile phases were prepared as described in Smyth et al (2010) where mobile phase A consisted of 99.95:0.05 water/TFA and mobile phase B consisted of 80:19.95:0.05 acetonitrile/water/TFA. A gradient elution was performed beginning with 100% of mobile phase A and changing to 100% mobile phase B over the course of 80 minutes. 1 ml of the fractions of purified sample was collected per minute using an automated fraction collector. The fractions that corresponded to areas under peaks observed in the HPLC profile were used for further testing. Fifteen fractions were tested for their ability to induce the production of chlamydospores in a culture of Fusarium sp. Ten microliters of each of the fractions was placed on filter paper disks and placed next to a culture of Fusarium sp. on a 6-well plate containing PDA. The area of the fungal colony nearest to the filter paper disk was excised, stained with aniline blue, and observed using light microscopy to determine if the diffused compound from the tested fractions induced chlamydospore production. Fractions that induced chlamydospore production in a Fusarium sp. culture were analyzed using MALDI-TOF as previously described to determine which lipopeptides were present.

Application of Bacillus amyloliquefaciens to cotton seeds to protect germinating seeds against Fusarium sp.

Bacillus amyloliquefaciens (strain Bamy) was applied to surface sterilized commercial cotton seeds to determine if applying the bacteria protected the germinating seeds against fungal infection. Seeds were surface sterilized using 4% NaOCl and were treated with a solution of ˜1×10⁸ cells/ml of Bacillus amyloliquefaciens (strain Bamy) for 2 hours. Seeds were placed in magenta vessels containing 20 g of twice-autoclaved soil. Seeds were subsequently treated with a solution containing ˜1×10⁸ spores/ml of Fusarium sp. Control treatments included seeds that were not treated with neither bacteria nor fungi, seeds treated with bacteria alone, and seeds treated only with fungi. A total of 36 seeds were included in each of 4 treatments for a total of 144 seeds. Seed germination counts were taken after 2, 4, and 6 days upon sowing.

### Results

Isolation and Identification of Bacteria and Fungi from Thespesia populnea and Gossypium hirsutum

Bacteria were isolated from leaf surfaces, green stems and seeds of Thespesia populnea. Seed-transmitted fungi were isolated from Gossypium hirsutum. Bacteria were identified using 16S DNA sequences. Bacillus amyloliquefaciens (strain Bamy) was isolated from leaf surfaces and green stems while Pseudomonas oleovorans (strain Poryz), Enterobacter cloacae (strain Entero), Pantoea dispersa (strain Pdisp), and Achromobacter xylosoxidans (strain Achromo) were isolated from surface sterilized seeds of Thespesia populnea. Fungi were isolated from surface sterilized seeds of both Thespesia populnea and Gossypium hirsutum that were inoculated on PDA. Lasiodiplodia theobromae, Diaporthe spp., Bionectria ochroleuca, Cladosporium sp., Curvularia lunata, Fusarium brachygibbosum, Setosphaeria rostrata, Neofusicoccum australe, Neofusicoccum parvum, Phoma spp., and Fusarium spp. were isolated and identified by their ITS sequences. Identification of fungi was complemented by observation of morphological features using light microscopy.

Screening for Bacterial Endophytes that Reduce the Growth of Seed-Transmitted Fungi

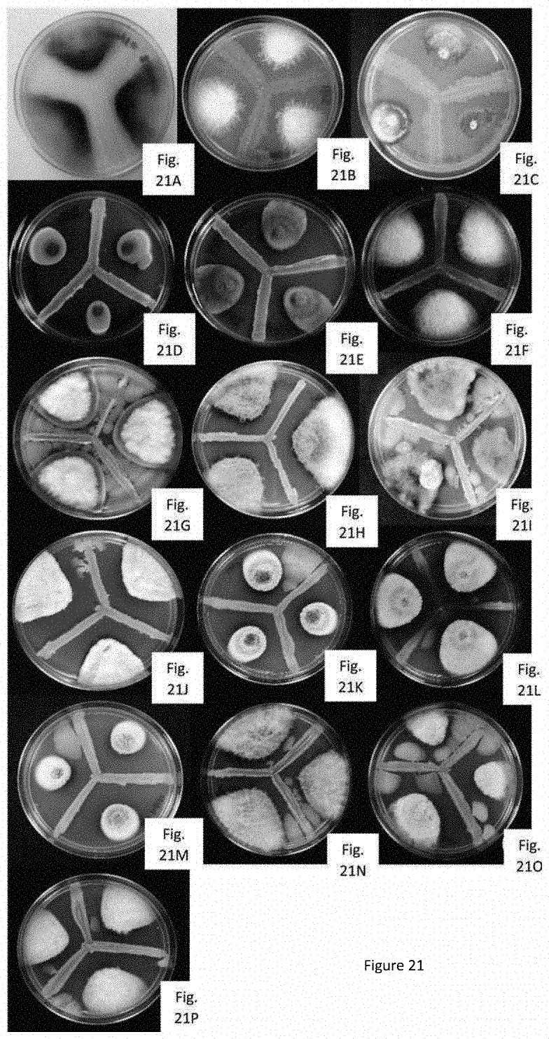

The bacterial endophytes Bacillus amyloliquefaciens (strain Bamy), Pseudomonas oleovorans (strain Poryz), Enterobacter cloacae (strain Entero), Pantoea dispersa (strain Pdisp), and Achromobacter xylosoxidans (strain Achromo) were co-cultured on PDA with various fungi including Lasiodiplodia theobromae, Bionectria ochroleuca, Diaporthe spp., Cladosporium sp., Curvularia lunata, Fusarium spp., Setosphaeria rostrata, Neofusicoccum australe, Neofusicoccum parvum, Phoma sp., and Fusarium brachygibbosum. The formation of inhibition zones between the bacterial and fungal colonies were observed when the fungi were co-cultured with Bacillus amyloliquefaciens (strain Bamy) (FIG. 21). When the fungi were co-cultured near a streak of the other bacterial species tested, the fungi grew through and over the bacteria and continued their growth throughout the Petri dish.

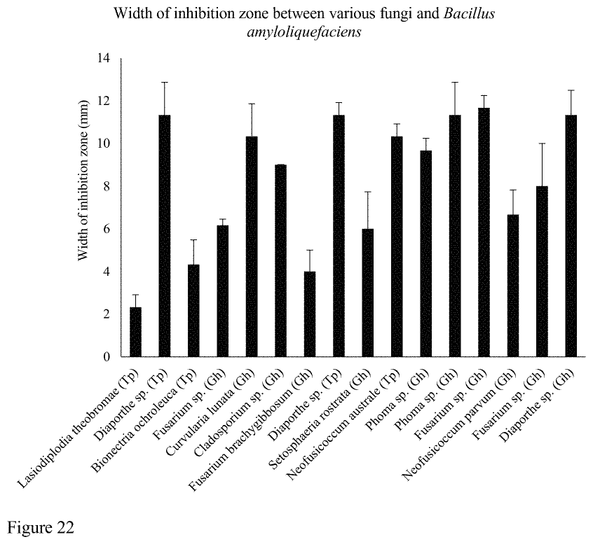

The average width of three inhibition zones produced between B. amyloliquefaciens (strain Bamy) and various fungi were measured (FIG. 22, Table 15). The widest inhibition zone formed after 7 days of growth on PDA was observed between Bacillus amyloliquefaciens (strain Bamy) and Fusarium sp. (11.67±0.58 mm) followed by two different isolates of Diaporthe sp. (both 11.33±1.53 mm), and Phoma sp. (11.33±1.53 mm). The narrowest inhibition zone was measured between Bacillus amyloliquefaciens (strain Bamy) and Lasiodiplodia theobromae (2.33±0.58 mm).

TABLE 15
Width of inhibition zones formed between Bacillus amyloliquefaciens
(strain Bamy) and fungal colonies. Values are the average of
three sample replicates ± the standard deviation.

| | Width of |
|---|---|
| | inhibition zone |
| Fungal isolate | (mm) |
| Lasiodiplodia theobromae | 2.3 ± 0.58 |
| Diaporthe sp. | 11.3 ± 1.53 |
| Bionectria ochroleuca | 4.3 ± 1.15 |
| Fusarium sp. | 6.2 ± 0.29 |
| Curvularia lunata | 10.3 ± 1.5 |
| Cladosporium sp. | 9 ± 0 |
| Fusarium brachygibbosum | 4 ± 1 |
| Diaporthe sp. | 11.3 ± 0.6 |
| Setosphaeria rostrata | 6 ± 1.7 |
| Neofusicoccum australe | 10.3 ± 0.6 |
| Phoma sp. | 9.7 ± 0.6 |
| Phoma sp. | 11.3 ± 1.5 |
| Fusarium sp. | 11.7 ± 0.6 |
| Neofusicoccum parvum | 6.7 ± 1.2 |
| Fusarium sp. | 8 ± 2 |
| Diaporthe sp. | 11.3 ± 1.2 |

Effects of Endophytic Bacteria on the Growth of Seed-Transmitted Fungi

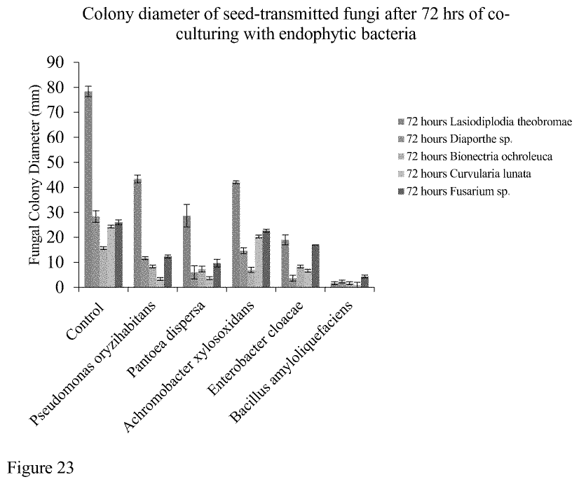

Bacteria and a subset of 5 fungal species were co-cultured on PDA and the diameter of fungal colonies was measured every 24 hours for three days. After 72 hours, all endophytic bacteria tested reduced the colony diameter of Lasiodiplodia theobromae, Diaporthe sp., Bionectria ochroleuca, Fusarium sp., and Curvularia lunata (Table 16, FIG. 23). Significant reduction in average fungal colony diameter was observed when fungi were co-cultured with endophytic bacteria (p<0.05). The average diameter of Lasiodiplodia theobromae colonies grown for 72 hours was smallest when co-cultured with Bacillus amyloliquefaciens (strain Bamy) (1.7 mm), followed by Enterobacter cloacae (strain Entero) (19 mm), Pantoea dispersa (strain Pdisp) (28.7 mm), Achromobacter xylosoxidans (strain Achromo) (42 mm), Pseudomonas oleovorans (strain Poryz) (43.3 mm) and the control without bacteria (78.3 mm). The average diameter of Diaporthe sp. colonies after 72 hours of growth was least when co-cultured with Bacillus amyloliquefaciens (strain Bamy) (2.3 mm) followed by Enterobacter cloacae (strain Entero) (3.7 mm), Pantoea dispersa (strain Pdisp) (6 mm), Pseudomonas oleovorans (strain Poryz) (11.7 mm), Achromobacter xylosoxidans (strain Achromo) (14.7 mm), and the control without bacteria (28.3 mm). The average colony diameter of Bionectria ochroleuca colonies was smallest when co-culturing with Bacillus amyloliquefaciens (strain Bamy) (1.7 mm) followed by Achromobacter xylosoxidans (strain Achromo) (7 mm), Pantoea dispersa (strain Pdisp) (7.3 mm), Enterobacter cloacae (strain Entero) (8.3 mm), Pseudomonas oryzihabitans (strain WCB2) (8.3 mm) and the control samples without bacteria (15.7 mm). The endophyte that reduced the colony diameter of Fusarium sp. the most was Bacillus amyloliquefaciens (strain Bamy) (4.3 mm), followed by Pantoea dispersa (strain Pdisp) (9.7 mm), Enterobacter cloacae (strain Entero) (17 mm), Achromobacter xylosoxidans (strain Achromo) (22.7 mm), and the control (26 mm). The colony diameter of the fungus Curvularia lunata was smallest when co-inoculated with Bacillus amyloliquefaciens (strain Bamy) (1 mm) followed by Pseudomonas oryzihabitans (strain Poryz) (3.3 mm), Pantoea dispersa (strain Pdisp) (3.7 mm), Enterobacter cloacae (strain Entero) (6.7 mm), Achromobacter xylosoxidans (strain Achromo) (20.3 mm) and the control (24.3 mm).

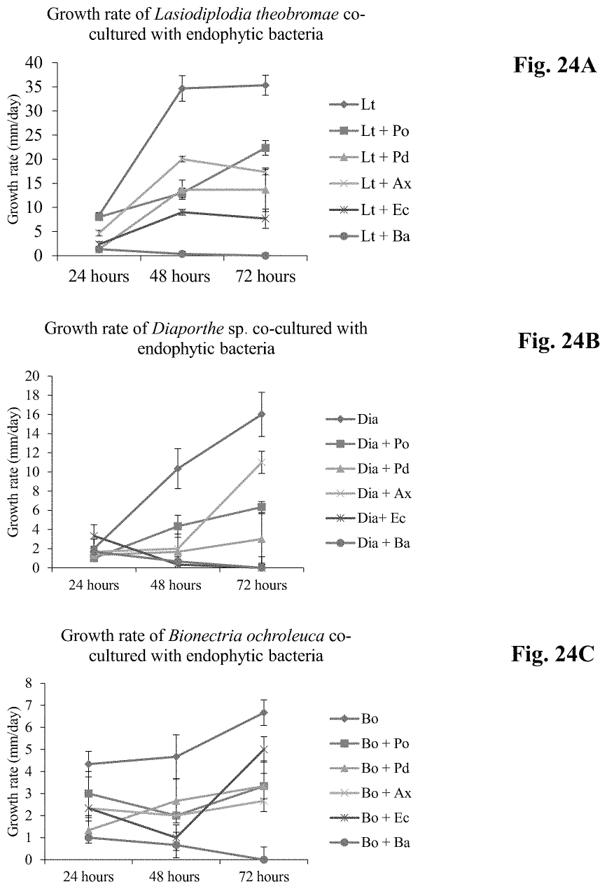

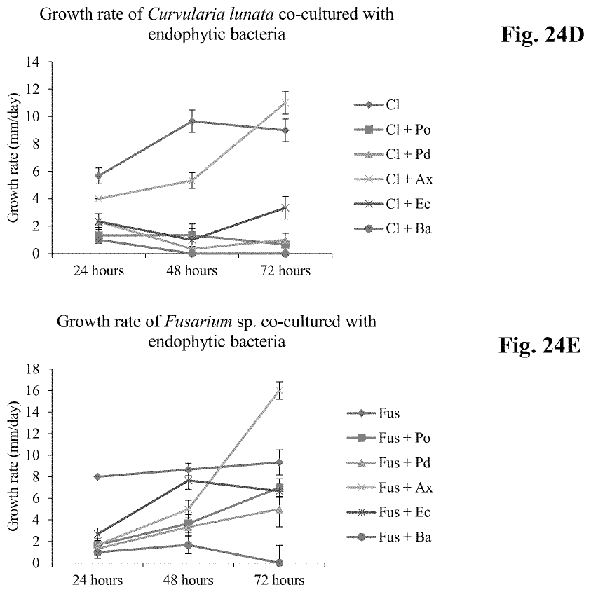

Growth rates (mm/day) of fungal colonies of Lasiodiplodia theobromae, Diaporthe sp., Bionectria ochroleuca, Fusarium sp., and Curvularia lunata co-cultured with various endophytic bacteria were calculated using the colony diameter data collected at 24, 48, and 72 hours (FIGS. 24A-24E). The bacterial endophyte that decreased the daily growth rate of filamentous fungi the most was Bacillus amyloliquefaciens (strain Bamy). After 72 hours, the average growth rate of Lasiodiplodia theobromae colonies were reduced 35-fold when co-cultured with Bacillus amyloliquefaciens (strain Bamy) compared to the growth of Lasiodiplodia theobromae without bacteria (FIG. 24A). The endophyte that decreased the growth rate of Lasiodiplodia theobromae the least was Pantoea dispersa (strain Pdisp), though the difference in growth rate was significant when compared to the control without bacteria. The growth rate of Diaporthe sp. colonies was reduced to 0 mm/day when co-culturing the fungus with Bacillus amyloliquefaciens (strain Bamy) for 48 hours. The growth rate was also reduced to 0 mm/day after co-culturing Diaporthe sp. with Enterobacter cloacae (strain Entero) for 72 hours (FIG. 24B). With the exception of Diaporthe sp. co-cultured with Bacillus amyloliquefaciens (strain Bamy) and Enterobacter cloacae (strain Entero), all other co-cultures with endophytic bacteria had positive growth rate values after 72 hours indicating that the rate of fungal growth was decreased and not entirely inhibited. After 72 hours, there was a significant reduction in the growth rate of Diaporthe sp. when co-cultured with any of the endophytic bacteria tested compared to fungal colonies without bacteria (p<0.0⁵). The growth rate of Bionectria ochroleuca was also significantly reduced by co-culturing the fungus with the endophytic bacteria after 72 hours (p<0.05). The species that least inhibited the growth of Bionectria ochroleuca after 72 hours was Enterobacter cloacae (strain Entero) (5 mm/day) while Bacillus amyloliquefaciens (strain Bamy) decreased the growth rate of this fungus the most after 72 hours (0 mm/day) (FIG. 24C). The average growth rate of Curvularia lunata was calculated when co-cultured with the various endophytic bacteria (FIG. 24D). The growth rate of Curvularia lunata after 72 hours of growth was reduced when co-cultured with endophytic bacteria. The growth rate was the least when co-cultured with Bacillus amyloliquefaciens (strain Bamy) (0 mm/day) compared to cultures without bacteria. The growth rate of Fusarium sp.co-cultured with various endophytic bacteria was also determined after 24, 48, and 72 hours. After 72 hours of growth, Fusarium sp. had a greater growth rate when co-cultured with the bacterial endophyte Achromobacter xylosoxidans (strain Achromo) compared to fungal colonies in the control treatment that were not co-cultured with any of the endophytic bacteria tested (16 mm/day vs 9.33 mm/day). The growth rate of Fusarium sp. co-cultured with Bacillus amyloliquefaciens (strain Bamy) for 72 hours was 0 mm/day (FIG. 24E). The most consistent bacterial endophyte at reducing the growth rate and diameter of different fungal species was Bacillus amyloliquefaciens (strain Bamy). The growth rate in all five fungi co-cultured with Bacillus amyloliquefaciens (strain Bamy) was reduced to 0 mm/day after 72 hours of growth.

TABLE 16
The effects of endophytic bacteria on the average colony diameter of
various filamentous fungi after 72 hours (n = 3). The values indicate
the average diameter of fungal colonies (mm) ± the standard deviation.

| | Lasiodiplodia | Diaporthe | Bionectria | Curvularia | Fusarium |
|---|---|---|---|---|---|
| Treatment | theobromae | sp. | ochroleuca | lunata | sp. |
| Control | 78.3 ± 2.1 | 28.3 ± 2.3 | 15.7 ± 0.6 | 24.3 ± 0.6 | 26.0 ± 1 |
| Pseudomonas | 43.3 ± 1.5 | 11.7 ± 0.6 | 8.3 ± 0.6 | 3.3 ± 0.6 | 12.3 ± 0.6 |
| oleovorans | | | | | |
| (Poryz) | | | | | |
| Pantoea dispersa | 28.7 ± 4.5 | 6.0 ± 2.6 | 7.3 ± 1.2 | 3.7 ± 0.6 | 9.7 ± 1.5 |
| (Pdisp) | | | | | |
| Achromobacter | 42.0 ± 0.6 | 14.7 ± 1.2 | 7.0 ± 1 | 20.3 ± 0.6 | 22.7 ± 0.6 |
| xylosoxidans | | | | | |
| (Achromo) | | | | | |
| Enterobacter | 19.0 ± 2 | 3.7 ± 1.2 | 8.3 ± 0.6 | 6.7 ± 0.6 | 17.0 ± 1 |
| cloacae (Entero) | | | | | |
| Bacillus | 1.7 ± 0.6 | 2.3 ± 0.6 | 1.7 ± 0.6 | 1.0 ± 0 | 4.3 ± 0.6 |
| amyloliquefaciens | | | | | |
| (Bamy) | | | | | |

Effects of Endophytic Bacteria on the Hyphal Width of Seed-Transmitted Fungi

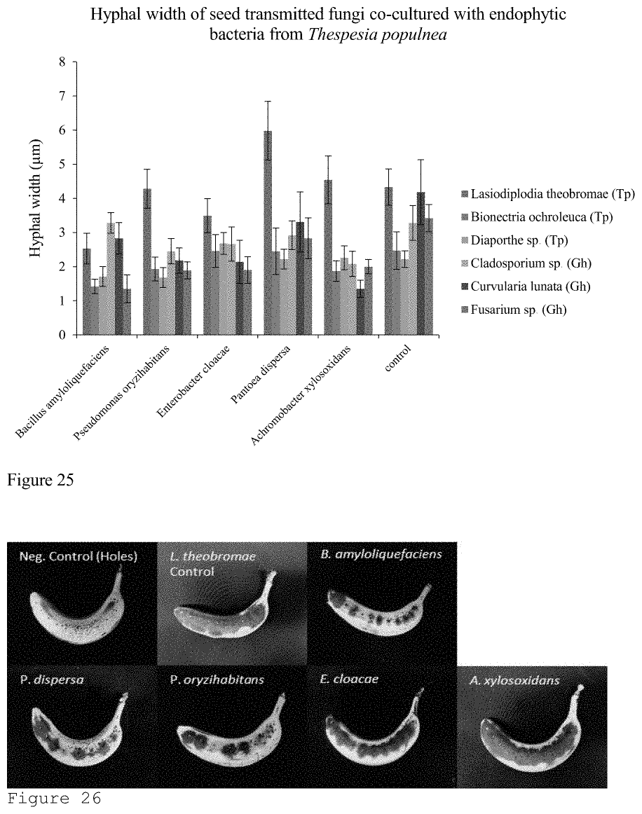

Some of the endophytic bacteria co-cultured with seed-transmitted fungi had an effect on the microscopic characteristics of fungal hyphae. The effects of the bacteria on the hyphae were quantified by measuring the hyphal width when co-cultured with the bacteria. Results showed that Bacillus amyloliquefaciens (strain Bamy) significantly decreased the hyphal width of Lasiodiplodia theobromae, Bionectria ochroleuca, Diaporthe sp., Curvularia lunata, and Fusarium sp. (p=0.00) (FIG. 25, Table 17). The bacterial species Pantoea dispersa (strain Pdisp) significantly increased the width of Lasiodiplodia theobromae hyphae (p=0.00). The bacterial endophyte Pseudomonas oleovorans (strain Poryz) significantly decreased the hyphal width of Diaporthe sp., Curvularia lunata, and Fusarium sp.

TABLE 17
Average hyphal width of fungi from Thespesia populnea (Tp) and Gossypium hirsutum
(Gh) co-cultured with different endophytic bacteria (n = 20).

| | Bacillus | Pseudomonas | Enterobacter | Pantoea | Achromobacter | |
|---|---|---|---|---|---|---|
| | amyloliquefaciens | oleovorans | cloacae | dispersa | xylosoxidans | |
| Treatment | (Bamy) | (Poryz) | (Entero) | (Pdisp) | (Achromo) | Control |
| Lasiodiplodia | 2.53 ± 0.45 | 4.28 ± 0.57 | 3.49 ± 0.5 | 5.98 ± 0.86 | 4.54 ± 0.7 | 4.33 ± 0.53 |
| theobromae (Tp) | | | | | | |
| Bionectria | 1.42 ± 0.21 | 1.93 ± 0.35 | 2.46 ± 0.48 | 2.45 ± 0.68 | 1.87 ± 0.3 | 2.47 ± 0.55 |
| ochroleuca (Tp) | | | | | | |
| Diaporthe sp. | 1.71 ± 0.29 | 1.68 ± 0.29 | 2.68 ± 0.32 | 2.22 ± 0.29 | 2.26 ± 0.35 | 2.22 ± 0.24 |
| (Tp) | | | | | | |
| Cladosporium | 3.28 ± 0.3 | 2.45 ± 0.37 | 2.66 ± 0.5 | 2.92 ± 0.42 | 2.08 ± 0.37 | 3.28 ± 0.51 |
| sp.(Gh) | | | | | | |
| Curvularia | 2.83 ± 0.46 | 2.18 ± 0.37 | 2.14 ± 0.63 | 3.31 ± 0.88 | 1.35 ± 0.25 | 4.18 ± 0.95 |
| lunata (Gh) | | | | | | |
| Fusarium | 1.35 ± 0.41 | 1.89 ± 0.25 | 1.9 ± 0.39 | 2.83 ± 0.6 | 2 ± 0.21 | 3.42 ± 0.4 |
| sp.(Gh) | | | | | | |

Effects of Endophytic Bacteriaon the Severity of Lasiodiplodia theobromae Fruit Rot in Bananas

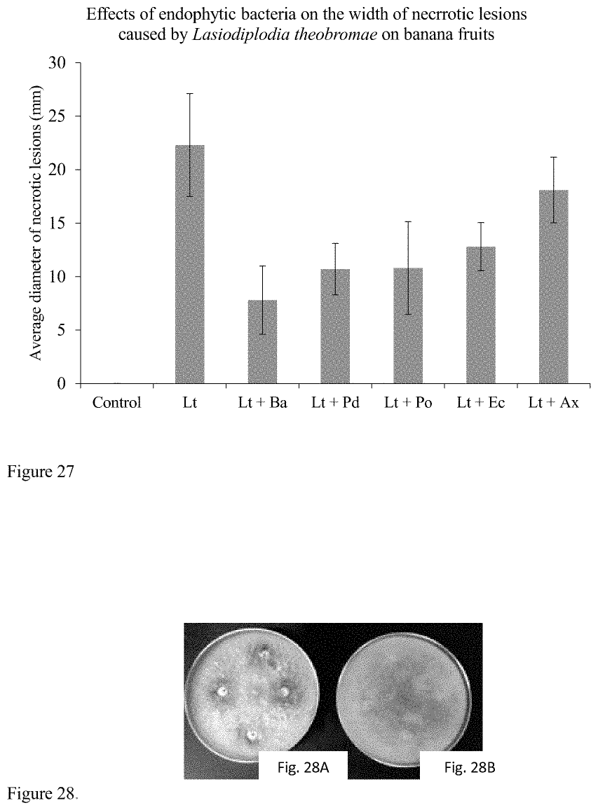

Lasiodiplodia theobromae was co-inoculated with endophytic bacteria on banana pericarps to evaluate if the bacteria reduced the severity of fruit rot in bananas. The endophytic bacteria Bacillus amyloliquefaciens (strain Bamy), Pantoea dispersa (strain Pdisp), Pseudomonas oleovorans (strain Poryz), Enterobacter cloacae (strain Entero), and Achromobacter xylosoxidans (strain Achromo) were inoculated into banana pericarps along with Lasiodiplodia theobromae. The diameter of necrotic lesions associated with the growth of L. theobromae on the banana pericarps was measured. After 3 days of inoculation, the average diameter of necrotic lesions inoculated with Lasiodiplodia theobromae alone was 22.3 mm. The average diameter of necrotic lesions when the fungus was co-inoculated with Bacillus amyloliquefaciens (strain Bamy), Pantoea dispersa (strain Pdisp), Pseudomonas oleovorans (strain Poryz), Enterobacter cloacae, and Achromobacter xylosoxidans were 7.8 mm, 10.7 mm, 10.8 mm, 12.8 mm, and 18.1 mm (FIG. 22). T-tests confirmed that all of the endophytic bacteria tested significantly reduced the severity of Lasiodiplodia theobromae fruit rot in bananas (p<0.0⁵). Bacillus amyloliquefaciens reduced the growth and necrosis caused by Lasiodiplodia theobromae in banana fruit pericarps the most (FIGS. 26 and 27).

Disk Diffusion Assay for Antifungal Activity of the Lipopeptides Produced by Bacillus amyloliquefaciens

Lipopeptides were extracted from a 2-day-old culture of Bacillus amyloliquefaciens (strain Bamy) in potato dextrose broth. The crude lipopeptides obtained showed evidence of antifungal activity towards Lasiodiplodia theobromae (FIG. 28A). The growth and height of aerial mycelia was reduced in the areas near the filter paper disks treated with the crude lipopeptide extract obtained from the liquid culture of Bacillus amyloliquefaciens (strain Bamy). Lasiodiplodia theobromae became more pigmented in the area closest to the lipopeptides diffusing into the medium. Antifungal activity was not observed in the control treated with methanol (FIG. 28B).

Production and Identification of Antifungal Lipopeptides by Bacillus amyloliquefaciens

The lipopeptide extract obtained from a 2-day-old culture of Bacillus amyloliquefaciens (strain Bamy) on PDB contained compounds with molecular weights similar to iturin A, kannurin, surfactin and fengycin using MALDI-TOF (Table 18). The highest peak observed from the extract was 1463.8 m/z which suggests that fengycin was produced the most among the lipopeptides produced by this strain of Bacillus amyloliquefaciens (strain Bamy). The second most produced lipopeptides were surfactins (1060.6 m/z) followed by iturin A (1044.7 m/z) and kannurins (1022.7 m/z). Additional smaller peaks surrounding the major peaks in the mass spectra differed by a factor of 14 indicating that they corresponded to the same group of lipopeptides but differed in the total number of carbon and hydrogen atoms (14 m/z).

Purification of Crude Lipopeptide Extract and Testing of Fractions Recovered by HPLC

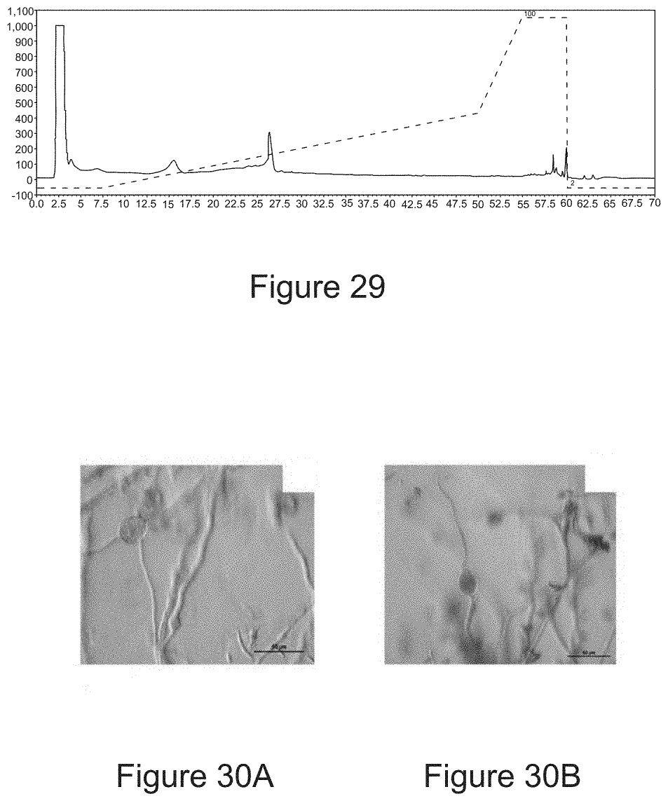

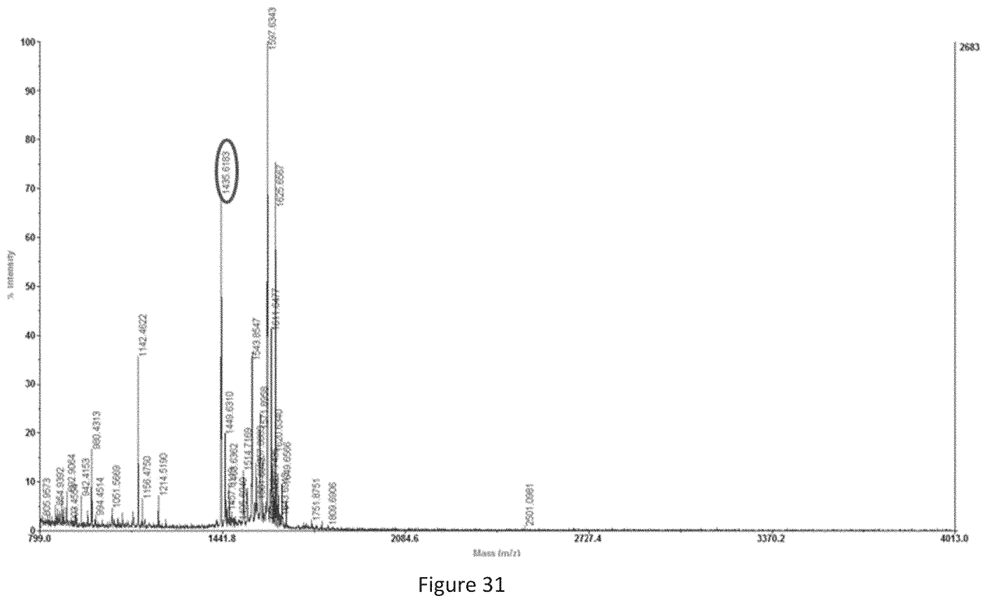

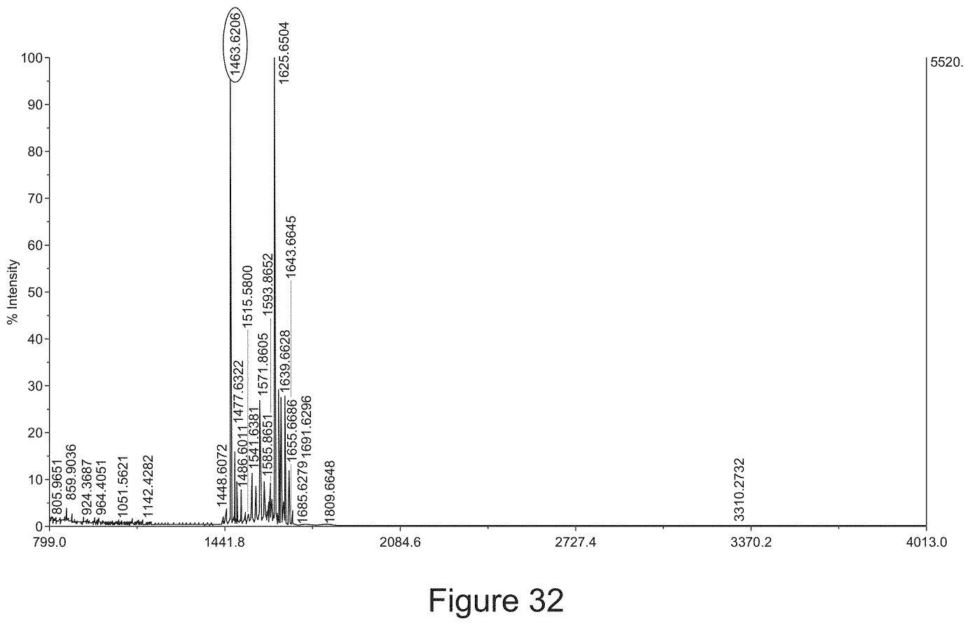

A crude lipopeptide extract was obtained from a 2-day-old culture of Bacillus amyloliquefaciens (strain Bamy) on potato dextrose broth. The sample was purified using HPLC and 80 fractions were obtained after the total run time. A number of peaks were detected using HPLC (FIG. 29). The contents of fifteen fractions corresponding to the areas under peaks detected were tested for their antimicrobial activity against Fusarium sp. and induction of chlamydospore formation. Only two of the fractions tested, #59 and #60, induced chlamydospore formation in Fusarium sp. on PDA using disk diffusion assays (FIG. 30A-30B). None of the other fractions tested induced chlamydospore production in Fusarium sp. Fractions #59 and #60 were analyzed by MALDI-TOF to determine which of the lipopeptides detected in the crude extract was responsible for the induction of chlamydospore formation (FIGS. 31 and 32). A peak of 1463 m/z was detected in both fractions along with another unidentified peak suggesting that fengycin plays an important role in inducing chlamydospore formation in fungi.

Protection of Germinating Cotton Seeds Against Fusarium sp. Using Bacillus amyloliquefaciens

Bacillus amyloliquefaciens (strain Bamy) and Fusarium sp. were individually inoculated and co-inoculated onto cotton seeds to determine if the bacteria enhanced the germination of seeds in the presence of a seed-borne pathogen. After 2, 4, and 6 days the germination of cotton seeds was greater when inoculated with Bacillus amyloliquefaciens than when seeds infected with Fusarium sp. were not inoculated with the bacteria (Table 19). After 6 days 44.5% of seeds inoculated with Fusarium sp. alone had germinated while 55.6% of seeds infected with Fusarium sp. and treated with Bacillus amyloliquefaciens (strain Bamy) germinated.

TABLE 19
Percent of seeds germinated after 2, 4, and 6 days of inoculating seeds
with Bacillus amyloliquefaciens and/or Fusarium sp. (n = 36).
Ba = Bacillus amyloliquefaciens (strain Bamy), Fus = Fusarium sp.

| Time | Control | Ba | Fus | Fus + Ba |
|---|---|---|---|---|
| 2 d | 52.8 | 61.1 | 33.3 | 47.2 |
| 4 d | 66.7 | 66.7 | 38.9 | 52.8 |
| 6 d | 80.6 | 80.6 | 44.4 | 55.6 |

Endophytic Bacteria Alter the Growth and Microscopic Characteristics of Fungal Colonies

The results presented herein reveal that plant endophytes from a non-cultivated relatives of cotton (T. populnea) are capable of altering the growth of seed-transmitted fungi that are potentially pathogenic. The growth in fungal colony diameter and growth rates over a 24 hour period of Lasiodiplodia theobromae, Diaporthe sp., Bionectria ochroleuca, Fusarium sp., and Curvularia lunata co-cultured with endophytic bacteria isolated from Thespesia populnea demonstrated that Bacillus amyloliquefaciens (strain Bamy) most consistently inhibited the growth and decreased the growth rate of the fungi tested. All of the bacteria decreased the colony diameter of the fungi tested. Pseudomonas oleovorans (strain Poryz), Pantoea dispersa (strain Pdisp), Achromobacter xylosoxidans (strain Achromo), and Enterobacter cloacae (strain Entero) decreased the growth rates of fungi but inhibition zones were not formed between the bacterial and fungal colonies. It appears that these bacteria were not secreting antifungal compounds into the culture medium during the incubation period under the growth conditions described here.

Effects of Bacillus amyloliquefaciens on Fungal Hyphae and Growth of Colonies

Bacillus amyloliquefaciens (strain Bamy) is detrimental to the growth of various fungi as demonstratedin this example. Fungal colonies that were co-cultured with Bacillus amyloliquefaciens (strain Bamy) had the greatest reduction in growth and hyphal width compared to fungal colonies co-cultured with other bacterial endophytes. Bacillus amyloliquefaciens (strain Bamy) was repeatedly observed to form inhibition zones and physically adhere to fungal hyphae of Lasiodiplodia theobromae and Fussarium sp. The data collected when co-culturing Bacillus amyloliquefaciens (strain Bamy) and various fungi suggested that Lasiodiplodia theobromae may be the least sensitive to lipopeptides produced by Bacillus amyloliquefaciens (strain Bamy). In addition, the data supports that the three Diaporthe sp. isolates tested were among the most sensitive to the lipopeptides produced by the bacteria. Bacillus spp. have been documented to adhere to cell surfaces and cause deformations in hyphae of fungi including Alternaria brassicae, Fusarium graminearum, Sclerotinia minor, Valsa mali, Aspergillus niger, Fusarium oxysporum, Dothiorella aromatica, and Phomopsis perseae (Sharma and Sharma 2008, Zhao et al 2014, Shrestha et al 2015, Zhang et al 2015, Benoit et al 2015, Vitullo et al 2012, Tesfagiorgis Demoz and Korsten 2005). The physical attachment of Bacillus amyloliquefaciens to fungal hyphae likely enhances its ability to control fungal growth and also suggests that the bacteria are chemotactic towards compounds produced and released by the fungi. Deformations in fungal hyphae were observed in Lasiodiplodia theobromae co-cultured with Bacillus amyloliquefaciens (strain Bamy).

Co-Production of Lipopeptides by Bacillus amyloliquefaciens

Of the various bacteria isolated and tested, Bacillus amyloliquefaciens (strain Bamy) was identified as a candidate bacteria to serve as a broad spectrum antifungal biocontrol agent for cultivated cotton seeds since it consistently inhibited growth of different fungal species. The MALDI-TOF analysis carried out on the crude lipopeptide extract supported that the isolate of Bacillus amyloliquefaciens used in this study co-produced a variety of lipopeptides such as iturin A, kannurin, surfactin, and fengycin. This study supports that kannurin could be produced by more than a single species within the genus Bacillus and is the first report of kannurin being detected in a liquid culture of an endophytic strain of Bacillus amyloliquefaciens.

Lipopeptides as Inducers of Chlamydospore Production in Fungi

The data presented suggest that lipopeptides are capable of inducing chlamydospore production in a wide range of filamentous fungi. The data presented herein appear to provide the first documented evidence of Bacillus amyloliquefaciens-derived lipopeptides inducing chlamydospore formation in cultures of Lasiodiplodia theobromae, Diaporthe sp., and Bionectria ochroleuca.

HPLC was used to separate the various components in the crude lipopeptide extract. It was evident in the HPLC profiles of the chlamydospore-inducing fractions #59 and #60 that a peak corresponding to fengycin was present. The peaks detected in these two fractions, did not correspond to surfactins or iturin, suggesting that fengycin is likely responsible for the induction of chlamydospore production in Fusarium sp. isolated from cotton seeds.

The formation of chlamydospores is associated with conditions of abiotic and biotic stress responses in fungi (Hood and Shew 1997, Kheng Goh et al 2009). The formation of chlamydospores is used by fungi as a mechanism to withstand periods of stress only to germinate once conditions are once again favorable for growth (Sitton and Cook 1981, Nyvall 1970, Hwang and Ko 1978). In some embodiments, lipopeptides produced by beneficial bacteria such as Bacillus spp. induce stress response pathways in filamentous fungi and eventually lead to the induction of chlamydospore production. Though Bacillus spp. and its lipopeptides are able to suppress fungal diseases by inhibiting fungal growth, the production of resistant chlamydospores by the fungus in response to lipopeptides allows it to persist in the environment.

Protection of Banana Fruits and Cotton Seedlings Against Fungal Diseases Using Bacillus amyloliquefaciens

In addition to being able to control the growth of fungal cultures through the production of lipopeptides, the isolate of Bacillus amyloliquefaciens (strain Bamy) used in this study decreased the severity of banana fruit rot caused by L. theobromae and increased the germination rates of cotton seeds infected with Fusarium sp., supporting that applying the bacteria provides a useful biocontrol agent at various stages of plant development and that it provides a benefit to a range of hosts.

Though other bacteria such as Pseudomonas oleovorans (strain Poryz), Pantoea dispersa (strain Pdisp), and Enterobacter cloacae (strain Entero) also decreased the severity of Lasiodiplodia theobromae banana fruit rot, these species were less effective than Bacillus amyloliquefaciens (strain Bamy). Among the bacteria tested, Bacillus amyloliquefaciens (strain Bamy) was the most effective in protecting fruits as well as seedlings and likely has the least threat of being pathogenic to humans.

### Conclusions

Bacterial endophytes interact with filamentous fungi and some are able to alter their growth. Certain bacterial endophytes such as Bacillus amyloliquefaciens (strain Bamy) have a greater ability to inhibit fungi through the co-production of antifungal lipopeptides. In addition to inhibiting growth, mixtures of these lipopeptides induce the production of chlamydospores in a wide range of filamentous fungi. The lipopeptide-producing Bacillus amyloliquefaciens (strain Bamy) can be used to advantage to increase host resistance to fungal pathogens and to be used as a biocontrol agent.

### Example IV

### In Vitro Antibiosis Assay

This example describes a procedure of in vitro antibiosis screenings of microbes against the crop pathogen Fusarium oxysporum, using the non-pathogenic Fusarium oxysporum Fo47 (ATCC, MYA-1198). Caspofungin diacetate (Sigma, SML0425-5MG) is a compound with antifungal activity that is used as a positive control. Caspofungin inhibits ß-1,3-D-glucan synthase and thereby disrupting fungal cell wall integrity. Amphotericin B is a compound with antifungal activity that was used as a positive control. All stock compounds were prepared in DMSO at a concentration of 5,120 μg/ml.

Preparation of Fo47 Spores

Fo47 was cultured on 2% potato dextrose agar (PDA) plates for 14 days at room temperature in a weak light condition. Three ml of 0.05% Silwett L-77 in 1× phosphate buffered saline (PBS) is added to each plate, then mycelium were scraped off and filtered through glass wool into a new 50 ml Falcon tube. Spores were then counted using a hemocytometer and adjusted to 5×106 CFU/ml with sterile 1×PBS.

Preparation of Endophytic Fungal Culture

Five glass beads (3 mm) were added to each well of a 24-deep well plate (VWR, Cat. No. 89080-534) and autoclaved. Fungal cultures were started by adding 5 μl of spore suspension normalized to 1×106 cfu/ml into 3 ml PDB culture into each well. The plates were incubated for 3 days at room temperature with vigorous shaking at 500 rpm.

Preparation of Endophytic Bacterial Culture

Bacterial cultures were started by looping one colony into 3 ml of tryptic soy agar (TSA) in each well of a 24-deep well plate (VWR, Cat. No. 89080-534). The plates were incubated for 3 days at room temperature with vigorous shaking at 500 rpm.

Antibiosis Assay

PDA plates were prepared as follows. PDA with 1% agar were autoclaved in a liquid cycle for 20 minutes with a magnetic stir bar in the flask and kept in a 50° C. water bath. When ready the PDA flask was taken to a sterile environment such as a biosafety cabinet and cooled at room temperature for 15-20 min. Then 2 ml of the prepared Fusarium spores were added per 1 liter of PDA. OmniTrays (ThermoFisher, Cat. No. 264728) were filled with 60 ml of the PDA/spore mixture. After the plates solidify, the plates were air dried for 30 min before covering with the lid.

For each OmniTray, 24 wells were drilled at once using the liquid handling system, BioMek Fx with the following setting: load pod1 (96 pin head) with 24 200-μl wide bore barrier tips (Beckman Coulter, Cat. No. B01110-AA), draw 165 μl well contents using the “Bacterial culture 100 μl technique” at 1.5 mm from the bottom of OmniTray using the “override technique”, dispense tips contents to reservoir plate using the “Reservoir technique” at 6 mm from bottom of OmniTray using “override technique”.

For each OmniTray, 7.5 μl of the prepared bacterial cultures were added into each of the 24 wells using BioMek Fx system, 3 replicated plates were prepared. A negative control (nothing added), a medium control, a DMSO control, a positive compound control (Amphotericin B) and a positive biological control of the same volume were included on each plate.

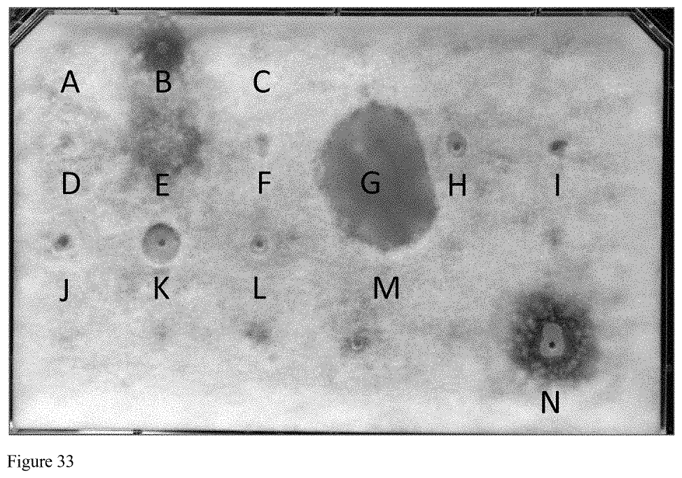

The plates were then incubated at room temperature in sterile conditions for 4 days. Photographs were taken of each plate and the zone of inhibition between the cultures and Fusarium growth were qualitatively scored using a 0-3 scale (3 denotes a strong inhibition) and quantitatively measure using the ImageJ program. An exemplary photograph is shown in FIG. 33 demonstrating the ability of the bacteria of the present invention to inhibit the growth of the fungal pathogen Fusarium oxysporum 47 (Fo47). Nothing was applied at position A as a negative control. The fungicidal compound Amphotericin B was applied at position B as a positive control. The carrier DMSO was applied at position C. The strain Achromo (Achromobacter xylosidans) was applied at position D. The strain Bamy (Bacillus amyloliquefaciens) was applied at position E; this strain produced a distinct zone of inhibition indicating its strong anti-Fusarium activity. The strain WCB1 (Curtobacterium oceanosedimentum) was applied at position F. The strain Entero (Enterobactger cloacae) was applied at position G and showed strong anti-Fusarium activity as denoted by the distinct and large zone of inhibition around the application site. The strain Pdisp (Pantoea dispersa) was applied at position H. The strain Poryz (Pseudomonas oleovorans from T. populnea) was applied at position I. The strain WCB2 (Pseduomonas oryzihabitans from G. hirsuta) was applied at position J. A strain with established anti-Fusarium activity was applied at position N as a positive biological control.

### Example V

### Method of Preparing Bacterial Biomass

The following methods are used to produce bacterial biomass for large scale seed treatments.

Sterile Reasoner's 2A (R2A) agar plates are prepared and strains are streaked for isolation and incubated at room temperature for 24 hours. Individual colonies are picked and re-suspended in adequate volume of 20% TSB to allow for spreading on enough plates for the amount of seeds used.

Sterile Tryptic Soy Agar (TSA) plates are prepared and inoculated with each strain using ˜400 μl of the TSB re-suspension per plate and spread over complete area with a sterile L-shaped plastic spreader. Plates are incubated at room temperature for 48 hours until a robust microbial lawn has grown.

Phosphate buffer is prepared by sterile filtration of 40 mM Phosphate Buffer and 6% wt./vol. Sucrose. A sterile solution of 2% wt./vol. sodium alginate is prepared by autoclaving at 121° C., 15 psi for 30 minutes.

The incubated TSA plates are scraped using an L-shaped spreader and ˜3-5 ml of the phosphate buffer solution, the bacterial suspensions should be as concentrated as possible. The bacterial suspension is then pipetted from the plate into a sterile 50 mL falcon tube using a 10 mL Sterile Serological Pipette and an automatic pipettor. An equal volume of the prepared 2% sodium alginate solution is added to the tube using a 50 mL Sterile Serological Pipette and an automatic pipettor and mixed thoroughly. The bacterial suspension should be stored at 4° C. until used.

### Example VI

### Stability of P. oleovorans in Liquid Culture

P. oleovorans (strain Poryz) was produced in a liquid shake flask in TSB (Tryptic Soy Broth). The initial titer of the biomass at the beginning of this experiment was 3.4×10⁸ CFU/ml. The biomass was stored in liquid culture at 4° C., and the titer of this biomass was checked again 14 days later. At this time, the titer decreased to 1.1×10⁸ CFU/ml. No contamination was detected on bulk biomass.

### Example VII

### Preparation of Sodium Alginate and Talc for Seed Treatments

A 2% weight/volume solution of sodium alginate for the seed coatings is prepared by the following method. An Erlenmeyer flask is filled with the appropriate volume of deionized water and warmed to 50 degrees Celsius on a heat plate with agitation using a stir bar. The appropriate mass of sodium alginate powder for the desired final concentration solution is slowly added until dissolved. The solution is autoclaved at 121 degrees Celsius at 15 PSI for 30 minutes to sterilize. Talcum powder for the powdered seed coatings is prepared by the following method. Talcum powder is aliquoted into Ziploc bags or 50 mL Falcon tubes, and autoclaved in dry cycle (121 degrees Celsius at 15 PSI for 30 minutes) to sterilize.

### Example VIII

### Inoculation of Wheat, Sorghum, Rice or Canola Seeds with Endophytes for Field Experiments

Seeds are heterologously disposed to each endophyte according to the following seed treatment protocol.

Equal parts of the bacterial suspension prepared in Example V and the 2% sodium alginate solution prepared in Example VI are mixed. The solution is applied so that an equivalent of 8.4 ml of bacterial suspension is applied per kg of seeds. Control treatments are prepared using equivalent volumes of 2% sodium alginate solution. The seeds are then agitated to disperse the solution evenly on the seeds.

Then 2 ml per kg of seed of Flo-Rite® 1706 (BASF, Ludwigshafen, Germany) is added and the seeds are agitated to disperse the powder evenly on the seeds. The final concentration of endophyte is targeted to be at least 10\hat{ }CFU. Treated seeds are allowed to dry overnight in a well-ventilated space before planting.

### Assessment of Improved Plant Characteristics: Vigor Assay

Assay of Wheat Seedling Vigor

Seed preparation: The lot of wheat seeds was first evaluated for germination by transfer of 100 seeds and with 8 mL of water to a filter paper lined petri dish. Seeds were incubated for 3 days at 24° C. The process should be repeated with a fresh seed lot if fewer than 95% of the seeds have germinated. Wheat seeds were then surface sterilized by co-incubation with chlorine gas in a 20×30 cm container in a chemical fume hood for 12 hours. Percent germination of 50 seeds, per sterilization batch, was tested as above and confirmed to be greater than 95%.

Preparation of endophyte treatments: Spore solutions were made by rinsing and scraping spores from agar slants which had been growing for about 1 month. Rinsing was done with 0.05% Silwet. Solutions were passed through Miracloth to filter out mycelia. Spores per ml were counted under a microscope using a hemocytometer. The stock suspension was then diluted into 10\hat{ }6 spores/ml utilizing water. 3 μl of spore suspension was used per wheat seed (˜10′CFUs/seed was obtained). Seeds and spores were combined a 50 mL falcon tube and gently shaken for 5-10 seconds until thoroughly coated. Control treatments were prepared by adding equivalent volumes of sterile water to seeds.

Assay of seedling vigor: Petri dishes were prepared by adding four sheets of sterile heavy weight seed germination paper, 50 mL of sterile water added. The sheets were positioned and then creased so that the back of the plate and one side wall were covered, two sheets were then removed and placed on a sterile surface. Along the edge of the plate across from the covered side wall 15 inoculated wheat seeds were placed evenly at least one inch from the top of the plate and half an inch from the sides. Seeds were placed smooth side up and with the pointed end of the seed pointing toward the side wall of the plate covered by germination paper. The seeds were then covered by the two reserved sheets, and the moist paper layers smoothed together to remove air bubbles and secure the seeds, and then the lid was replaced.

For each treatment, three plates with 15 seeds per plate were prepared. The plates were then randomly distributed into stacks of 8-12 plates and a plate without seeds was placed on the top. The stacks were incubated at 60% relative humidity, and 22° C. day, 18° C. night with 12 hours light and 12 hours dark for 24 hours, then each plate was turned to a semi-vertical position with the side wall covered by paper at the bottom. The plates were incubated for an additional 5 days, then wheat seeds were scored manually for germination, root and shoot length.

Statistical analysis was performed using R (R Core Team, 2016. R: A language and environment for statistical computing. R Foundation for Statistical Computing, Vienna, Austria. R-project.org/).

Wheat % Gain Over NT Control

| Strain ID | Root_Length | Shoot_length |
|---|---|---|
| Achromo | −2 | 6 |
| Bamy | 4 | 5 |
| WCB1 | 4 | 5 |
| Entero | −3 | −13 |
| Pdisp | 3 | 7 |
| Poryz | 15 | 12 |

### Example IX

### Inoculation of Soy, Corn, Cotton, or Peanut Seeds with Endophytes for Field Experiments

Seeds are heterologously disposed to each endophyte according to the following seed treatment protocol.

Equal parts of the bacterial suspension prepared in Example V and the 2% sodium alginate solution prepared in Example VI are mixed. The solution is applied so that an equivalent of 8.4 ml of bacterial suspension is applied per kg of seeds. Control treatments are prepared using equivalent volumes of 2% sodium alginate solution. The seeds are then agitated to disperse the solution evenly on the seeds.

Then 2.7 ml per kg of seed of Flo-Rite® 1706 (BASF, Ludwigshafen, Germany) is added and the seeds are agitated to disperse the powder evenly on the seeds. The final concentration of endophyte is targeted to be at least 10\hat{ }CFU. Treated seeds are allowed to dry overnight in a well-ventilated space before planting.

### Assay of Soy Seedling Vigor

Seed preparation: The lot quality of soybean seeds was first assessed by testing germination of 100 seeds. Seeds were placed, 8 seeds per petri dish, on filter paper in petri dishes, 12 mL of water was added to each plate and plates are incubated for 3 days at 24° C. The process should be repeated with a fresh seed lot if fewer than 95% of the seeds have germinated. One thousand soybean seeds were then surface sterilized by co-incubation with chlorine gas in a 20×30 cm container placed in a chemical fume hood for 16 hours. Percent germination of 50 seeds, per sterilization batch, was tested as above and confirmed to be greater than 95%.

Preparation of endophyte treatments: Spore solutions were made by rinsing and scraping spores from agar slants which have been growing for about 1 month. Rinsing was done with 0.05% Silwet. Solutions were passed through Miracloth to filter out mycelia. Spores per ml were counted under a microscope using a hemocytometer. The stock suspension was then diluted into 10\hat{ }6 spores/ml utilizing water. 3 μl of spore suspension was used per soy seed (˜10\hat{ }CFUs/seed is obtained). Control treatments were prepared by adding equivalent volumes of sterile water to seeds.

Assay of seedling vigor: Two rolled pieces of germination paper were placed in a sterile glass gar with 50 mL sterile water, then removed when completely saturated. Then the papers are separated, and inoculated seeds were placed at approximately 1 cm intervals along the length of one sheet of moistened germination paper, at least 2.5 cm from the top of the paper and 3.8 cm from the edge of the paper. The second sheet of was placed on top of the soy seeds and the layered papers and seeds were loosely rolled into a tube. Each tube was secured with a rubber band around the middle and placed in a single sterile glass jar and covered loosely with a lid. For each treatment, three jars with 15 seeds per jar were prepared. The position of jars within the growth chamber was randomized. Jars were incubated at 60% relative humidity, and 22° C. day, 18° C. night with 12 hours light and 12 hours dark for 4 days and then the lids were removed, and the jars incubated for an additional 7 days. Then the germinated soy seedlings were weighed and photographed, and root length and root surface area were scored as follows.

Dirt, excess water, seed coats and other debris was removed from seedlings to allow accurate scanning of the roots. Individual seedlings were laid out on clear plastic trays and trays are arranged on an Epson Expression 11000XL scanner (Epson America, Inc., Long Beach CA). Roots were manually arranged to reduce the amount of overlap. For root measurements, shoots were removed if the shape of the shoot caused it to overlap the roots.

The WinRHIZO software version Arabidopsis Pro2016a (Regents Instruments, Quebec Canada) was used with the following acquisition settings: greyscale 4000 dpi image, speed priority, overlapping (1 object), Root Morphology: Precision (standard), Crossing Detection (normal). The scanning area was set to the maximum scanner area. When each scan was completed, the root area was selected, and root length and root surface area were measured.

Statistical Analysis was Performed Using R.

| | Soy % Gain Over NT Control | | | | |
|---|---|---|---|---|---|
| Strain | Mass of | Root | Root | Seedling | Seedling |
| ID | seedling | Length | Surface Area | Length | Surface Area |
| Achromo | −29 | 19 | 21 | | |
| Bamy | 5 | 9 | 7 | 5 | 10 |
| WCB1 | −18 | 24 | 38 | | |
| Entero | −22 | 22 | 26 | | |
| Pdisp | −8 | 24 | 37 | | |
| Poryz | −9 | 31 | 24 | | |

### Assay of Corn Seedling Vigor

Seed preparation: The lot quality of corn seeds is first evaluated for germination by transfer of 100 seeds and with 3.5 mL of water to a filter paper lined petri dish. Seeds are incubated for 3 days at 24° C. The process should be repeated with a fresh seed lot if fewer than 95% of the seeds have germinated. One thousand corn seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% PEG 6000 (Calbiochem, San Diego, CA) is prepared by adding 75 g of PEG to 1000 mL of water, then stirred on a warm hot plate until the PEG is fully dissolved. The solution is then autoclaved.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per corn seed (˜10\hat{ }3 CFUs/seed is obtained). Control treatments are prepared by adding equivalent volumes of sterile water to seeds.

Assay of seedling vigor: Either 25 ml of sterile water or, optionally, 25 ml of PEG solution as prepared above, is added to each Cyg™ germination pouch (Mega International, Newport, MN) and place into pouch rack (Mega International, Newport, MN). Sterile forceps are used to place corn seeds prepared as above into every other perforation in the germination pouch. Seeds are fitted snugly into each perforation to ensure they did not shift when moving the pouches. Before and in between treatments forceps are sterilized using ethanol and flame and workspace wiped down with 70% ethanol. For each treatment, three pouches with 15 seeds per pouch are prepared. The germination racks with germination pouches are placed into plastic tubs and covered with perforated plastic wrap to prevent drying. Tubs are incubated at 60% relative humidity, and 22° C. day, 18° C. night with 12 hours light and 12 hours dark for 6 days to allow for germination and root length growth. Placement of pouches within racks and racks/tubs within the growth chamber is randomized to minimize positional effect. At the end of 6 days the corn seeds are scored manually for germination, root and shoot length.

Statistical analysis is performed using R or a similar statistical software program.

### Example X

### Assessment of Improved Plant Characteristics: Field Conditions

Wheat

The trial was conducted in an area with sufficient rainfall and access to supplemental irrigation when necessary. Soil information prior to fertilization was collected and any fertilizer applied before and after planting needs is reported by type, grade, and amounts.

Seed varieties were blocked by variety in the field with a border surrounding each trial. The location for the trial was in a relatively flat area with good drainage. Whenever possible, trials are not conducted in drainage areas or areas where water collects. A planter/drill was employed to place seeds in a uniform manner while also ensuring good seed to soil contact. When irrigation was needed and available, the crop was maintained at a rate to target approximately 25% reduction in yield unless otherwise directed. All weed, disease, and insect pressure was also controlled as needed and as appropriate. When doing winter wheat trials, it is preferred that the trial not be planted in a field where the previous crop was corn.

Appropriate harvest equipment was employed to minimize harvest loss. In certain embodiments, the trial is crop destruct wherein all grain is destroyed, and all residue tilled after harvest.

In some embodiments, a 1 kg sample of the harvested grain is collected from each plot and data generated.

Information collected included the date of planting, GPS coordinates of the four corners of each trial, plot maps for the planted plant species and varieties for each trial (range, row, plot number, treatment number), soil moisture levels, seeding rate, herbicides used and row width.

It is also useful to have weather station data. In certain embodiments, a Davis weather station is placed on or near the trial location. In other embodiments, weather data can be requested monthly from publicly accessible sites such as NOAA, Mesonet, etc. This information can include air temperature, relative humidity, solar radiation, rainfall/snowfall, soil temperature at planting (required) and through the season if available.

Soil information was also collected and includes, for example, soil texture, soil pH, organic matter content, cation exchange capacity and quantitation of nitrogen, phosphate and potassium levels.

Emergence was also assessed. Emerging plants were counted at least twice and the date at which the entire field has reached full emergence was recorded.

Seedling vigor and quality rating was also assessed as described above. Seedling vigor/quality for each plot is assessed by surveying the entire plot and rating the apparent health and quality of the plot in the fall before dormancy and just after dormancy in early spring. The rating was done on a scale of 1-5 and exemplary ratings include:
1.) Failed plot, do not use;
2.) Heavily compromised plot, recommended do not use;
3.) Marginally compromised plot;
4.) Mildly compromised plot, but acceptable; and
5.) High quality uncompromised plot.

It is also preferred that normalized difference vegetation index (NFVI) be recorded. A Greenseeker was utilized for this purpose. Greenseeker readings were taken at growth stages Feekes 3.0, 6.0, 8.0 and 10.0. One pass per row was made over each of the two interior rows with the device while maintaining a height of 36 inches (0.91 m) above the crop canopy. In some embodiments, a string 36 inches (0.91 m) long with a weight is attached to the device to assist in maintaining a consistent height over the crop canopy. At least one rating was reported per plot.

Lodging Scores were also determined. In cases where significant levels of lodging occur in the trial prior to harvest, each plot is rated on a scale of 1-10 for plant lodging in increments of 10%, where 1=10% lodging and 10=100% lodging.

Harvest gap count is determined by measurement (in cm) of gaps in plant density in rows to be harvested. It is preferred that only count gaps greater than 45 cm be measured.

The number of harvestable head in 1 m² is also recorded.

Recorded harvest data includes, for example, test weight (lb./bushel), moisture (%), One Thousand (1000) kernel weight, yield (lb./plot), yield (bu/A), and grain protein levels.

Disease, weed, or insect assessment were performed. All pesticides used and all fertilizers (types and analysis) applied were also recorded.

In an exemplary winter wheat field trial, evaluation of microbial seed treatment to detect improvements in yield and crop quality in the presence of environmental stress conditions was performed.

Winter wheat rating descriptions used in this trial are provided below.

| | | |
|---|---|---|
| Yield | bu/acre | Crop yield per plot |
| % Moisture | % | Moisture content of harvested grain |
| Emergence | Plants/Plot | Number of plants counted in a plot, |
| | | taken 10 days after planting |
| Vigor Score | 1-10 | Rating of overall plant health made by |
| | | cooperator |
| Harvest Heads | heads/square | Number of heads contributing to yield |
| | yard | |
| NDVI | | Measure of live green vegetation. Taken |
| | | at Feekes 3.0, 6.0, 8.0, and 10.0 |

The data obtained included the following.

| | Observation | | | Percent Delta |
|---|---|---|---|---|
| Observation Name | Avg | n= | StdDev | from Control |
| Yield (bu/ac) | 37.1 | 36 | 19.06 | −2.82% |
| % Moisture | 11.9 | 36 | 0.80 | 1.63% |
| Emergence (plants/plot) | 26.8 | 8 | 6.18 | 2.10% |
| Fan Vigor Score | 9.1 | 37 | 1.14 | −0.48% |
| Harvest Heads (yd2) | 331.3 | 23 | 284.68 | −3.83% |
| NDVI1 | 0.37 | 37 | 0.11 | 0.31% |
| NDVI2 | 0.53 | 37 | 0.15 | −0.55% |
| NDVI3 | 0.6 | 30 | 0.13 | −4.31% |
| NDVI4 | 0.47 | 23 | 0.23 | −13.17% |
| Spring Vigor Score | 8.9 | 30 | 1.16 | −2.08% |

The results from this trial testing a bacterial endophyte P. oleovorans (strain Poryz) reveal positive yield gains which were observed in two trials experiencing drought stress.

| | Observation | | | Percent Delta |
|---|---|---|---|---|
| Observation Name | Avg | StdDev | N= | from Control |
| Yield (bu/ac) | 45.05 | 4.19 | 8 | 1.04% |
| Yield (bu/ac) | 11.46 | 3.76 | 8 | 6.62% |

Soy

Field trials are conducted under non-irrigated (dryland) conditions at multiple locations, preferably in diverse geographic regions. Seeds are prepared with the endophyte formulations and formulation control (lacking any endophyte) as described in Example VIII. Seeds are sown in regularly spaced rows in soil at 40,000 seeds/acre seeding density. At each location, at least 3 replicate plots are planted per endophyte or control treatment in a randomized complete block design. Each plot consisted of four 15.24 m (40 ft.) rows, each separated by 76.2 cm (30 in).

At the end of the field trial employing endophyte treatment and control treatment plants, plots are machine harvested with a 5-ft research combine and yield calculated by the on-board computer. Only the middle two rows of the 4 row plots are harvested to prevent border effects.

Corn

Field trials are conducted at multiple locations, preferably in diverse geographic regions. Plots are non-irrigated (dryland) or maintained with suboptimal irrigation at a rate to target approximately 25% reduction in yield. Seeds are prepared with the endophyte formulations and formulation control (lacking any endophyte) as described in Example VIII. Seeds are sown in regularly spaced rows in soil at planting densities typical for each region. At each location 3 replicate plots are planted per endophyte or control treatment in a randomized complete block design. Each plot consisted of four 15.24 m (40 ft.) rows, each separated by 76.2 cm (30 in).

At the end of the field trial employing endophyte treatment and control treatment plants, plots are machine harvested with a 5-ft research combine and yield calculated by the on-board computer. Only the middle two rows of the 4 row plots are harvested to prevent border effects.

Field trials for corn can be conducted to assess the results of endophyte formulation application as described above for wheat. When conducting field trials on corn, different parameters to be assessed can include, without limitation, the effect of the formulation on differenent varieties of corn, the effect of different fertilizer applications, soil content, herbicide use, etc. The data to be collected are those similar to that described above. Aspects of the treated corn to be assessed include, early emergence, vigor rating, NDVI measurements, weather station data, effects on tasseling/silking, lodging, stay green, early disease rating, late disease rating, plot assessments at harvest (e.g., stand, lodging, ear drop etc.), maturity date, and final stand count at harvest.

Nutrient analysis can also be performed in tissues sampled at V5 and at tasseling for example.

Cotton

Field trials are conducted under non-irrigated (dryland) conditions at multiple locations, preferably in diverse geographic regions. Seeds are prepared with the endophyte formulations and formulation control (lacking any endophyte) as described in Example VIII. Seeds are sown in regularly spaced rows in soil at 40,000 seeds/acre seeding density. At each location, at least 3 replicate plots are planted per endophyte or control treatment in a randomized complete block design. Each plot consisted of four 15.24 m (40 ft.) rows.

At the end of the field trial employing endophyte treatment and control treatment plants, the middle two rows are harvested and yield calculated. Only the middle two rows of the 4 row plots are harvested to prevent border effects.

Field trials for cotton can be conducted to assess the results of endophyte formulation application, essentially as described above for wheat and soybean. When conducting field trials on cotton, different parameters to be assessed can include, without limitation, the effect of the formulation on different varieties of cotton, the effect of different fertilizer applications, soil content, herbicide use, etc. The data to be collected are also those similar to that described above for wheat and soybean. Aspects of the treated cotton to be assessed include, early emergence, vigor rating, NDVI measurements, weather station data, effects on flowering, harvest gap count, lodging, disease and insect rating, plot assessments at, and final stand count at harvest. Data to be recorded at final harvest include, for example, total plot weight (lb/plot), lint yield (post ginning (lb/plot), gin turnout and fiber quality.

In certain cotton field trials, resistance to root knot nematode (RKN) induced by application to endophyte formulations will be assessed. Roots will be analyzed from treated and untreated nematode exposed plants and examined for RKN damage or reduction of the same in response to endophyte formulation application. Assessments can be performed at early flower and damage rated on a 1 to 5 scale, with 5 being severe damage.

Rice

Field trials are conducted under flood conditions at multiple locations, preferably in diverse geographic regions. Seeds are prepared with the endophyte formulations and formulation control (lacking any endophyte) as described in Example VII. Treated seeds are planted at a standard seeding rate, plots are flooded, weeds and insects are controlled with local standard practices, fertilizer are applied using standard local practices. Plots are machine harvested with a 5-ft research combine and yield calculated by the on-board computer.

Field trials for rice can be conducted to assess the results of endophyte formulation application, essentially as described above for wheat. When conducting field trials on rice, different parameters to be assessed can include, without limitation, the effect of the formulation on different varieties of rice, the effect of different fertilizer applications, soil content, herbicide use, etc. The data to be collected are also those similar to that described above for wheat. Aspects of the treated rice to be assessed include, early emergence, vigor rating, NDVI measurements, weather station data, effects on flowering, harvest gap count, lodging, disease and insect rating, plot assessments at, and final stand count at harvest. Data to be recorded at final harvest include, for example, total plot weight (lb/plot), lint yield (post ginning (lb/plot), gin turnout and fiber quality.

### Example XI

### Greenhouse Assessment of Improved Plant Characteristics Under Biotic Stress and No Stress

Biotic Stress

This example describes a procedure of greenhouse screening of microbes against a crop pathogen Rhizoctonia solani, one of the causal agents of seedling damping off disease. The Rhizoctonia solani isolate R9 was originally isolated from field-grown plants in Alabama and was re-isolated in the laboratory in 2016 to maintain the virulence. Chemically treated soybean varieties are used.

Preparation of Rhizoctonia R9 Inoculum

The permanent stock of R9 is maintained on corn meal agar slants at room temperature. R9 is subcultured in a PDA plate for a week, then 5 plugs of mycelium are transferred into one liter of PDB broth in a 3-liter flask. The culture is grown at room temperature with vigorous shaking for 5 days. The entire one liter of the culture is poured into and mixed well 4 pounds of doubly autoclaved millet seeds. The mixture is sealed in a large plastic bag and incubated for 2 weeks at room temperature with gentle mixing every other day followed by a 2-day air drying inside a biosafety cabinet. Dried infected millet seeds are aliquoted into smaller bags and are usually used to set up disease assay in greenhouse within a week.

Greenhouse Assay Setup

This greenhouse assay is conducted in 6.5 inch diameter plastic pots. The pots are first filled with 400 cc of mildly moistured Sunshine potting mix, followed by another layer of 400 cc potting mix uniformly blended with 2 tablespoons of R9-infected millet seeds. The pots are generally two third full with 800 cc of potting mix. The pots are left sit at room temperature under dark condition for two nights before placing seeds to ensure a thick layer of aggressively grown pathogen mycelium in the soil.

This greenhouse assay is conducted using chemically treated soy seeds coated with the endophyte biomaterials and formulation control (no endophyte) and untreated controls (no endophyte and no formulation) as described in Example VII or VIII. Five seeds are evenly placed onto each pot on top of the inoculum layer and the pots are filled up with another 400 cc potting mix. Ten replicated pots of each treatment are set up and placed on a greenhouse bench using a random block design. The following growth and vigor metrics are measured: percentage emergence at Day 7 and percentage standing at Days 14 and 21, top view images at Day 7 and side view images of pulled and washed seedlings at Day 21, plant height at Day 21, plant dry weight at Day 21, and root crown disease rating at Day 21 using a 0-5 scale with 5 denotes the strongest necrosis and collapse of stem at the root crown region.

At Day 21 post planting, seedlings are gently pulled off the pot, washed with tap water to remove dirt, and kept in plastic bags at 4° C. for further data measurement. The severity of root crown necrosis is first independently rated by multiple persons using the scale described above, followed by plant height measurement before being laid on to a fluted plastic board for side view imaging. After side view imaging, seedlings from the same pot are put into a paper bag and dried in an oven. Plant dry weight of each individual seedling is recorded.

No Stress

In additional greenhouse experiments performed on wheat, it was demonstrated that P. oleovorans (strain Poryz) was effective to increase median shoot length.

Further green house trials with strain Poryz were performed on wheat, soybean, corn and cotton with strain Poryz. Plant tissues (roots, shoots) were separated and oven-dried and assessed at day 4, day 5 and day 7 emergence. Data were fit to Bayesian regression models in R to identify significant interactions.

### Results

| | shoot dry weight (g) | | |
|---|---|---|---|
| Poryz | corn | Cotton | soybean |
| % change relative | 15% | −5% | 0% |
| to Control | | | |
| % confidence the | 99.9% | 6.9% | 42.6% |
| observed effect is | | | |
| positive | | | |

While shoot dry weight was increased in corn exposed to Poryz, this result was not observed in cotton or soy.

### Example XII

### Assessment of Improved Biotic Stress Tolerance of Soy in Field Conditions

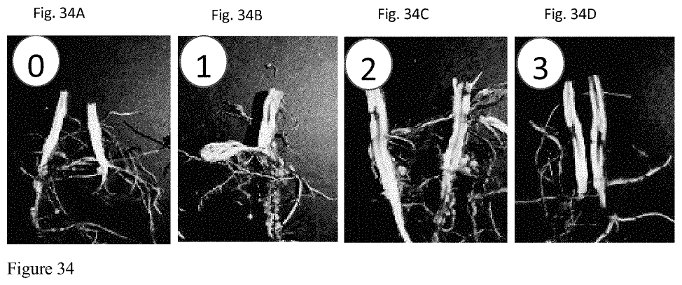

Field trials were conducted using chemically treated soy seeds coated with the endophyte biomaterials and formulation control (no endophyte) and untreated controls (no endophyte and no formulation) as described in Example IX. The two plots used in field assessment harbour populations root knot nematode (Meloidogyne incognita) and Reniform nematode (Rotylenchulus reniformis), respectively, at an approximately 1.0+E04 eggs per gram of fresh root weight. Opportunistically, these plots were infected with natural inoculum of Fusarium virguliforme, the causal agent of Fusarium Sudden Death Syndrome (SDS). The trials followed standard Agriculture Resource Management (ARM) design. Five replicate plots were planted per endophyte or control treatment in a randomized complete block design. Each plot consisted of a 7.62 m (25 ft.) by 0.76 m (2.5 ft) row. The following early growth metrics were measured: percent emergence at 14 days post planting, standing count at 28 and 45 days post planting, plant vigor at 14, 28, and 45 days post planting, plant height at 45 days post planting, fresh shoot weight, fresh root weight, disease rating at a 0-3 scale (3 denotes strong disease symptoms) using the split-root scoring system at 45 days post planting, nematode count at 45 days post planting, and yield parameters. FIG. 34 shows exemplary soy roots which were scored using the split-root scoring system at 45 days post planting.

At the end of the field trial employing endophyte treatment and control treatment plants, 4 plants were randomly dig out from each row, kept in a plastic bag, and brought back to lab for metric measurements. For each seedling, shoot and root were separated by cutting at 3 cm from the first branch of the root. The heights of the separated shoot of each plant were measured, followed by fresh shoot weight, and fresh root weight. The main root was vertically split into two halves and discoloration of xylem was scored as described above. To extract and count nematode eggs on root, roots were place in a container prefilled with 100 ml 10% sucrose and incubated on a shaker at room temperature overnight. The supernatant was then collected, and nematode eggs are counted under a stereomicroscope.

Data were manually curated and entered into ARM database before being analyzed. The percentage of survival plants, fresh root weight, and nematode egg count were plotted as bar graph of mean±95% confidence interval from the mean using the ggplot2 package of R (R Core Team, 2016. R: A language and environment for statistical computing. R Foundation for Statistical Computing, Vienna, Austria. R-project.org/). Plant heights, fresh shoot weight, and disease scores were plotted as jittered dot of mean±nonparametric bootstrap (1000) of 95% confidence interval from the mean using the ggplot2 package of R. ARM ST analyses were also performed for all data metrics to assess the treatment stability and trial clusters using aggregated data.

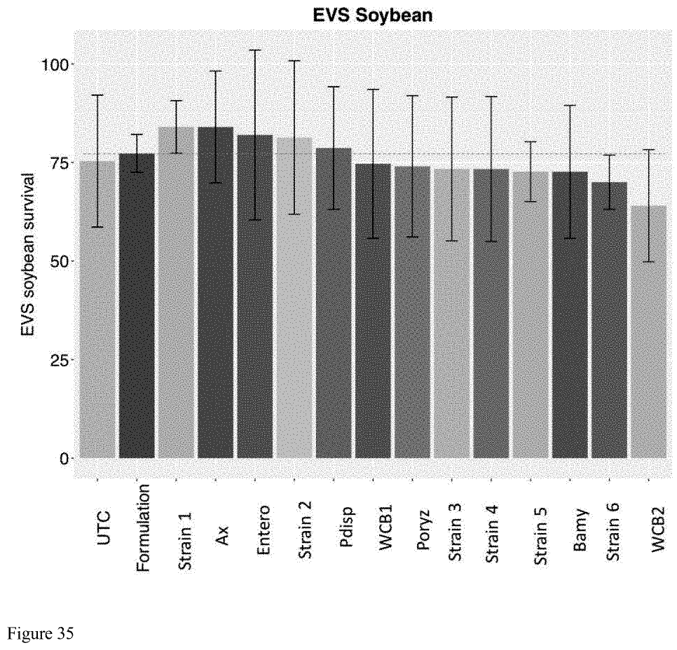

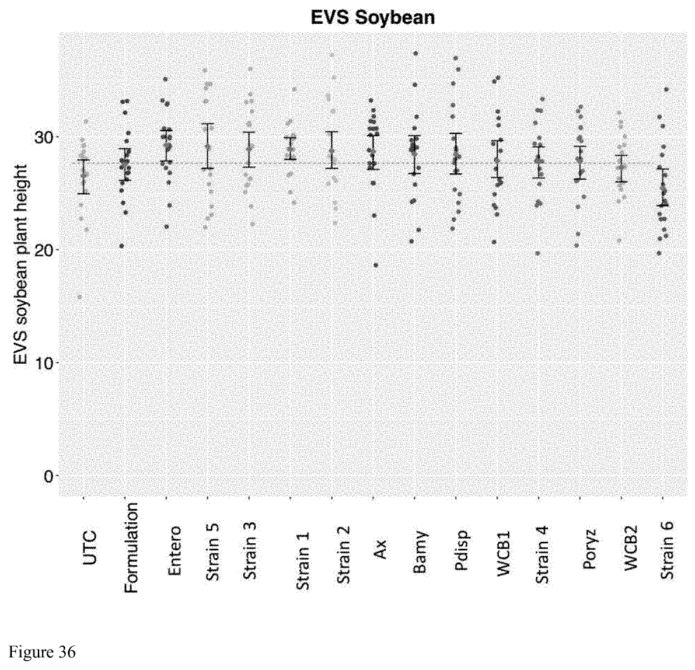

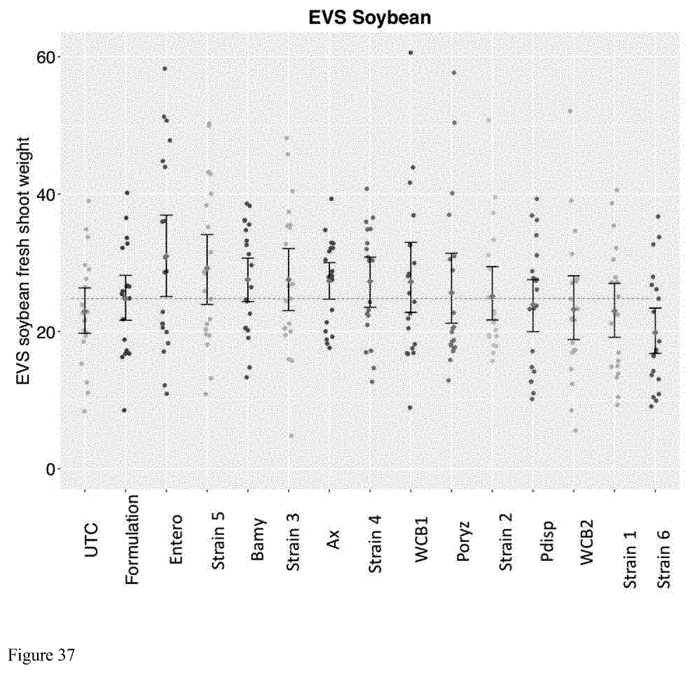

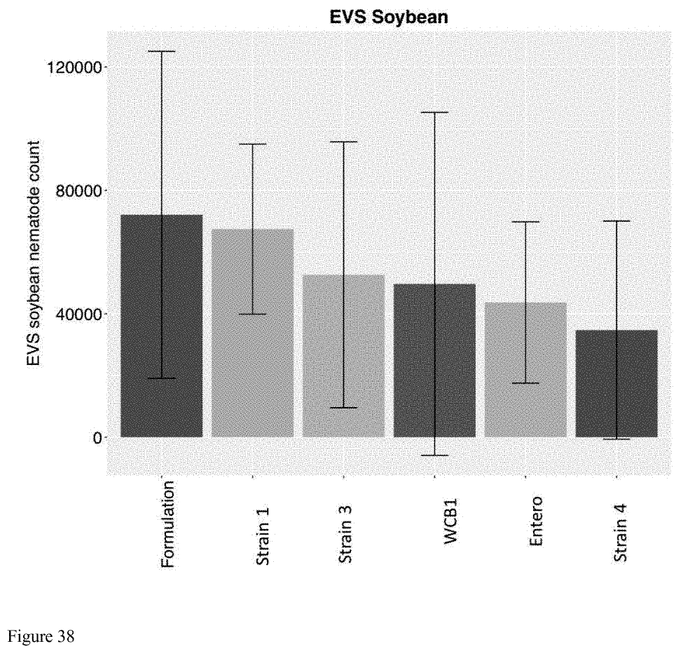

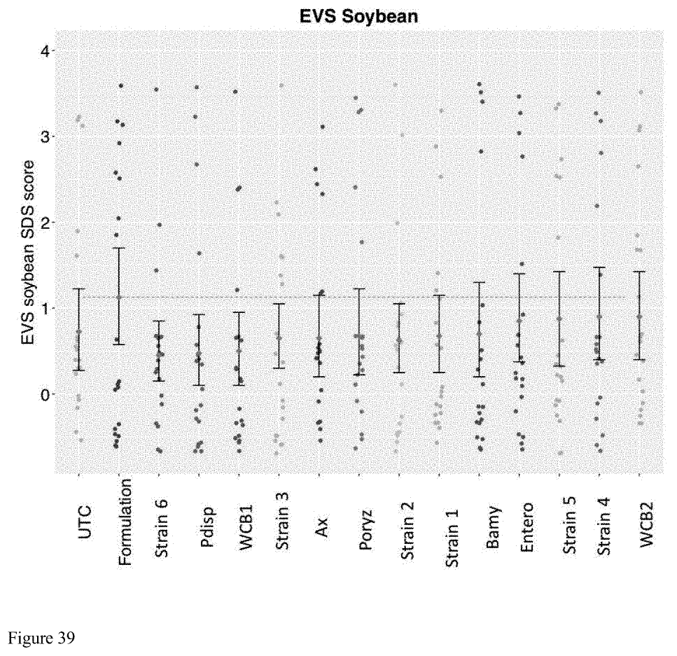

Exemplary results are shown in FIGS. 35-39. Percent survival of soybean plants in the presence of Fusarium virguliforme and Rotylenchulus reniformis are depicted in FIG. 35. Soybean plants treated with Achromobacter xylosidans (strain Achromo) showed a 10% increase in survival relative to formulation controls. FIG. 36 shows the height of soybean plants in the presence of Fusarium virguliforme and Rotylenchulus reniformis. Soybean plants treated with Enterobacter cloacae (strain Entero) and showed a greater than 10% increase height relative to formulation controls. FIG. 37 shows the fresh shoot weight of soybean plants in the presence of Fusarium virguliforme and Rotylenchulus reniformis. Soybean plants treated with Enterobacter cloacae (strain Entero), Bacillus amyloliquefaciens (strain Bamy), and Achromobacter xylosidans (strain Achromo) showed a greater than 10% increase in fresh shoot weight relative to formulation controls. FIG. 38 shows the number of Rotylenchulus reniformis (Reniform) eggs detected per plant for 20 plants selected from each replicate for each treatment. Soybean plants treated with Enterobacter cloacae (strain Entero) and Curobacterium oceanosedimentum (strain WCB1) showed reduced numbers of Reniform eggs per plant compared the formulation control and similar reductions to Strain 3 and Strain 4 which are known to have biocontrol activity against Reniform. FIG. 39 shows the soybean sudden death syndrome (SDS) disease score of soybean plants grown in the presence of Fusarium virguliforme and Rotylenchulus reniformis. Treatment with Pantoea dispersa (strain Pdisp) or Curtobacterium oceanosedimentum (strain WCB1) conferred improved SDS resistance.

### Example XIII

### Assessment of Improved Biotic Stress Tolerance or Increased Yield of Cotton and Corn in Field Conditions

Field trials are conducted using chemically treated cotton seeds coated with the endophyte biomaterials and formulation control (no endophyte) and untreated controls (no endophyte and no formulation) as described in Example VIII. The plots for field assessment harbour of root knot nematode (Meloidogyne incognita) and Reniform nematode (Rotylenchulus reniformis), respectively, at an approximately 1.0+E04 eggs per gram of fresh root weight. Opportunistically, these plots are infected with natural inoculum of Fusarium virguliforme, the causal agent of Fusarium SDS. The trials follow standard ARM design. Five replicate plots are planted per endophyte or control treatment in a randomized complete block design. Each plot consists of a 7.62 m (25 ft.) by 0.76 m (2.5 ft) row. The following early growth metrics are measured: percent emergence at 14 days post planting, standing count at 28 and 45 days post planting, plant vigor at 14, 28, and 45 days post planting, plant height at 45 days post planting, fresh shoot weight, fresh root weight, disease rating at a 0-3 scale (3 denotes strong disease symptoms) using the split-root scoring system at 45 days post planting, nematode count at 45 days post planting, and yield parameters.

At the end of the field trial employing endophyte treatment and control treatment plants, 4 plants are randomly dig out from each row, kept in a plastic bag, and brought back to lab for metric measurements. For each seedling, shoot and root are separated by cutting at 3 cm from the first branch of the root. The heights of the separated shoot of each plant are measured, followed by fresh shoot weight, and fresh root weight. The main root is vertically split into two halves and discoloration of xylem are scored as described above. To extract and count nematode eggs on root, roots are placed in a container prefilled with 100 ml 10% sucrose and incubated on a shaker at room temperature overnight. The supernatant is then collected, and nematode eggs are counted under a stereomicroscope.

Data are manually curated and entered into ARM database before being analyzed. The percentage of survival plants, fresh root weight, and nematode egg count are plotted as bar graph of mean±95% confidence interval from the mean using the ggplot2 package of R (R Core Team, 2016. R: A language and environment for statistical computing. R Foundation for Statistical Computing, Vienna, Austria. R-project.org/). Plant heights, fresh shoot weight, and disease scores are plotted as jittered dot of mean±nonparametric bootstrap (1000) of 95% confidence interval from the mean using the ggplot2 package of R. ARM ST analyses are also performed for all data metrics to assess the treatment stability and trial clusters using aggregated data.

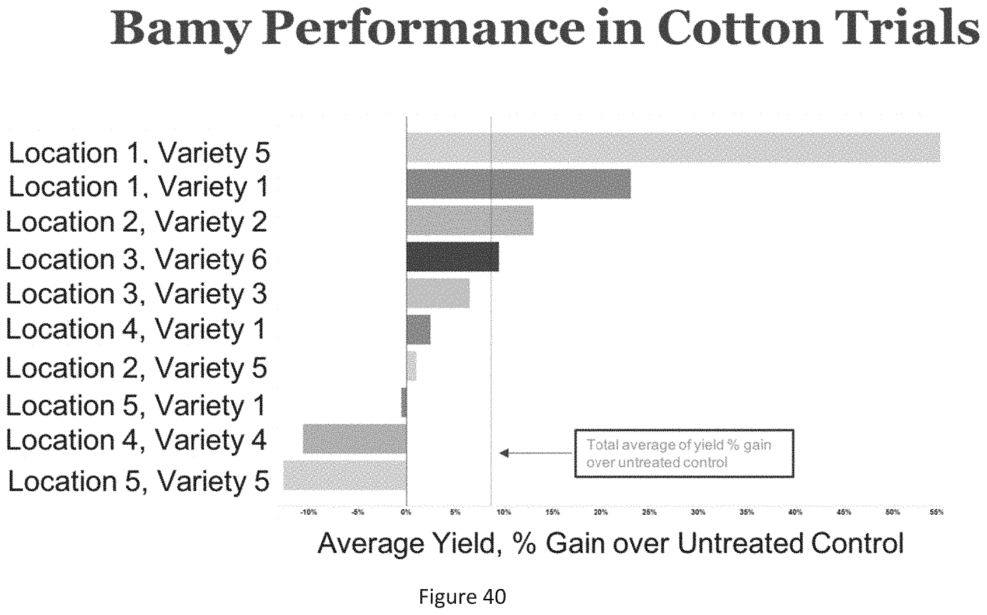

In another field trial for cotton, seeds were coated with endophyte combinations containing strains Bamy or M. oxidans B2. All trials were run as randomized blocks split by genotype with 4 reps for each trial except Seed Variety Interaction. Notably, a positive effect on yield was observed for both treatments in cotton. All seed was first chemically treated, followed by microbial treatment, followed by a polymer formulation. FIG. 40 provides the results showing increased yield obtained when strain Bamy was employed in a cotton field trial.

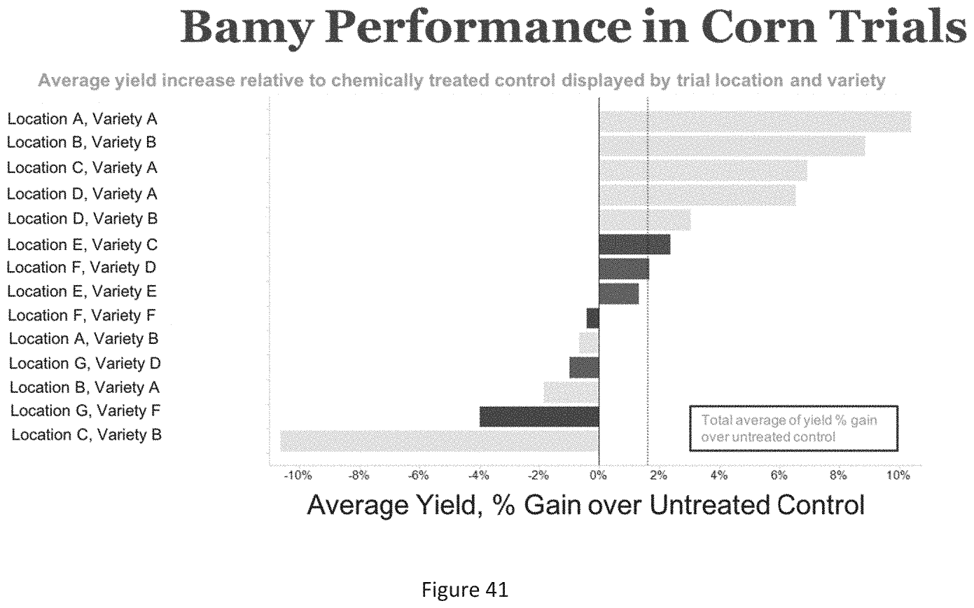

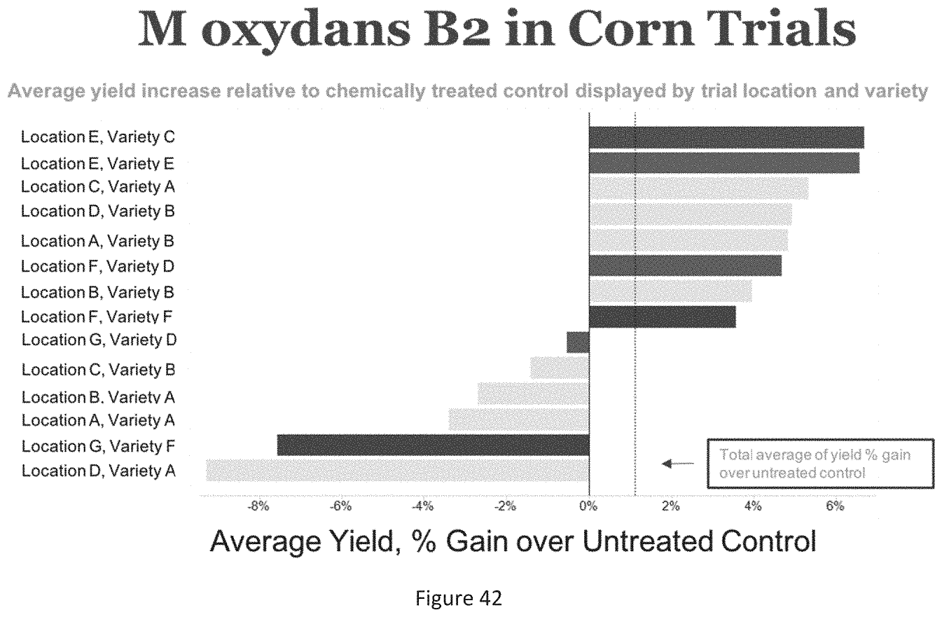

Corn was also treated in a field trial with strains Bamy or M. oxidans B2. As with the cotton trial described, increased yield was obtained in corn treated with each of these endophytes. See FIG. 41 (Bamy) and FIG. 42 (M. oxidans B2).

### Example XIV

### Chemical Compatibility

The strains Bamy, Pdisp, and Poryz were assayed for compatibility with commercial seed treatment chemicals to identify the chemicals that have the least amount of inhibition and thus the most commercial relevance for use in conjunction with these strains.

The following protocol was utilized to assay the chemical compatibility of each strain: A colony or cluster of colonies of each strain was picked from agar plates on which the stains had grown, and transferred into a well of a 24-well plate containing 3 mL of 20% TSB. The plate was then covered with a sterile breathable membrane and incubated at room temperature with agitation for 2 days. 100× dilutions of the bacterial stocks were then made. 100 uL of the 100× bacterial dilutions were pipetted onto 20% tryptic soy agar (TSA) plates and a cell spreader was used to evenly disperse the liquid to create a bacterial lawn on the plates. The plates were then left in a biosafety cabinet until dried, between 20-60 minutes.

Chemically treated seeds prepared so as to coincide with standard commercial practice were placed in the center of the dried plates. The plates were then placed in the dark and incubated at room temperature for two days. On the third day plates were photographed and manually scored using the scale described in as described in the following table:

| Score | Definition |
|---|---|
| 0 | No Inhibition, the bacterium in the |
| | presence of a chemically treated |
| | seed grows equal to that of the |
| | control. |
| 1 | Slight Inhibition, a small zone of |
| | inhibition has formed around the |
| | chemically treated seed or the |
| | colony size/number has been |
| | slightly reduced compared to the |
| | control, or otherwise the strain |
| | appears only slightly altered |
| | compared to control. |
| 2 | Moderate Inhibition, a clearly |
| | defined zone of inhibition has |
| | formed around the chemically |
| | treated seed or the |
| | colony/size/number has been |
| | moderately reduced compared to |
| | the control. |
| 3 | Strong Inhibition, a large zone of |
| | inhibition has formed around the |
| | chemically treated seed or the |
| | colony size/number has been |
| | greatly reduced compared to the |
| | control. |
| 4 | Complete Inhibition, wells |
| | containing a chemically treated |
| | seed show no growth at all, while |
| | the control well shows full growth. |

The following results were obtained
Bamy: scored 0.0 (no inhibition) for the following chemistry and crop combinations:
CruiserMaxx 250 (Corn)
CruiserMaxx Rice (Rice)
Albaugh A (Cotton)
TMI50 (Wheat)
Metalaxyl/ST Methyl Blend (Soy)
Pdisp: scored 0.0 (no inhibition) for the following chemistry and crop combinations:
Albaugh A (Cotton)
Metalaxyl/ST Methyl Blend (Soy)
Poryz: scored 0.0 (no inhibition) for the following chemistry and crop combinations:
CruiserMaxx 250 (Corn)
CruiserMaxx Rice (Rice)
Albaugh A (Cotton)
TMI50 (Wheat)
Metalaxyl/ST Methyl Blend (Soy)

### Example XV

### Method of Determining Seed Nutritional Quality Trait Component: Fat

Seed samples are harvested from mature plants. Analysis of fat is conducted on replicate samples (5×) according to the Association of Official Agricultural Chemists Reference Method AOAC 920.39, of the Official Methods of Analysis of AOAC International, 20th Edition (2016), herein incorporated by reference in its entirety. Samples are weighed onto filter paper, dried, and extracted in hot hexane for 4 hrs using a Soxhlet system. Oil is recovered in pre-weighed glassware, and percent Fat is measured gravimetrically. Mean percent changes between the treatment (endophyte-treated seed) and control (seed treated with the formulation but no endophyte) are calculated.

### Example XVI

### Method of Determining Seed Nutritional Quality Trait Component: Ash

Seed samples are harvested from mature plants. Analysis of fat is conducted on replicate samples (5×) according to the Association of Official Agricultural Chemists Reference Method AOAC 920.39, of the Official Methods of Analysis of AOAC International, 20th Edition (2016). Samples are weighed into pre-weighed crucibles, and ashed in a furnace at 600° C. for 3 hr. Weight loss on ashing is calculated as percent Ash. Mean percent changes between the treatment (endophyte-treated seed) and control (seed treated with the formulation but no endophyte) are calculated.

### Example XVII

### Method of Determining Seed Nutritional Quality Trait Component: Fiber

Seed samples are harvested from mature plants. Analysis of fat is conducted on replicate samples (5×) according to the Association of Official Agricultural Chemists Reference Method AOAC 920.39, of the Official Methods of Analysis of AOAC International, 20th Edition (2016). Samples are weighed into filter paper, defatted and dried, and hydrolyzed first in acid, then in alkali solution. The recovered portion is dried, weighed, ashed at 600°, and weighed again. The loss on ashing is calculated as percent Fiber. Mean percent changes between the treatment (endophyte-treated seed) and control (seed treated with the formulation but no endophyte) are calculated.

### Example XVIII

### Method of Determining Seed Nutritional Quality Trait Component: Moisture

Seed samples are harvested from mature plants. Analysis of fat is conducted on replicate samples (5×) according to the Association of Official Agricultural Chemists Reference Method AOAC 920.39, of the Official Methods of Analysis of AOAC International, 20th Edition (2016). Samples are weighed into pre-weighed aluminum dishes, and dried at 135° C. for 2 hrs. Weight loss on drying is calculated as percent Moisture. Mean percent changes between the treatment (endophyte-treated seed) and control (seed treated with the formulation but no endophyte) are calculated.

### Example XIX

### Method of Determining Seed Nutritional Quality Trait Component: Protein

Seed samples are harvested from mature plants. Analysis of fat is conducted on replicate samples (5×) according to the Association of Official Agricultural Chemists Reference Method AOAC 920.39, of the Official Methods of Analysis of AOAC International, 20th Edition (2016). Samples are combusted, and nitrogen gas is measured using a combustion nitrogen analyzer (Dumas). Nitrogen is multiplied by 6.25 to calculate % protein. Mean percent changes between the treatment (endophyte-treated seed) and control (seed treated with the formulation but no endophyte) are calculated.

### Example XX

### Method of Determining Seed Nutritional Quality Trait Component: Carbohydrate

Seed samples are harvested from mature plants. Analysis of carbohydrate is determined for replicate samples (5×) as a calculation according to the following formula:

Total Carbohydrate=100%−% (Protein+Ash+Fat+Moisture+Fiber)

Where percent Protein is determined according to the method of Example 17, percent Ash is determined according to the method of Example 14, percent Fat is determined according to the method of Example 13, percent Moisture is determined according to the method of Example 16, and percent Fiber is determined according to the method of Example 15. Mean percent changes between the treatment (endophyte-treated seed) and control (seed treated with the formulation but no endophyte) are calculated.

### Example XXI

### Method of Determining Seed Nutritional Quality Trait Component: Calories

Seed samples are harvested from mature plants. Analysis of Calories is determined for replicate samples (5×) as a calculation according to the following formula: Total Calories=(Calories from protein)+(Calories from carbohydrate)+Calories from fat) Where Calories from protein are calculated as 4 Calories per gram of protein (as determined according to the method of Example 17), Calories from carbohydrate are calculated as 4 Calories per gram of carbohydrate (as determined according to the method of Example 18), and Calories from fat are calculated as 9 Calories per gram of fat (as determined according to the method of Example 13). Mean percent changes between the treatment (endophyte-treated seed) and control (seed treated with the formulation but no endophyte) are calculated.

### Example XXII

### Additional Methods for Creating Synthetic Compositions

Osmopriming and Hydropriming

A fungal or bacterial endophyte is inoculated onto seeds during the osmopriming (soaking in polyethylene glycol solution to create a range of osmotic potentials) and/or hydropriming (soaking in de-chlorinated water) process. Osmoprimed seeds are soaked in a polyethylene glycol solution containing a bacterial and/or fungal endophyte for one to eight days and then air dried for one to two days. Hydroprimed seeds are soaked in water for one to eight days containing a bacterial and/or fungal endophyte and maintained under constant aeration to maintain a suitable dissolved oxygen content of the suspension until removal and air drying for one to two days. Talc and/or flowability polymer are added during the drying process.

Foliar Application

A fungal or bacterial endophyte is inoculated onto aboveground plant tissue (leaves and stems) as a liquid suspension in dechlorinated water containing adjuvants, sticker-spreaders and UV protectants. The suspension is sprayed onto crops with a boom or other appropriate sprayer.

Soil Inoculation

A fungal or bacterial endophyte is inoculated onto soils in the form of a liquid suspension either; pre-planting as a soil drench, during planting as an in furrow application, or during crop growth as a side-dress. A fungal or bacterial endophyte is mixed directly into a fertigation system via drip tape, center pivot or other appropriate irrigation system.

Hydroponic and Aeroponic Inoculation

A fungal or bacterial endophyte is inoculated into a hydroponic or aeroponic system either as a powder or liquid suspension applied directly to the rockwool substrate, or applied to the circulating or sprayed nutrient solution.

Vector-Mediated Inoculation

A fungal or bacterial endophyte is introduced in power form in a mixture containing talc or other bulking agent to the entrance of a beehive (in the case of bee-mediation) or near the nest of another pollinator (in the case of other insects or birds. The pollinators pick up the powder when exiting the hive and deposit the inoculum directly to the crop's flowers during the pollination process.

Root Wash

The method includes contacting the exterior surface of a plant's roots with a liquid inoculant formulation containing a purified bacterial population, a purified fungal population, or a mixture of purified bacteria and fungi. The plant's roots are briefly passed through standing liquid microbial formulation or liquid formulation is liberally sprayed over the roots, resulting in both physical removal of soil and microbial debris from the plant roots, as well as inoculation with microbes in the formulation.

Seedling Soak

The method includes contacting the exterior surfaces of a seedling with a liquid inoculant formulation containing a purified bacterial population, a purified fungal population, or a mixture of purified bacteria and fungi. The entire seedling is immersed in standing liquid microbial formulation for at least 30 seconds, resulting in both physical removal of soil and microbial debris from the plant roots, as well as inoculation of all plant surfaces with microbes in the formulation. Alternatively, the seedling can be germinated from seed in or transplanted into media soaked with the microbe(s) of interest and then allowed to grow in the media, resulting in soaking of the plantlet in microbial formulation for much greater time totaling as much as days or weeks. Endophytic microbes likely need time to colonize and enter the plant, as they explore the plant surface for cracks or wounds to enter, so the longer the soak, the more likely the microbes will successfully be installed in the plant.

Wound Inoculation

The method includes contacting the wounded surface of a plant with a liquid or solid inoculant formulation containing a purified bacterial population, a purified fungal population, or a mixture of purified bacteria and fungi. Plant surfaces are designed to block entry of microbes into the endosphere, since pathogens attempt to infect plants in this way. In order to introduce beneficial endophytic microbes to plant endospheres, a way to access the interior of the plant is needed, which can be done by opening a passage by wounding. This wound can take a number of forms, including pruned roots, pruned branches, puncture wounds in the stem breaching the bark and cortex, puncture wounds in the tap root, puncture wounds in leaves, and puncture wounds seed allowing entry past the seed coat. Wounds can be made using needles, hammer and nails, knives, drills, etc. Into the wound can then be contacted the microbial inoculant as liquid, as powder, inside gelatin capsules, in a pressurized capsule injection system, in a pressurized reservoir and tubing injection system, allowing entry and colonization by microbes into the endosphere. Alternatively, the entire wounded plant can be soaked or washed in the microbial inoculant for at least 30 seconds, giving more microbes a chance to enter the wound, as well as inoculating other plant surfaces with microbes in the formulation—for example pruning seedling roots and soaking them in inoculant before transplanting is a very effective way to introduce endophytes into the plant.

Injection

The method includes injecting microbes into a plant in order to successfully install them in the endosphere. Plant surfaces are designed to block entry of microbes into the endosphere, since pathogens attempt to infect plants in this way. In order to introduce beneficial endophytic microbes to endospheres, a way to access the interior of the plant is needed, which can be done by puncturing the plant surface with a need and injecting microbes into the inside of the plant. Different parts of the plant can be inoculated this way including the main stem or trunk, branches, tap roots, seminal roots, buttress roots, and even leaves. The injection can be made with a hypodermic needle, a drilled hole injector, or a specialized injection system, and through the puncture wound can then be contacted the microbial inoculant as liquid, as powder, inside gelatin capsules, in a pressurized capsule injection system, in a pressurized reservoir and tubing injection system, allowing entry and colonization by microbes into the endosphere.

### Example XXIII

### Identification of Sequence Variants Across Core Genes

Phylogenomic analysis of whole genome sequences of endophytes can be used to identify distinguishing sequence variants. Sets of genes suitable for phylogenomic analysis as well as methods for identifying the same are well known in the art, for example Floutas et al. (2012) The Paleozoic origin of enzymatic lignin decomposition reconstructed from 31 fungal genomes. Science, 336(6089):1715-9. doi: 10.1126/science.1221748 and James TY, Pelin A, Bonen L, Ahrendt S, Sain D, Corradi N, Stajich J E. Shared signatures of parasitism and phylogenomics unite Cryptomycota and microsporidia. Curr Biol. 2013; 23(16):1548-53. doi: 10.1016/j.cub.2013.06.057. Orthologous genes to the reference set are identified in protein data bases derived from the genome of each species. Orthologous genes can be identified in the genomes using methods well known including reciprocal best hits (Ward N, Moreno-Hagelsieb G. Quickly Finding Orthologs as Reciprocal Best Hits with BLAT, LAST, and UBLAST: How Much Do We Miss? de Crdcy-Lagard V, ed. PLoS ONE. 2014; 9(7):e101850. doi:10.1371/journal.pone.0101850) and Hidden Markov Models (HMMs). The best hits are extracted and a multiple sequence alignment generated for each set of orthologous genes. The alignments are used to build phylogenetic trees using methods well known in the art including Bayesian inference and maximum likelihood methods, for example using software tools MrBayes (Huelsenbeck, J. P. & Ronquist (2001) MRBAYES: Bayesian inference of phylogenetic trees. Bioinformatics, 17(8):754-755) and RAxML (Stamatakis, A. (2014) RAxML version 8: a tool for phylogenetic analysis and post-analysis of large phylogenies. Bioinformatics, 30 (9): 1312-1313. doi: 10.1093/bioinformatics/btu033). Sequence variants which distinguish between closely related species are identified.

In some cases, bacterial species have been reassigned due to various reasons (such as, but not limited to, the evolving field of whole genome sequencing), and it is understood that such nomenclature reassignments are within the scope of any claimed species. For example, the Poryz strain was initially identified as a Pseudomonas oryzihabitans where further in depth sequencing revealed that the species is actually Pseudomonas oleovorans.

### Example XXIV

### Identification of Unique Genes in an Endophyte of Interest

Whole genome analysis of endophytes can be used to identify genes whose presence, absence or over or under representation (“differential abundance”) are associated with desirable phenotypes. To identify genes with differential abundance in the genome of an endophyte of interest, protein sequences predicted from the genomes of the endophyte and closely related species are compared in an all-vs-all pairwise comparison (for example, using BLAST) followed by clustering of the protein sequences based on alignment scores (for example, using MCL: Enright A. J., Van Dongen S., Ouzounis C. A. An efficient algorithm for large-scale detection of protein families. Nucleic Acids Research 30(7):1575-1584 (2002)). Additional software tools useful for this analysis are well known in the art and include OMA, OrthoMCL and TribeMCL (Roth A C, Gonnet G H, Dessimoz C. Algorithm of OMA for large-scale orthology inference. BMC Bioinformatics. 2008; 9:518. doi: 10.1186/1471-2105-9-518, Enright A J, Kunin V, Ouzounis C A. Protein families and TRIBES in genome sequence space. Nucleic Acids Res. 2003; 31(15):4632-8; Chen F, Mackey A J, Vermunt J K, Roos D S. Assessing performance of orthology detection strategies applied to eukaryotic genomes. PLoS One. 2007; 2(4):e383.). The protein clusters are queried to identify clusters with differential abundance of proteins derived from endophytes having desirable phenotypes. Proteins of these clusters define the unique properties of these endophytes, and the abundance of genes encoding these proteins may be used to identify endophytes of the present invention.

### Example XXV

### Application of Combinations of Heterologous Endophytic Bacteria to Improve Agronomic Traits

As described in the previous example, the endophytic bacteria listed in Table 1 can be used alone or in combination with other bacteria or agents to confer beneficial phenotypic changes to host plants. These changes include one or more of an increase in root growth promotion, shoot growth promotion, resistance to salt stress, competition with undesirable plant species and resistance to plant pathogens such as fungus, nematodes, weevils etc., in a plant produced from the seed, as compared to a reference plant which is not treated with the inventive bacterial combination. The bacteria can act synergistically in combination or their effects may be additive in achieving the advantages set forth above. Such combinations can include 1, 2, 3, 4, 5, 6, 7, or all of the strains listed in Table 1. Combinations can include one or more strains in Table 1 and other strains known to confer beneficial growth properties to plants. The strains may be present in differing amounts or ratios, e.g., 1:1, 1:2, 1:3, 1:4, 1:2:1, 1:5:1, etc. Exemplary combinations include, but are not limited to:

Bamy + WCB1
Bamy + WCB2
Bamy + Poryz
Bamy + Achromo
Bamy + Pdisp
Bamy + Entero
WCB1 + WCB2
WCB1 + Poryz
WCB1 + Achromo
WCB1 + Pdisp
WCB1 + Entero
WCB2 + Poryz
WCB2 + Achromo
WCB2 + Pdisp
WCB2 + Entero
Poryz + Achromo
Poryz + Pdisp
Poryz + Entero

Additionally, exemplary combinations include, but are not limited to:

Bamy + WCB1 + WCB2
Bamy + WCB1 + Poryz
Bamy + WCB1 + Achromo
Bamy + WCB1 + Pdisp
Bamy + WCB1 + Entero
Bamy + WCB2 + Poryz
Bamy + WCB2 + Achromo
Bamy + WCB2 + Pdisp
Bamy + WCB2 + Entero
Bamy + Poryz + Achromo
Bamy + Poryz + Pdisp
Bamy + Poryz + Entero
Bamy + Achromo + Pdisp
Bamy + Achromo + Entero
Bamy + Pdisp + Entero
WCB1 + WCB2 + Poryz
WCB1 + WCB2 + Achromo
WCB1 + WCB2 + Pdisp
WCB1 + WCB2 + Entero
WCB2 + Poryz + Achromo
WCB2 + Poryz + Pdisp
WCB2 + Poryz + Entero
Poryz + Achromo + Pdisp
Poryz + Achromo + Entero

Exemplary combinations include, but are not limited to:

Bamy + WCB1 + WCB2 + Poryz
Bamy + WCB1 + WCB2 + Achromo
Bamy + WCB1 + WCB2 + Pdisp
Bamy + WCB1 + WCB2 + Entero
Bamy + WCB1 + Poryz + Achromo
Bamy + WCB1 + Poryz + Pdisp
Bamy + WCB1 + Poryz + Entero
Bamy + WCB1 + Achromo + Pdisp
Bamy + WCB1 + Achromo + Entero
Bamy + WCB1 + Pdisp + Entero
Bamy + WCB2 + Poryz + Achromo
Bamy + WCB2 + Poryz + Pdisp
Bamy + WCB2 + Poryz + Entero
Bamy + WCB2 + Achromo + Pdisp
Bamy + WCB2 + Achromo + Entero
Bamy + WCB2 + Pdisp + Entero
Bamy + Poryz + Achromo + Pdisp
Bamy + Poryz + Achromo + Entero
Bamy + Poryz + Pdisp + Entero
Bamy + Achromo + Pdisp + Entero
WCB1 + WCB2 + Poryz + Achromo
WCB1 + WCB2 + Poryz + Pdisp
WCB1 + WCB2 + Poryz + Entero
WCB1 + WCB2 + Achromo + Pdisp
WCB1 + WCB2 + Achromo + Entero
WCB1 + WCB2 + Pdisp + Entero
WCB2 + Poryz + Achromo + Pdisp
WCB2 + Poryz + Achromo + Entero
WCB2 + Poryz + Pdisp + Entero
Poryz + Achromo + Pdisp + Entero

Other exemplary combinations include, without limitation:
combination 1: Bacillus amyloliquefaciens (strain Bamy), 2) Curtobacterium oceanosedimentum (strain WCB1) and 3) Pseudomonas oryzihabitans (strain WCB2) where strains compliment one another, and the mixture shows maximum capability in auxin production, protease production and host plant stress tolerance induction;
combination 2: Bacillus amyloliquefaciens (strain Bamy) and any of the strains Achromo, Pdisp, Entero or Pseudomonas oleovorans, (strain Poryz) that show antifungal activity in order to produce mixtures for fungal disease control; and
combination 3: Bacillus amyloliquefaciens (strain Bamy), M. oxidans B2, and Porzy;

These mixtures should enhance disease protection capability as well as other processes effective to increase of plant growth and yield.

The combined bacteria may be formulated to facilitate administration to target plants of interest, using methods described herein above. They may be lyophilized, and optionally formulated into synthetic alginate beads for distribution into soil. Alternatively, they can be formulated as an aerosol for spraying on areas to be treated. Such methods are known to those of skill in the art of plant and crop propagation.

The formulations described above can also be added to certain fertilizer compositions, such as the controlled release fertilizer composition described in U.S. Pat. No. 9,266,787. Such fertilizer compositions can optionally comprise one or more reagents selected from urea, ammonia, ammonium nitrate, ammonium sulfate, calcium nitrate, diammonium phosphate, monoammonium phosphate, potassium nitrate and sodium nitrate. monopotassium phosphate, dipotassium phosphate, tetrapotassium pyrophosphate, and potassium metaphosphate and optionally a macronutrient selected from the group consisting of sulfur, calcium and magnesium and/or micronutrients including boron, copper, iron, manganese, molybdenum and zinc provided that such reagent does not interfere with the growth promoting action of the endophytic bacteria described herein.

These aforementioned formulations and compositions can further comprise a dispersing agent, such as those disclosed in U.S. Pat. No. 8,241,387.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of improving a plant phenotype, comprising inoculating a plant element with a formulation comprising an effective amount of one or more biologically pure endophyte strains isolated from Thespesia populnea and Gossypium hirsutum which are heterologously disposed to said plant element, and modulate a trait of agronomic importance in a plant comprising or derived from said plant element, as compared to a reference plant grown under the same conditions, wherein said plant is a monocot or dicot, wherein said one or more endophyte strains are selected from
Bacillus amyloliquefaciens (strain Bamy) having a 16S rDNA sequence with at least 97% identity to SEQ ID NO: 1,
Bacillus amyloliquefaciens (strain Bamy) having a 16S rDNA sequence with at least 97% identity to SEQ ID NO: 2,
Curtobacterium oceanosedimentum (strain WCB1) having a 16S rDNA sequence with at least 97% identity to SEQ ID NO: 7,
Curtobacterium oceanosedimentum (strain WCB1) having a 16S rDNA sequence with at least 97% identity to SEQ ID NO: 8,
Pseudomonas oryzihabitans (strain WCB2) having a 16S rDNA sequence with at least 97% identity to SEQ ID NO: 9,
Pseudomonas oryzihabitans (strain WCB2) having a 16S rDNA sequence with at least 97% identity to SEQ ID NO: 10,
Pseudomonas oleovorans (strain Poryz) having a 16S rDNA sequence with at least 97% identity to SEQ ID NO: 11,
Pseudomonas oleovorans (strain Poryz) having a 16S rDNA sequence with at least 97% identity to SEQ ID NO: 12,
Achromobacter xylosidans (strain Achromo) having a 16S rDNA sequence with at least 97% identity to SEQ ID NO: 13,
Achromobacter xylosidans (strain Achromo) having a 16S rDNA sequence with at least 97% identity to SEQ ID NO: 14,
Pantoea dispersa (strain Pdisp) as having a 16S rDNA sequence with at least 97% identity to SEQ ID NO: 15,
Pantoea dispersa (strain Pdisp) as having a 16S rDNA sequence with at least 97% identity to SEQ ID NO: 16,
Enterobacter cloacae (strain Entero) having a 16S rDNA sequence with at least 97% identity to SEQ ID NO: 17, and
Enterobacter cloacae (strain Entero) having a 16S rDNA sequence with at least 97% identity to SEQ ID NO: 18.

2. The method of claim 1, wherein said plant is selected from the group of plants consisting of cotton, okra, soybean, cacao, kenaf and kola nut, coffee, tobacco, potato, tomato, sweet potato, sunflower, rapeseed, wheat, corn, rice, barley, sorghum, grass, sugarcane, bamboo, buckwheat, snap bean, dry bean, canola, peas, peanuts, safflower, sunflower, alfalfa hay, clover, vetch, and trefoil, blackberry, blueberry, currant, elderberry, gooseberry, huckleberry, loganberry, raspberry, strawberry, grape, garlic, leek, onion, shallot, citrus hybrid, grapefruit, kumquat, lime, orange, pummelo, cucumber, melon, gourd, pumpkin, squash, eggplant, sweet pepper, hot pepper, tomatillo, herb, spice, mint, arugula, celery, chervil, endive, fennel, lettuce, parsley, radicchio, rhubarb, spinach, swiss chard, broccoli, brussels sprout, cabbage, cauliflower, collard, kale, kohlrabi, mustard green, asparagus, pear, quince, beet, sugarbeet, red beet, carrot, celeriac, chicory, horseradish, parsnip, radish rutabaga, salsify, and turnips, maple, pine, rye, wheat, sorghum, millet, apricot, cherry, nectarine, peach, plum, prune, almond, beech nut, Brazil nut, butternut, cashew, chestnut, filbert, hickory nut, macadamia nut, pecan, pistachio, walnut, artichoke, cassava, and ginger plants.

3. The method of claim 1, wherein said one or more endophyte strains comprises a nucleic acid sequence having at least 99% identity to a nucleic acid sequence selected from the group consisting of SEQ ID NO: 1, 2 and 7 to 18.

4. The method of claim 1, wherein the trait of agronomic importance is at least one trait selected from suppression of growth of soil borne fungal pathogens, increased resistance to nematode disease, increased resistance to salt stress, increased biotic stress resistance, and increased abiotic stress resistance.

5. The method of claim 1, wherein the plant is selected from soy, cotton, and okra and the trait of agronomic importance is selected from resistance to nematode disease relative to untreated host plants and increased resistance to salts relative to untreated host plants.

6. The method of claim 5, wherein the one or more endophyte strains in at least one of said formulations is selected from a synthetic seed ball, a seed treatment, and a liquid formulation.

7. A synthetic combination comprising an effective amount of a plant element and a formulation comprising one or more biologically pure endophyte strains isolated from Thespesia populnea and Gossypium hirsutuma, which modulate a trait of agronomic importance in the plant comprising or derived from said plant element, as compared to a reference plant grown under the same conditions wherein said one or more endophyte strains are selected from
Bacillus amyloliquefaciens (strain Bamy) having a 16S rDNA sequence with at least 97% identity to SEQ ID NO: 1,
Bacillus amyloliquefaciens (strain Bamy) having a 16S rDNA sequence with at least 97% identity to SEQ ID NO: 2,
Curtobacterium oceanosedimentum (strain WCB1) having a 16S rDNA sequence with at least 97% identity to SEQ ID NO: 7,
Curtobacterium oceanosedimentum (strain WCB1) having a 16S rDNA sequence with at least 97% identity to SEQ ID NO: 8,
Pseudomonas oryzihabitans (strain WCB2) having a 16S rDNA sequence with at least 97% identity to SEQ ID NO: 9,
Pseudomonas oryzihabitans (strain WCB2) having a 16S rDNA sequence with at least 97% identity to SEQ ID NO: 10,
Pseudomonas oleovorans (strain Poryz) having a 16S rDNA sequence with at least 97% identity to SEQ ID NO: 11,
Pseudomonas oleovorans (strain Poryz) having a 16S rDNA sequence with at least 97% identity to SEQ ID NO: 12,
Achromobacter xylosidans (strain Achromo) having a 16S rDNA sequence with at least 97% identity to SEQ ID NO: 13,
Achromobacter xylosidans (strain Achromo) having a 16S rDNA sequence with at least 97% identity to SEQ ID NO: 14,
Pantoea dispersa (strain Pdisp) as having a 16S rDNA sequence with at least 97% identity to SEQ ID NO: 15,
Pantoea dispersa (strain Pdisp) as having a 16S rDNA sequence with at least 97% identity to SEQ ID NO: 16,
Enterobacter cloacae (strain Entero) having a 16S rDNA sequence with at least 97% identity to SEQ ID NO: 17, and
Enterobacter cloacae (strain Entero) having a 16S rDNA sequence with at least 97% identity to SEQ ID NO: 18.

8. The synthetic combination of claim 7, wherein said plant is selected from the group of plants consisting of cotton, okra, soybean, cacao, kenaf and kola nut, coffee, tobacco, potato, tomato, sweet potato, sunflower, rapeseed, wheat, corn, rice, barley, sorghum, grass, sugarcane, bamboo, buckwheat, snap bean, dry bean, canola, peas, peanuts, safflower, sunflower, alfalfa hay, clover, vetch, and trefoil, blackberry, blueberry, currant, elderberry, gooseberry, huckleberry, loganberry, raspberry, strawberry, grape, garlic, leek, onion, shallot, citrus hybrid, grapefruit, kumquat, lime, orange, pummelo, cucumber, melon, gourd, pumpkin, squash, eggplant, sweet pepper, hot pepper, tomatillo, herb, spice, mint, arugula, celery, chervil, endive, fennel, lettuce, parsley, radicchio, rhubarb, spinach, swiss chard, broccoli, brussels sprout, cabbage, cauliflower, collard, kale, kohlrabi, mustard green, asparagus, pear, quince, beet, sugarbeet, red beet, carrot, celeriac, chicory, horseradish, parsnip, radish rutabaga, salsify, and turnips, maple, pine, rye, wheat, sorghum, millet, apricot, cherry, nectarine, peach, plum, prune, almond, beech nut, Brazil nut, butternut, cashew, chestnut, filbert, hickory nut, macadamia nut, pecan, pistachio, walnut, artichoke, cassava, and ginger plants.

9. The synthetic combination of claim 7, wherein said one or more endophyte strains comprises a nucleic acid sequence having at least 99% identity to a nucleic acid sequence selected from the group consisting of SEQ ID NO: 1, 2 and 7 to 18.

10. The synthetic combination of claim 7, wherein said one or more endophyte strains comprises a nucleic acid sequence having at least 100% identity to a nucleic acid sequence selected from the group consisting of SEQ ID NO: 1, 2 and 7 to 18.

11. The synthetic combination of claim 7, wherein the synthetic combination confers at least one trait of agronomic importance is selected from suppression of growth of soil borne fungal pathogens, increased resistance to nematode disease, increased resistance to salt stress, increased biotic stress resistance, and increased abiotic stress resistance.

12. The synthetic combination of claim 7, wherein the plant is selected from soy, cotton, and okra and the trait of agronomic importance is selected from resistance to nematode disease relative to untreated host plants and increased resistance to salts relative to untreated host plants.

13. The synthetic combination of claim 7, wherein the one or more endophyte strains in at least one of the formulations selected from synthetic seed ball, seed treatment, liquid formulation, controlled release fertilizer formulation, lyophilized formulation, and alginate bead formulation.

14. The synthetic combination of claim 7, wherein the formulation comprises an insecticide or fungicide.

15. The synthetic combination of claim 7, wherein said one or more endophyte strains are selected from Bacillus amyloliquefaciens (strain Bamy), Curtobacterium oceanosedimentum (strain WCB1) and Pseudomonas oryzihabitans.

16. The synthetic combination of claim 7, wherein said one or more endophyte strains are selected from
i) Bacillus amyloliquefaciens (strain Bamy), Curtobacterium oceanosedimentum (strain WCB1) and Pseudomonas oryzihabitans (strain WCB2) said strains being effective to modulate auxin production, protease production and plant stress tolerance induction; and one or more endophyte strains are selected from the group of endophyte strains consisting of:
ii) Bacillus amyloliquefaciens (strain Bamy) and one or more of Achromo, Pdisp, Entero or Poryz for enhancement of fungal disease control.

17. The synthetic combination of claim 7, wherein said one or more endophyte strains are Bacillus amyloliquefaciens (strain Bamy) and one or more of Pseudomonas oleovorans (strain Poryz), Enterobacter cloacae (strain Entero), Pantoea dispersa (strain Pdisp), and Achromobacter xylosoxidans (strain Achromo), said strains being effective to suppress the growth of sol borne fungal pathogens.

18. The synthetic combination of claim 7, wherein said one or more endophyte strains are Achromobacter xyloidans (strain Acbromo), Enterobacter cloacae (strain Entero), Bacillus amyloliquefaciens (strain Bany), and Curobacterium oceanosedimnentun (strain WCB1) said strains being effective to increase resistance to nematode disease or decrease biotic stress.
